(19) United States
(12) Patent Application Publication
(10) Pub. No.: US 2010/0041642 A1
(43) Pub. Date: Feb. 18, 2010

(54) UREA INHIBITORS OF MAP KINASES

(75) Inventors: Duyan NGUYEN, Lower Gwynedd, PA (US); Martha KELLY, Collegeville, PA (US); Kristofer MOFFETT, Blue Bell, PA (US); Enrique Luis MICHELOTTI, Ft. Washington, PA (US); Bin Liu, Dayton, NJ (US)

Correspondence Address: PEPPER HAMILTON LLP, ONE MELLON CENTER, 50TH FLOOR, 500 GRANT STREET, PITTSBURGH, PA, 15219, US

(73) Assignee: LOCUS PHARMACEUTICALS, INC., Blue Bell, PA (US)

(21) Appl. No.: 12/541,922

(22) Filed: Aug. 15, 2009

Related U.S. Application Data
(60) Provisional application No. 61/089,264, filed on Aug. 15, 2008.

(51) Int. Cl. A61K 31/496; C07D 409/14; C07D 413/14; A61K 31/5377; A61K 31/551; A61P 43/00

(52) U.S. Cl. 514/218; 544/364; 544/121; 540/575; 514/253.11; 514/235.8

(57) ABSTRACT

Urea containing compounds that inhibit MAP kinases, pharmaceutical compositions including such compounds and methods for using these compounds to treat inflammatory diseases and cancer are described herein.

### CROSS-REFERENCE TO RELATED APPLICATIONS

This application claims benefit of priority to U.S. Provisional patent application Ser. No. 61/089,264 filed Aug. 15, 2008, which is hereby incorporated by reference.

### GOVERNMENT INTERESTS

Not applicable

### PARTIES TO A JOINT RESEARCH AGREEMENT

Not applicable

### INCORPORATION BY REFERENCE OF MATERIAL SUBMITTED ON A COMPACT DISC

Not applicable

### BACKGROUND

1. Field of Invention

Not applicable

2. Description of Related Art

Intracellular signal transduction is the means by which cells respond to extracellular stimuli. Protein kinases are involved in signal transduction. Protein kinases are usually categorized into five classes with the two major classes being tyrosine kinases and serine/threonine kinases. For many biological responses, multiple intracellular kinases are involved and an individual kinase can be involved in more than one signaling event. These kinases are often cytosolic and can translocate to the nucleus or the ribosomes where they can affect transcriptional and translational events respectively.

Overproduction of cytokines such as interleukin-1 (IL-1) and tumor necrosis factor-alpha (TNF-α) is implicated in a wide variety of inflammatory diseases, including rheumatoid arthritis (RA), psoriasis, multiple sclerosis, inflammatory bowel disease, endotoxin shock, osteoporosis, Alzheimer's disease, and congestive heart failure among others (see, e.g., Henry et al., Drugs Future, 24:1345-1354 (1999) Salituro et al., Curr. Med. Chem., 6:807-823 (1999)). There is convincing evidence in human patients that protein antagonists of cytokines, such as for example, monoclonal antibody to TNF-oa, soluble TNF-α receptor-Fc fusion protein and IL-1 receptor antagonists can provide effective treatment for chronic inflammatory diseases.

TNF-α is a protein whose synthesis occurs in many cell types in response to an external stimulus such as for example, a mitogen, an infectious organism, or trauma. Signaling from the cell surface to the nucleus proceeds via several intracellular mediators including kinases that catalyze phosphorylation of proteins downstream in the signaling cascade. Important mediators for the production of TNF-(include the mitogen-activated protein (MAP) kinases, and in particular, p38 kinase.

p38 kinases are activated in response to various stress stimuli, including, but not limited to, proinflammatory cytokines, endotoxin, ultraviolet light, and osmotic shock. At least four isoforms of p38 have been identified to date including: p38α, p38β, p38γ and p38δ, and p38 MAP kinase may be referred to by other names including, for example, cytokine suppressive anti-inflammatory drug-binding protein (CSBP), CSBP kinase, and stress activated protein kinase (SAPK). The sequences of p38 MAP kinases have been disclosed in the following U.S. Pat. Nos. 5,783,664; 5,777,097; 5,955,366; 6,033,873; 5,869,043; 6,444,455 B1; 5,948,885; and 6,376,214, each of which are hereby incorporated by reference in their entireties. The α and β forms of p38 appear to be expressed in inflammatory cells and are considered to be key mediators of TNF-α production, and inhibition of p38 α and β in cells has been shown to result in reduced levels of expression of TNF-α in animal models of inflammatory disease.

Small molecule inhibitors of p38 are expected to have several advantages over protein inhibitors of TNF-α or IL-1. p38 inhibitors cannot only block the production of TNF-α and IL-1, but can also directly interfere with many of their secondary biological effects. In addition, small molecule inhibitors are unlikely to induce immune reactions commonly associated with the administration of proteins. A small molecule inhibitor of p38 would have less chance of being inactivated after oral administration; a major drawback of peptides is their degradation upon oral administration. Thus, there remains a need for compounds which are inhibitors of a p38 kinase, in particular a p38α kinase.

### BRIEF SUMMARY OF THE INVENTION

Not applicable

### DESCRIPTION OF DRAWINGS

Not applicable

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims.

It must be noted that, as used herein, and in the appended claims, the singular forms “a”, “an” and “the” include plural reference unless the context clearly dictates otherwise. Unless defined otherwise, all technical and scientific terms used herein have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

As used herein, the term “about” means plus or minus 10% of the numerical value of the number with which it is being used. Therefore, about 50% means in the range of 45%-55%.

The term “alkyl” as employed herein by itself or as part of another group refers to both straight and branched chain radicals of up to 10 carbons, unless the chain length is otherwise limited, such as methyl, ethyl, propyl, isopropyl, butyl, s-butyl, t-butyl, isobutyl, pentyl, hexyl, isohexyl, heptyl, 4,4-dimethylpentyl, octyl, 2,2,4-trimethylpentyl, nonyl, or decyl.

The term “alkenyl” is used herein to mean a straight or branched chain radical of 2-10 carbon atoms, unless the chain length is otherwise limited, wherein there is at least one double bond between two of the carbon atoms in the chain, including, but not limited to, ethenyl, 1-propenyl, 2-propenyl, 2-methyl-1-propenyl, 1-butenyl, 2-butenyl, and the like. Preferably, the alkenyl chain is 2 to 8 carbon atoms in length, most preferably from 2 to 4 carbon atoms in length.

The term “alkynyl” is used herein to mean a straight or branched chain radical of 2-10 carbon atoms, unless the chain length is otherwise limited, wherein there is at least one triple bond between two of the carbon atoms in the chain, including, but not limited to, ethynyl, 1-propynyl, 2-propynyl, and the like. Preferably, the alkynyl chain is 2 to 8 carbon atoms in length, most preferably from 2 to 4 carbon atoms in length.

In all instances herein where there is an alkenyl or alkynyl moiety as a substituent group, the unsaturated linkage, i.e., the vinyl or ethenyl linkage, is preferably not directly attached to a nitrogen, oxygen or sulfur moiety.

The term “alkoxy” or “alkyloxy” refers to any of the above alkyl groups linked to an oxygen atom. Typical examples are methoxy, ethoxy, isopropyloxy, sec-butyloxy, and t-butyloxy.

The term “aryl” as employed herein by itself or as part of another group refers to monocyclic or bicyclic aromatic groups containing from 6 to 12 carbons in the ring portion, preferably 6-10 carbons in the ring portion. Typical examples include phenyl, biphenyl, naphthyl or tetrahydronaphthyl.

The term “aralkyl” or “arylalkyl” as employed herein by itself or as part of another group refers to C₁₋₆ alkyl groups as discussed above having an aryl substituent, such as benzyl, phenylethyl or 2-naphthylmethyl.

The term “heterocycle” may refer to a “heteroaryl.” “Heteroaryl” as employed herein refers to groups having 5 to 14 ring atoms; 6, 10 or 14 pi electrons shared in a cyclic array; and containing carbon atoms and 1, 2, 3, or 4 oxygen, nitrogen or sulfur heteroatoms (where examples of heteroaryl groups are: thienyl, benzo[b]thienyl, naphtho[2,3-b]thienyl, thianthrenyl, furyl, pyranyl, isobenzofuranyl, benzoxazolyl, chromenyl, xanthenyl, phenoxathiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, 4αH-carbazolyl, carbazolyl, β-carbolinyl, phenanthridinyl, acridinyl, perimidinyl, phenanthrolinyl, phenazinyl, isothiazolyl, phenothiazinyl, isoxazolyl, furazanyl, phenoxazinyl, and tetrazolyl groups).

The term “heterocycle” may also refer to a “heterocycloalkyl.” “Heterocycloalkyls” as used herein may refer to any saturated or partially unsaturated heterocycle. By itself or as part of another group, “heterocycle” may refer to a saturated or partially unsaturated ring system having 5 to 14 ring atoms selected from carbon atoms and 1, 2, 3, or 4 oxygen, nitrogen, or sulfur heteroatoms. Typical saturated examples include pyrrolidinyl, imidazolidinyl, pyrazolidinyl, tetrahydrofuranyl, tetrahydropyranyl, piperidyl, piperazinyl, quinuclidinyl, morpholinyl, and dioxacyclohexyl. Typical partially unsaturated examples include pyrrolinyl, imidazolinyl, pyrazolinyl, dihydropyridinyl, tetrahydropyridinyl, and dihydropyranyl. Either of these systems can be fused to a benzene ring. When a substituent is oxo (i.e., ═O), then 2 hydrogens on the atom are replaced. When aromatic moieties are substituted by an oxo group, the aromatic ring is replaced by the corresponding partially unsaturated ring. For example a pyridyl group substituted by oxo results in a pyridone.

The terms “heteroarylalkyl” or “heteroaralkyl” as employed herein both refer to a heteroaryl group attached to an alkyl group. Typical examples include 2-(3-pyridyl)ethyl, 3-(2-furyl)-n-propyl, 3-(3-thienyl)-n-propyl, and 4-(1-isoquinolinyl)-n-butyl.

The term “cycloalkyl” as employed herein by itself or as part of another group refers to cycloalkyl groups containing 3 to 9 carbon atoms. Typical examples are cyclopropyl, cyclobutyl, cyclopentyl, cyclohexyl, cycloheptyl, cyclooctyl and cyclononyl.

The term “cycloalkylalkyl” or “cycloalkyl(alkyl)” as employed herein, by itself or as part of another group, refers to a cycloalkyl group attached to an alkyl group. Typical examples are 2-cyclopentylethyl, cyclohexylmethyl, cyclopentylmethyl, 3-cyclohexyl-n-propyl, and 5-cyclobutyl-n-pentyl.

The term “cycloalkenyl” as employed herein, by itself or as part of another group, refers to cycloalkenyl groups containing 3 to 9 carbon atoms and 1 to 3 carbon-carbon double bonds. Typical examples include cyclopropenyl, cyclobutenyl, cyclopentenyl, cyclohexenyl, cyclohexadienyl, cycloheptenyl, cycloheptadienyl, cyclooctenyl, cyclooctadienyl, cyclooctatrienyl, cyclononenyl, and cyclononadienyl.

The term “halogen” or “halo” as employed herein by itself or as part of another group refers to chlorine, bromine, fluorine or iodine.

The term “monoalkylamine” or “monoalkylamino” as employed herein by itself or as part of another group refers to the group NH₂ wherein one hydrogen has been replaced by an alkyl group, as defined above.

The term “dialkylamine” or “dialkylamino” as employed herein by itself or as part of another group refers to the group NH₂ wherein both hydrogens have been replaced by alkyl groups, as defined above.

The term “hydroxyalkyl” as employed herein refers to any of the above alkyl groups wherein one or more hydrogens thereof are substituted by one or more hydroxyl moieties.

The term “haloalkyl” as employed herein refers to any of the above alkyl groups wherein one or more hydrogens thereof are substituted by one or more halo moieties. Typical examples include fluoromethyl, difluoromethyl, trifluoromethyl, trichloroethyl, trifluoroethyl, fluoropropyl, and bromobutyl.

The term “carboxyalkyl” as employed herein refers to any of the above alkyl groups wherein one or more hydrogens thereof are substituted by one or more carboxylic acid moieties.

The term “heteroatom” is used herein to mean an oxygen atom (“O”), a sulfur atom (“S”) or a nitrogen atom (“N”). It will be recognized that when the heteroatom is nitrogen, it may form an NR^{a}R^{b} moiety, wherein R^{a} and R^{b} are, independently from one another, hydrogen or C₁ to C₈ alkyl, or together with the nitrogen to which they are bound form a saturated or unsaturated 5-, 6-, or 7-membered ring.

The terms “hydroxy” and “hydroxyl” are used interchangeably to refer to the radical —OH. The terms “pyridyl” and “pyridinyl” are used interchangeably to refer to a monovalent radical of pyridine. The terms “carbamoyl” and “aminocarbonyl” are used interchangeably to refer to the radical NH₂—C(O)—. The terms “ureido” and “aminocarbonylamino” are used interchangeably to refer to the radical NH₂—C(O)—NH—.

“Optional” or “optionally” may be taken to mean that the subsequently described structure, event or circumstance may or may not occur, and that the description includes instances where the event occurs and instances where it does not.

The phrase “optionally substituted” when not explicitly defined refers to a group or groups being optionally substituted with one or more substituents independently selected from the group consisting of hydroxy, nitro, trifluoromethyl, halogen, C₁₋₆ alkyl, C₁₋₆ haloalkyl, C₁₋₆ alkoxy, C₁₋₆ alkylenedioxy, C₁₋₆ aminoalkyl, C₁₋₆ hydroxyalkyl, C₂₋₄ alkenyl, C₂₋₄ alkynyl, C₆₋₁₀ aryl, phenoxy, benzyloxy, 5-10 membered heteroaryl, C₁₋₆ aminoalkoxy, amino, mono(C₁₋₄)alkylamino, di(C₁₋₄)alkylamino, C₂₋₆ alkylcarbonylamino, C₂₋₆ alkoxycarbonylamino, C₂₋₆ alkoxycarbonyl, C₂₋₆ alkoxycarbonylalkyl, carboxy, C₂₋₆ hydroxyalkoxy, (C₁₋₆)alkoxy(C₂₋₆)alkoxy, mono(C₁₋₄)alkylamino(C₂₋₆)alkoxy, di(C₁₋₄)alkylamino(C₂₋₆)alkoxy C₂₋₁₀ mono(carboxyalkyl)amino, bis(C₂₋₁₀ carboxyalkyl)amino, C₂₋₆ carboxyalkoxy, C₂₋₆ carboxyalkyl, carboxyalkylamino, guanidinoalkyl, hydroxyguanidinoalkyl, cyano, trifluoromethoxy, perfluoroethoxy, aminocarbonylamino, mono(C₁₋₄)alkylaminocarbonylamino, di(C₁₋₄)alkylaminocarbonylamino, N—(C₁₋₄)alkyl-N-aminocarbonyl-amino, N—(C₁₋₄)alkyl-N-mono(C₁₋₄)alkylaminocarbonyl-amino or N—(C₁₋₄)alkyl-N-di(C₁₋₄)alkylaminocarbonyl-amino.

The invention described herein provides compounds that are selective inhibitors of p38 MAP kinase including, for example, p38α. As p38 MAP kinase inhibitors the compounds of the invention are also inhibitors of TNF-α expression in human cells. Thus, the compounds presented herein may be useful for treating diseases associated with p38 and TNF-α expression such as, for example, inflammatory disorders as well as other disorders, including, but not limited to, bone resorption, graft vs. host reaction, atherosclerosis, arthritis, osteoarthritis, rheumatoid arthritis, gout, psoriasis, topical inflammatory disease states, adult respiratory distress syndrome, asthma, chronic pulmonary inflammatory disease, chronic obstructive pulmonary disorder, cardiac reperfusion injury, renal reperfusion injury, thrombus, glomerulonephritis, Crohn's disease, ulcerative colitis, inflammatory bowel disease, multiple sclerosis, endotoxin shock, osteoporosis, Alzheimer's disease, congestive heart failure, allergy, cancer, and cachexia.

Compounds of various embodiments of the invention include those of general formula I:

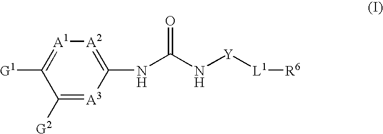

wherein:

A¹ may be N or CR¹;

A² may be N or CR²;

A³ may be N or CR³;

G¹ may be hydrogen, halogen, C₁₋₆ alkyl, C₁₋₆ alkoxy or OR^{a};

G² may be 5- or 6-membered heteroaryl, 5- or 6-membered heterocycloalkyl, C₂₋₆ alkynyl or cyano each of which may be substituted by one or more independently selected R^{A} groups;

R¹, R², and R³ may each, independently, be hydrogen, halogen, hydroxyl, cyano, nitro, C₁₋₄ alkyl, C₁₋₄ haloalkyl, C₁₋₄ alkoxy, C₁₋₄ haloalkoxy, amino, C₁₋₄alkylamino, di-C₁₋₄alkylamino, or C₁₋₆ alkynyl;

Y may be phenyl or 5- or 6-membered heteroaryl, either of which may be substituted with one or more independently selected R^{Y} groups;

L¹ may be a bond, CH₂ or —C(O)—;

R⁶ is

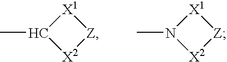

Z may be —NR⁷—, —CHR⁷—, —C(O)—, —SR⁷—, —S(O)—, S(O₂)—, —NH—SO₂—;

X¹ and X² may each, independently, be C₁₋₃ alkyl, which may be substituted with one or more independently selected groups selected from oxo, hydroxyl, cyano, nitro, C₁₋₄ alkyl, C₁₋₄ haloalkyl, C₁₋₄ alkoxy, C₁₋₄ haloalkoxy, amino, C₁₋₄alkylamino, and di-C₁₋₄alkylamino;

each R⁷ may, independently, be hydrogen, cyano, C(O)R^{m}, C(O)C(O)R^{m}, C(O)C(O)NRⁿR^{o}, C(O)NRⁿR^{o}, C(O)OR^{p}, S(O)R^{p}, S(O)₂R^{p}, S(O)NRⁿR^{o}, S(O)₂R^{p}, C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀ aryl, C₆₋₁₀aryl-C₁₋₆ alkyl, C₅₋₉ heteroaryl, or C₅₋₉ heteroaryl-C₁₋₆ alkyl; wherein said C₁₋₆ alkyl, C₂₋₆ alkenyl, and C₂₋₆ alkynyl are each substituted with 1, 2, 3, or 4 independently selected R^{7′} groups; and wherein said C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀ aryl, C₆₋₁₀ aryl-C₁₋₆ alkyl, C₅₋₉ heteroaryl, or C₅₋₉ heteroaryl-C₁₋₆ alkyl may each be substituted with 1, 2, 3, or 4 independently selected R^{7″} groups;

each R^{Y} may, independently, be halogen, hydroxyl, cyano, nitro, C₁₋₆ alkyl, C₁₋₆ haloalkyl, C₁₋₆ alkoxy, C₁₋₆ haloalkoxy, amino, amino, C₁₋₆ alkylamino, di-C₁₋₆ alkylamino, amino(C₁₋₆ alkyl), C₁₋₆ alkylamino(C₁₋₆ alkyl), di(C₁₋₆ alkyl)amino(C₁₋₆ alkyl), C₃₋₉ cycloalkyl, C₃₋₉ heterocycloalkyl, C₁₋₆ acyl, formyl, C₁₋₆ carbamyl, C₁₋₆ alkylcarbamoyl, di(C₁₋₆ alkyl)carbamyl, C₁₋₆ alkylcarbamoyloxy, di(C₁₋₆ alkyl)carbamyloxy, C₁₋₆ acyloxy, carboxy, C₁₋₆ alkylsulfonyl, C₁₋₆ alkylsulfonylamido, or di(C₁₋₆ alkyl)sulfonamide; wherein C₁₋₆ alkyl, C₂₋₆ alkenyl, and C₂₋₆ alkynyl each may be substituted with 1, 2, 3, or 4 independently selected R^{Y′} groups;

each R^{A} may, independently, be halogen, cyano, nitro, tri(C₁₋₆)alkylsilyl, OR^{a}, C(O)R^{a}, C(O)NR^{e}R^{f}, C(O)OR^{a}, OC(O)R^{b}, OC(O)NR^{e}R^{f}, NR^{c}R^{d}, NR^{e}C(O)R^{f}, NR^{e}C(O)OR^{f}, NR^{e}C(O)NR^{f}, S(O)R^{a}, S(O)₂R^{a}, S(O)NR^{e}R^{f}, S(O)₂R^{a}, NR^{e}S(O)₂R^{f}, NR^{g}S(O)₂NR^{e}R^{f}, C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₅₋₁₀ aryl, C₅₋₁₀ aryl-C₁₋₆ alkyl, C₅₋₉ heteroaryl, or C₅₋₉ heteroaryl-C₁₋₆ alkyl; wherein C₁₋₆ alkyl, C₂₋₆ alkenyl, and C₂₋₆ alkynyl may each optionally be substituted with 1, 2, 3, or 4 independently selected R^{A′} groups; and wherein said C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₅₋₁₀ aryl, C₅₋₁₀ aryl-C₁₋₆ alkyl, C₅₋₉ heteroaryl, or C₅₋₉ heteroaryl-C₁₋₆ alkyl are each optionally substituted by 1, 2, or 3 independently selected R^{A″} groups;

each R^{Y′}, R^{7′}, R^{7″}, R^{A′}, and R^{A″} may, independently, be halogen, cyano, nitro, hydroxyl, C₁₋₆ alkyl, C₁₋₆ haloalkyl, C₁₋₆ alkoxy, C₁₋₆ haloalkoxy, amino, C₁₋₆ alkylamino, di-C₁₋₆alkylamino, amino(C₁₋₆ alkyl), C₁₋₆ alkylamino(C₁₋₆alkyl), di(C₁₋₆alkyl)amino(C₁₋₆ alkyl), C₅₋₁₀ aryl, C₅₋₁₀ aryl-C₁₋₆ alkyl, C₅₋₉ heteroaryl, or C₅₋₉ heteroaryl-C₁₋₆ alkyl C₁₋₆ acyl, formyl, carboxy, C₁₋₆ alkyloxycarbonyl, C₁₋₆ carbamyl, C₁₋₆ alkylcarbamoyl, di(C₁₋₆ alkyl)carbamyl, C₁₋₆ alkylcarbamoyloxy, di(C₁₋₆ alkyl)carbamyloxy, C₁₋₆ acyloxy, C₁₋₆ alkylsulfonyl, C₁₋₆ alkylsulfonylamido, or di(C₁₋₆ alkyl)sulfonamide;

each R^{a}, R^{c}, R^{d}, R^{e}, and R^{f} may, independently, be hydrogen, C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀aryl, C₆₋₁₀aryl-C₁₋₆alkyl, C₅₋₉ heteroaryl, or C₅₋₉heteroaryl-C₁₋₆alkyl; wherein said C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀ aryl, C₆₋₁₀ aryl-C₁₋₆alkyl, C₅₋₉ heteroaryl, and C₅₋₉heteroaryl-C₁₋₆alkyl are each optionally substituted with 1, 2 or 3 independently selected R^{g} groups;

each R^{b} may, independently, be C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀aryl, C₆₋₁₀ aryl-C₁₋₆ alkyl, C₅₋₉ heteroaryl, or C₅₋₉ heteroaryl-C₁₋₆ alkyl; wherein said C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀ aryl, C₆₋₁₀ aryl-C₁₋₆ alkyl, C₅₋₉ heteroaryl, and C₅₋₉ heteroaryl-C₁₋₆ alkyl are each optionally substituted with 1, 2 or 3 independently selected R^{h} groups;

each R^{m}, Rⁿ, R^{o}, and R^{p} may, independently, be hydrogen, C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀ aryl, C₆₋₁₀ aryl-C₁₋₆ alkyl, C₅₋₉ heteroaryl, or C₅₋₉ heteroaryl-C₁₋₆ alkyl; wherein said C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀ aryl, C₆₋₁₀ aryl-C₁₋₆ alkyl, C₅₋₉ heteroaryl, and C₅₋₉ heteroaryl-C₁₋₆ alkyl are each optionally substituted with 1, 2 or 3 independently selected Rⁱ groups;

each R^{g}, R^{h}, and Rⁱ may, independently, be halogen, C₁₋₆ alkyl, C₁₋₆ haloalkyl, hydroxyl, C₁₋₆ alkoxy, C₁₋₆ haloalkoxy, cyano, nitro, amino, C₁₋₆alkylamino, or di-C₁₋₆alkylamino.

Each of the compounds described herein may provided as a free base or as a pharmaceutically acceptable salt of the compound, and in additional embodiments, compounds of formula I may be provided as an N-oxide or pharmaceutically acceptable salt of an N-oxide.

In some embodiments, each R¹, R² and R³ may, independently, be hydrogen, halogen, cyano, C₁₋₄ alkyl, C₁₋₄ haloalkyl or C₁₋₄ alkoxy, and in other embodiments, R¹, R² and R³ may each, independently, be hydrogen, methyl, fluoro, chloro, or methoxy. In particular embodiments, R¹ may be hydrogen, halogen or methyl and/or R² may be hydrogen, halogen or methyl and/or R³ may be hydrogen, halogen, methyl or methoxy.

In certain embodiments, G² may be a 6-membered heteroaryl, 5-membered heteroaryl, 5-membered heterocycloalkyl or C₂₋₆ alkynyl each of which may be substituted by 1, 2, 3, or 4 independently selected R^{A} groups. For example, in some embodiments, G² may be a pyridine ring, a pyrimidine ring, an oxaxole ring, an isoxazole ring, a thiazole ring, or an imidazolidine-2,4-dione ring, each of which may optionally be substituted by 1 or more independently selected R^{A} groups, and in particular embodiments, G² may be pyridin-4-yl, pyridin-3-yl, pyridin-2-yl, N-oxo-pyridin-2-yl, N-oxo-pyridin-4-yl, pyrimidin-5-yl, oxazol-5-yl, thiazol-4-yl, isoxazol-4-yl, 2,4-dioxoimidazolidin-3-yl each of which may optionally be substituted by 1 or more independently selected R^{A} groups.

In various embodiments, each R^{A} may, independently, be halogen, cyano, nitro, tri(C₁₋₆)alkylsilyl, OR^{a}, C(O)R^{a}, C(O)NR^{e}R^{f}, C(O)OR^{b}, NR^{c}R^{d}, C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀aryl, C₆₋₁₀aryl-C₁₋₆alkyl, C₅₋₉ heteroaryl, or C₅₋₉heteroaryl-C₁₋₆alkyl each of which may optionally be substituted with 1 or more independently selected R^{A′} groups. In such embodiments, each R^{A′} may, independently, be C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀aryl, C₆₋₁₀aryl-C₁₋₆alkyl, C₅₋₉ heteroaryl, or C₅₋₉heteroaryl-C₁₋₆alkyl each of which may optionally be substituted by 1 or more independently selected R^{A″} groups, where each R^{A″} may, independently, be halogen, cyano, nitro, hydroxyl, C₁₋₆ alkyl, C₁₋₆ haloalkyl, C₁₋₆ alkoxy, C₁₋₆ haloalkoxy, amino, C₁₋₆ alkylamino, di-C₁₋₆alkylamino, or C₁₋₆ alkylsulfonyl. For example, in some embodiments, each R^{A} may, independently, be fluoro, trimethylsilyl, methoxy, ethoxy, isopropoxy, 2-(methoxy)ethoxy, 2-(dimethylamino)ethoxy, 2-(morpholin-4-yl)ethoxy, 3-(morpholin-4-yl)-n-propoxy, 3-(pyridin-2-yl)-n-propoxy, 3-(4-methylpiperazinyl)-n-propoxy, 3-(piperazinyl)-ethoxy, 3-(dimethylamino)-n-propoxy, 2-(piperidinyl)ethoxy, tetrahydropyran-4-yl-oxy, ethylaminocarbonyl, isobutylamino, dimethylamino, 2-(methoxy)ethylamino, 2,3-(dihydroxyl)-n-propylamino, methyl, ethyl, 1-aminomethyl, morpholin-4-yl, (morpholin-4-yl)methyl, (morpholin-4-yl)ethyl, 4-methylpiperazinyl, piperidinyl, 3-(amino)pyrrolinyl, 3-(dimethylamino)-pyrrolidinyl, or 3-aminopyrrolinyl. In still other embodiments, R^{A} or R^{A′} may be:

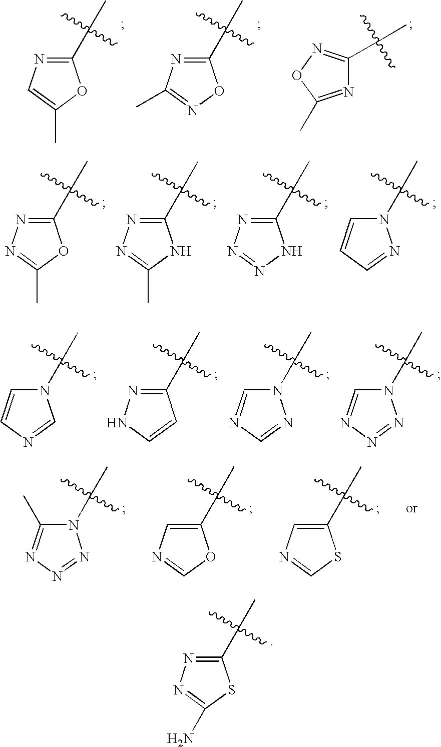

In various embodiments, each R^{a}, R^{c}, R^{d}, R^{e}, and R^{f} may, independently, be hydrogen, C₁₋₆ alkyl, C₁₋₆ haloalkyl, C₃₋₇ cycloalkyl, C₃₋₇ cycloalkyl-C₁₋₁₆alkyl, C₃₋₇ heterocycloalkyl, C₃₋₇ heterocycloalkyl-C₁₋₆ alkyl, C₆₋₁₀aryl, C₆₋₁₀aryl-C₁₋₆alkyl, C₅₋₉ heteroaryl, or C₅₋₉heteroaryl-C₁₋₆alkyl; wherein said C₁₋₆ alkyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀aryl, C₆₋₁₀aryl-C₁₋₆alkyl, C₅₋₉ heteroaryl, and C₅₋₉heteroaryl-C₁₋₆alkyl, and each of these may optionally be substituted with 1, 2 or 3 independently selected R^{g} groups. Each R^{b} may, independently, be C₁₋₆ alkyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀aryl, C₆₋₁₀aryl-C₁₋₆alkyl, C₅₋₉ heteroaryl, or C₅₋₉heteroaryl-C₁₋₆alkyl, and each C₁₋₆ alkyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀aryl, C₆₋₁₀aryl-C₁₋₆alkyl, C₅₋₉ heteroaryl, and C₅₋₉heteroaryl-C₁₋₆alkyl, and each of these may be substituted with 1, 2 or 3 independently selected R^{h} groups. In such embodiments, each R^{g} and R^{h} may, independently, be halogen, C₁₋₆ alkyl, C₁₋₆ haloalkyl, hydroxyl, C₁₋₆ alkoxy, C₁₋₆ haloalkoxy, amino, C₁₋₆alkylamino, or di-C₁₋₆alkylamino.

Y of some embodiments, may be phenyl or 5-membered heteroaryl, each of which may be substituted with 1 or more independently selected R^{Y} groups, and in certain embodiments, Y may be thiophene, thioazole, furan, or pyrazole, each of which may be substituted with 1 or more independently selected R^{Y} groups. In such embodiments, each R^{Y} may, independently, be halogen, C₁₋₆ alkyl, C₁₋₆ haloalkyl, C₅₋₇ heterocycloalkyl, or 5- or 6-membered heterocycle, and in certain such embodiments, each R^{Y} may be tert-butyl or trifluoromethyl. For example, in some embodiments, Y may be:

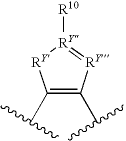

where R^{Y′}, R^{Y″} and R^{Y′″} may each independently be C, CH, O, N, NH, S, SH, or SH₂ and at least one of R^{Y′}, R^{Y″} and R^{Y′″} is a heteroatom and R¹⁰ is tert-butyl, trifluoromethyl, morpholinyl or 5-membered heterocycloalkyl. In further exemplary embodiments, Y may include, but is not limited to:

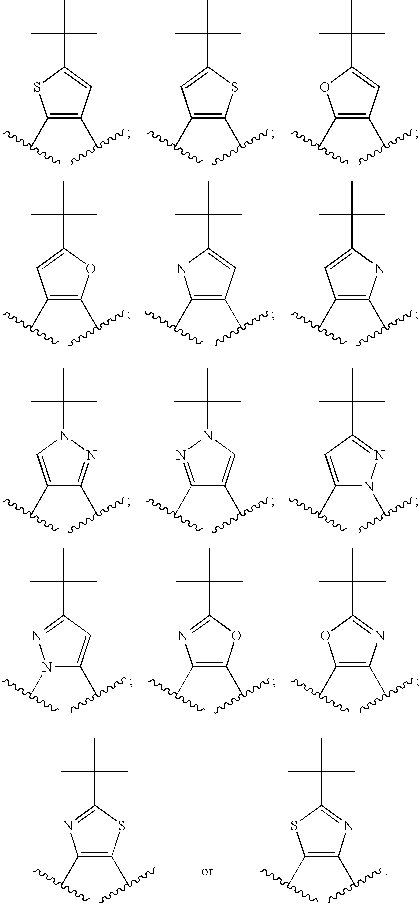

In some embodiments, Y is 5- or 6-membered heteroaryl, either of which may be substituted with one or more independently selected R^{Y} groups as described above.

In particular embodiments, Z of R⁶ may be —NR⁷—, and each X¹ and X² of R⁶ may, independently, be C₁₋₃ alkyl in which each carbon atom may be optionally substituted with one or more oxo or C₁₋₄ alkyl. Thus, in such embodiments, R⁶ may be a heterocycloalkyl having one or more nitrogen heteroatoms and which may include one or more carbon that is substituted by, for example, an oxo or methyl. For example, the R⁶ of some exemplary embodiments, may be:

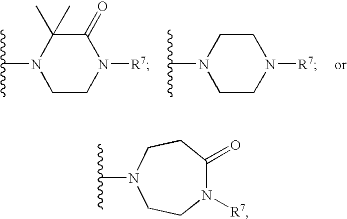

and in particular embodiments, R⁶ may be 3,3-dimethyl-piperazin-2-one.

R⁷ of various embodiments may be hydrogen, C(O)R^{m}, C(O)C(O)R^{m}, C(O)C(O)NRⁿR^{o}, C(O)NRⁿR^{o}, C(O)OR^{p}, S(O)R^{p}, S(O)₂R^{p}, S(O)NRⁿR^{o}, S(O)₂R^{p}, C₁₋₆ alkyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀aryl, C₆₋₁₀aryl-C₁₋₆alkyl, C₅₋₉ heteroaryl, or C₅₋₉heteroaryl-C₁₋₆alkyl, and in such embodiments, each C₁₋₆ alkyl may be optionally substituted with 1 or more independently selected R^{7′} groups, and each C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀aryl, C₆₋₁₀aryl-C₁₋₆alkyl, C₅₋₉ heteroaryl, or C₅₋₉heteroaryl-C₁₋₆alkyl which may be optionally substituted with 1, 2, 3, or 4 independently selected R^{7″} groups. In such embodiments, R^{m}, Rⁿ, R^{o}, and R^{p} may, independently, be hydrogen, C₁₋₆ alkyl, C₁₋₆ haloalkyl, C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀ aryl, C₆₋₁₀ aryl-C₁₋₆ alkyl, C₅₋₉ heteroaryl, or C₅₋₉ heteroaryl-C₁₋₆ alkyl each of which may optionally be substituted with 1, 2 or 3 independently selected R¹ groups that may be halogen, C₁₋₆ alkyl, C₁₋₆ haloalkyl, hydroxyl, C₁₋₆ alkoxy, C₁₋₆ haloalkoxy, amino, C₁₋₆alkylamino, or di-C₁₋₆alkylamino. Each R^{7′} and R^{7″} may, independently, be hydroxyl, C₁₋₆ alkyl, C₁₋₆ haloalkyl, C₁₋₆ alkoxy, C₁₋₆ haloalkoxy, amino, C₁₋₆ alkylamino, di-C₁₋₆alkylamino, or C₁₋₆ alkyloxycarbonyl, such as, but not limited to, hydroxyl, methyl, dimethylamino, or methyloxycarbonyl. For example, in some embodiments, R⁷ may be hydrogen, methyl, (2-methyl-1,3,4-oxadiazol-5-yl)-methyl, (methyloxycarbonyl)methyl, 2-(dimethyl)aminoethyl, 2-(dimethylamino)acetyl, acetyl, 1-methylpyrrolidin-3-yl, (1-methylpyrrolidin-3-yl)methyl, 2-hydroxyethyl, methyloxycarbonyl, methylsulfonyl, 2-(N,N-dimethyl)-2-oxo-acetyl, (1-methyl-1H-imidazol-4-yl)methyl, or (piperidin-4-yl)methyl.

Further embodiments of the invention are directed to compounds of general Formula I where: A¹ may be N or CR¹; A² may be CR²; and A³ may be CR³. Such embodiments, therefore, include those compounds of Formulae II or III:

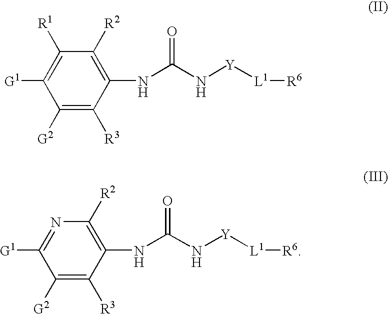

In such embodiments, R¹, R², R³, G², G², Y, L¹ and R³ may be as described above, and in some embodiments, of Formulae I, II or III:

G¹ may be a CH₃, Cl, F, or OCH₃;

G² may be a 6-membered heteroaryl, 5-membered heteroaryl or C₁₋₆ alkynyl, each of which may optionally be substituted by one or more independently selected R^{A} groups;

Y may be a 5-membered heteroaryl which may be substituted with 1 or more independently selected R^{Y} groups;

L¹ is CH₂ or —C(O)—;

R⁶ is

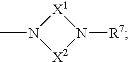

X¹ and X² may each, independently, be C₁₋₃ alkyl, which is optionally substituted with one or more independently selected groups selected from oxo and C₁₋₄ alkyl;

R⁷ may be hydrogen, C(O)R^{m}, C(O)C(O)R^{m}, C(O)C(O)NRⁿR^{o}, C(O)NRⁿR^{o}, C(O)OR^{p}, S(O)R^{p}, S(O)₂R^{p}, S(O)NRⁿR^{o}, S(O)₂R^{p}, C₁₋₆ alkyl which may be substituted with 1 or more independently selected R^{7′} groups, or C₁₋₆ haloalkyl, C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀ aryl, C₆₋₁₀ aryl-C₁₋₆ alkyl, C₅₋₉ heteroaryl, or C₅₋₉ heteroaryl-C₁₋₆ alkyl, and C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀ aryl, C₆₋₁₀ aryl-C₁₋₆ alkyl, C₅₋₉ heteroaryl, or C₅₋₉ heteroaryl-C₁₋₆ alkyl may each be substituted with 1 or more independently selected R^{7″} groups;

each R^{7′} and R^{7″} may, independently, be hydroxyl, C₁₋₆ alkyl, C₁₋₆ haloalkyl, C₁₋₆ alkoxy, C₁₋₆haloalkoxy, amino, C₁₋₆ alkylamino, or di-C₁₋₆alkylamino; and

each R^{A} may, independently, be halogen, cyano, nitro, tri(C₁₋₆)alkylsilyl, OR^{a}, C(O)R^{a}, C(O)NR^{e}R^{f}, C(O)OR^{b}, NR^{c}R^{d}, C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀aryl, C₆₋₁₀aryl-C₁₋₆alkyl, C₅₋₉ heteroaryl, or C₅₋₉heteroaryl-C₁₋₆alkyl, and each C₁₋₆ alkyl, C₂₋₆ alkenyl, and C₂₋₆ alkynyl may be substituted with 1, 2, 3, or 4 independently selected R^{A′} groups and each C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀aryl, C₆₋₁₀aryl-C₁₋₆alkyl, C₅₋₉ heteroaryl, or C₅₋₉heteroaryl-C₁₋₆alkyl may be substituted by 1, 2, or 3 independently selected R^{A″} groups;

each R^{A′} and R^{A″} may, independently, be halogen, cyano, nitro, hydroxyl, C₁₋₆ alkyl, C₁₋₆ haloalkyl, C₁₋₆ alkoxy, C₁₋₆ haloalkoxy, amino, C₁₋₆ alkylamino, di-C₁₋₆alkylamino, or C₁₋₆ alkylsulfonyl;

each R^{a}, R^{c}, R^{d}, R^{e}, and R^{f} may, independently, be hydrogen, C₁₋₆ alkyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀aryl, C₆₋₁₀aryl-C₁₋₆alkyl, C₅₋₉ heteroaryl, or C₅₋₉heteroaryl-C₁₋₆alkyl each which may be substituted with 1, 2 or 3 independently selected R^{g} groups;

each R^{b} may, independently, be C₁₋₆ alkyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀aryl, C₆₋₁₀aryl-C₁₋₆alkyl, C₅₋₉ heteroaryl, or C₅₋₉heteroaryl-C₁₋₆alkyl each of which may be substituted with 1, 2 or 3 independently selected R^{h} groups;

each R^{g} and R^{h} may, independently, be halogen, C₁₋₆ alkyl, C₁₋₆ haloalkyl, hydroxyl, C₁₋₆ alkoxy, C₁₋₆ haloalkoxy, amino, C₁₋₆alkylamino, or di-C₁₋₆alkylamino;

R^{Y} may be halogen, C₁₋₆ alkyl, or C₁₋₆ haloalkyl or C₅₋₇ heterocycloalkyl;

each R^{m}, Rⁿ, R^{o}, and R^{p} may, independently, be hydrogen, C₁₋₆ alkyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀aryl, C₆₋₁₀aryl-C₁₋₆alkyl, C₅₋₉ heteroaryl, or C₅₋₉heteroaryl-C₁₋₆alkyl where each C₁₋₆ alkyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀aryl, C₆₋₁₀aryl-C₁₋₆alkyl, C₅₋₉ heteroaryl, and C₅₋₉heteroaryl-C₁₋₆alkyl may be substituted with 1, 2 or 3 independently selected Rⁱ groups; and

each Rⁱ may, independently, be halogen, C₁₋₆ alkyl, C₁₋₆ haloalkyl, hydroxyl, C₁₋₆ alkoxy, C₁₋₆haloalkoxy, amino, C₁₋₆alkylamino, or di-C₁₋₆alkylamino.

Still other embodiments of the invention are directed to compounds of general Formula IV:

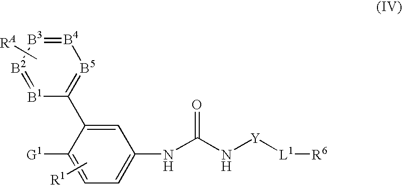

where:

B¹ is N or CR^{1′};

B² is N or CR^{2′};

B³ is N or CR^{3′};

B⁴ is N or CR^{4′};

B⁵ is N or CR^{5′};

R^{1′}, R^{2′}, R^{3′}, R^{4′} and R^{5′} may each, independently, be hydrogen, halogen, cyano, C₁₋₄ alkyl, C₁₋₄ haloalkyl, C₁₋₄ alkoxy, and at least one of R^{1′}, R^{2′}, R^{3′}, R^{4′} and R^{5′} is R^{A};

G¹ may be CH₃, Cl, F, or OCH₃;

R¹ may be absent and may be halogen or C₁₋₄ alkyl; and

R^{A}, Y, L¹ and R⁶ may be as described above.

Yet other embodiments of the invention include compounds of general Formula V:

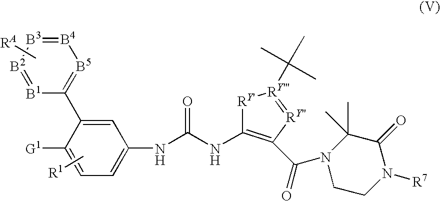

where:

B¹ is N or CR^{1′};

B² is N or CR^{2′};

B³ is N or CR^{3′};

B⁴ is N or CR^{4′};

B⁵ is N or CR^{5′};

R^{1′}, R^{2′}, R^{3′}, R^{4′} and R^{5′} may each, independently, be hydrogen, halogen, cyano, C₁₋₄ alkyl, C₁₋₄ haloalkyl, C₁₋₄ alkoxy, and at least one of R^{1′}, R^{2′}, R^{3′}, R^{4′} and R^{5′} is R^{A};

R^{A} may be as described above; or

R^{A} may be L²-R⁸ where L² may be a bond, C₁₋₆ alkyl, C₁₋₆ alkenyl, C₁₋₆ alkynyl, —C(O)—, —O—, OR^{q} or R^{r}OR^{q}; where each R^{q} and R^{r} may independently be C₁₋₆ alkyl, C₁₋₆ alkenyl, C₁₋₆ alkynyl, OR^{s}, R^{t}OR^{s}, NR^{s}, R^{t}NR^{s}; and each R^{s} and R^{t} may each independently be C₁₋₆ alkyl, C₁₋₆ alkenyl, C₁₋₆ alkynyl; and R⁸ may be absent or a 6-membered aryl, 5- or 6-membered heteroaryl, C₅₋₇ cycloalkyl or C₅₋₇ heterocycloalkyl, each of which may optionally be substituted by 1, 2, 3, or 4 independently selected R^{B} groups where each R^{B} may, independently, be halogen, cyano, nitro, C₁₋₆ alkyl, C₂₋₆ alkenyl, C₂₋₆ alkynyl, C₁₋₆ haloalkyl;

R^{Y′}, R^{Y″} and R^{Y′″} may each independently be C, CH, O, N, NH, S, SH, or SH₂ and at least one of R^{Y′}, R^{Y″} and R^{Y′″} is a heteroatom;

G¹ may be CH₃, Cl, F, or OCH₃;

R¹ may be absent and may be halogen or C₁₋₄ alkyl; and

R⁷ is as described above.

In certain embodiments, L² may be a bond, methyl, ethyl, propyl, butyl, C₃₋₅ alkyne, —C(O)—, —O—, —OCH₂, —OCH₂CH₂—, —OCH₂CH₂CH₂, —NH—, —NHCH₂, —NHCH₂CH₂ and —NHCH₂CH₂CH₂, and R⁸ may be a morpholino, thiomorpholino, piperizino, or pyrrolidino each of which may optionally be substituted with one or more halogen, amino, nitro, cyano, C₁₋₄ alkyl, C₁₋₄ haloalkyl, C₁₋₄ alkoxy.

Embodiments of the invention encompass stereoisomers and optical isomers of the compounds described above including, e.g., mixtures of enantiomers, individual enantiomers and diastereomers, which can arise as a consequence of structural asymmetry of atoms in the compounds of the invention. Such embodiments further include the purified enantiomers which may or may not contain trace amounts of a non-selected enantiomer or diastereomer.

Some embodiments of the invention include salts of the compounds described above. In general, the term salt can refer to an acid and/or base addition salt of a compound. For example, an acid addition salt can be formed by adding an appropriate acid to a free base form of any of the compounds embodied above. Similarly, a base addition salts can be formed by adding an appropriate base to a free base form of any of the compounds described above. Examples of suitable salts include, but are not limited to, sodium, potassium, carbonate, methylamine, hydrochloride, hydrobromide, acetate, furmate, maleate, oxalate, and succinate salts. Methods for preparing free base forms of compounds such as those described herein and acid addition or base addition salts of such compounds are well known in the art, and any such method may be used to prepare the acid or base addition salts of embodiments of the invention.

Other embodiments of the invention include solvates or hydrates of the compounds of the invention. In some cases, hydration of a compound may occur during manufacture of the compounds or compositions including the compounds as a consequence of the method for preparing the compound or as a result of a specific step used to create a hydrate or solvate of the compound. In other cases, hydration may occur over time due to the hygroscopic nature of the compounds. Such hydrated compounds whether intentionally prepared or naturally produced are encompassed by the invention.

Embodiments of the invention also include derivatives of the compounds of the invention which may be referred to as “prodrugs.” The term “prodrug” as used herein denotes a derivative of a known drug that may have enhanced delivery characteristics, enhanced therapeutic value as compared to the active form of the drug, sustained release characteristics, reduced side-effects, or combinations thereof. For example, in some embodiments, a prodrug form of a compound of the invention may be administered in an inactive form or a form having reduced activity that is transformed into an active or more active form of the drug by an enzymatic or chemical process. For instance, in some embodiments, a prodrug form of a compound such as those described above may include one or more metabolically cleavable groups that are removed by solvolysis, hydrolysis or physiological metabolisms to release the pharmaceutically active form of the compound. In other embodiments, prodrugs may include acid derivatives of the compounds of the invention. Acid derivatives are well known in the art and include, but are not limited to, esters or double esters such as, for example, (acyloxy) alkyl esters or ((alkoxycarbonyl)oxy)alkyl esters prepared by reaction of an acid on the parent molecule with a suitable alcohol. Without wishing to be bound by theory, the compounds of the invention may have activity in both their acid and acid derivative forms. However, the acid derivative form may exhibit enhanced solubility, tissue compatibility or delayed release in the mammalian organism (see, e.g., Bundgard, H., Design of Prodrugs, pp. 7-9, 21-24, Elsevier, Amsterdam 1985). In still other embodiments, prodrugs that include an amide may be prepared by reacting a parent compound containing an acid with an amine, and in yet other embodiments, simple aliphatic or aromatic esters derived from acidic groups pendent on a compound of this invention may be prepared as prodrugs.

Embodiments of the invention also include pharmaceutical compositions or formulations including at least one compound embodied hereinabove, an acid or base addition salt, hydrate, solvate or prodrug of the at least one compound and one or more pharmaceutically acceptable carriers or excipients. Pharmaceutical formulations and pharmaceutical compositions are well known in the art, and can be found, for example, in Remington's Pharmaceutical Sciences, Mack Publishing Co., Easton, Pa., USA, which is hereby incorporated by reference in its entirety. Any formulations described therein or otherwise known in the art are embraced by embodiments of the invention.

Pharmaceutical excipients are well known in the art and include, but are not limited to, saccharides such as, for example, lactose or sucrose, mannitol or sorbitol, cellulose preparations, calcium phosphates such as tricalcium phosphate or calcium hydrogen phosphate, as well as binders, such as, starch paste such as, for example, maize starch, wheat starch, rice starch, potato starch, gelatin, tragacanth, methyl cellulose, hydroxypropylmethylcellulose, sodium carboxymethylcellulose, polyvinyl pyrrolidone or combinations thereof.

In particular embodiments, pharmaceutical formulations may include the active compound described and embodied above, a pharmaceutically acceptable carrier or excipient and any number of additional or auxiliary components known in the pharmaceutical arts such as, for example, binders, fillers, disintegrating agents, sweeteners, wetting agents, colorants, sustained release agents, and the like, and in certain embodiments, the pharmaceutical composition may include one or more secondary active agents. Disintegrating agents, such as starches as described above, carboxymethyl-starch, cross-linked polyvinyl pyrrolidone, agar, alginic acid or a salt thereof, such as sodium alginate and combinations thereof. Auxiliary agents may include, for example, flow-regulating agents and lubricants, such as silica, talc, stearic acid or salts thereof, such as magnesium stearate or calcium stearate, polyethylene glycol and combinations thereof. In certain embodiments, dragee cores may be prepared with suitable coatings that are resistant to gastric juices, such as concentrated saccharide solutions, which may contain, for example, gum arabic, talc, polyvinyl pyrrolidone, polyethylene glycol, titanium dioxide, lacquer solutions and suitable organic solvents or solvent mixtures and combinations thereof. In order to produce coatings resistant to gastric juices, solutions of suitable cellulose preparations, such as acetylcellulose phthalate or hydroxypropylmethyl-cellulose phthalate may also be used. In still other embodiments, dye stuffs or pigments may be added to the tablets or dragee coatings, for example, for identification or in order to characterize combinations of active compound doses.

Pharmaceutical compositions of the invention can be administered to any animal, and in particular, any mammal, that may experience a beneficial effect as a result of being administered a compound of the invention including, but not limited to, humans, canines, felines, livestock, horses, cattle, sheep, and the like. The dosage or amount of at least one compound according to the invention provided pharmaceutical compositions of embodiments may vary and may depend, for example, on the use of the pharmaceutical composition, the mode of administration or delivery of the pharmaceutical composition, the disease indication being treated, the age, health, weight, etc. of the recipient, concurrent treatment, if any, frequency of treatment, and the nature of the effect desired and so on. Various embodiments of the invention include pharmaceutical compositions that include one or more compounds of the invention in an amount sufficient to treat or prevent disease indication such as an inflammatory condition, an inflammatory disease, rheumatoid arthritis, psoriatic arthritis or cancer. An effective amount of the one or more compounds may vary and may be, for example, from about 0.001 mg to about 1000 mg or, in other embodiments, from about 0.01 mg to about 10 mg.

The pharmaceutical compositions of the invention can be administered by any means that achieve their intended purpose. For example, routes of administration encompassed by the invention include, but are not limited to, subcutaneous, intravenous, intramuscular, intraperitoneal, buccal, or ocular routes, rectally, parenterally, intrasystemically, intravaginally, topically (as by powders, ointments, drops or transdermal patch), oral or nasal spray are contemplated in combination with the above described compositions.

Embodiments of the invention also include methods for preparing pharmaceutical compositions as described above by, for example, conventional mixing, granulating, dragee-making, dissolving, lyophilizing processes and the like. For example, pharmaceutical compositions for oral use can be obtained by combining the one or more active compounds with one or more solid excipients and, optionally, grinding the mixture. Suitable auxiliaries may then be added and the mixture may be processed to form granules which may be used to form tablets or dragee cores. Other pharmaceutical solid preparations include push-fit capsules containing granules of one or more compound of the invention that can, in some embodiments, be mixed, for example, with fillers, binders, lubricants, stearate, stabilizers or combinations thereof. Push-fit capsules are well known and may be made of gelatin alone or gelatin in combination with one or more plasticizer such as glycerol or sorbitol to form a soft capsule. In embodiments in which soft capsules are utilized, compounds of the invention may be dissolved or suspended in one or more suitable liquids, such as, fatty oils or liquid paraffin and, in some cases, one or more stabilizers.

Liquid dosage formulations suitable for oral administration are also encompassed by embodiments of the invention. Such embodiments, may include one or more compounds of the invention in pharmaceutically acceptable emulsions, solutions, suspensions, syrups, and elixirs that may contain, for example, one or more inert diluents commonly used in the art such as, but not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (for example, cottonseed, groundnut, corn, germ, olive, castor, and sesame oils), glycerol, fatty acid derivatives of glycerol (for example, labrasol), tetrahydrofurfuryl alcohol, polyethylene glycols and fatty acid esters of sorbitan, and mixtures thereof. Suspensions may further contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Formulations for parenteral administration may include one or more compounds of the invention in water-soluble form, for example, water-soluble salts, alkaline solutions, and cyclodextrin inclusion complexes in a physiologically acceptable diluent which may be administered by injection. Physiologically acceptable diluent of such embodiments, may include, for example, sterile liquids such as water, saline, aqueous dextrose, other pharmaceutically acceptable sugar solutions; alcohols such as ethanol, isopropanol or hexadecyl alcohol; glycols such as propylene glycol or polyethylene glycol; glycerol ketals such as 2,2-dimethyl-1,3-dioxolane-4-methanol; ethers such as poly(ethyleneglycol)400; pharmaceutically acceptable oils such as fatty acid, fatty acid ester or glyceride, or an acetylated fatty acid glyceride. In some embodiments, formulations suitable for parenteral administration may additionally include one or more pharmaceutically acceptable surfactants, such as a soap or detergent; suspending agent such as pectin, carbomers, methylcellulose, hydroxypropylmethylcellulose, or carboxymethylcellulose; an emulsifying agent; pharmaceutically acceptable adjuvants or combinations thereof. Additional pharmaceutically acceptable oils which may be useful in such formulations include those of petroleum, animal, vegetable or synthetic origin including, but not limited to, peanut oil, soybean oil, sesame oil, cottonseed oil, olive oil, sunflower oil, petrolatum, and mineral oil; fatty acids such as oleic acid, stearic acid, and isostearic acid; and fatty acid esters such as ethyl oleate and isopropyl myristate. Additional suitable detergents include, for example, fatty acid alkali metal, ammonium, and triethanolamine salts; cationic detergents such as dimethyl dialkyl ammonium halides, alkyl pyridinium halides, and alkylamine acetates; and anionic detergents, such as alkyl, aryl, and olefin sulfonates, alkyl, olefin, ether and monoglyceride sulfates, and sulfosuccinates. In some embodiments, non-ionic detergents including, but not limited to, fatty amine oxides, fatty acid alkanolamides and polyoxyethylenepolypropylene copolymers or amphoteric detergents such as alkyl-β-aminopropionates and 2-alkylimidazoline quaternary salts, and mixtures thereof may be useful in parenteral formulations of the invention.

In particular embodiments, alkaline salts such as ammonium salts of compounds of the invention may be prepared by the addition of, for example, Tris, choline hydroxide, Bis-Tris propane, N-methylglucamine, or arginine to a free base form of the compound. Such alkaline salts may be particularly well suited for use as parenterally administered forms of the compounds of the invention. Buffers, preservatives, surfactants and so on may also be added to formulations suitable for parenteral administration. For example, suitable surfactants may include polyethylene sorbitan fatty acid esters, such as sorbitan monooleate, and the high molecular weight adducts of ethylene oxide with a hydrophobic base, formed by the condensation of propylene oxide with propylene glycol.

Pharmaceutical compositions for parenteral administration may contain from about 0.5 to about 25% by weight of one or more of the compounds of the invention and from about 0.05% to about 5% suspending agent in an isotonic medium. In various embodiments, the injectable solution should be sterile and should be fluid to the extent that it can be easily loaded into a syringe. In addition, injectable pharmaceutical compositions may be stable under the conditions of manufacture and storage and may be preserved against the contaminating action of microorganisms such as bacteria and fungi.

Topical administration includes administration to the skin or mucosa, including surfaces of the lung and eye. Compositions for topical administration, may be prepared as a dry powder which may be pressurized or non-pressurized. In non-pressurized powder compositions, the active ingredients in admixture are prepared as a finely divided powder. In such embodiments, at least 95% by weight of the particles of the admixture may have an effective particle size in the range of 0.01 to 10 micrometers. In some embodiments, the finely divided admixture powder may be additionally mixed with an inert carrier such as a sugar having a larger particle size, for example, of up to 100 micrometers in diameter. Alternatively, the composition may be pressurized using a compressed gas, such as nitrogen or a liquefied gas propellant. In embodiments, in which a liquefied propellant medium is used, the propellant may be chosen such that the compound and/or an admixture including the compound do not dissolve in the propellant to any substantial extent. In some embodiments, a pressurized form of the composition may also contain a surface-active agent. The surface-active agent may be a liquid or solid non-ionic surface-active agent or may be a solid anionic surface-active agent which in certain embodiments, may be in the form of a sodium salt.

Topical formulations for administration to the eye may be delivered in a pharmaceutically acceptable ophthalmic vehicle, such that the compounds are maintained in contact with the ocular surface for a sufficient time period to allow the compounds to penetrate the corneal and internal regions of the eye such as, for example, the anterior chamber, posterior chamber, vitreous body, aqueous humor, vitreous humor, cornea, iris/ciliary, lens, choroid/retina and sclera. A pharmaceutically acceptable ophthalmic vehicle may, for example, be an ointment, vegetable oil or an encapsulating material.

Compositions for rectal or vaginal administration may be prepared by mixing the compounds or compositions of the invention with suitable non-irritating excipients or carriers such as for example, cocoa butter, polyethylene glycol or a suppository wax. Such carriers may be solid at room temperature but liquid at body temperature and therefore melt in the rectum or vaginal cavity and release the drugs.

In still other embodiments, the compounds or compositions of the invention can be administered in the form of liposomes. Liposomes are generally derived from phospholipids or other lipid substances that form mono- or multi-lamellar hydrated liquid crystals when dispersed in an aqueous medium. Any non-toxic, physiologically acceptable and metabolizable lipid capable of forming liposomes can be used, and in particular embodiments, the lipids utilized may be natural and/or synthetic phospholipids and phosphatidyl cholines (lecithins). Methods to form liposomes are known in the art (see, for example, Prescott, Ed., Meth. Cell Biol. 14:33 (1976)). Compositions including one or more compounds of the invention in liposome form can contain, for example, stabilizers, preservatives, excipients and the like.

In yet other embodiments, one or more compounds of the invention may be formulated for in vitro use in, for example, an assay for inhibition of p38 or an assay that requires inhibition of p38. In such embodiments, the composition of the invention may include one or more compounds presented herein above in a carrier that is suitable for an assay. Such carriers may be in solid, liquid or gel form and may or may not be sterile. Examples of suitable carriers include, but are not limited to, dimethylsulfoxide, ethanol, dichloromethane, methanol and the like.

Embodiments of the invention are further directed to methods for using the compounds and compositions described herein above. For example, in some embodiments, the compounds or compositions of the invention may be used in the treatment or prevention of a p38-mediated condition. Methods of such embodiments may generally include the step of administering to a subject in need of such treatment an effective amount of a compound or a composition selected from one or more of the embodiments described above to treat, prevent or ameliorate a p38-mediated compound, and in particular embodiments, the condition or disease may be mediated by p38α. In other embodiments, methods of the invention may include the step of administering to a subject in need of such treatment an effective amount of a compound or composition selected from one or more of the embodiments described above to treat, prevent or ameliorate an inflammatory condition or disease.

In such embodiments, the subject may be an animal, preferably a mammal, including but not limited, a cow, horse, sheep, pig, chicken, turkey, quail, cat, dog, mouse, rat, rabbit or guinea pig, and in particular embodiments, a human.

The term “p38-mediated condition,” as used herein means any disease or other deleterious condition in which p38 is known to play a role, and include those conditions known to be caused by interleukins or TNFs and, in particular, TNF-α overproduction. Such conditions include, for example, inflammatory diseases including, but are not limited to, acute pancreatitis, chronic pancreatitis, asthma, allergies, and adult respiratory distress; autoimmune diseases, including but are not limited to, glomerulonephritis, rheumatoid arthritis, systemic lupus erythematosus, scleroderma, chronic thyroiditis, Graves' disease, autoimmune gastritis, diabetes, autoimmune hemolytic anemia, autoimmune neutropenia, thrombocytopenia, atopic dermatitis, chronic active hepatitis, myasthenia gravis, multiple sclerosis, inflammatory bowel disease, ulcerative colitis, Crohn's disease, psoriasis, or graft vs. host disease; chronic obstructive pulmonary disorder, destructive bone disorders including but are not limited to, osteoporosis, osteoarthritis and multiple myeloma-related bone disorder; proliferative disorders including but are not limited to, acute myelogenous leukemia, chronic myelogenous leukemia, metastatic melanoma, Kaposi's sarcoma, and multiple myeloma; cancer; infectious diseases including but are not limited to, sepsis, septic shock, and Shigellosis and viral diseases including but are not limited to, acute hepatitis infection (including hepatitis A, hepatitis B and hepatitis C), and CMV; retinitis; neurodegenerative diseases including but are not limited to, Alzheimer's disease, Parkinson's disease, and cerebral ischemias or neurodegenerative disease caused by traumatic injury; allergies; reperfusion/ischemia in stroke; heart attacks; angiogenic including but not limited to, solid tumors, ocular neovasculization, infantile haemangiomas disorders, organ hypoxia, vascular hyperplasia, cardiac hypertrophy, thrombin-induced platelet aggregation, and conditions associated with prostaglandin endoperoxidase synthase-2.

In one embodiments one or more compound and composition of the invention may be used for the treatment and/or prevention of allergies or may be used to treat or prevent inflammatory symptoms of an allergic reaction. In another embodiment, the compound or composition is used to treat or prevent a respiratory inflammatory response evoked by an allergen.

In other embodiments, one or more compounds or compositions of the invention may be used to treat cancer, such as, for example, cancer associated with chronic inflammation, including but not limited to colorectal cancer, colon cancer, esophageal cancer, mesothelioma, ovarian cancer, and gastric cancer. In still other embodiments, the compound or composition of the invention may be used to treat cancer by blocking tumorigenesis, inhibiting metastasis or inducing apoptosis.

Other embodiments of the invention include methods in which one or more of the compounds or compositions described herein may be administered to a subject to inhibit or prevent a healthy subject from developing an inflammatory condition or a p38-mediated condition. As such, the compounds and compositions of the invention may be used as a prophylactic that prevents or inhibits the development of an inflammatory or p38-mediated condition or disease. In such embodiments, the compound or composition may be administered to a subject who does not have an inflammatory or p38-mediated condition or is not exhibiting the symptoms of an inflammatory of p38-mediated condition but may be at risk of developing one to prevent or inhibit the onset of such a disorder. For example, the individual may be genetically predisposed to an inflammatory disorder or p38-mediated condition or has increased likelihood of developing such a disorder as a result of, for instance, an injury, surgery or other medical condition.

In general, methods of embodiments of the invention may include the step of administering or providing an “effective amount” or a “therapeutically effective amount” of a compound or composition of the invention to an individual. In such embodiments, an effective amount of the compounds of the invention may be any amount which produces the desired effect. As described above, this amount may vary depending on, for example, the circumstances under which the compound or composition is administered (e.g., to incite treatment or prophylactically), the type of individual, the size, health, etc. of the individual and so on. The dosage may further vary based on the severity of the condition. For example, a higher dose may be administered to treat an individual with a well-developed inflammatory condition, compared to the amount used to prevent a subject from developing the inflammatory condition. Those skilled in the art can discern the proper dosage based on such factors. For example, in some embodiments, the dosage may be within the range of about 0.01 mg/kg body weight to about 300 mg/kg body weight or between about 0.1 mg/kg body weight and about 100 mg/kg body weight, and in particular embodiments, the dosage may be from about 0.1 mg/kg body weight to about 10 mg/kg body weight.

The administration schedule may also vary. For example, in some embodiments, the compounds or compositions of the invention may be administered in a single dose once per day or once per week. In other embodiments, the compounds or compositions of the invention may be administered in two, three, four or more doses per day or per week. For example, in one embodiment, an effective amount for a single day may be divided into separate dosages that may contain the same or a different amount of the compound or composition and may be administered several times throughout a single day. Without wishing to be bound by theory, the dosage per administration and frequency of administration may depend, for example, on the specific compound or composition used, the condition being treated, the severity of the condition being treated, and the age, weight, and general physical condition of the individual to which the compound or composition is administered and other medications which the individual may be taking. In another exemplary embodiment, treatment may be initiated with smaller dosages that are less than the optimum dose of the compound, and the dosage may be increased incrementally until a more optimum dosage is achieved.

In each of the embodiments above, the compound administered can be provided as a pharmaceutical composition including compound as described above and a pharmaceutically acceptable excipient, or a pure form of the compound may be administered.

In additional embodiments, the compound or composition of the invention may be used alone or in combination with one or more additional agents. For example, in some embodiments, a compound or composition of invention may be formulated with one or more additional anti-inflammatory agents, anti-cancer agents or combinations thereof such that the pharmaceutical composition obtained including the compound or composition of the invention and the one or more additional agents can be delivered to an individual in a single dose. In other embodiments, the compound or composition of the invention may be formulated as a separate pharmaceutical composition that is delivered in a separate dose from pharmaceutical compositions including the one or more additional agents. In such embodiments, two or more pharmaceutical compositions may be administered to deliver effective amounts of a compound or composition of the invention and the one or more additional agents.

Method of certain embodiments of the invention may include the step of selectively inhibiting a p38 kinase by, for example, contacting p38 kinase with a compound or composition according to the invention. In such embodiments, the p38 kinase may be contained within a living organism, living tissue or one or more living cells to provide in vivo inhibition, or the p38 kinase may be isolated to provide in vitro inhibition. For example, compounds or compositions described herein may be useful in in vitro drug discovery assays in which the efficacy and/or potency of other anti-inflammatory or p38 kinase inhibitors. The amount of the compound or composition of the invention used to inhibit p38 is not necessarily the same when used in vivo compared to in vitro. For example, factors such as pharmacokinetics and pharmacodynamics of a particular compound may require that a larger or smaller amount of the compound be used for in vivo applications. In another embodiment, a compound or composition according to the invention may be used to form a co-crystallized complex with p38 protein.

By “selectively” is meant that the compounds and compositions described herein inhibit the activity of p38 kinase without interfering with the activity of the other kinases. For example, compounds or compositions of the invention can be administered to a cell that contains a p38 kinase as well as other kinases such as, for example, c-RAF, Flt3, JNK2α2, JNK3, Lck, Lyn, Tie2, TrkB, IGF-R, ERK1, ERK2, MEK1, PRAK, Yeo and/or ZAP-70. For instance, in some embodiments, the method of the invention can inhibit greater than about 80% of the activity of a p38 kinase while inhibiting less than about 5%, about 10%, about 20% or about 30% of the activity of other kinases such as those listed above. Additionally, it is noted that the compounds or compositions of the invention can selectively inhibit a p38α or p38β kinase without substantially inhibiting the activity of a p38γ or p38δ kinase. For example, in certain embodiments, compounds or compositions of the invention can inhibit greater than about 80% of the activity of a p38α or p38β kinase while inhibiting less than about 30%, about 40% or about 50% of the activity of a p38γ or p38δ kinase.

One skilled in the art can evaluate the ability of a compound to inhibit or modulate the activity of a p38 kinase and/or prevent, treat, or inhibit an inflammatory condition by one or more assays known in the art. For example, in one embodiment, inhibition of the p38-catalyzed phosphorylation of EGF receptor peptide as described in U.S. Publication No. 2003/0149037 to Salituro et al., which is hereby incorporated by reference can be used to test a compound or composition of the invention. The inhibitory activity of the test compound can be determined by comparing the extent of phosphorylation of the EGF receptor peptide in the presence of test compound compared to phosphorylation in the absence of test compound. In another embodiment, p38-inhibitory activity can be tested by observing or quantifying inhibition of ATPase activity of activated p38 using HPLC or TLC analysis. In still another embodiment, inhibition of p38 kinase activity can be determined by the incorporation of ³³P/³²P from γ-[³³P/³²P]ATP into the GST-ATF-2 substrate which is catalyzed by p38. In yet another embodiment, inhibition of p38 can be measured by determining the activation kinetics of p38 by MKK6 using, e.g., ELISA. Similarly, in certain embodiments, a compound or composition of the invention may be tested for the ability to inhibit TNFα secretion caused by lipopolysaccharide (LPS).

The compounds of the invention can be synthesized by any method known in the art, and embodiments of the invention further include methods for preparing or the compounds described above. For example, a compound of Formula I, wherein L¹ is C(O) or CH₂, can be prepared using general Method I as follows:

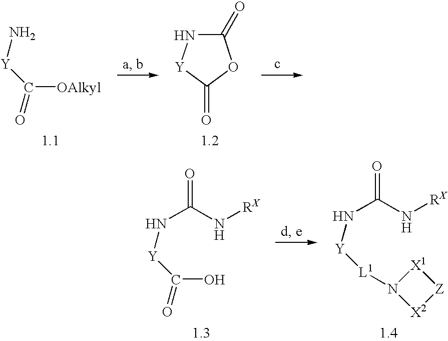

where L¹ is CH₂ or C(O), Y, X¹, X², and Z are as defined for Formula I and R^{X} represents the aryl:

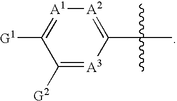

In such embodiments, Step (a) may use a base such as sodium hydroxide or potassium hydroxide to hydrolyze the ester and the resulting acid may be reacted with phosgene or triphosgene to form Compound 1.2 in Step (b). Compound 1.2 may then be reacted with a suitable amine such as, for example, R^{X}—NH₂, in Step (c) to form Compound 1.3 which may then be coupled with another suitable amine of Formula VI:

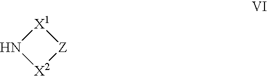

to form Compound 1.4 in Step (d). In such exemplary embodiments, L¹ is C(O). In certain embodiments, a coupling agent such as, for example, EDCI and 1-hydroxybenzotriazole may be used in Step (d). If desired, Compound 1.4 may be further reacted with a reducing agent such as, for example, BH₃-THF, which can reduce the C(O) to CH₂ to create a compound where L¹ is CH₂.

In an exemplary embodiment, a compound according to Formula I, where L¹ is either C(O) or CH₂, can be prepared as follows:

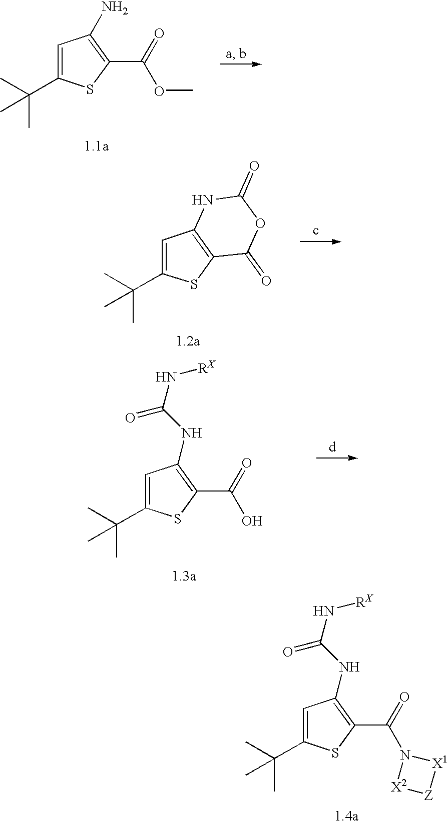

where X¹, X² and Z are defined for Formula I and R^{X} is defined as above. In particular embodiments, potassium hydroxide may be used to convert the ester of Compound 1.1a to an acid in Step (a), COCl₂, phosgene or triphosgene may be used to catalyze formation of the substituted cyclohexane as shown in Compound 1.2a in Step (b), R^{X}—NH₂ is used to from an amide in Step (c) to form Compound 1.3a and in Step (d) an amine of Formula VI and, optionally, a catalyst or coupling agent, e.g., EDCI and 1-hydroxybenzotriazole (HOBt), can be used to effect the formation of the amide as illustrated in Compound 1.4a in Step (d).

In other embodiments, a compounds of the invention wherein L¹ is C(O) and R⁶ is:

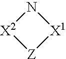

can be prepared using general Method II as follows:

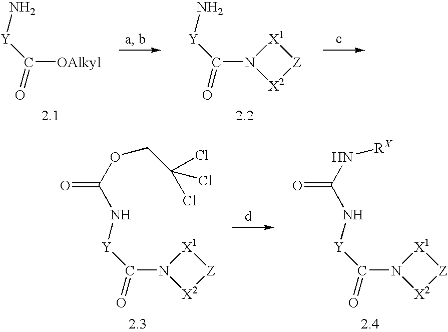

where Y, X¹ and X² are as defined for Formula I and R^{X} is defined as above. In Method II, Step (a) may include the steps of reacting Compound 2.1 with a base, e.g., NaOH or K₂CO₃ to form the acid, which is then reacted with an appropriate amine to form the amide shown in Compound 2.2. In Step (c), the amine associated with Y may be reacted with, for example, 2,2,2-trifluoroethylchloroformate to form the carbamate shown in Compound 2.3, and the carbamate may then be reacted with amine R^{X}—NH₂ to form compound 2.4 in Step (d).

For example, a compound according to Formula I can be prepared according to Method II as follows:

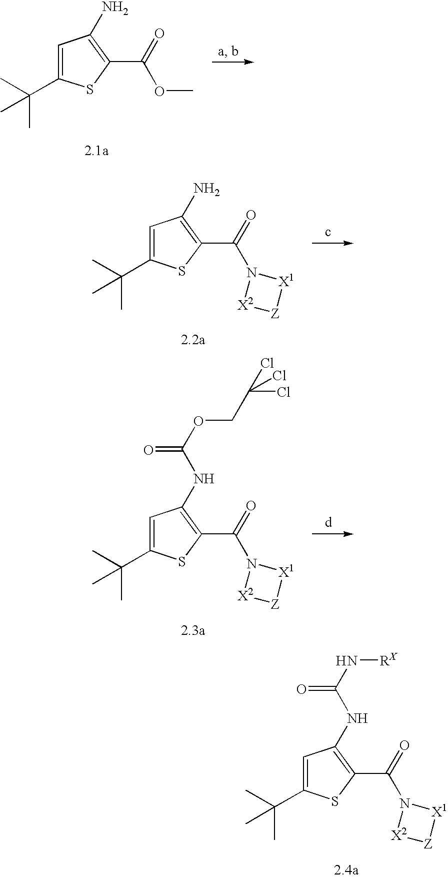

where in Step (a) the ester of Compound 2.1a may react with KOH and an amide of Formula V, EDCI and HOBt to form Compound 2.2a. The amide associated with the thiophene may then be reacted with a carbamate, for example:

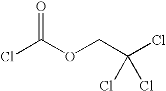

to form Compound 2.3a and a urea may be formed by reacting and amine, R^{X}—NH₂ with the carbamate. In some embodiments, the resultant Compound 2.4a can be further reacted with a reducing agent such as, for example, BH₃-THF, which can reduce the C(O) to CH₂ to create a compound where L¹ is CH₂.

In still other embodiments, compounds according to Formula I can be prepared as shown in Method III:

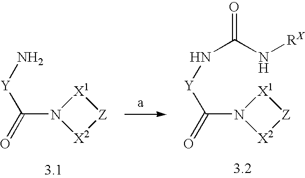

where Y, X¹, X² and Z are as defined as in Formula I and R^{X} is defined as above. Step (a) may include reacting the amine of Compound 3.1 with an appropriate isocyanate R^{X}—NCO or carbamoyl chloride under microwave conditions.

For example, a compound of Formula I can be prepared according to Method III as follows:

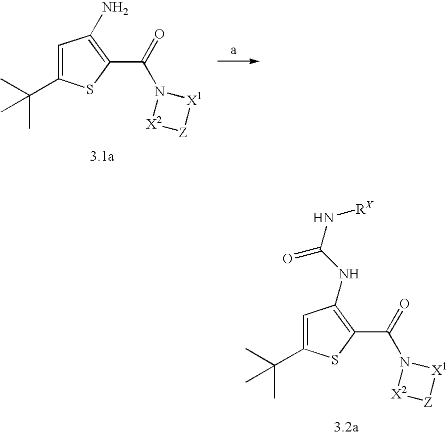

wherein X¹, X² and Z are defined as for Formula I and R^{X} is defined as above. In this method, Step (a) reacts a compound R^{X}—NCO with the amino thiophene shown as Compound 3.1a while subjecting the reaction components to microwave conditions to produce Compound 3.2a. In some embodiments, the resultant Compound 3.2a can be further reacted with a reducing agent such as, for example, BH₃-THF, which can reduce the C(O) to CH₂ to create a compound where L¹ is CH₂.

Further embodiments of the invention include a method for preparing compounds of Formula I using Method IV as follows:

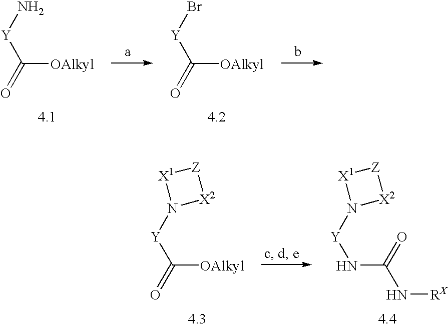

where Y, X¹, X² and Z are as defined for Formula I and R^{X} is defined as above. In Step (a), the amino ester (4.1) may be reacted with, for example, NaNO₂/CuBr₂, to produce a halogen containing (4.2) may then be reacted with an appropriate amine of Formula VI in the presence of a catalyst (e.g., Pd) to form the amine containing group (4.3) in Step (b). In Step (c), the resulting compound (4.3) may be the hydrolyzed with a base to form the corresponding acid. The resulting acid may then be reacted with DPPA/TEA in Step (d), and the resulting compound may be reacted with an amine of, for example, the formula R^{X}—NH₂ to produce a compound of Formula I (4.4).

For example, a compound of Formula I having a thiophene at Y can be prepared according to Method IV as follows:

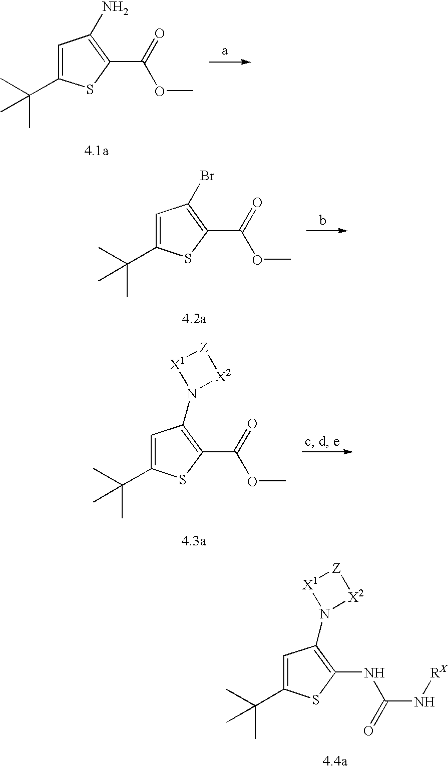

where X¹, X² and Z are defined as described for Formula I and R^{X} is defined as above. In Step (a), as amino thiophene (4.1a) may be reacted the with NaNO₂/CuBr₂ to produce a brominated thiophene (4.2a). In Step (b) an amine of Formula VI may be reacted with the brominated thiophene (4.2a) to produce compound 4.3. This ester of Compound 6.3 is then hydrolyzed with a base such as, for example, NaOH, in Step (c). DPPA/TEA is added to the reaction in Step (c), and an amine of formula R^{X}NH₂ is reacted in Step (e) to form a compound or Formula I (4.4a).

Still other methods for preparing compounds of Formula I include Method V as follows:

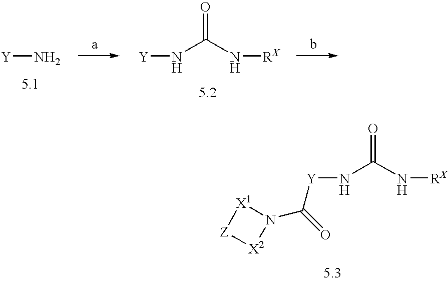

where Y may be a nitrogen-containing heteroaryl, and X¹, X² and Z are as defined for Formula I and R^{X} is defined as above. In Step (a), an amine containing compound (5.1) may be reacted with and isocyanate of, for example, formula R^{X}—NCO to produce a urea containing compound (5.2). In Step (b), the resulting urea compound (5.2) may than be reacted with a compound of formula:

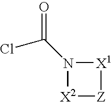

such that a carbamate forms a bond with a nitrogen atom of Y, thereby forming a compound according to Formula I (5.3).

For example, a compound of Formula I where Y is a 1,2-diazole can be prepared according to Method V as follows:

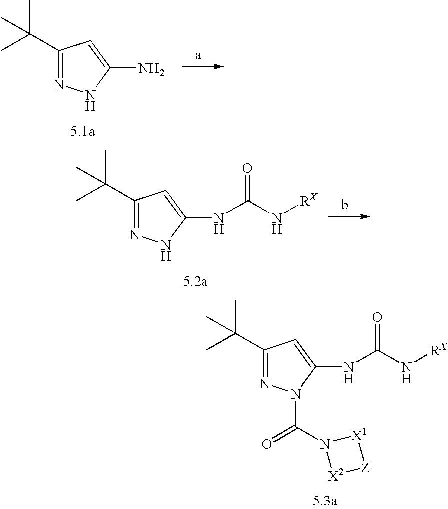

wherein X¹, X² and Z are defined as for Formula I and R^{X} is defined as above. In Step (a), an isocyanate of formula R^{X}—NCO may be reacted with the 1,2-diazole (5.1a) to form a urea containing 1,2-diazole (5.2a). In Step (b), the urea containing 1,2-diazole is reacted with an amine compound of Formula VI to produce a compound of Formula I (5.3a).

In still other embodiments, compounds of Formula I can be prepared using Method VI as follows:

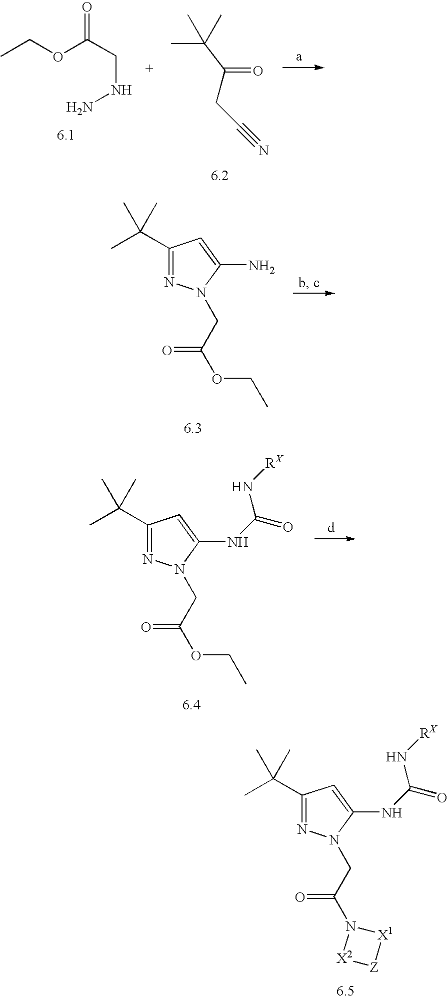

where X¹, X² and Z are defined as for Formula I and R^{X} is defined as above. In Step (a), a hydrazinylactate (6.1) and nitrile containing compound (6.2) may be heated in an organic solvent such as, for example, toluene, to produce a substituted amine-1,2-diazole containing compound 6.3. In Step (b), the product of Step (a) (6.3) may be reacted with an isocyanate of, for example, formula R^{X}—NCO followed by addition of a base such as, for example, NaOH, in Step (c) to produce a urea containing (6.4). In Step (d), the product of Step (c) (6.4) may be reacted with an amine of Formula VI in the presence of DIEA/EDCI to produce a compound of Formula I (6.5).

In further embodiments, compounds of the invention can be prepared using Method VII as follows:

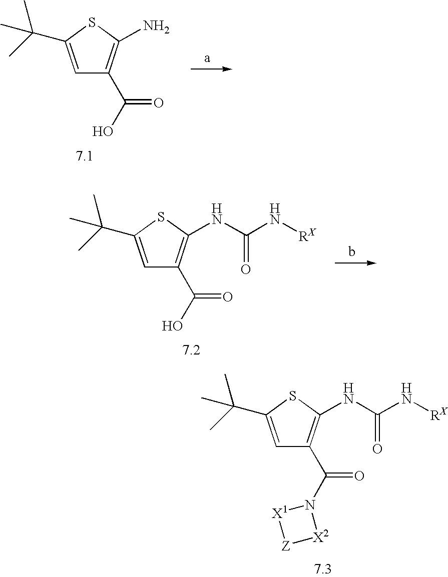

where X¹, X² and Z are defined as for Formula I and R^{X} is defined as above. In Step (a), an amino thiophene (7.1) may be reacted with an isocyanate of formula R^{X}—NCO to form a urea containing compound (7.2). This urea containing compound (7.2) may then be reacted with an amine of Formula VI in the presence of EDCI and HOBt to form a compound according to Formula I (7.3).

In yet other embodiments, compounds of the invention can be prepared using Method VIII as follows:

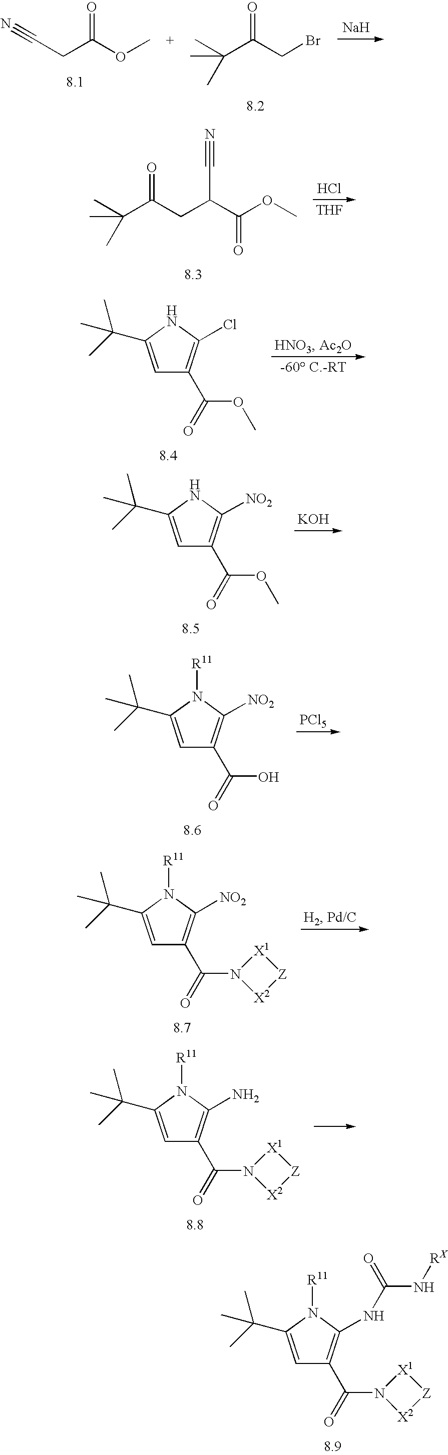

where X¹, X² and Z are as defined as for Formula I, R^{X} is defined as above and R¹¹ may be H or methyl. In such embodiments, Y may be an aminopyrrole and L¹ may be C(O) as illustrated above, however, the method provided above various reactants may be modified or exchanged to produce compounds in which Y is, for example, thiophene, furan, 1,2-diazole and the like. In the method above, 2-chloropyrrol (8.4) may be prepared by reacting a cyano containing compound (8.1) with a brominated oxo containing compound (8.2) to from a oxo and cyano containing acetic acid (8.3). The 2-chloropyrrol (8.4) can be converted in 2 steps to 2-nitropyrrole (8.5) as shown. In a further step, an ester associated with the 2-nitropyrrole (8.5) can be hydrolyzed using, for example, KOH, to the corresponding carboxylic acid (8.6), which may then be reacted with an amine of Formula VI to yield an amide (8.7). In another step, a reducing agent may be used to reduce the nitro group of the 2-nitropyrrole to an amine (8.8) which can then be reacted with an isocyanate to yield a urea containing compound of Formula I (8.9).

Further embodiments of the invention include methods for preparing compound of Formula I using Method IX as follows:

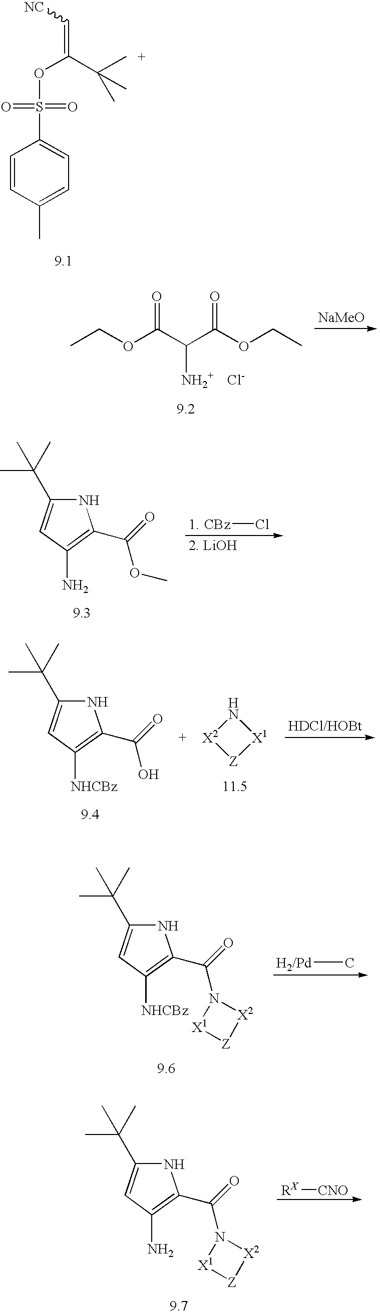

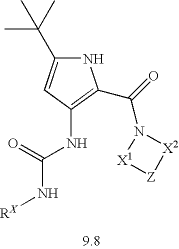

where X¹, X² and Z are defined as for Formula I and R^{X} is defined as above. In such embodiments, a 3-aminopyrrole (9.3) may be prepared from a cyano containing tosylate (9.1) and a bis-ethoxycarbonyl and ammonium containing compound (9.2). The 3-aminopyrrole (9.3) may then be reacted with, for example, benzylcarbamate chloride, to provide a benzylcarbamate protecting group on the amine and the ester may be hydrolyzed to a carboxylic acid using, for example, LiOH, to yield, for example, compound (9.4). The acid is then reacted with appropriate amine of Formula VI (9.5) to yield amide an amide containing compound (9.6), and, for example, Pd on carbon (H2/Pd—C) may be used to remove the protected benzylcarbamate to give an amine containing compound (9.7). The amine containing compound can then be reacted with the appropriate isocyanate (R¹—CNO) to yield compound of Formula I (9.8).

Still further embodiments certain compounds of Formula I can be prepared using Method X as follows:

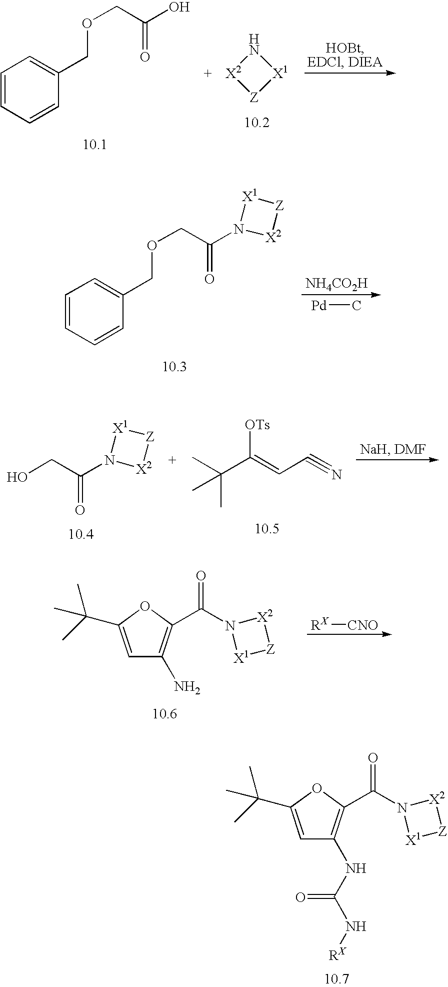

wherein X¹, X², and Z are defined as for Formula I and R^{X} is defined as above. In such embodiments, 2-benzyloxyacetic acid (10.1) can be reacted with the appropriate amine Formula VI (10.2) to yield an amide (10.3), which may then be reacted with ammonium formate and Pd—C to remove the benzyl group to yield a hydroxyacetaldehyde containing amide (10.4). The hydroxyacetaldehyde containing amide may then be reacted with an appropriate tosylate (10.5) to give an aminofuran (10.6) which can be reacted with the appropriate isocyanate (R^{X}—CNO) to produce an exemplary compound of Formula I (10.7). In some embodiments, compounds according to Formula I can be prepared using Method XI as follows:

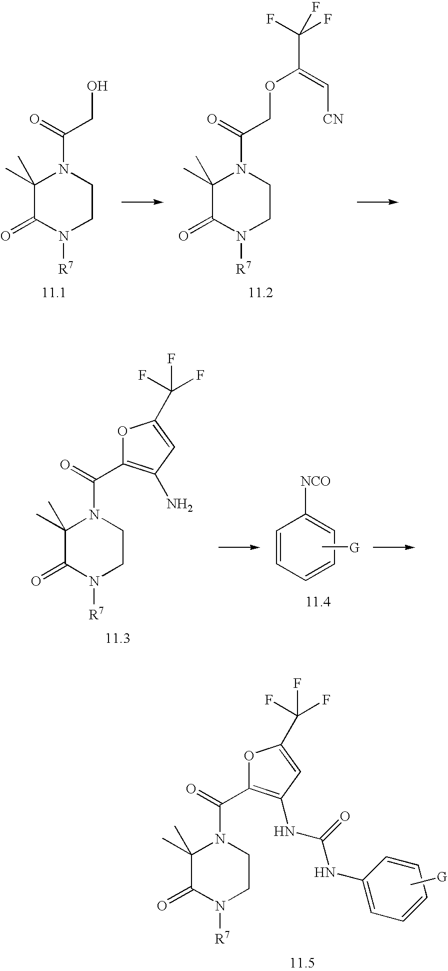

where R⁷ is defined as for Formula I. In such embodiments, the acid of the hydroxyacetaldehyde containing compound (11.1) may be converted to an ester using, for example, NaH, DMF, and reacted with a cyano containing compound to yield a compound such as compound (11.2) which may then be heated to produce a aminofuran compound (11.3). The aminofuran (11.3) may then be reacted with an appropriate isocyanate (11.4) to give an exemplary compound of Formula I. In particular embodiments, G may be a substituted or unsubstituted phenyl or a substituted or unsubstituted 5- or 6-membered heteroaryl.

In other embodiments, compounds according to Formula I can be prepared according to Method XII as follows:

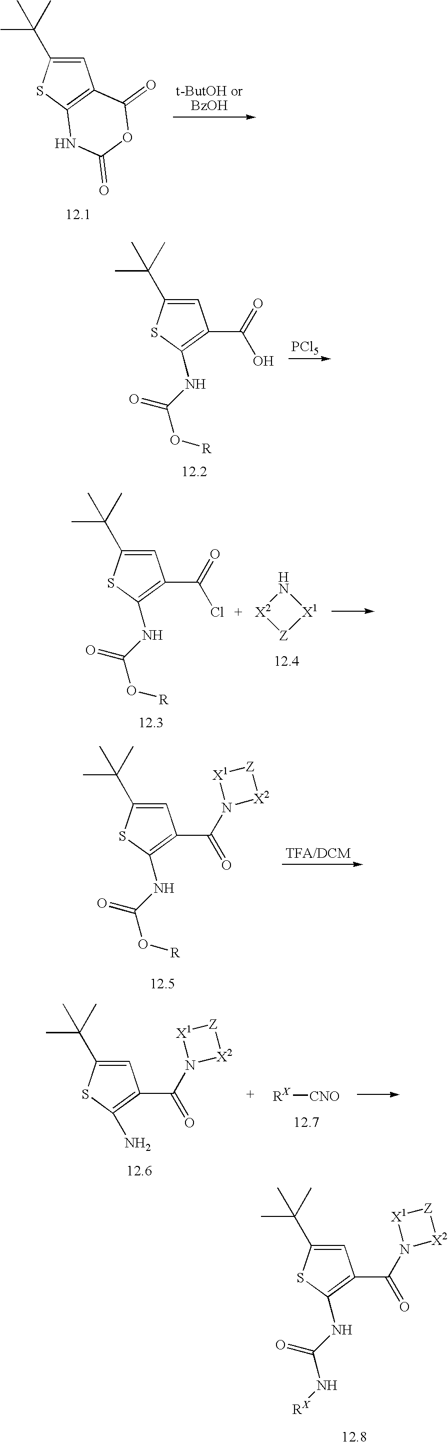

where X¹, X² and Z are as defined above and R^{X} is defined as above. In such embodiments, a thienooxazine dione (12.1) can be reacted with, for example, t-butanol or benzyl alcohol, to produce an aminothiophene substituted with a carboxylic acid (12.2) in which the amine may be protected with, for example, a t-Boc or a benzylcarbamate. The carboxylic acid of compound (12.2) may then be converted to acid chloride (12.3) which may then be reacted with an appropriate amine (12.4) to produce an amide (12.5). The t-Boc or BzC protecting group may then be removed by conventional means to produce an amine containing compound (12.6), and the amine (12.6) may be reacted with appropriate isocyanate (12.7) to produce an exemplary compound of Formula I (12.8).

In still other embodiments, compounds according to Formula I can be prepared using Method XIII as follows:

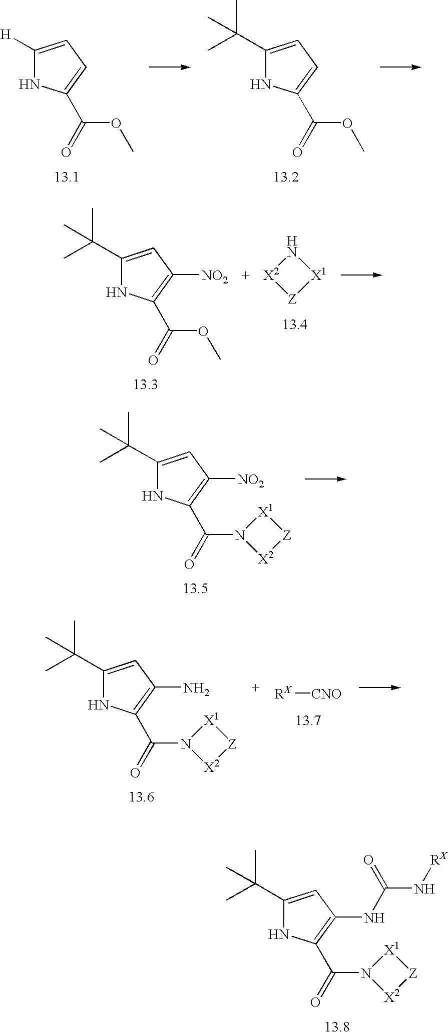

wherein X¹, X² and Z are defined as for Formula I and R^{X} is defined as above. In such embodiments, a pyrrole (13.1) can reacted with tert-butylchloride under Friedel-Crafts alkylation conditions to give the tert-butylpyrrol (13.2), which can then be nitrated to form a nitro tert-butylpyrrol (13.3). The ester may then be hydrolyzed using, for example, LiOH, and the nitro tert-butylpyrrole can be coupled to an appropriate amine of Formula VI (13.4) to produce an amide containing compound (13.5). The nitro group of compound (13.5) may then be reduced to produce an amine containing compound (13.6) which can be reacted with isocyanate (13.7) to give an exemplary compound of Formula I (13.8).

The corresponding starting amines are either commercially available or can be prepared by methods available in the art. Non-commercially available starting materials and production of non-commercially available compounds are described below. Of course, other methods and procedures well known in the art may be used to prepare certain compounds of Formula I.

Several procedures are described below related to preparation of the named compounds. Those of skill in the art will recognize that analogous compounds can be made through procedures analogous to either the general schemes or specific procedures discussed herein.

Procedures

### Synthesis of 1-(5-tert-butyl-3-(2,2-dimethyl-3-oxopiperazine-1-carbonyl)thiophen-2-yl)-3-(4-methyl-3-(5-(morpholinomethyl)pyridin-2-yl)phenyl)urea

### Preparation of 4-methyl-3-(5-(morpholinomethyl)pyridin-2-yl)aniline

To a RB flask containing formylpyridine (9.347 g, 1 equiv.) in 175 mL 1,2-dichloroethane (3.5 mL/mmol) was added morpholine (4.7 mL, 1.07 equiv.) followed by NaBH(OAc)₃ (14.819 g, 1.4 equiv.) and acetic acid (3.1 mL, 1.07 equiv.). The flask was loosely capped and the mixture was stirred at r.t. Mixture gets slightly warm. After 40 min, the reaction was quenched with saturated NaHCO₃. When the gas evolution was greatly reduced, 1M NaOH was added to bring the pH to 8-9. The two layers were separated and the aqueous layer was extracted with DCM (×3). The organic layer was dried over Na₂SO₄, filtered and solvent was removed in vacuo. The product was filtered through silica with 1000 mL 100:1 EtOAc:NH₄OH to remove baseline material. The material was dissolved in 15 mL EtOAc and 150 mL hexanes was added. The mixture was allowed to sit overnight at 4° C. to crystallize. The supernatant was decanted off and the crystals were washed with a little hexanes which was decanted off. The crystals were transferred to another flask using DCM. LC-MS showed only product. The solvent from the supernatant was removed in vacuo. The remaining mixture was purified by flash column (6.5×8.5 cm silica) using 500 mL 8:2 EtOAc; 1400 mL 100:1 EtOAc:NH₄OH. All product fractions were combined giving 10.931 g (85%) of the 4-((6-bromopyridin-3-yl)methyl)morpholine as a yellow solid.

In a RB flask, a mixture of 4-((6-bromopyridin-3-yl)methyl)morpholine (10.959 g, 1 equiv.), 4-methyl-3-(4,4,5,5-tetramethyl-1,3,2-dioxaborolan-2-yl)aniline (10.478 g, 1.05 equiv.), 2M potassium phosphate solution (42.5 mL, 2 equiv.) and Pd(PPh₃)₄ (2.479, 0.050 equiv.)) in 210 mL dioxane (5 mL/mmol) was sparged with argon for 5 min. The flask was fitted with a septum and argon balloon and the mixture was stirred at 100° C. (amber solution). After 27 h, the mixture was allowed to cool then volume was reduced by at least half in vacuo. The remainder was diluted with water and extracted with EtOAc (×3). The organic layers were washed with brine, dried over Na₂SO₄, filtered and solvent was removed in vacuo. The material was purified by column chromatography on silica eluting with DCM:MeOH to give 10.268 g (85%) of 4-methyl-3-(5-(morpholinomethyl)pyridin-2-yl)aniline as a very viscous dark amber oil.

### Preparation of 3,3-dimethylpiperazine-2-one

To a solution of ethylenediamine (2.3 L, 1.92 Kg, 32 mol) in distilled toluene (8 L) was added ethyl-2-bromoisobutyrate (1 Kg, 5.11 mol) in toluene (2 L) drop wise over a period of 2 hours under nitrogen. The reaction mixture was stirred for 3 hours at room temperature and KOH (290 g, 5.11 mol) powder was added and refluxed at 110° C. overnight. The reaction mixture was cooled and filtered. The filtrate was concentrated to get a yellow residue. The residue was washed with ethyl acetate (3*1 L) until the impurities were removed by TLC. It was filtered and dried to obtain 3,3-dimethylpiperazine-2-one as a white crystalline solid (240 g, 36%).

### Preparation of 1-(5-tert-butyl-3-(2,2-dimethyl-3-oxopiperazine-1-carbonyl)thiophen-2-yl)-3-(4-methyl-3-(5-(morpholinomethyl)pyridin-2-yl)phenyl)urea

To a 1 L flask containing a mixture of methyl cyanoacetate (MW=99.08, 19.9 g, 201 mmol) and pre-ground sulfur (MW=32.06, 6.45 g, 201 mmol) in 100 mL DMF at r.t. was added TEA (MW=101.19, 10.9 g, 15 mL, 108 mmol, 0.535 eq). The mixture turns brown immediately upon TEA addition. The mixture was stirred until the sulfur had dissolved (ca 30 min). 3,3-dimethylbutraldehyde (MW=100.15, 20.15 g, 201 mmol, d 0.783, 25.2 mL) was added (5 mL DMF used to wash over remaining aldehyde) and the mixture was stirred at r.t. Precipitate formed shortly after aldehyde addition. At ca. 30 min the precipitate was dissolved by mild heating of the reaction with a heat gun, and the solution was stirred for 3 h at rt. Water (1 L) was added slowly to the reaction mixture to form cloudy yellow precipitate, and the mixture was stirred for 30 min. The cloudy orange solution (600 mL) was decanted, and the remaining solution with most of the precipitate was filtered. The solid was washed with water, dried, to give methyl 2-amino-5-tert-butylthiophene-3-carboxylate (MW=213.3) as a yellow solid (30 g, 70%) References: Patent: WO 98/52558

To a 250 mL flask containing methyl 2-amino-5-tert-butylthiophene-3-carboxylate (45 g, 210 mmol) in 1:1 mixture of MeOH/water (600 mL) was added 45% KOH (75 g g, 600 mmol), and the reaction mixture was heated to 80° C. for 5 h. The methanol evaporated as the reaction proceeded. The solvent was removed in vacuo and the volume was brought to 600 mL with water and a small amount of insoluble material was filtered. To the vigorously stirred solution was added 20% phosgene in toluene (150 mL, 1.45 eq) over a period of 5 min. A gooey solid was formed during addition of phosgene. The mixture was stirred for 1 h at rt, then filtered and washed with water. The solid was dried in vacuo to provide 6-tert-butyl-1H-thieno[2,3-d][1,3]oxazine-2,4-dione (39 g, 82%).

A solution of 6-tert-butyl-1H-thieno[2,3-d][1,3]oxazine-2,4-dione (43 gm, 190.9 mmol) in t-BuOH (635 mL) was brought to reflux (90° C.), stirring under N₂ overnight. The reaction mixture was concentrated by rotovap to remove t-BuOH and the crude product was purified by trituration/recrystallization with ca. 6:1 Hexanes/Acetone in four batches. The solid was vacuum filtered and washed sparingly with ca. 12:1 Hexanes/Acetone to give 37.28 g of white solid, pure by LC. The mother liquor is concentrated to give 11.3 g of beige solid (80% pure by LC), which was recrystallized again to give an additional 3.66 g (95% purity).

To a pressure vessel containing a DMF (120 mL) solution of 2-(tert-butoxycarbonyl)-5-tert-butylthiophene-3-carboxylic acid (11.91 gm, 39.78 mmol, 1 eq.) and 3,3-dimethylpiperazine-2-one (5.61 gm, 43.76 mmol, 1.1 eq.) was added TEA (6.11 mL, 43.83 mmol, 1.1 eq.) followed by HATU (16.60 gm, 43.66 mmol, 1.1 eq.). The vessel was sealed and the mixture stirred at 70° C. overnight.

The reaction was returned to ambient, diluted with EtOAc, and washed with sat'd. aq. NaHCO₃, H₂O, and brine. The organics were separated and dried over MgSO₄, filtered and concentrated to give an orange solid. The crude material was triturated/partially recrystallized 3× from hot Acetone/Hexanes (ca 1:2), and the solids are washed with hexane. The mother liquor is concentrated and the crystallization procedure is repeated a few times to give 10.28 gm of tert-butyl 5-tert-butyl-3-(2,2-dimethyl-3-oxopiperazine-1-carbonyl)thiophen-2-ylcarbamate (63% yield), as a white solid.

To a flask containing tert-butyl 5-tert-butyl-3-(2,2-dimethyl-3-oxopiperazine-1-carbonyl)thiophen-2-ylcarbamate (11.27 gm, 27.52 mmol) was added 50% TFA/DCM (88 mL). The reaction was stirred for 45 min at r.t. and the solvent was removed in vacuo. The residue was dissolved in EtOAc, washed 3× with 1N NaOH, water and brine. The organics were separated, dried over MgSO₄, filtered and concentrated. The product precipitated from the mother liquor on the rotovap during the concentration of the organic portion, was filtered and washed copiously with Hexanes to give to off white solid 4-(2-amino-5-tert-butylthiophene-3-carbonyl)-3,3-dimethylpiperazine-2-one.

On standing overnight, additional product was observed in the reserved aq. portion. The solid is filtered under vacuum and washed sequentially with water, and Hexanes mixed with a small amount of acetone. The combined off-white solids were dried under vacuum to give a total of 7.27 gm, 85% yield of highly pure 4-(2-amino-5-tert-butylthiophene-3-carbonyl)-3,3-dimethylpiperazine-2-one.

To a RB flask containing triphosgene (1.776 g, 0.34 equiv.) in 35 mL DCM, cooled in an ice bath, was added a solution of 4-methyl-3-(5-(morpholinomethyl)pyridin-2-yl)aniline (4.989 g, 1 equiv.) and TEA (2.45 mL, 1 equiv.) in 15 mL DCM over 3 min. An additional 5 mL DCM was used to wash over any remaining material. The ice bath was removed and the mixture was allowed to stir at room temperature for 15 minutes to give a solution of the carbamoyl chloride.

To the solution of carbamoyl chloride (1.07 equiv.) in 55 mL DCM, cooled in an ice bath, was added a mixture of ert-butyl 5-tert-butyl-3-(2,2-dimethyl-3-oxopiperazine-1-carbonyl)thiophen-2-ylcarbamate (5.092 g, 1 equiv.) in 50 mL DCM over 3 minutes. A precipitate formed. An additional 10 mL DCM was used to wash over any remaining material (7 mL/mmol DCM total). The ice bath was removed and the solution was stirred at r.t. At 3 min, TEA was added slowly dropwise. After 20.5 h, the reaction volume was reduced by half in vacuo then the mixture was filtered. The solids were washed with a minimal amount of DCM. The solids were taken up in DCM then the mixture was heated while adding MeOH until solids dissolved. The solution was quickly washed with 1M NaOH before solids reformed. The aqueous layer was extracted with DCM (×1). Upon standing, solids precipitated out of the organic layer. More MeOH and heating were applied to dissolve solids then the organic layer was dried over Na₂SO₄, filtered and solvent removed in vacuo to give 6.984 g (69%) of 1-(5-tert-butyl-3-(2,2-dimethyl-3-oxopiperazine-1-carbonyl)thiophen-2-yl)-3-(4-methyl-3-(5-(morpholinomethyl)pyridin-2-yl)phenyl)urea.

The original filtrate from the reaction was washed with 1M NaOH. Remaining product can then be removed by crystallization from DCM or chromatography as 0.5 g or more of product may remain in the filtrate.

### Synthesis of 1-(5-tert-butyl-3-(2,2,4-trimethyl-3-oxopiperazine-1-carbonyl)thiophen-2-yl)-3-(4-methyl-3-(6-(morpholinomethyl)pyridin-3-yl)phenyl)urea

### Synthesis of 4-methyl-3-(6-(morpholinomethyl)pyridin-3-yl)aniline

To a RB flask containing 5-bromopicolinaldehyde (2.024 g, 10.88 mmol) in 35 mL DCE (3.5 mL/mmol) was added morpholine (0.996 g, 11.4 mmol) followed by NaBH(OAc)₃ (93.23 g, 15.24 mmol) and 0.65 mL (11.3 mmol) acetic acid. The flask was loosely capped and the mixture was stirred at room temperature. The mixture got slightly warm. At 30 min, LC-MS showed product and reduced aldehyde. The reaction was quenched with saturated NaHCO₃. After stirring until gas evolution was greatly reduced, 1M NaOH was added to bring pH to 8-9. The two layers were separated and the aqueous layer was extracted with methylene chloride (×3). The organic layers were dried over Na2SO4, filtered and solvent was removed in vacuo. The material was purified by flash column (6.5×8.5 cm silica) using 500 mL 8:2 EtOAc:hexane, then 1400 mL 100:1 EtOAc:NH4OH to give 2.388 g (85%) of 4-((5-bromopyridin-2-yl)methyl)morpholine as an amber oil.

In a 2 L flask placed the 4-((5-bromopyridin-2-yl)methyl)morpholine (33.55 g, 130 mmol), 4-methyl-3-(4,4,5,5-tetramethyl-1,3,2-dioxaborolan-2-yl)aniline (31.37 g, 135 mmol), 2M aqueous solution of K₃PO₄ (0.135 mL, 269 mmol) and the catalyst Pd(PPh₃)₄ (7.78 g, 6.73 mmol). To this was added 450 mL of non anhydrous-stabilized 1,4-dioxane to give a yellow solution. The system was flushed/sparged with nitrogen for 5 minutes, capped with a rubber stopper with a nitrogen filled balloon. It was left to heat overnight at 95° C. in an oil bath (eventually turned dark brown and the LCMS indicated no more starting material remaining). The reaction mixture was cooled down to room temperature and filtered through a pad of celite. Water (ca. 300 mL) was added to the filtrate and the organic layer was separated. The aqueous layer was extracted twice with EtOAc. The combined organic layers were dried using Na₂SO₄, filtered and concentrated on the rotavapor until a dark oil remained. Diethyl ether was added to obtain a precipitate. The solids were collected by vacuum filtration, washed with diethyl ether several times and dried under high vacuum for 16 hours to afford 4-methyl-3-(6-(morpholinomethyl)pyridin-3-yl)aniline as a dark beige solid (29.4 g, 77%).

### Preparation of 1,3,3-trimethylpiperazin-2-one hydrochloride salt

To a 22-L, round-bottom flask equipped with overhead stirrer, temperature probe, condenser, and nitrogen inlet/outlet were charged 3,3-dimethylpiperazine-2-one (763.5 g) and THF (9.16 L, 12 vol) to form a clear solution. Boc anhydride (1.50 kg, 1.1 equiv) was added in one portion and the resulting mixture was stirred at 24° C. Precipitation occurred during the reaction time. After 24 h, the reaction mixture was cooled to 5° C. and filtered. The filter cake was washed with heptane (1.5 L) and dried under vacuum at 45° C. for 17 h to afford tert-butyl 2,2-dimethyl-3-oxopiperazine-1-carboxylate in 47% yield (634 g). The mother liquid was concentrated to approximately 2.1 L and cooled to 5° C. After filtration and drying, another crop of tert-butyl 2,2-dimethyl-3-oxopiperazine-1-carboxylate was obtained in 35% yield (470 g).

To a 5-L jacketed reactor equipped with overhead stirrer, J-KEM temperature probe, and nitrogen inlet/outlet were charged compound tert-butyl 2,2-dimethyl-3-oxopiperazine-1-carboxylate (320 g) and DMF (1.28 L, 4 vol). The mixture was cooled to −10° C. To another 5-L jacketed reactor was added 1.28 L of DMF (5 vol) and then KHMDS (350 g, 1.25 equiv) was added portionwise maintaining temperature below 10° C. The KHMS solution was cooled to −4° C. and added to the slurry of tert-butyl 2,2,4-trimethyl-3-oxopiperazine-1-carboxylate slowly maintaining temperature below 0° C. Then dimethyl sulfate (174 mL, 1.3 equiv) was added slowly maintaining temperature below 6° C. After stirring at −4° C. for 3 h, the mixture was quenched with water (3.2 L) maintaining temperature below 10° C. The product was extracted with EtOAc (4×2.78 L). The combined EtOAc layers were washed with water (2×2.38 L) and then brine (1.7 L). The organic layer was then concentrated to approximately 1 L and IPA (1.5 L) was added. The solution was concentrated to approximately 1 L again. The IPA addition (1.5 L) and concentration (to 1 L) was repeated to give compound tert-butyl 2,2,4-trimethyl-3-oxopiperazine-1-carboxylate as a solution in IPA. To this solution was added 5-6 N HCl in IPA (1 L) at ambient temperature and the mixture was heated to 45° C. After 3 h, a large quantity of precipitate was observed. The mixture was stirred at 45° C. for additional 2 h until NMR indicated no tert-butyl 2,2,4-trimethyl-3-oxopiperazine-1-carboxylate remained in the mixture. The mixture was then cooled to 30° C. and MTBE (2.5 L) was added slowly over a period of 30 min. The resulting mixture was cooled to 8° C. and filtered. The filter cake was washed with MTBE (0.5 L) and dried under vacuum at 40° C. for 15 h to afford 1,3,3-trimethylpiperazin-2-one hydrochloride salt in 82% yield (206 g).

### Preparation of 1-(5-tert-butyl-3-(2,2,4-trimethyl-3-oxopiperazine-1-carbonyl)thiophen-2-yl)-3-(4-methyl-3-(6-(morpholinomethyl)pyridin-3-yl)phenyl)urea

2-(tert-butoxycarbonylamino)-5-tert-butylthiophene-3-carboxylic acid (9.0 g, 30.1 mmol) was dissolved in 200 ml DCM and cooled to −25° C. via acetone/dry ice bath. PCl₅ (6.5 g, 31.6 mmol) was added portion-wise and stirring was continued at −25° C. until all PCl₅ dissolved (15 min). In a separate 1000 ml flask, 1,3,3-trimethylpiperazin-2-one (4.92 g, 34.6 mmol) was dissolved in 200 ml DCM and treated with DIEA (6.28 ml, 36.1 mmol) for 10 min, then cooled to −5° C. in an ice water bath. To this solution was poured portion-wise the pre-formed acid chloride described above. 10 min after addition was completed, LC-MS analysis showed reaction was complete. The reaction mixture was washed with saturated sodium bicarbonate, water, brine, then solvent was removed in vacuo. The product was crystallized from hexane/EtOAc and filtered to give tert-butyl 5-tert-butyl-3-(2,2,4-trimethyl-3-oxopiperazine-1-carbonyl)thiophen-2-ylcarbamate.

tert-Butyl 5-tert-butyl-3-(2,2,4-trimethyl-3-oxopiperazine-1-carbonyl)thiophen-2-ylcarbamate was dissolved in 200 ml of 3:2 DCM/TFA mixture and stirred for 1 hour at room temperature. The solvent was removed in vacuo and the residue was dissolved in DCM and washed with 1N NaOH. The product was crashed out from hexane/EtOAc to afford 6.40 g of 4-(2-amino-5-tert-butylthiophene-3-carbonyl)-1,3,3-trimethylpiperazin-2-one as a yellow solid. (66% yield over 2 steps).

Phosgene solution (20% in toluene, 29.6 ml, 65.9 mmol) was added to 200 ml DCM and this solution was cooled to 0° C. in an ice water bath. A solution of 4-(2-amino-5-tert-butylthiophene-3-carbonyl)-1,3,3-trimethylpiperazin-2-one (7.1 g, 21.95 mmol) was added drop-wise to the phosgene solution over 5 minutes. After the addition was completed, the flask was removed from the ice bath and stirring was continued for 20 minutes. The solvents and phosgene were evaporated under a nitrogen stream. The resulting carbamoyl chloride residue was dissolved in 200 ml DCM and added portion-wise to a solution of 4-methyl-3-(6-(morpholinomethyl)pyridin-3-yl)aniline (6.03 g, 21.29 mmol) and DIEA (4.40 ml, 25.2 mmol) in 200 ml DCM at −5° C. LC/MS analysis was taken 5 minutes after addition was completed, showing complete conversion to the desired product.

The reaction solution was washed with saturated sodium bicarbonate solution, water, brine, and solvent was removed in vacuo.

The crude oil was dissolved in 100 ml ethyl acetate and added drop-wise to a cooled solution of 8:2 hexane/ethyl acetate (500 ml combined) with vigorous stirring. After 3 hours, the solids were filtered, then suspended in minimal amount of cold ethyl acetate (100 ml) and stirred for 3 hours. The solids were filtered and dried in the vacuum oven. The process was repeated for the mother liquor and in total yielded 11.9 g of 1-(5-tert-butyl-3-(2,2,4-trimethyl-3-oxopiperazine-1-carbonyl)thiophen-2-yl)-3-(4-methyl-3-(6-(morpholinomethyl)pyridin-3-yl)phenyl)urea (86% yield) as an off white solid.

¹H NMR (400 MHz, CDCl₃) δ 9.98 (s, 1H), 8.49 (d, J=2.2 Hz, 1H), 8.25 (s, 1H), 7.61 (dd, J=7.9, 2.2 Hz, 1H), 7.41 (d, J=7.9 Hz, 1H), 7.32 (d, J=2.3 Hz, 1H), 7.24 (dd, J=8.2, 2.3 Hz, 1H), 7.15 (d, J=8.3 Hz, 1H), 6.37 (s, 1H), 3.70 (m, 8H), 3.44 (t, J=5.0 Hz, 2H), 3.00 (s, 3H), 2.52 (t, J=4.5 Hz, 4H), 2.17 (s, 3H), 1.68 (s, 6H), 1.29 (s, 9H).

### Synthesis of 1-(1-tert-butyl-3-(2,2-dimethyl-3-oxopiperazine-1-carbonyl)-1H-pyrazol-4-yl)-3-(3-(6-isopropylpyridin-3-yl)-4-methylphenyl)urea

### Preparation of 3-(6-isopropylpyridin-3-yl)-4-methylaniline

In a 2 L round-bottomed flask was mixed 2,5-dibromopyridine (54 g, 228 mmol) in toluene (600 ml) to give a colorless solution. The reaction was cooled to −78° C. and n-Butyllithium 2.5M hexanes (100 ml, 251 mmol) was added at a rate that the temperature did not exceed −70° C. The reaction was stirred for 30 minutes and then acetone (20.08 ml, 274 mmol) was added quickly. The reaction was stirred for 30 minutes and then quenched with saturated ammonium chloride. The organic layer was washed with brine, dried over sodium sulfate and solvent removed under reduced pressure. The crude residue was purified by column chromatography 20-50% ethyl acetate/heptane to give 42.5 g of 2-(5-bromopyridin-2-yl)propan-2-ol in 86% yield.

In a 1 L round-bottomed flask was mixed compound 2-(5-bromopyridin-2-yl)propan-2-ol (10 g, 46.3 mmol) and 4-methyl-3-(4,4,5,5-tetramethyl-1,3,2-dioxaborolan-2-yl)aniline (11.87 g, 50.9 mmol) in dioxane (300 ml). To this was added saturated sodium bicarbonate (150 mL). The reaction mixture was degassed via purging with a stream of nitrogen and then Pd(PPh₃)₄ (2.67 g, 2.314 mmol) was added. The mixture was heated to reflux becoming very thick then finally going into solution. The reaction was heated for 2 hours, cooled to room temperature and the solvent was removed under reduced pressure. The residue was partitioned between ethyl acetate and water. The organic layer was dried over sodium sulfate and solvent removed under reduced pressure to give 2-(5-(5-amino-2-methylphenyl)pyridin-2-yl)propan-2-ol.

The crude product from above was dissolved in dioxane (30 mL) and cooled to 0° C. Sulfuric acid was added to the solution through an addition funnel with manual stirring necessary at the beginning of addition, finally going to solution. The reaction was allowed to exotherm up to ˜30° C. and stirred for 30 minutes. Upon completion of the reaction as determined by LC/MS, the reaction was poured onto ice, extracted with ethyl acetate (2×200 mL) and the pH of the aqueous was adjusted to 9-10 by addition of 50% sodium hydroxide solution. The mixture was then extracted with ethyl acetate, the organic was washed with brine and dried over sodium sulfate. After removal of solvent the crude product was isolated by column chromatography eluting 0-100% ethyl acetate/heptane to give 4-methyl-3-(6-(prop-1-en-2-yl)pyridin-3-yl)aniline. Yield 9 g, 86% over 2 steps.

4-Methyl-3-(6-(prop-1-en-2-yl)pyridin-3-yl)aniline (9 g, 40.1 mmol) was dissolved in ethanol (100 mL) and 10% palladium on carbon (0.5 g) was added. The mixture was hydrogenated at 30 psi for 2 hours. Filtration and concentration of the product afforded clean 3-(6-isopropylpyridin-3-yl)-4-methylaniline. Yield 8 g, 88%

### Preparation of 1-(1-tert-butyl-3-(2,2-dimethyl-3-oxopiperazine-1-carbonyl)-1H-pyrazol-4-yl)-3-(3-(6-isopropylpyridin-3-yl)-4-methylphenyl)urea

To a 1 L R.B. flask containing 4-nitro-3-pyrazolecarboxylic acid (40 g, 0.255 mol), and t-BuOH (94 g, 122 mL, 1.275 mol, 5 eq.) was added conc. H₂SO₄ (25 g, 13.5 mL, 0.255 mol); and the reaction mixture was stirred at 100° C. for 2 h. After cooling to rt, the reaction mixture was diluted with EtOAc and water. Then added saturated aq NaHCO₃ solution until pH was around 3-4. The aqueous layer was extracted with excess ethyl acetate. The combined ethyl acetate part was dried over any Na₂SO₄, filtered and distilled off to give 28 g of 1-tert-butyl-4-nitro-1H-pyrazole-3-carboxylic acid as solid.

To a vial containing 1-tert-butyl-4-nitro-1H-pyrazole-3-carboxylic acid, (214 mg, 1.0 mmol, 1 eq.) was added PCl₅ (240 mg, 1.15 mmol, 1.5 eq.) in chilled toluene (2 mL). The mixture was stirred until all solids dissolved (ca. 5-10 min.). The pre-formed acid chloride solution was then added to 3,3-dimethylpiperazine-2-one (141 mg, 1.10 mmol, 1.1 eq.) and TEA (0.300 mL, 2.15 mmol, 2.15 eq) in DCM (4 mL) and stirred for 45 min. at room temperature. The reaction was quenched with NaHCO₃, and the organics extracted with EtOAc, washed with water, and brine. The aqueous portions were back-extracted and the combined organics were dried over MgSO₄, filtered and concentrated to an off-white solid, 4-(1-tert-butyl-4-nitro-1H-pyrazole-3-carbonyl)-3,3-dimethylpiperazine-2-one, 310.4 mg.

To a rb flask containing 4-(1-tert-butyl-4-nitro-1H-pyrazole-3-carbonyl)-3,3-dimethylpiperazine-2-one (303 mg, 0.937 mmol, 1 eq.) in MeOH (9 mL) was added Pd/C (105 mg, 0.1 eq.). A balloon filled with H₂ was attached and the atmosphere of the vessel purged with H₂. The contents of the flask were stirred at r.t. overnight. The

mixture was filtered through Celite®, eluting with MeOH. The solvent was removed in vacuo to give 4-(4-amino-1-tert-butyl-1H-pyrazole-3-carbonyl)-3,3-dimethylpiperazine-2-one 259.7 mg, as a purple solid.

The urea was formed from 4-(4-Amino-1-tert-butyl-1H-pyrazole-3-carbonyl)-3,3-dimethylpiperazine-2-one and 3-(6-isopropylpyridin-3-yl)-4-methylaniline to give 1-(1-tert-butyl-3-(2,2-dimethyl-3-oxopiperazine-1-carbonyl)-1H-pyrazol-4-yl)-3-(3-(6-isopropylpyridin-3-yl)-4-methylphenyl)urea.

### Synthesis of 1-(5-tert-butyl-3-(2,2,4-trimethyl-3-oxopiperazine-1-carbonyl)thiophen-2-yl)-3-(3-(6-(2-hydroxypropan-2-yl)pyridin-3-yl)-4-methylphenyl)urea

4-(2-Amino-5-tert-butylthiophene-3-carbonyl)-1,3,3-trimethylpiperazin-2-one and 2-(5-(5-amino-2-methylphenyl)pyridin-2-yl)propan-2-ol were coupled to give 1-(5-tert-butyl-3-(2,2,4-trimethyl-3-oxopiperazine-1-carbonyl)thiophen-2-yl)-3-(3-(6-(2-hydroxypropan-2-yl)pyridin-3-yl)-4-methylphenyl)urea.

H-1 NMR (400 MHz, CDCl₃): □ (ppm) 10.00 (s, 1H), 8.44 (d, J=1.6 Hz, 1H), 7.80 (s, 1H), 7.66 (dd, J=8.0, 2.1 Hz, 1H), 7.36-7.40 (m, 2H), 7.22 (dd, J=8.4, 2.3 Hz, 1H), 7.17 (d, J=8.4 Hz, 1H), 6.38 (s, 1H), 5.05 (s, 1H), 3.73 (t, J=5.0 Hz, 2H), 3.46 (t, J=5.0 Hz, 2H), 3.02 (s, 3H), 2.20 (s, 3H), 1.73 (s, 6H), 1.58 (s, 6H), 1.30 (s, 9H).

### Synthesis of 1-(5-tert-butyl-2-(2,2,4-trimethyl-3-oxopiperazine-1-carbonyl)thiophen-3-yl)-3-(4-methyl-3-(6-(morpholinomethyl)pyridin-3-yl)phenyl)urea

To a round bottom flask was added a solution of 3,3-dimethylpiperazine-2-one (3.85 gm, 30 mmol, 1 eq.) in chilled DCM (55 mL), followed by TEA (5.05 mL, 36 mmol, 1.2 eq.) and 2-chloroacetyl chloride (4.07 gm, 36 mmol, 1.2 eq.). The reaction was stirred at r.t. overnight. The volatiles were removed under reduced pressure and the residue was taken up in acetone to precipitate TEA salts. The salts were filtered off and the filtrate was treated with Hexanes to the cloud point then stripped to precipitate beige solid which was collected to give 3.60 g of 4-(2-chloroacetyl)-3,3-dimethylpiperazine-2-one.

To a flask containing 4-(2-chloroacetyl)-3,3-dimethylpiperazine-2-one, 3.60 g, 17.53 mmol, 1 eq.) in chilled 50:50 THF/DCM (62 mL) was added potassium thioacetate (2.40 gm, 21.01 mmol, 1.2 eq.) in one portion. The mixture was stirred at r.t. for 7 hr. The salts were filtered and rinsed copiously with DCM. The filtrate was concentrated to give a pale beige solid. NMR analysis indicated that the reaction wasn't complete, so the crude solid was resuspended in 60 mL 50:50 DCM/THF to which potassium thioacetate (1.4 gm, 0.7 eq.) was added. The contents of the flask were stirred for 4 hr. The salts were filtered off rinsing with DCM and concentrating the filtrate to give 3.90 g S-2-(2,2-dimethyl-3-oxopiperazine-1-yl)-2-oxoethyl ethanethiolate as a beige solid.

To a vial containing S-2-(2,2-dimethyl-3-oxopiperazine-1-yl)-2-oxoethyl ethanethiolate, (3.90 g, 15.96 mmol, 1 eq.) in MeOH (70 mL), warmed to dissolve the solids, under N₂, was added freshly prepared NaOMe (Na, 1.25 gm, 54.4 mmol, 3.4 eq. in 20 mL MeOH). The resulting solution was stirred for 30 min. at room temperature. 3-Chloro-4,4-dimethylpent-2-enenitrile (2.43 gm, 16.92 mmol, 1.06 eq.) in MeOH (10 mL) was added, and the solution was stirred for 90 min. The precipitate was filtered and washed copiously with MeOH and the filtrate was reduced in vacuo. The crude product was dissolved in EtOAc, washed with H₂O, brine, dried over MgSO₄, filtered and concentrated. The resultant solid was triturated/recrystallized from hot acetone/hexanes (four crops) to give 2.462 g of 4-(3-amino-5-tert-butylthiophene-2-carbonyl)-3,3-dimethylpiperazine-2-one as a white solid.

4-(3-Amino-5-tert-butylthiophene-2-carbonyl)-3,3-dimethylpiperazine-2-one (0.050 g, 0.16 mmol) was dissolved in 3 ml DMF and the mixture was cooled to 0° C. NaH (60%, 0.013 g, 2 eq.) was added over 15 minutes and stirring was continued for an additional 10 minutes. A solution of methyl iodide (0.025 g, 1.1 eq.) in 1 ml DMF was subsequently added at 0° C. and the mixture was gradually warmed to RT over 1 hour. After aqueous workup, the product was crashed out from hexane/EtOAc to afford 4-(3-amino-5-tert-butylthiophene-2-carbonyl)-1,3,3-trimethylpiperazin-2-one.

The urea was formed from 4-(3-Amino-5-tert-butylthiophene-2-carbonyl)-1,3,3-trimethylpiperazin-2-one and 4-methyl-3-(6-(morpholinomethyl)pyridin-3-yl)aniline, giving 1-(5-tert-butyl-2-(2,2,4-trimethyl-3-oxopiperazine-1-carbonyl)thiophen-3-yl)-3-(4-methyl-3-(6-(morpholinomethyl)pyridin-3-yl)phenyl)urea.

H-1 NMR (400 MHz, CDCl₃): □ (ppm) 9.75 (s, 1H), 8.52 (d, J=1.6 Hz, 1H), 7.74 (s, 1H), 7.63 (dd, J=7.8, 2.0 Hz, 1H), 7.42 (d, J=8.0 Hz, 1H), 7.34 (s, 1H), 7.31 (d, J=2.1 Hz, 1H), 7.28 (dd, J=8.2, 2.1 Hz, 1H), 7.20 (d, J=8.4 Hz, 1H), 3.90 (t, J=4.9 Hz, 2H), 3.74 (t, J=4.3 Hz, 4H), 3.69 (s, 2H), 3.50 (t, J=5.0 Hz, 2H), 3.01 (s, 3H), 2.53 (t, J=3.9 Hz, 4H), 2.20 (s, 3H), 1.69 (s, 6H), 1.34 (s, 9H).

### Synthesis of 1-(5-tert-butyl-2-(2,2,4-trimethyl-3-oxopiperazine-1-carbonyl)furan-3-yl)-3-(4-methyl-3-(5-(morpholinomethyl)pyridin-2-yl)phenyl)urea

A 200 mL RB was charged with PPh₃ (15 g, 56 mmol) and THF (60 mL). The solution was stirred under N2 at 0° C. and treated with DEAD ((E)-diethyl diazene-1,2-dicarboxylate, 24 g, 40%, 56 mmol). After 10 min. ethyl 2-hydroxyacetate (5.8 g, 56 mmol) was added. After 5 min. the crystalline 4,4-dimethyl-3-oxopentanenitrile (5.0 g, 40 mmol) was added all at once. The mixture was stirred overnight, the quenched with 20 mL sat. aq. NaHCO3 and THF was removed in vacuo. The residue was diluted with 40 mL water and extracted w/60 mL EtOAc, washed with 45 mL brine, dried over Na2SO4 and concentrated to give an orange residue. The residue was treated with ca 80 mL hexanes and 10 mL EtOAc. The mixture was filtered and the filtrate was concentrated to give 4.01 g of ethyl 2-(1-cyano-3,3-dimethylbut-1-en-2-yloxy)acetate. To this ester was added 100 mL of a mixture of THF:MeOH:H₂O in 6:3:1 ratio. Lithium hydroxide hydrate (1.022 g, 1.2 equivalents) was added and the reaction mixture was heated at 70° C. for 4 h, then cooled. The reaction mixture was neutralized to pH 7, and extracted with DCM. The organic phase was concentrated to give 3.5 g of 2-(1-cyano-3,3-dimethylbut-1-en-2-yloxy)acetic acid.

To a vial containing 1,3,3-trimethylpiperazin-2-one (0.10 g, 0.70 mmol) was added 0.15 mL TEA (1.5 eq.) and 2-(1-cyano-3,3-dimethylbut-1-en-2-yloxy)acetic acid (0.13 g, 0.70 mmol). A solution of 0.16 g PCl₅ (0.77 mmol) in 2 mL DCM was then added slowly. The solution was stirred at r.t. for 1 h. The reaction was quenched with MeOH and the solution was diluted with DCM and washed with 1M NaOH (×2). The aqueous layers were washed with DCM. The combined organic layers were dried over Na2SO4, filtered and solvent was removed in vacuo to give 4,4-dimethyl-3-(2-oxo-2-(2,2,4-trimethyl-3-oxopiperazine-1-yl)ethoxy)pent-2-enenitrile.

4,4-Dimethyl-3-(2-oxo-2-(2,2,4-trimethyl-3-oxopiperazine-1-yl)ethoxy)pent-2-enenitrile (0.20 g, 0.65 mmol) was dissolved in 5 mL THF and treated with 30 mg NaH (60%). The reaction mixture was heated at 60° C. for overnight. The mixture was stripped, diluted with DCM and quenched with Sat'd NaHCO3. The DCM layer was washed with brine and dried over Na2SO4, filtered through Celite and concentrated to dryness to give 0.20 g of 4-(2-amino-5-tert-butylfuran-3-carbonyl)-1,3,3-trimethylpiperazin-2-one.

The urea was formed from 4-(2-Amino-5-tert-butylfuran-3-carbonyl)-1,3,3-trimethylpiperazin-2-one and 4-methyl-3-(5-(morpholinomethyl)pyridin-2-yl)aniline giving 1-(5-tert-butyl-2-(2,2,4-trimethyl-3-oxopiperazine-1-carbonyl)furan-3-yl)-3-(4-methyl-3-(5-(morpholinomethyl)pyridin-2-yl)phenyl)urea.

H-1 NMR (400 MHz, CDCl₃): □ (ppm) 9.25 (s, 1H), 8.59 (d, J=1.6 Hz, 1H), 7.72 (d, J=7.9 Hz, 1H), 7.46 (d, J=2.1 Hz, 1H), 7.41 (d, J=8.0 Hz, 1H), 7.30 (dd, J=8.2, 2.3 Hz, 1H), 7.19 (d, J=8.4 Hz, 1H), 7.05 (s, 1H), 7.00 (s, 1H), 3.89 (t, J=5.0 Hz, 2H), 3.72 (t, J=4.1 Hz, 4H), 3.56 (s, 2H), 3.50 (t, J=4.9 Hz, 2H), 3.02 (s, 3H), 2.49 (s, 4H), 2.29 (s, 3H), 1.72 (s, 6H), 1.26 (s, 9H).

### Synthesis of 1-(5-tert-butyl-3-(2,2,4-trimethyl-3-oxopiperazine-1-carbonyl)thiophen-2-yl)-3-(4-methyl-3-(6-(morpholine-4-carbonyl)pyridin-3-yl)phenyl)urea

5-Bromopicolinic acid (1.60 g, 7.92 mmol) was suspended in 10 ml DCM and a solution of oxalyl chloride (2M, 7.92 mL, 2 equivalents) was added. The mixture was heated at 60° C. for 4 hours, then the solvents were removed in vacuo. The residue was redissolved in 10 ml THF and was added drop-wise to a solution of morpholine (0.828 g, 9.5 mmol) in DCM. The reaction mixture was diluted with water and extracted with DCM. The product was crashed out from hexane/EtOAc to afford (5-bromopyridin-2-yl)(morpholino)methanone in good yield.

Suzuki coupling of 4-methyl-3-(4,4,5,5-tetramethyl-1,3,2-dioxaborolan-2-yl)aniline and (5-bromopyridin-2-yl)(morpholino)methanone as previously described provided (5-(5-amino-2-methylphenyl)pyridin-2-yl)(morpholino)methanone in 78% yield.

The urea was formed from 4-(2-Amino-5-tert-butylthiophene-3-carbonyl)-1,3,3-trimethylpiperazin-2-one and (5-(5-amino-2-methylphenyl)pyridin-2-yl)(morpholino)methanone to give 1-(5-tert-butyl-3-(2,2,4-trimethyl-3-oxopiperazine-1-carbonyl)thiophen-2-yl)-3-(4-methyl-3-(6-(morpholine-4-carbonyl)pyridin-3-yl)phenyl)urea.

H-1 NMR (400 MHz, CDCl₃): □ (ppm) 10.04 (s, 1H), 8.52 (d, J=1.4 Hz, 1H), 7.76 (dd, J=8.0, 2.1 Hz, 1H), 7.71 (d, J=7.9 Hz, 1H), 7.61 (s, 1H), 7.48 (d, J=2.0 Hz, 1H), 7.18 (d, J=8.2 Hz, 1H), 7.13 (dd, J=8.2, 2.1 Hz, 1H), 6.41 (s, 1H), 3.81-3.85 (m, 4H), 3.73-3.75 (m, 6H), 3.47 (t, J=5.0 Hz, 2H), 3.03 (s, 3H), 2.19 (s, 3H), 1.75 (s, 6H), 1.31 (s, 9H).

### Synthesis of 5-(5-(3-(5-tert-butyl-3-(2,2,4-trimethyl-3-oxopiperazine-1-carbonyl)thiophen-2-yl)ureido)-2-methylphenyl)-N-(2-methoxyethyl)pyridine-2-carboxamide

In a similar manner to the synthesis of (5-bromopyridin-2-yl)(morpholino)methanone, 5-bromopicolinic acid was coupled with 2-methoxyethanamine to give 5-bromo-N-(2-methoxyethyl)picolinamide. Suzuki coupling with 4-methyl-3-(4,4,5,5-tetramethyl-1,3,2-dioxaborolan-2-yl)aniline gave 5-(5-amino-2-methylphenyl)-N-(2-methoxyethyl)picolinamide. The ureas was formed from this aniline and 4-(2-Amino-5-tert-butylthiophene-3-carbonyl)-1,3,3-trimethylpiperazin-2-one to provide 5-(5-(3-(5-tert-butyl-3-(2,2,4-trimethyl-3-oxopiperazine-1-carbonyl)thiophen-2-yl)ureido)-2-methylphenyl)-N-(2-methoxyethyl)pyridine-2-carboxamide.

H-1 NMR (400 MHz, CDCl₃): □ (ppm) 9.97 (s, 1H), 8.48 (d, J=1.4 Hz, 1H), 8.34-8.38 (m, 2H), 8.18 (d, J=8.0 Hz, 1H), 7.74 (dd, J=8.0, 2.1 Hz, 1H), 7.36 (dd, J=8.2, 2.3 Hz, 1H), 7.30 (d, J=2.3 Hz, 1H), 7.17 (d, J=8.4 Hz, 1H), 6.37 (s, 1H), 3.67-3.72 (m, 4H), 3.58 (t, J=5.1 Hz, 2H), 3.45 (t, J=4.9 Hz, 2H), 3.38 (s, 3H), 3.01 (s, 3H), 2.16 (s, 3H), 1.69 (s, 6H), 1.29 (s, 9H).

### Synthesis of 1-(5-tert-butyl-3-(2,2,4-trimethyl-3-oxopiperazine-1-carbonyl)thiophen-2-yl)-3-(4-methyl-3-(6-(5-methyl-1,3,4-oxadiazol-2-yl)pyridin-3-yl)phenyl)urea

A suspension of 5-bromo-2-pyridinecarboxylic acid (1.0 g, 4.95 mmol) and thionyl chloride (1.4 mL, 19.8 mmol) was heated at 70° C. for 2 hours. The reaction was cooled to room temperature and the excess thionyl chloride was removed under a stream of nitrogen. Toluene (5 mL) and ethanol (2.91 mL, 49.5 mmol) were added and heated at 80° C. for 18 hours. Excess ethanol was removed under reduced pressure. Ethyl acetate was added and the mixture was neutralized with sat. NaHCO3. The organic layer was washed with water and sat. NaCl. Extract was dried over MgSO4, filtered and solvent removed under reduced pressure to give 1.07 g of ethyl 5-bromopicolinate.

Ethyl 5-bromopicolinate (1.0 g, 4.35 mmol) was mixed with 4.3 mL hydrazine and 4 mL ethanol and heated at 90° C. for 70 minutes then the excess reagent and ethanol was removed with a stream of nitrogen. The wet solid was triturated with ether then 90:10 Ether:MeOH to give a gray/green solid which was dried on the vacuum pump. The yield of 5-bromopicolinohydrazide was 0.536 g.

5-Bromopicolinohydrazide (0.2 g, 0.926 mmol) was mixed with 0.4 mL acetic acid and 4.0 mL phosphorus oxychloride and heated at 100° C. for 1 hour then at 120° C. for 1.5 hours. Excess reagents were removed with a stream of nitrogen. The residue was dissolved in ethyl acetate and sat. NaHCO3 was added until the mixture was slightly basic. The organic layer was washed again with sat. NaHCO3, water and finally sat. NaCl. The organic layer was dried over MgSO4, filtered and solvent was removed under reduced pressure to give 0.236 g of 2-(5-bromopyridin-2-yl)-5-methyl-1,3,4-oxadiazole.

2-(5-Bromopyridin-2-yl)-5-methyl-1,3,4-oxadiazole e (0.222 g, 0.925 mmol) was mixed with 4-methyl-3-(4,4,5,5-tetramethyl-1,3,2-dioxaborolan-2-yl)aniline (0.226 g, 0.971 mmol), palladium tetrakis triphenyl phosphine (0.012 g, 0.046 mmol), 2M K3PO4 (0.957 mL), and 3.2 mL dioxane. The reaction was purged with nitrogen, sealed in a tube and put in an oil bath at 95° C. under a balloon filled with nitrogen for 18 hours. The reaction was cooled to room temperature and filtered through a plug of Celite washing with water and ethyl acetate. The layers were separated and the water layer was extracted with ethyl acetate (2×25 mL). The combined organic layers were washed with 20 mL sat. NaCl, dried with MgSO4, filtered and solvent removed under reduced pressure. The crude product was purified by column chromatography on 100 mL Silica Gel eluting with 100% ethyl acetate followed by 90:10 ethyl acetate:MeOH to give 0.147 g of 4-methyl-3-(6-(5-methyl-1,3,4-oxadiazol-2-yl)pyridin-3-yl)aniline.

Phosgene solution (0.146 mL, 0.278 mmol) in 1 mL DCM was cooled to 0° C. A solution of 4-(2-amino-5-tert-butylthiophene-3-carbonyl)-1,3,3-trimethylpiperazin-2-one (0.030 g, 0.093 mmol) in 1 mL DCM was added to the cooled phosgene solution over 5 minutes. The ice bath was removed and after 20 minutes the reaction was checked by LC/MS. The excess phosgene and solvents were removed in a stream of Nitrogen and the residue was taken up in 1 mL of DCM. 4-Methyl-3-(6-(5-methyl-1,3,4-oxadiazol-2-yl)pyridin-3-yl)aniline (0.024 g, 0.090 mmol) and DIEA (0.019 mL, 0.107 mmol) were mixed with 1 mL DCM and cooled in salt ice bath. The carbamoyl chloride solution was added dropwise to the amine solution. LC/MS of the reaction mixture 20 minutes later showed completion of the reaction. The reaction was diluted with DCM and washed with sat. NaHCO3, water and sat. NaCl. The combined organic layers were dried over MgSO4, filtered and solvent removed under reduced pressure. Purification by column chromatography on silica gel eluting with 90:10 ethyl acetate:Hex then 100% ethyl acetate gave the material contaminated with some residual aniline. This material was dissolved in DCM and two drops of Acetyl chloride were added to form the acetate of the aniline impurity. The reaction was run for 30 minutes then it was diluted with DCM and washed with sat. NaHCO3 and sat. NaCl. The organic layers were dried over MgSO4, filtered and solvent removed under reduced pressure. The solid was purified by column chromatography on silica gel eluting with 100% ethyl acetate to give 0.0168 g of 1-(5-tert-butyl-3-(2,2,4-trimethyl-3-oxopiperazine-1-carbonyl)thiophen-2-yl)-3-(4-methyl-3-(6-(5-methyl-1,3,4-oxadiazol-2-yl)pyridin-3-yl)phenyl)urea.

H-1 NMR (400 MHz, CDCl₃): □ (ppm) 10.05 (s, 1H), 8.70 (d, J=1.4 Hz, 1H), 8.24 (d, J=8.2 Hz, 1H), 7.85 (dd, J=8.2, 2.1 Hz, 1H), 7.75 (s, 1H), 7.45 (d, J=2.1 Hz, 1H), 7.30 (dd, J=8.2, 2.3 Hz, 1H), 7.21 (d, J=8.4 Hz, 1H), 6.40 (s, 1H), 3.73 (t, J=5.0 Hz, 2H), 3.46 (t, J=5.0 Hz, 2H), 3.02 (s, 3H), 2.67 (s, 3H), 2.22 (s, 3H), 1.72 (s, 6H), 1.31 (s, 9H).

### Synthesis of 1-(2-tert-butyl-4-(2,2,4-trimethyl-3-oxopiperazine-1-carbonyl)thiazol-5-yl)-3-(4-methyl-3-(6-(morpholinomethyl)pyridin-3-yl)phenyl)urea

A flask containing ethyl 2-cyano-2-(hydroxyimino)acetate (5.048 g, 35.5 mmol) and 10% Pd/C (0.942 g) in 100 mL EtOAc was placed on a Parr shaker hydrogenator. The atmosphere was purged several times then the mixture was shaken under hydrogen (50 psi) at room temperature. After 16 h, the mixture was filtered through Celite, eluting with EtOAc. Solvent was removed in vacuo to give ethyl 2-amino-2-cyanoacetate. This was combined in a vial with DIEA in 100 mL DCM and cooled under nitrogen to 0° C. Pivaloyl chloride (4.70 g, 39 mmol) was added and the solution was allowed to warm slowly to r.t. At 5 h, TLC (1:1 hexanes:EtOAc) showed no SM. The reaction solution was washed with 1M HCl, saturated NaHCO3, and brine. The organic layer was dried over Na2SO4 and filtered. The solvent was removed in vacuo. Purification by flash column on silica with 7:3 hexanes:EtOAc gave 4.229 g (56% over two steps) of ethyl 2-cyano-2-pivalamidoacetate.

Lawsesson's Reagent (1.16 g, 2.87 mmol) was added to a solution of ethyl 2-cyano-2-pivalamidoacetate (1.0 g, 4.71 mmol) in 13 mL Toluene. The mixture was heated at 70° C. under nitrogen for 20 hours then cooled to room temperature. The product was chromatographed on silica ge eluting with 30:70 EA:Hex to give 0.24 g of ethyl 5-amino-2-tert-butylthiazole-4-carboxylate.

Ethyl 5-amino-2-tert-butylthiazole-4-carboxylate (0.24 g, 1.05 mmol) was combined with 3 mL dioxane and then 50% NaOH (0.252 g, 3.15 mmol) in 0.75 mL water was added. The mixture was heated at 85° C. for 6.5 hours. The reaction was acidified to pH 5 with 1M HCl then extracted with EtOAc (3×20 mL). The extract was dried over MgSO4, filtered and solvent removed under reduced pressure to give 0.150 g of 5-amino-2-tert-butylthiazole-4-carboxylic acid.

5-Amino-2-tert-butylthiazole-4-carboxylic acid e (0.15 g, 0.749 mmol) in 2 mL THF were treated with trichloroethyl chloroformate (0.113 mL, 0.824 mmol). The mixture was heated at 80° C. for 2.5 hours. The solvent was removed in vacuo. The yellow solid was triturated with hexanes. The hexanes were removed and the solids were dried under vacuum to give 0.170 g of 2-tert-butyl-5-((2,2,2-trichloroethoxy)carbonylamino)thiazole-4-carboxylic acid.

2-tert-Butyl-5-((2,2,2-trichloroethoxy)carbonylamino)thiazole-4-carboxylic acid (0.05 g, 0.133 mmol) in 1 mL DCM was cooled to −25° C. and then add phosphorus pentachloride (0.030 g, 0.146 mmol) was added portionwise and the reaction mixture was stirred 10 until all solids dissolved. A mixture of 1,3,3-trimethylpiperazin-2-one (0.021 g, 0.146 mmol) and Hunig's base (0.028 mL, 0.160 mmol) in 1 mL DCM was stirred at room temperature for 10 minutes then cooled to 0° C. The base solution was added to the acid chloride over 2 minutes. After 5 minutes the reaction was quenched with water and sat. NaHCO3 then extracted with DCM. The extract was dried over MgSO4, filtered and solvent was removed under reduced pressure. Column chromatography on silica gel eluting with 50:50 EA:Hex provided 0.032 g of 2,2,2-trichloroethyl 2-tert-butyl-4-(2,2,4-trimethyl-3-oxopiperazine-1-carbonyl)thiazol-5-ylcarbamate.

4-Methyl-3-(6-(morpholinomethyl)pyridin-3-yl)aniline (0.019 g, 0.067 mmol) was dissolved in 0.5 mL DMF and cooled in an ice bath. Sodium hydride (0.003 g, 0.083 mmol) was added then the reaction was stirred at 0° C. for ten minutes. 2,2,2-Trichloroethyl 2-tert-butyl-4-(2,2,4-trimethyl-3-oxopiperazine-1-carbonyl)thiazol-5-ylcarbamate (0.032 g, 0.064 mmol) was mixed with 1.0 mL DMF and warmed with a heat gun to completely dissolve the material. The thiazole solution was added to the amine mixture and the reaction put in a sealed tube and heated at 90° C. for 15 minutes. The reaction was cooled then quenched with water and extracted with EtOAc (2×25 mL). The organic layer was backwashed with sat. NaHCO3 (15 mL). The extract was dried over MgSO4, filtered and solvent removed under reduced pressure. Prep-TLC on silica with 95:5 DCM:MeOH provided a product which was dissolved in a minimum amount of ethyl acetate then precipitated with hexanes three times to give 1.9 mg of 1-(2-tert-butyl-4-(2,2,4-trimethyl-3-oxopiperazine-1-carbonyl)thiazol-5-yl)-3-(4-methyl-3-(6-(morpholinomethyl)pyridin-3-yl)phenyl)urea.

H-1 NMR (400 MHz, CDCl₃): □ (ppm) 10.90 (s, 1H), 8.54 (d, J=1.8 Hz, 1H), 7.64 (dd, J=7.9, 2.2 Hz, 1H), 7.44 (d, J=8.2 Hz, 1H), 7.40 (d, J=2.1 Hz, 1H), 7.27 (dd, J=8.2, 2.2 Hz, 1H), 7.24 (d, J=8.1 Hz, 1H), 7.01 (s, 1H), 4.13 (t, J=4.8 Hz, 2H), 3.75 (t, J=4.7 Hz, 4H), 3.70 (s, 2H), 3.55 (t, J=4.8 Hz, 2H), 3.02 (s, 3H), 2.55 (t, J=4.5 Hz, 4H), 2.22 (s, 3H), 1.76 (s, 6H), 1.37 (s, 9H).

### Synthesis of 1-(5-tert-butyl-3-(2,2-dimethyl-3-oxopiperazine-1-carbonyl)thiophen-2-yl)-3-(3-(6-ethylpyridin-3-yl)-4-methylphenyl)urea

In a 2 L flask was added 2,5-dibromopyridine (60 g, 235 mmol) and 800 mL of triethylamine. The solution was degassed via purging with a stream of nitrogen through the solution for 30 minutes. The reaction was charged with trimethylsilylacetylene (36 mL, 255 mmol) followed by PdCl₂(PPh₃)₂ (3 g, 4.27 mmol) and cuprous iodide (1 g, 5.26 mmol). The reaction was stirred for 10 minutes and an exotherm began. The temperature of the reaction was not allowed to exceed 30° C. by cooling in a water bath. The thick reaction mixture was stirred for 2 hours and LC showed completion. The reaction was poured into water and extracted with ethyl acetate (2×400 mL) The combined organic layers were washed with water (3×500 mL), dried over sodium sulfate, filtered and solvent removed under reduced pressure. The residue was purified by passing through a plug of silica gel (200 g) eluting with heptane followed by 5% ethyl acetate heptane to give 5-bromo-2-((trimethylsilyl)ethynyl)pyridine. Yield 58 g, 97%

In a flask was added 5-bromo-2-((trimethylsilyl)ethynyl)pyridine (30 g, 118 mmol), 200 mL of ethanol, and solid sodium hydroxide (5 g, 125 mmol). The reaction was stirred for 2 hours, and then poured into water and the pH was adjusted to ˜6 by the addition of 1 N hydrochloric acid. The mixture was washed with diethyl ether (2×300 mL) and the combined organic layers were dried over sodium sulfate and solvent removed under reduced pressure. The product, 5-bromo-2-ethylnylpyridine, was used without further purification. Yield 20 g, 93%

5-Bromo-2-ethylnylpyridine (10 g, 54.9 mmol) was dissolved in ethanol (100 mL) and Adam's Catalyst (PtO₂, 75%, 1 g) was added. The mixture was hydrogenated at 3 psi of hydrogen, continually checking the progress of the reaction by LC and ¹H NMR between each charge of hydrogen. After ˜10 psi was consumed, the data showed completion of the reaction with <5% of reduction of the bromine. The catalyst was filtered off and the solvent was removed under reduced pressure at 20° C. to give 5-bromo-2-ethylpyridine. Yield 8.7 g, 85%.

In a 1 L round-bottomed flask was 5-bromo-2-ethylpyridine (10 g, 53.8 mmol) and 4-methyl-3-(4,4,5,5-tetramethyl-1,3,2-dioxaborolan-2-yl)aniline (13.83 g, 59.1 mmol) in Dioxane (300 ml) followed by saturated sodium bicarbonate (150 ml). The mixture was degassed by passing a stream of nitrogen through the mixture for 20 minutes. Tetrakis(triphenylphosphine) palladium(0) (3.36 g, 2.91 mmol) was added and the mixture was heated to reflux becoming very thick then finally going to solution. The reaction was heated for 2 hours, cooled to room temperature and the solvent removed under reduced pressure. The residue was partitioned between ethyl acetate and water. The organic layer was dried over sodium sulfate and solvent removed under reduced pressure. The residue was purified by column chromatography 0-100% ethyl acetate/heptane to give 5-(6-ethylpyridin-3-yl)-2-methylaniline. Yield 6.7 g, 58.8%

The urea was formed from 5-(6-Ethylpyridin-3-yl)-2-methylaniline and 4-(2-amino-5-tert-butylthiophene-3-carbonyl)-3,3-dimethylpiperazine-2-one.

### Synthesis of 1-(5-tert-butyl-3-(2,2-dimethyl-3-oxopiperazine-1-carbonyl)thiophen-2-yl)-3-(3-(5-ethylpyridin-2-yl)-4-methylphenyl)urea

In a 2 L round-bottomed flask was charged n-butyllithium 2.5M hexanes (99 ml, 248 mmol) in Et2O (1680 ml) to give a colorless solution. The reaction was cooled to −78° C. n-Butyllithium 2.5M hexanes (99 ml, 248 mmol) was added dropwise keeping the temperature below −70° C. The reaction was stirred for 1 hour and N,N-dimethylformamide (36.6 ml, 473 mmol) was added keeping the temperature below −70° C. The reaction was stirred for 1 hour and quenched with saturated ammonium chloride solution (2 L). The organic layer was washed with brine, dried over sodium sulfate and solvent removed under reduced pressure. The crude residue was purified by column chromatography eluting with 0-30% ethyl acetate to give 6-bromonicotinaldehyde. Yield 19.7 g, 44.8%

In a 2 L round-bottomed flask was added Methyltriphenylphosphonium bromide (42.9 g, 120 mmol) in THF (600 ml) and cooled to −20° C. n-Butyllithium 2.5M hexanes (48.0 ml, 120 mmol) was added dropwise keeping the temperature below 0° C. The reaction was warmed to room temperature for 20 minutes and cooled back to 0° C. A solution of 6-bromonicotinaldehyde (18.6 g, 100 mmol) in THF (40 mL) was added. The reaction was warmed to room temperature and stirred overnight. The reaction was partitioned between water and diethyl ether (1 L) and the organic layer was dried over sodium sulfate, filtered and solvent removed at room temperature under reduced pressure. The product, 2-bromo-5-vinylpyridine, was purified by bulb to bulb distillation (˜600 mTorr, 80-100° C.). Yield 17.2 g, 93%

2-Bromo-5-vinylpyridine (17.2 g, 40.1 mmol) was dissolved in ethanol (150 mL) and Adam's Catalyst (PtO2, 75%, 1.4 g) was added. The mixture was hydrogenated at 3 psi of hydrogen, continually checking the progress of the reaction by LC and ¹H NMR between each charge of hydrogen. After ˜10 psi was consumed, the data showed completion of the reaction with <5% of reduction of the bromine. The catalyst was filtered off and the solvent was removed under reduced pressure at 20° C. to give 2-bromo-5-ethylpyridine. Yield 17 g, 98%.

In a 1 L round-bottomed flask was compound 6 (10 g, 53.8 mmol) and 4-methyl-3-(4,4,5,5-tetramethyl-1,3,2-dioxaborolan-2-yl)aniline (13.83 g, 59.1 mmol) in Dioxane (300 ml) followed by saturated sodium bicarbonate (150 ml). The mixture was degassed by passing a stream of nitrogen through the mixture for 20 minutes. Tetrakis(triphenylphosphine) palladium(0) (3.36 g, 2.91 mmol) was added and the mixture was heated to reflux becoming very thick then finally going to solution. The reaction was heated for 2 hours, cooled to room temperature and the solvent removed under reduced pressure. The residue was partitioned between ethyl acetate and water. The organic layer was dried over sodium sulfate and solvent removed under reduced pressure. The residue was purified by column chromatography 0-100% ethyl acetate/heptane to give 3-(5-ethylpyridin-2-yl)-4-methylaniline. Yield 8.7 g, 77%

The urea was formed from 3-(5-ethylpyridin-2-yl)-4-methylaniline and 4-(2-amino-5-tert-butylthiophene-3-carbonyl)-3,3-dimethylpiperazine-2-one.

Protein Kinase Assay. The protein kinase activity of p38 was determined by measuring the incorporation of ³³P from γ-[³³P]ATP into the GST-ATF-2 substrate, amino acids 19-96 (Upstate, N.Y. USA). The reactions were carried out in a final volume of 50 μL of 24 mM Tris-HCl buffer, pH 7.5, containing 13 mM MgCl₂, 12% Glycerol, 2% DMSO, 2 mM DTT, 2.5 Ci of γ-[³³P]ATP (1000 Ci/mmol; 1 Ci=37 GBq) (GE Healthcare, Piscataway, N.J.), 10 uM cold ATP (GE Healthcare, Piscataway, N.J.), and 2 uM GST-ATF2. Compounds were preincubated with 2 nM p38 for 20 min at 30° C.; the reactions were initiated by the addition of GST-ATF2 and ATP and incubated for 70 min at 30° C. before being stopped by the addition of 10 μL of 600 mM phosphoric acid. The phosphorylated substrate was captured on phosphocellulose 96-well plate (Millipore MAPHNOB 10), washed with 100 mM phosphoric acid, and counted in a BeckmenCoulter LS6500 liquid scintillation counter. For this test compounds where broken down into categories, where IC50<0.10 μM is listed as A; IC50<1 μM is listed at B; and IC50<10 μM is listed at C, in the table below under the heading Act. (Activity).

Some exemplary compounds made through the methods outline above or analogous methods are listed in the table below, which also contains some NMR data and activity in the Protein Kinase Assay.

In some instances, NMR data was not available in proper form for submission herewith; in such instances, MS data has been provided.

| NAME | | |
|---|---|---|
| STRUCTURE | NMR or MS | ACT. |
| 1-(5-tert-butyl-3-(2,2-dimethyl-3- oxopiperazine-1-carbonyl)thiophen-2-yl)-3- (3-(2-methylthiazol-4-yl)phenyl)urea 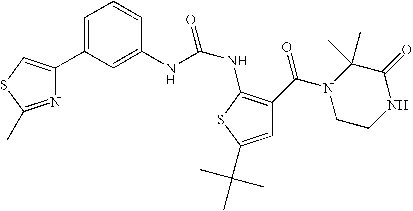 | ¹H NMR (400 MHz, acetone-d₆): δ 9.99 (s, 1H), 9.42 (s, 1H), 8.28 (t, J = 2.0 Hz, 1H), 7.68 (s, 1H), 7.63 (d, J = 7.6 Hz, 1H), 7.58-7.54 (m, 1H), 7.35 (t, J = 7.9 Hz, 1H), 7.24 (s, 1H), 6.67 (s, 1H), 3.79 (t, J = 5.0 Hz, 2H), 3.59-3.52 (m, 2H), 2.73 (s, 3H), 1.75 (s, 6H), 1.37 (s, 9H). | A |
| 1-(5-tert-butyl-3-(2,2-dimethyl-3- oxopiperazine-1-carbonyl)thiophen-2-yl)-3- (3-(pyridin-4-yl)phenyl)urea 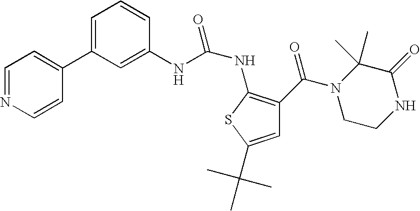 | ¹H NMR (400 MHz, CD₂Cl₂): δ 9.82 (s, 1H), 8.74 (s, 1H), 8.61 (d, J = 6.1 Hz, 2H), 7.91 (t, J = 1.9 Hz, 1H), 7.57 (d, J = 6.1 Hz, 2H), 7.52-7.48 (m, 1H), 7.43 (t, J = 7.8 Hz, 1H), 7.37-7.33 (m, 1H), 6.67 (s, 1H) 6.46 (s, 1H), 3.73 (t, J = 5.1 Hz, 2H), 3.50-3.45 (m, 2H), 1.79 (s, 6H), 1.36 (s, 9H). | A |
| 1-(5-tert-butyl-3-(2,2-dimethyl-3- oxopiperazine-1-carbonyl)thiophen-2-yl)-3- (3-(pyridin-3-yl)phenyl)urea 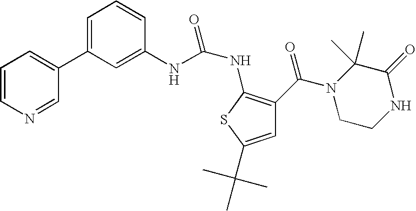 | ¹H NMR (400 MHz, (CD₃)₂SO): δ 10.01 (s, 1H), 9.73 (s, 1H), 8.76 (d, J = 1.8 Hz, 1H), 8.50 (dd, J = 4.8, 1.5 Hz, 1H), 7.95 (t, J = 11.6 Hz, 2H), 7.77 (s, 1H), 7.45- 7.24 (m, 4H), 6.49 (s, 1H), 3.53-3.48 (m, 2H), 3.28-3.24 (m, 2H), 1.62 (s, 6H), 1.24 (s, 9H). | A |
| 1-(5-tert-butyl-3-(2,2-dimethyl-3- oxopiperazine-1-carbonyl)thiophen-2-yl)-3- (4-methyl-3-(pyridin-4-yl)phenyl)urea 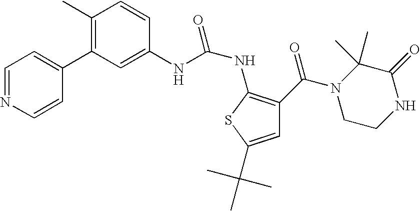 | ¹H NMR (400 MHz, acetone-d₆): δ 9.94 (s, 1H), 9.33 (s, 1H), 8.64 (d, J = 6.1 Hz, 2H), 7.57 (d, J = 2.3 Hz, 1H), 7.48 (dd, J = 8.4, 2.3 Hz, 1H), 7.36 (d, J = 6.1 Hz, 2H), 7.25 (d, J = 8.2 Hz, 1H), 7.14 (s, 1H), 6.64 (s, 1H), 3.76 (t, J = 5.1 Hz, 2H), 3.56-3.49 (m, 2H), 2.20 (s, 3H), 1.72 (s, 6H), 1.34 (s, 9H). | A |
| 1-(5-tert-butyl-3-(2,2-dimethyl-3- oxopiperazine-1-carbonyl)thiophen-2-yl)-3- (3-(oxazol-5-yl)phenyl)urea 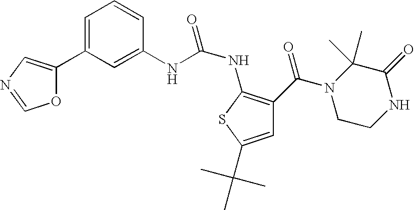 | M + H 496 | A |
| 1-(5-tert-butyl-3-(2,2-dimethyl-3- oxopiperazine-1-carbonyl)thiophen-2-yl)-3- (3-(2-fluoropyridin-4-yl)phenyl)urea 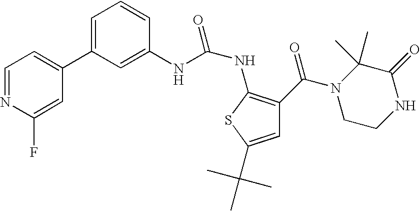 | ¹H NMR (400 MHz, acetone-d₆): δ 10.02 (s, 1H), 9.53 (s, 1H), 8.30 (d, J = 5.3 Hz, 1H), 8.13-8.10 (m, 1H), 7.65-7.58 (m, 2H), 7.48-7.44 (m, 2H), 7.34-7.31 (m, 1H), 7.16 (s, 1H), 6.66 (s, 1H), 3.77 (t, J = 5.0 Hz, 2H), 3.56-3.51 (m, 2H), 1.73 (s, 6H), 1.36 (s, 9H). | A |
| 1-(5-tert-butyl-3-(2,2-dimethyl-3- oxopiperazine-1-carbonyl)thiophen-2-yl)-3- (3-(6-(2-morpholinoethoxy)pyridin-3-yl)phenyl)urea 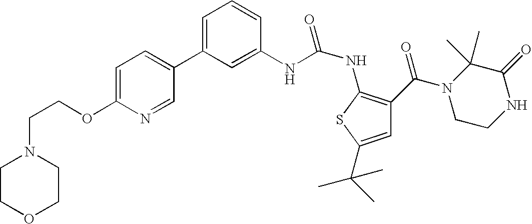 | ¹H NMR (400 MHz, CDCl₃/CD₃OD): δ 8.30 (d, J = 2.5 Hz, 1H), 7.83 (dd, J = 8.8, 2.5 Hz, 1H), 7.74-7.72 (m, 1H), 7.37- 7.28 (m, 2H), 7.16 (dt, J = 4.4, 2.4 Hz, 1H), 6.80 (d, 1H), 6.42 (s, 1H), 4.45 (t, J = 5.7 Hz, 2H), 3.71 (t, J = 4.7 Hz, 4H), 3.64-3.70 (m, 2H), 3.41 (t, J = 4.9 Hz, 2H), 2.83 (t, J = 5.6 Hz, 2H), 2.61 (t, J = 4.6 Hz, 4H), 1.76 (s, 6H), 1.31 (s, 9H). | A |
| 1-(5-tert-butyl-3-(2,2-dimethyl-3- oxopiperazine-1-carbonyl)thiophen-2-yl)-3- (4-methyl-3-(6-(2-morpholinoethoxy)pyridin-3-yl)phenyl)urea 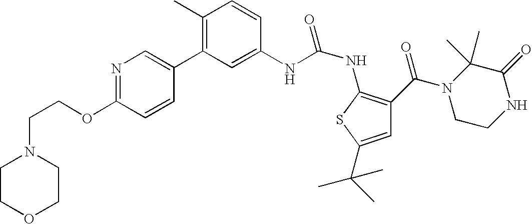 | ¹H NMR (400 MHz, CDCl₃/CD₃OD): δ 8.04 (dd, J = 2.4, 0.7 Hz, 1H), 7.60 (dd, J = 8.5, 2.4 Hz, 1H), 7.37 (d, J = 2.3 Hz, 1H), 7.28 (dd, J = 8.2, 2.3 Hz, 1H), 7.16 (d, J = 8.2 Hz, 1H), 6.81 (dd, J = 8.5, 0.7 Hz, 1H), 6.45 (s, 1H), 4.47 (t, J = 5.6 Hz, 2H), 3.72 (t, J = 4.7 Hz, 4H), 3.70-3.62 (m, 2H), 3.44-3.36 (m, 2H), 2.88 (t, J = 5.6 Hz, 2H), 2.66 (t, J = 4.5 Hz, 4H), 2.17 (s, 3H), 1.76 (s, 6H), 1.31 (s, 9H). | A |
| 1-(5-tert-butyl-3-(2,2-dimethyl-3- oxopiperazine-1-carbonyl)thiophen-2-yl)-3- (4-fluoro-3-(6-(2-morpholinoethoxy)pyridin-3-yl)phenyl)urea 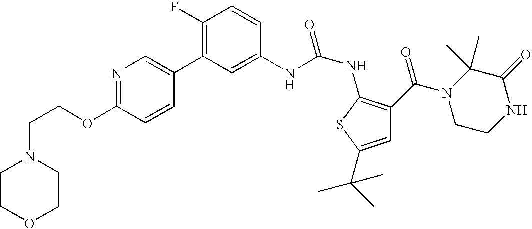 | ¹H NMR (400 MHz, CDCl₃/CD₃OD): δ 8.27 (s, 1H), 7.83-7.77 (m, 1H), 7.60 (dd, J = 6.8, 2.7 Hz, 1H), 7.35-7.29 (m, 1H), 7.05 (dd, J = 10.1, 8.9 Hz, 1H), 6.82 (d, J = 8.6 Hz, 1H), 6.42 (s, 1H), 4.52-4.42 (t, J = 5.6 Hz, 2H), 3.71 (t, J = 4.8 Hz, 4H), 3.68 (t, J = 5.0 Hz, 2H), 3.41 (t, J = 5.0 Hz, 2H), 2.83 (t, J = 5.7 Hz, 2H), 2.61 (t, J = 4.5 Hz, 4H), 1.76 (s, 6H), 1.31 (s, 9H). | A |
| 1-(5-tert-butyl-3-(2,2-dimethyl-3- oxopiperazine-1-carbonyl)thiophen-2-yl)-3- (4-chloro-3-(6-(2-morpholinoethoxy)pyridin-3-yl)phenyl)urea 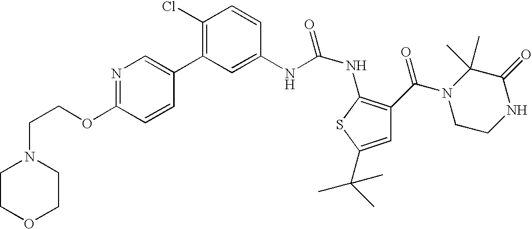 | M + H 669 | |
| 1-(5-tert-butyl-3-(2,2-dimethyl-3- oxopiperazine-1-carbonyl)thiophen-2-yl)-3- (3-(6-(3-morpholinopropoxy)pyridin-3-yl)phenyl)urea 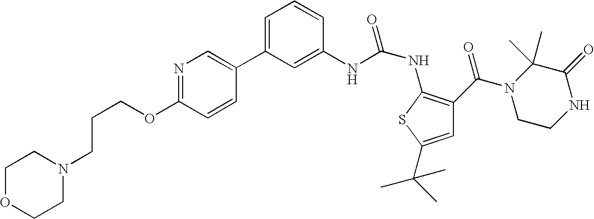 | ¹H NMR (400 MHz, CDCl₃/CD₃OD): δ 8.29 (d, J = 2.5 Hz, 1H), 7.82 (dd, J = 8.6, 8.6, 2.5 Hz, 1H), 7.71 (t, J = 1.7 Hz, 1H), 7.38-7.28 (m, 2H), 7.15 (dt, J = 4.4, 2.4 Hz, 1H), 6.77 (d, J = 8.6 Hz, 1H), 6.40 (s, 1H), 3.73-3.65 (m, 7H), 3.41 (t, J = 4.9 Hz, 2H), 3.31-3.27 (m, 1H), 2.60-2.46 (m, 7H), 2.04-1.93 (m, 2H), 1.76 (s, 6H), 1.30 (s, 9H). | A |
| 1-(5-tert-butyl-3-(2,2-dimethyl-3- oxopiperazine-1-carbonyl)thiophen-2-yl)-3- (4-methyl-3-(6-(3-morpholinopropoxy)pyridin-3-yl)phenyl)urea 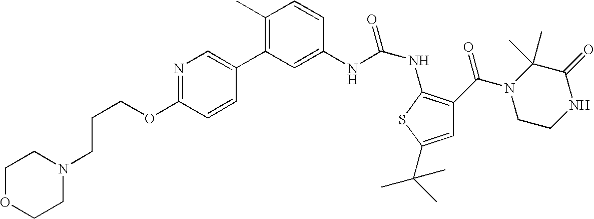 | ¹H NMR (400 MHz, CDCl₃/CD₃OD): δ 8.02 (d, J = 2.5 Hz, 1H), 7.58 (dd, J = 8.5, 2.4 Hz), 7.37 (d, J = 2.3 Hz, 1H), 7.28 (dd, J = 8.4, 2.3 Hz, 1H), 7.15 (d, J = 8.4 Hz, 1H), 6.77 (d, J = 8.4 Hz, 1H), 6.41 (s, 1H), 4.32 (t, J = 6.2 Hz, 2H), 3.72 (t, J = 4.7 Hz, 4H), 3.70-3.65 (m, 2H), 3.41 (t, J = 4.9 Hz, 2H), 2.61 (t, J = 7.7 Hz, 2H), 2.56 (s, 4H), 2.16 (s, 3H), 2.07- 1.97 (m, 2H), 1.75 (s, 6H), 1.30 (s, 9H). | A |
| 1-(5-tert-butyl-3-(2,2-dimethyl-3- oxopiperazine-1-carbonyl)thiophen-2-yl)-3- (3-morpholinophenyl)urea 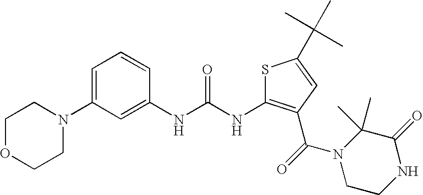 | | C |
| 1-(5-tert-butyl-3-(2,2-dimethyl-3- oxopiperazine-1-carbonyl)thiophen-2-yl)-3- (3-(6-(3-(pyridin-2-yl)propoxy)pyridin-3-yl)phenyl)urea 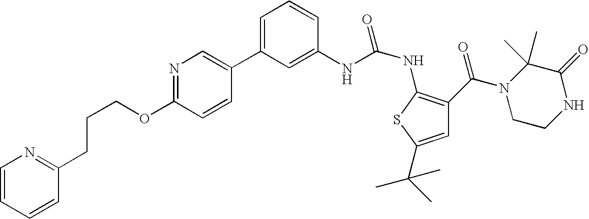 | ¹H NMR (400 MHz, CDCl₃/CD₃OD): δ 8.42-8.38 (m, 1H), 8.27 (d, J = 2.3 Hz, 1H), 7.82 (dd, J = 8.6, 2.5 Hz, 1H), 7.70 (t, J = 1.8 Hz, 1H), 7.65 (td, J = 10.8, 3.9 Hz, 1H), 7.39-7.35 (m, 1H), 7.30 (t, J = 7.8 Hz, 1H), 7.24 (d, J = 7.8 Hz, 1H), 7.18-7.13 (m, 2H), 6.75 (d, J = 8.6 Hz, 1H), 6.40 (s, 1H), 4.29 (t, J = 6.3 Hz, 2H), 3.66 (t, J = 4.9 Hz, 2H), 3.41 (t, J = 4.8 Hz, 2H), 2.95 (t, J = 7.7 Hz, 2H), 2.24- 2.13 (m, 2H), 1.56 (s, 6H), 1.30 (s, 9H). | A |
| 1-(5-tert-butyl-3-(2,2-dimethyl-3- oxopiperazine-1-carbonyl)thiophen-2-yl)-3- (3-(6-(3-(4-methylpiperazin-1-yl)propoxy)pyridin-3-yl)phenyl)urea 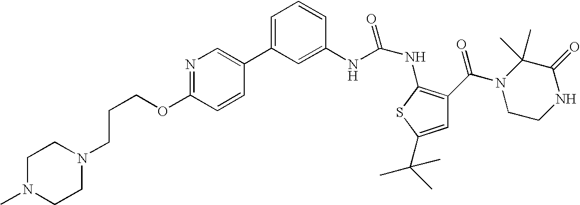 | M + H 662 | A |
| 1-(5-tert-butyl-3-(3,3-dimethyl-1-((5- methyl-1,3,4-oxadiazol-2-yl)methyl)-2- oxopiperazine-4-carbonyl)thiophen-2-yl)-3-(4-methyl-3- (pyridin-4-yl)phenyl)urea 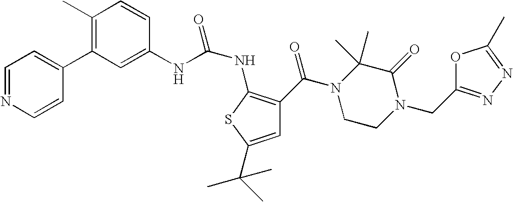 | ¹H NMR (400 MHz, CDCl₃): δ 9.86 (s, 1H), 8.15 (s, 1H), 7.25-7.20 (m, 2H), 7.13 (t, J = 8.1 Hz, 1H), 6.81 (dd, J = 8.1, 3.1 Hz, 1H), 6.60 (dd, J = 8.2, 2.0 Hz, 1H), 6.39 (s, 1H), 3.80-3.79 (m, 2H), 3.72 (t, J = 4.9 Hz, 2H), 3.52-3.43 (m, 2H), 3.20 (t, J = 5.0 Hz, 2H), 2.53 (t, J = 5.0 Hz, 3H), 2.31 (s, 3H), 1.79 (s, 6H), 1.32 (s, 9H). | A |
| 1-(5-tert-butyl-3-(2,2-dimethyl-3- oxopiperazine-1-carbonyl)thiophen-2-yl)-3- (3-(4-methylpiperazin-1-yl)phenyl)urea 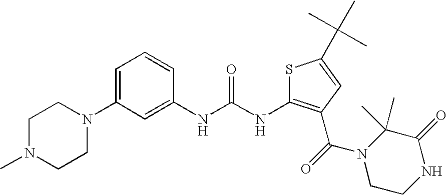 | | C |
| 1-(5-tert-butyl-3-(2,2-dimethyl-3- oxopiperazine-1-carbonyl)thiophen-2-yl)-3-(4-methyl-3- (6-(3-(4-methylpiperazin-1-yl)propoxy)pyridin-3-yl)phenyl)urea 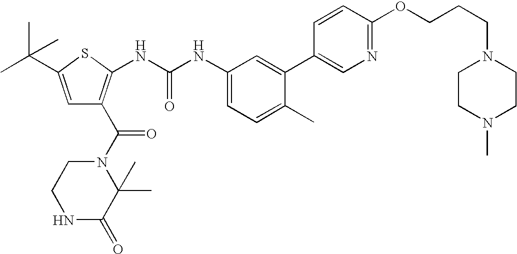 | | A |
| 1-(5-tert-butyl-3-(2,2-dimethyl-3- oxopiperazine-1-carbonyl)thiophen-2-yl)-3-(3-(6- (3-(dimethylamino)propoxy)pyridin-3-yl)phenyl)urea 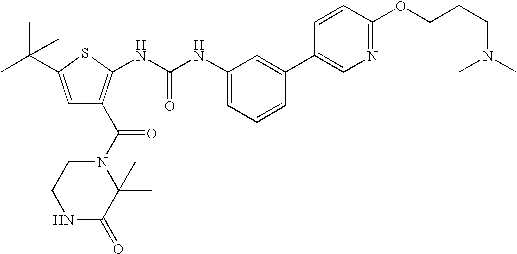 | ¹H NMR (400 MHz, CDCl₃) δ 9.95 (s, 1H), 9.09 (s, 1H), 8.31 (dd, J = 2.5 Hz, 0.6 Hz, 1H), 7.78 (dd, J = 8.6, 2.5 Hz, 1H), 7.68 (t, J = 1.8 Hz, 1H), 7.54-7.49 (m, 1H), 7.46 (s, 1H), 7.32 (t, J = 7.8 Hz, 1H), 7.15 (d, J = 7.8 Hz, 1H), 6.73 (d, J = 7.8 Hz, 1H), 6.38 (s, 1H), 4.34 (t, J = 6.1 Hz, 2H), 3.72 (t, J = 4.7 Hz, 2H), 3.48 (s, 2H), 2.80 (t, J = 8.0 Hz, 2H), 2.49 (s, 6H), 2.13-2.05 (m, 2H), 1.79 (s, 6H), 1.31 (s, 9H). | A |
| methyl 2-(4-(2-tert-butyl-5-(3-(4-methyl-3- (pyridin-4-yl)phenyl)ureido)thiophene-4- carbonyl)-3,3-dimethyl-2-oxopiperazin-1-yl)acetate 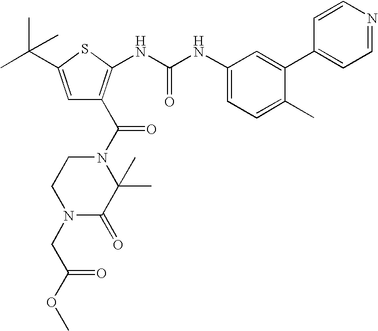 | ¹H NMR (400 MHz, CD₃OD) δ 8.57 (d, J = 6.1 Hz, 2H), 7.51 (s, 1H), 7.42 (s, J = 6.1 Hz, 3H), 7.34 (dd, J = 8.2, 2.3 Hz, 1H), 7.23 (d, J = 8.2 Hz, 1H), 4.16 (s, 2H), 3.93 (t, J = 4.9 Hz, 2H), 3.72 (s, 3H), 3.60 (t, J = 4.9 Hz, 2H), 2.22 (s, 3H), 1.75 (s, 6H), 1.37 (s, 9H). | A |
| 1-(5-tert-butyl-3-(2,2-dimethyl-3- oxopiperazine-1-carbonyl)thiophen-2-yl)-3- (3-(6-fluoropyridin-3-yl)-4-methylphenyl)urea 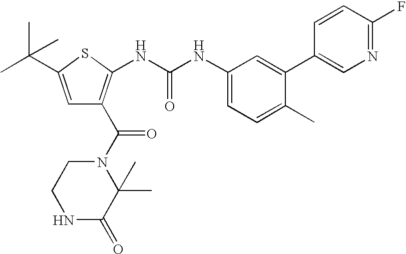 | ¹H NMR (400 MHz, CD₃OD) δ 8.15-8.12 (m, 1H), 7.90-7.83 (m, 1H), 7.40 (d, J = 2.3 Hz, 1H), 7.36-7.31 (m, 1H), 7.22 (d, J = 8.4 Hz, 1H), 7.08-7.04 (m, 1H), 6.46 (d, J = 1.2 Hz, 1H), 3.68 (t, J = 4.8 Hz, 2H), 3.41 (t, J = 4.9 Hz, 2H), 2.18 (s, 3H), 1.76 (s, 6H), 1.33 (s, 9H). | A |
| 1-(5-tert-butyl-3-(2,2-dimethyl-3- oxopiperazine-1-carbonyl)thiophen-2-yl)-3- (4-methoxy-3-(pyridin-4-yl)phenyl)urea 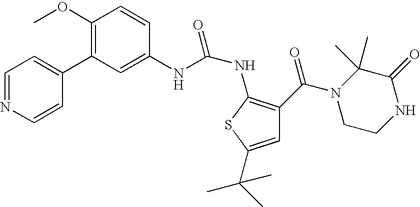 | ¹H NMR (400 MHz, CD₃OD): δ 8.52 (d, J = 6.2 Hz, 2H), 7.61 (dd, J = 4.6, 1.7 Hz, 2H), 7.54 (d, J = 2.7 Hz, 1H), 7.42 (dd, J = 8.8, 2.7 Hz, 1H), 7.09 (d, 1H), 6.56 (s, 1H), 3.81 (s, 3H), 3.74-3.67 (m, 2H), 3.45-3.40 (m, 2H), 1.78 (s, 6H), 1.35 (s, 9H). | A |
| 1-(5-tert-butyl-3-(2,2-dimethyl-3- oxopiperazine-1-carbonyl)thiophen-2-yl)-3- (4-methoxy-2-methyl-3-(pyridin-4-yl)phenyl)urea 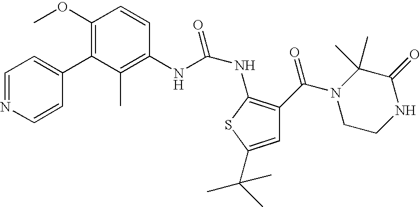 | ¹H NMR (400 MHz, CD₃Cl): δ 9.95 (s, 1H), 8.61 (s, 2H), 7.31 (t, J = 4.8 Hz, 3H), 7.18 (d, J = 2.5 Hz, 1H), 6.62-6.50 (m, 2H), 6.34 (s, 1H), 3.75 (s, 3H), 3.67 (t, J = 5.1 Hz, 2H), 3.49-3.39 (m, 2H), 2.02 (s, 3H), 1.68 (s, 6H), 1.26 (s, 9H). | A |
| 1-(5-tert-butyl-3-(2,2-dimethyl-3- oxopiperazine-1-carbonyl)thiophen-2-yl)-3- (3-(6-(2-(dimethylamino)ethoxy)pyridin-3-yl)phenyl)urea 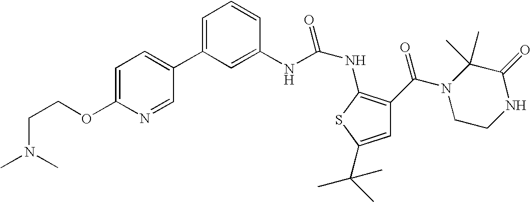 | ¹H NMR (400 MHz, CDCl₃): δ 9.94 (s, 1H), 9.00 (s, 1H), 8.31 (dd, J = 2.5, 0.6 Hz, 1H), 7.77 (dd, J = 8.6, 2.5 Hz, 1H), 7.66 (t, J = 1.8 Hz, 1H), 7.55-7.48 (m, 2H), 7.32 (t, J = 7.9 Hz, 1H), 7.18-7.12 (m, 1H), 6.77 (dd, J = 8.6, 0.6 Hz, 1H), 6.38 (s, 1H), 4.50 (t, J = 5.6 Hz, 2H), 3.72 (t, J = 4.7 Hz, 2H), 3.48 (s, 2H), 2.89 (t, J = 5.6 Hz, 2H), 2.42 (s, 6H), 1.79 (s, 6H), 1.30 (s, 9H). | A |
| 1-(5-tert-butyl-3-(2,2-dimethyl-3- oxopiperazine-1-carbonyl)thiophen-2-yl)-3- (4-methyl-3-(6-(3-(pyridin-2-yl)propoxy)pyridin-3-yl)phenyl)urea 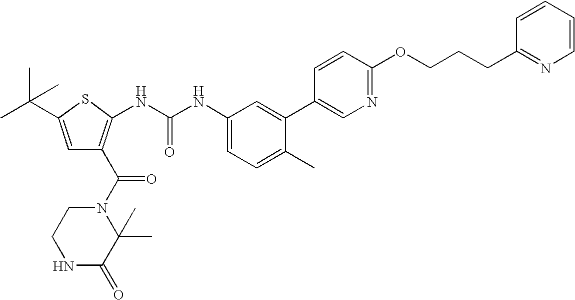 | ¹H NMR (400 MHz, CDCl₃/CD₃OD): δ 8.44-8.39 (m, 1H), 8.01 (dd, J = 2.3, 0.6 Hz, 1H), 7.68 (td, J 10.8, 3.9 Hz, 1H), 7.56 (dd, J = 8.5, 2.4 Hz, 1H), 7.35 (d, J = 2.3 Hz, 1H), 7.32-7.25 (m, 2H), 7.21-7.12 (m, 2H), 6.73 (dd, J = 8.5, 0.7 Hz, 1H), 6.39 (s, 1H), 4.30 (t, J = 6.2 Hz, 2H), 3.67 (t, 4.9 Hz, 2H), 3.40 (t, J = 4.9 Hz, 2H), 2.97 (t, J = 7.6 Hz, 2H), 2.25-2.16 (m, 2H), 2.16 (s, 3H), 1.74 (s, 6H), 1.29 (s, 9H). | A |
| 1-(5-tert-butyl-3-(2,2-dimethyl-3- oxopiperazine-1-carbonyl)thiophen-2-yl)-3- (3-(6-(2-(dimethylamino)ethoxy)pyridin-3-yl)-4-methylphenyl)urea 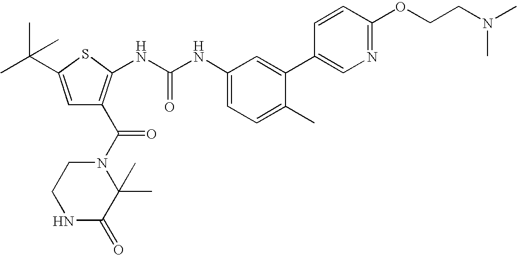 | ¹H NMR (400 MHz, CDCl₃): δ 9.79 (s, 1H), 8.71 (s, 1H), 8.02 (d, J = 2.0 Hz, 1H), 7.54-7.43 (m, 2H), 7.26 (d, J = 2.0 Hz, 1H), 7.15-7.05 (m, 2H), 6.75 (d, J = 8.4 Hz, 1H), 6.32 (s, 1H), 4.62-4.52 (m, 2H), 3.67 (d, J = 4.1 Hz, 2H), 3.43 (s, 2H), 3.06 (d, J = 4.7 Hz, 2H), 2.56 (d, J = 3.3 Hz, 6H), 2.13 (s, 3H), 1.71 (s, 6H), 1.26 (s, 9H). | A |
| 1-(5-tert-butyl-3-(2,2-dimethyl-3- oxopiperazine-1-carbonyl)thiophen-2-yl)-3- (3-(6-(3-(dimethylamino)propoxy)pyridin-3-yl)- 4-methylphenyl)urea 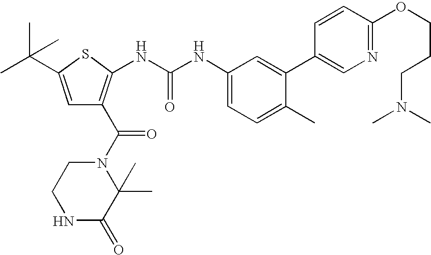 | ¹H NMR (400 MHz, CDCl₃) δ 9.90 (s, 1H), 8.56 (s, 1H), 8.07 (d, J = 2.4 Hz, 1H), 7.52 (dd, J = 2.5 Hz, 8.5, 1H), 7.41 (s, 1H), 7.38 (dd, J = 2.3 Hz, 8.3, 1H), 7.29 (d, J = 2.3 Hz, 1H), 7.15 (d, J = 8.4 Hz, 1H), 6.72 (d, J = 8.5 Hz, 1H), 6.35 (s, 1H), 4.34 (t, J = 6.5 Hz, 2H), 3.74-3.68 (m, 2H), 3.53-3.42 (m, 2H), 2.44 (t, J = 8.5 Hz, 2H), 2.23 (s, 3H), 2.18 (s, 3H), 2.00-1.88 (m, 2H), 1.75 (s, 6H), 1.28 (s, 9H). | A |
| 1-(5-tert-butyl-3-(2,2-dimethyl-3-oxopiperazine-1- carbonyl)furan-2-yl)-3-(4-methyl-3-(pyridin-4-yl)phenyl)urea 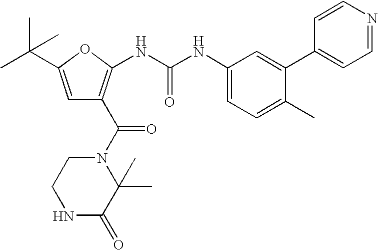 | ¹H NMR (400 MHz, CD₃OD) δ 8.54 (d, J = 5.9 Hz, 2H), 7.45-7.32 (m, 4H), 7.22 (d, J = 8.7 Hz, 1H), 6.95 (s, 1H), 3.96-3.83 (m, 2H), 3.55-3.45 (m, 2H), 3.33 (s, 3H), 3.30 (s, 3H), 1.75 (s, 6H), 1.27 (s, 9H). | A |
| 1-(5-tert-butyl-3-(2,2-dimethyl-3- oxopiperazine-1-carbonyl)thiophen-2-yl)-3- (4-methyl-3-(pyridin-3-yl)phenyl)urea 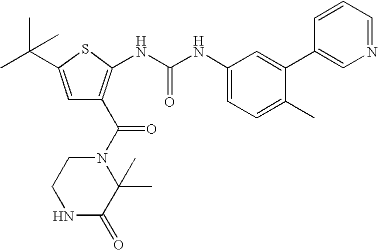 | ¹H NMR (400 MHz, (CD₃)₂SO) δ 9.97 (s, 1H), 9.74 (s, 1H), 8.60 (dd, J = 1.6, 4.8 Hz, 2H), 8.58-8.56 (m, 1H), 8.11-8.05 (m, 1H), 7.83-7.79 (m, 1H), 7.50 (dd, J = 4.8, 7.8 Hz, 2H), 7.46 (d, J = 2.3 Hz, 1H), 7.35 (dd, J = 2.3, 8.2 Hz, 1H), 7.26 (d, J = 8.3 Hz, 1H), 6.57 (s, 1H), 3.63- 3.55 (m, 2H), 3.37-3.33 (m, 2H), 2.19 (s, 3H), 1.70 (s, 6H), 1.32 (s, 9H). | A |
| 1-(5-tert-butyl-3-(2,2-dimethyl-3- oxopiperazine-1-carbonyl)thiophen-2-yl)-3- (3-(3,5-dimethylisoxazol-4-yl)-4-methylphenyl)urea 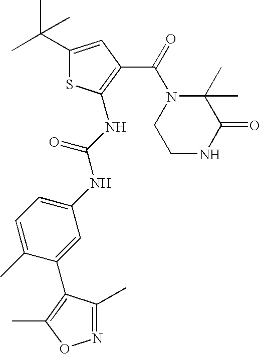 | | A |
| 1-(5-tert-butyl-3-(2,2-dimethyl-3- oxopiperazine-1-carbonyl)thiophen-2-yl)-3- (4-methyl-3-(pyridin-2-yl)phenyl)urea 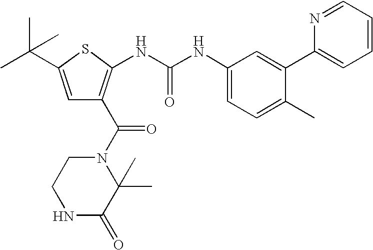 | ¹H NMR (400 MHz, CD₃OD) δ 8.61-8.50 (m, 1H), 7.87 (td, J = 1.8, 7.7 Hz, 1H), 7.46 (dd, J = 2.6, 5.1 Hz, 2H), 7.41-7.33 (m, 2H), 7.19 (d, J = 8.3, 1H), 6.53 (s, 1H), 3.72-3.60 (m, 2H), 3.46-3.34 (m, 2H), 2.19 (s, 3H), 1.76 (s, 6H), 1.31 (s, 9H). | A |
| 1-(5-tert-butyl-3-(2,2-dimethyl-3- oxopiperazine-1-carbonyl)thiophen-2-yl)-3- (4-methyl-3-(6-morpholinopyridin-3-yl)phenyl)urea 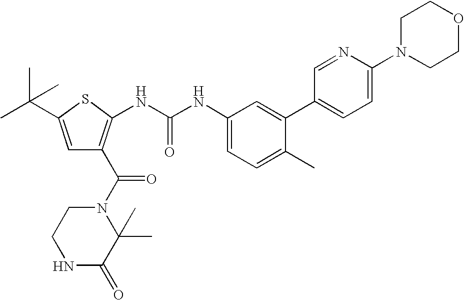 | ¹H NMR (400 MHz, CDCl₃) δ 9.97 (s, 1H), 8.21-8.08 (m, 2H), 7.49 (dd, J = 2.4, 8.7 Hz, 1H), 7.40 (dd, J = 2.3, 8.2 Hz, 1H), 7.28 (d, J = 2.3 Hz, 1H), 7.18 (d, J = 8.3 Hz, 1H), 6.94 (s, 1H), 6.66 (d, J = 8.7 Hz, 1H), 6.38 (s, 1H), 3.84 (t, J = 8.7 Hz, 4H), 3.73 (t, J = 4.0 Hz, 2H), 3.55 (t, J = 8.7 Hz, 4H), 3.52-3.46 (m, 2H), 2.22 (s, 3H), 1.75 (s, 6H), 1.31 (s, 9H). | A |
| 1-(5-tert-butyl-3-(2,2-dimethyl-3- oxopiperazine-1-carbonyl)thiophen-2-yl)-3-(4-methyl-3- (6-(2-(piperazin-1-yl)ethoxy)pyridin-3-yl)phenyl)urea 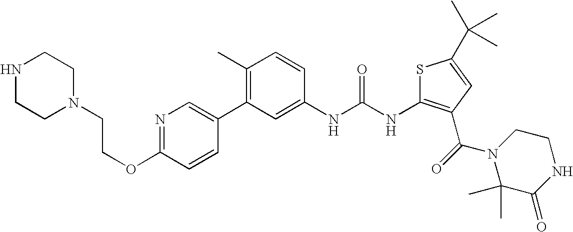 | | A |
| 1-(5-tert-butyl-3-(2,2-dimethyl-3- oxopiperazine-1-carbonyl)thiophen-2-yl)-3-(3- (6-methoxypyridin-3-yl)-4-methylphenyl)urea 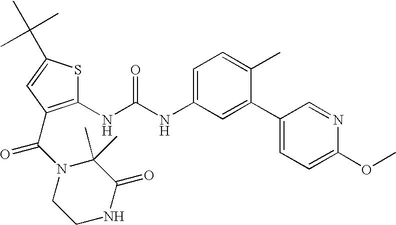 | | A |
| 1-(5-tert-butyl-3-(2,2-dimethyl-3- oxopiperazine-1-carbonyl)thiophen-2-yl)-3-(4-methyl-3- (6-(4-methylpiperazin-1-yl)pyridin-3-yl)phenyl)urea 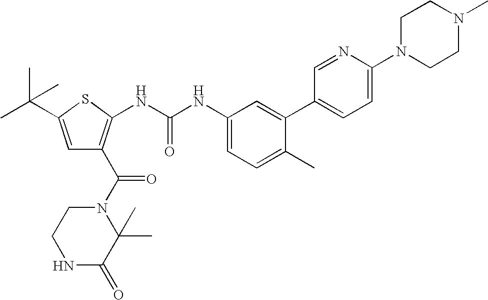 | ¹H NMR (400 MHz, CDCl₃) δ 9.92 (s, 1H), 8.27-8.11 (m, 2H), 7.47 (dd, J = 2.5, 8.7 Hz, 1H), 7.39 (dd, J = 2.3, 8.2 Hz, 1H), 7.17 (d, J = 8.4 Hz, 1H), 7.03 (s, 1H), 6.67 (d, J = 8.8 Hz, 1H), 6.37 (s, 1H), 3.74-3.70 (m, 3H), 3.60-3.55 (m, 4H), 3.53-3.43 (m, 2H), 2.54-2.51 (m, 4H), 2.33 (s, 3H), 2.22 (s, 3H), 1.76 (s, 6H), 1.30 (s, 9H). | A |
| 1-(5-tert-butyl-3-(2,2-dimethyl-3- oxopiperazine-1-carbonyl)thiophen-2-yl)-3-(3- (6-(dimethylamino)pyridin-3-yl)-4-methylphenyl)urea 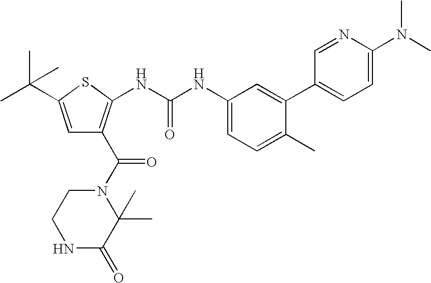 | ¹H NMR (400 MHz, CD₃OD) δ 7.99 (d, J = 2.4 Hz, 1H), 7.51-7.43 (m, 1H), 7.31 (dd, J = 2.4, 8.3 Hz, 1H), 7.27 (d, J = 2.3 Hz, 1H), 7.14 (d, J = 8.4 Hz, 1H), 6.59 (d, J = 8.6 Hz, 1H), 6.40 (s, 1H), 3.70- 3.64 (m, 2H), 3.43-3.38 (m, 2H), 3.08 (s, 6H), 2.19 (s, 3H), 1.75 (s, 6H), 1.30 (s, 9H). | A |
| 2-(5-(3-(5-tert-butyl-3-(2,2-dimethyl-3- oxopiperazine-1-carbonyl)thiophen-2-yl)ureido)-2- methylphenyl)pyridine 1-oxide 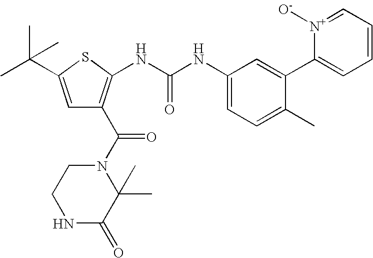 | ¹H NMR (400 MHz, (CD₃)₂SO) δ 9.88 (s, 1H), 9.65 (s, 1H), 8.30-8.23 (m, 1H), 7.98 (s, 1H), 7.42-7.29 (m, 3H), 7.25 (dd, J = 2.3, 8.3 Hz, 1H), 7.13 (d, J = 8.5 Hz, 1H), 6.48 (s, 1H), 3.52-3.48 (m, 3H), 3.29-3.19 (m, 2H), 1.98 (s, 3H), 1.61 (s, 6H), 1.22 (s, 9H). | A |
| 1-(5-tert-butyl-3-(2,2-dimethyl-3- oxopiperazine-1-carbonyl)thiophen-2-yl)-3- (4-methyl-3-(2-(trimethylsilyl)ethynyl)phenyl)urea 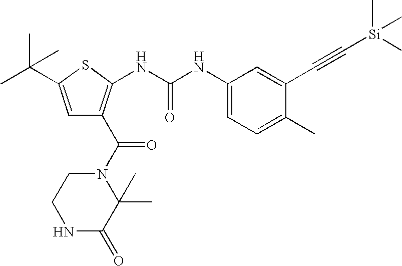 | ¹H NMR (400 MHz, (CD₃)₂SO) δ 9.92 (s, 1H), 9.73 (s, 1H), 8.07 (s, 1H), 7.79 (s, 1H), 7.20 (s, 2H), 6.57 (s, 1H), 3.65- 3.50 (m, 2H), 3.35-3.27 (m, 2H), 2.30 (s, 3H), 1.70 (s, 6H), 1.32 (s, 9H), 0.25 (s, 9H). | A |
| 1-(5-tert-butyl-3-(2,2-dimethyl-3- oxopiperazine-1-carbonyl)thiophen-2-yl)-3- (3-(6-methoxypyridin-3-yl)-4-methylphenyl)urea 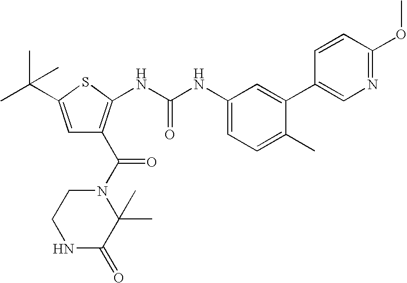 | ¹H NMR (400 MHz, CD₃OD) δ 8.56 (dd, J = 2.4, 9.0 Hz, 1H), 8.47 (d, J = 2.3 Hz, 1H), 7.92 (d, J = 8.2 Hz, 2H), 7.83-7.79 (m, 1H), 7.73 (d, J = 9.0 Hz, 1H), 7.53- 7.43 (m, 2H), 7.38 (d, J = 7.9 Hz, 2H), 6.68 (s, 1H), 4.45 (s, 3H), 3.91 (dd, J = 5.2, 10.3 Hz, 2H), 3.68-3.62 (m, 2H), 2.43 (s, 3H), 2.00 (s, 6H), 1.55 (s, 9H). | A |
| 1-(5-tert-butyl-3-(2,2-dimethyl-3- oxopiperazine-1-carbonyl)thiophen-2-yl)-3- (3-ethynyl-4-methylphenyl)urea 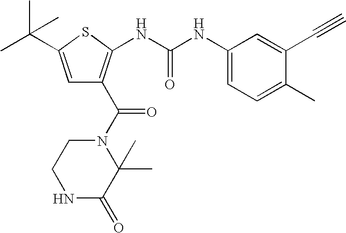 | ¹H NMR (400 MHz, (CD₃)₂SO) δ 9.88 (s, 1H), 9.69 (s, 1H), 8.04 (s, 1H), 7.61 (d, J = 2.3 Hz, 1H), 7.26 (dd, J = 2.3, 8.3 Hz, 1H), 7.16 (d, J = 8.4 Hz, 1H), 6.54 (s, 1H), 3.65-3.50 (m, 2H), 3.33-3.25 (m, 2H), 2.28 (s, 3H), 1.66 (s, 6H), 1.29 (s, 9H). | A |
| 1-(5-tert-butyl-3-(2,2-dimethyl-3- oxopiperazine-1-carbonyl)thiophen-2-yl)-3- (3-(6-(3-(dimethylamino)pyrrolidin-1-yl)pyridin-3- yl)-4-methylphenyl)urea 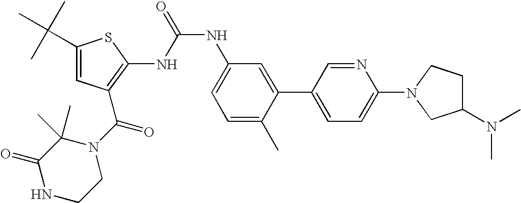 | | A |
| 1-(3-(6-(aminomethyl)pyridin-3-yl)-4- methylphenyl)-3-(5-tert-butyl-3-(2,2-dimethyl-3- oxopiperazine-1-carbonyl)thiophen-2-yl)urea 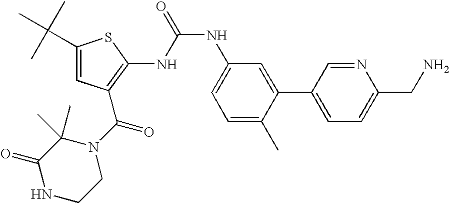 | | A |
| 1-(5-tert-butyl-3-(4-methyl-5-oxo-1,4- diazepane-1-carbonyl)thiophen-2-yl)-3-(4- methyl-3-(5-morpholinopyridin-2-yl)phenyl)urea 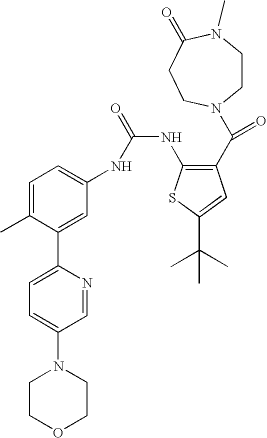 | M + H 605 | A |
| 1-(5-tert-butyl-2-(2,2-dimethyl-3- oxopiperazine-1-carbonyl)thiophen-3-yl)-3- (4-methyl-3-(5-morpholinopyridin-2-yl)phenyl)urea 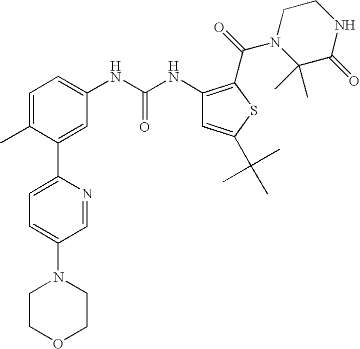 | M + H 605 | A |
| 1-(5-tert-butyl-2-(2,2-dimethyl-3- oxopiperazine-1-carbonyl)thiophen-3-yl)-3- (2-methyl-5-(5-morpholinopyridin-2-yl)phenyl)urea 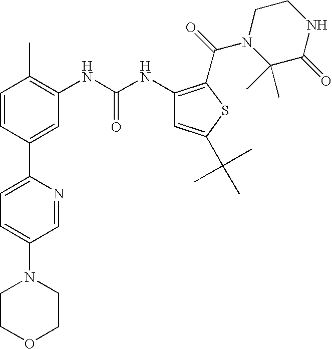 | M + H 605 | A |
| 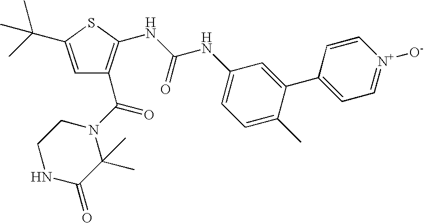 4-(5-(3-(5-tert-butyl-3-(2,2-dimethyl-3-oxopiperazine-1- carbonyl)thiophen-2-yl)ureido)-2-methylphenyl)pyridine 1-oxide | M + H 536 | A |
| 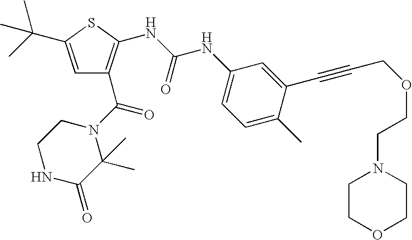 1-(5-tert-butyl-3-(2,2-dimethyl-3- oxopiperazine-1-carbonyl)thiophen-2-yl)-3-(4-methyl-3- (3-(2-morpholinoethoxy)prop-1-ynyl)phenyl)urea | M + H 610 | A |
| 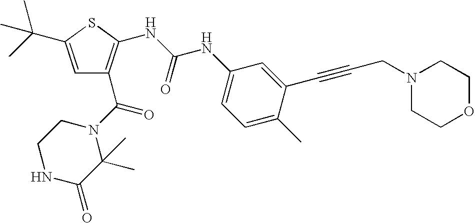 1-(5-tert-butyl-3-(2,2-dimethyl-3- oxopiperazine-1-carbonyl)thiophen-2-yl)-3-(4-methyl-3- (3-morpholinoprop-1-ynyl)phenyl)urea | M + H 566 | A |
| 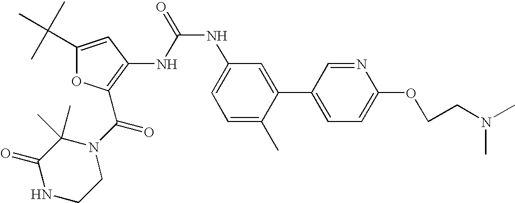 1-(5-tert-butyl-2-(2,2-dimethyl-3- oxopiperazine-1-carbonyl)furan-3-yl)-3-(3- (6-(2-(dimethylamino)ethoxy)pyridin-3-yl)-4-methylphenyl)urea | | A |
| 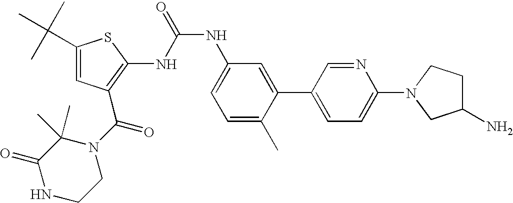 1-(3-(6-(3-aminopyrrolidin-1-yl)pyridin-3- yl)-4-methylphenyl)-3-(5-tert-butyl-3-(2,2-dimethyl-3- oxopiperazine-1-carbonyl)thiophen-2-yl)urea | M + H 604 | A |
| 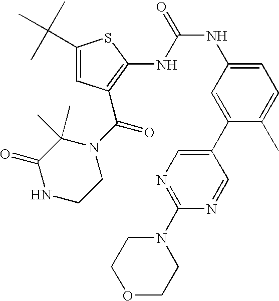 1-(5-tert-butyl-3-(2,2-dimethyl-3- oxopiperazine-1-carbonyl)thiophen-2-yl)-3-(4-methyl-3- (2-morpholinopyrimidin-5-yl)phenyl)urea | M + H 606 | A |
| 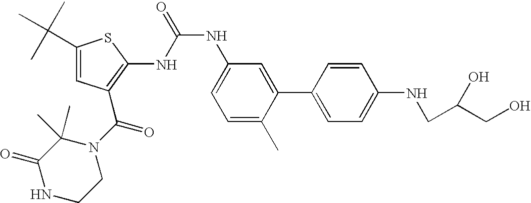 1-(5-tert-butyl-3-(2,2-dimethyl-3- oxopiperazine-1-carbonyl)thiophen-2-yl)-3-(3-(6- (2,3-dihydroxypropylamino)pyridin-3-yl)-4-methylphenyl)urea | M + H 609 | A |
| 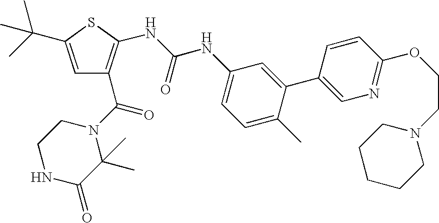 1-(5-tert-butyl-3-(2,2-dimethyl-3- oxopiperazine-1-carbonyl)thiophen-2-yl)-3-(4-methyl-3- (6-(2-(piperidin-1-yl)ethoxy)pyridin-3-yl)phenyl)urea | M + H 647 | A |
| 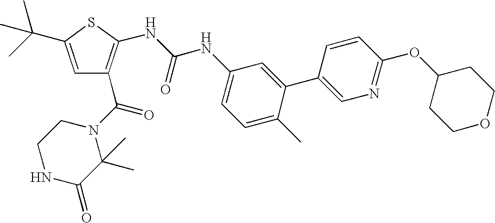 1-(5-tert-butyl-3-(2,2-dimethyl-3- oxopiperazine-1-carbonyl)thiophen-2-yl)-3-(4-methyl-3- (6-(tetrahydro-2H-pyran-4-yloxy)pyridin-3-yl)phenyl)urea | M + H 620 | A |
| 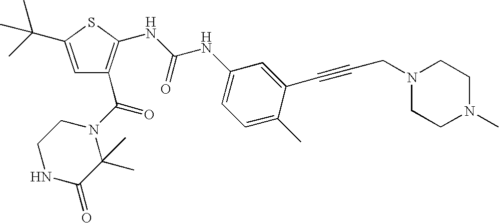 1-(5-tert-butyl-3-(2,2-dimethyl-3- oxopiperazine-1-carbonyl)thiophen-2-yl)-3-(4-methyl-3- (3-(4-methylpiperazin-1-yl)prop-1-ynyl)phenyl)urea | M + H 579 | A |
| 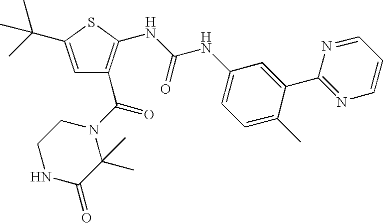 1-(5-tert-butyl-3-(2,2-dimethyl-3- oxopiperazine-1-carbonyl)thiophen-2-yl)-3-(4- methyl-3-(pyrimidin-2-yl)phenyl)urea | M + H 521 | A |
| 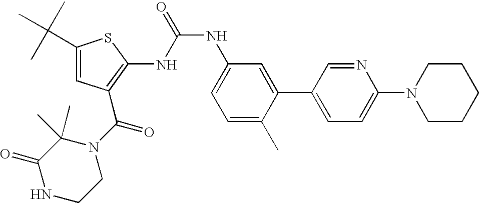 1-(5-tert-butyl-3-(2,2-dimethyl-3- oxopiperazine-1-carbonyl)thiophen-2-yl)-3-(4-methyl-3- (6-(piperidin-1-yl)pyridin-3-yl)phenyl)urea | M + H 603 | A |
| 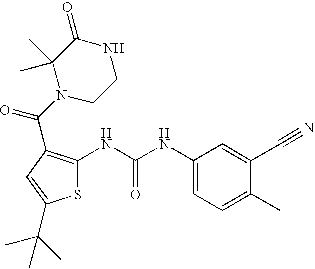 1-(5-tert-butyl-3-(2,2-dimethyl-3- oxopiperazine-1-carbonyl)thiophen-2-yl)-3- (3-cyano-4-methylphenyl)urea | M + H 468 | A |
| 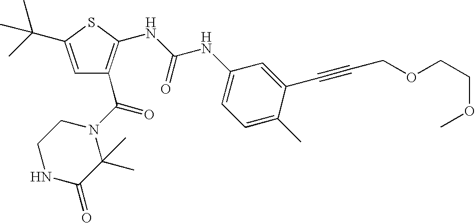 1-(5-tert-butyl-3-(2,2-dimethyl-3- oxopiperazine-1-carbonyl)thiophen-2-yl)-3-(3-(3- (2-methoxyethoxy)prop-1-ynyl)-4-methylphenyl)urea | M + H 555 | A |
| 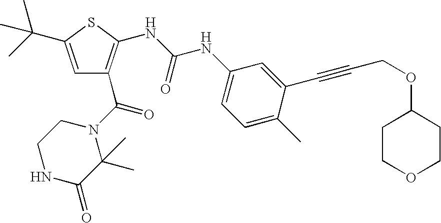 1-(5-tert-butyl-3-(2,2-dimethyl-3- oxopiperazine-1-carbonyl)thiophen-2-yl)-3-(4-methyl-3- (3-(tetrahydro-2H-pyran-4-yloxy)prop-1-ynyl)phenyl)urea | M + H 581 | A |
| 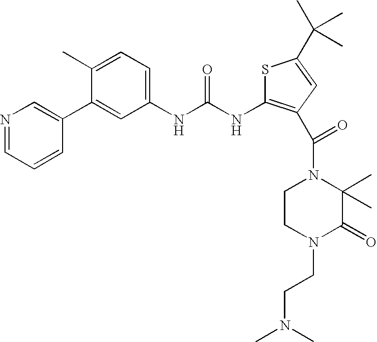 1-(5-tert-butyl-3-(4-(2-(dimethylamino)ethyl)- 2,2-dimethyl-3-oxopiperazine-1-carbonyl)thiophen- 2-yl)-3-(4-methyl-3-(pyridin-3-yl)phenyl)urea | M + H 591 | A |
| 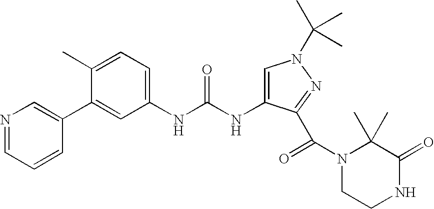 1-(1-tert-butyl-3-(2,2-dimethyl-3- oxopiperazine-1-carbonyl)-1H-pyrazol-4-yl)-3- (4-methyl-3-(pyridin-3-yl)phenyl)urea | M + H 504 | A |
| 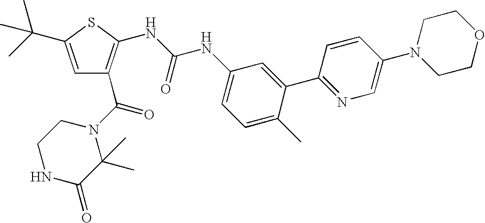 1-(5-tert-butyl-3-(2,2-dimethyl-3- oxopiperazine-1-carbonyl)thiophen-2-yl)-3-(4- methyl-3-(5-morpholinopyridin-2-yl)phenyl)urea | M + H 605 | A |
| 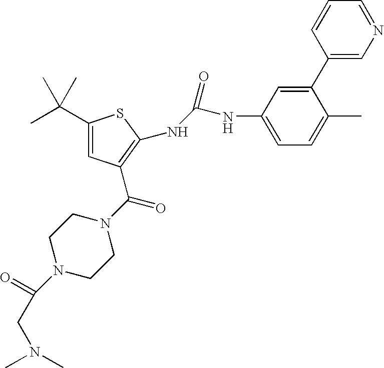 1-(5-tert-butyl-3-(4-(2-(dimethylamino)ethanoyl)piperazine- 1-carbonyl)thiophen-2-yl)-3-(4-methyl-3-(pyridin-3-yl)phenyl)urea | M + H 563 | B |
| 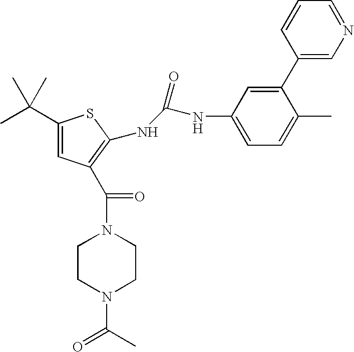 1-(5-tert-butyl-3-(4-ethanoylpiperazine-1-carbonyl)thiophen- 2-yl)-3-(4-methyl-3-(pyridin-3-yl)phenyl)urea | M + H 520 | A |
| 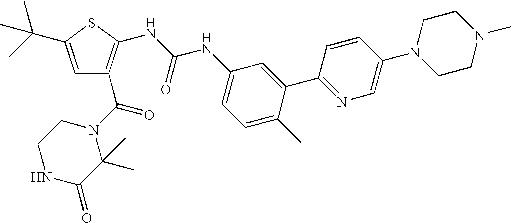 1-(5-tert-butyl-3-(2,2-dimethyl-3- oxopiperazine-1-carbonyl)thiophen-2-yl)-3-(4- methyl-3-(5-(4-methylpiperazin-1-yl)pyridin-2-yl)phenyl)urea | M + H 618 | A |
| 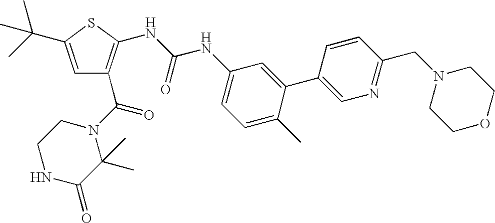 1-(5-tert-butyl-3-(2,2-dimethyl-3- oxopiperazine-1-carbonyl)thiophen-2-yl)-3-(4- methyl-3-(6-(morpholinomethyl)pyridin-3-yl)phenyl)urea | M + H 619 | A |
| 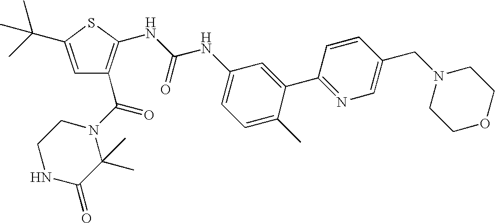 1-(5-tert-butyl-3-(2,2-dimethyl-3- oxopiperazine-1-carbonyl)thiophen-2-yl)-3-(4- methyl-3-(5-(morpholinomethyl)pyridin-2-yl)phenyl)urea | M + H 619 | A |
| 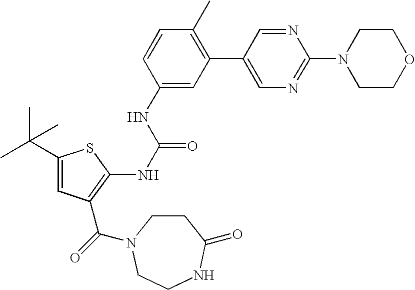 1-(5-tert-butyl-3-(5-oxo-1,4-diazepane-1- carbonyl)thiophen-2-yl)-3-(4-methyl-3-(2- morpholinopyrimidin-5-yl)phenyl)urea | M + H 592 | A |
| 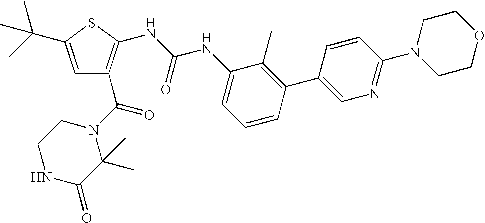 1-(5-tert-butyl-3-(2,2-dimethyl-3- oxopiperazine-1-carbonyl)thiophen-2-yl)-3- (2-methyl-3-(6-morpholinopyridin-3-yl)phenyl)urea | M + H 605 | A |
| 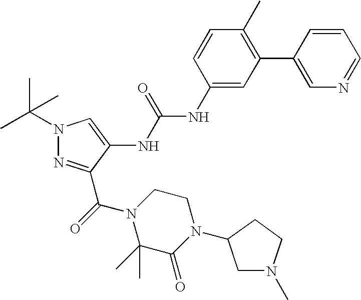 1-(1-tert-butyl-3-(2,2-dimethyl-4-(1- methylpyrrolidin-3-yl)-3-oxopiperazine-1-carbonyl)- 1H-pyrazol-4-yl)-3-(4-methyl-3-(pyridin-3-yl)phenyl)urea | M + H 587 | B |
| 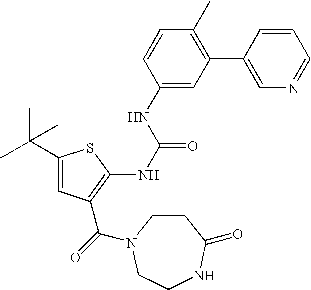 1-(5-tert-butyl-3-(5-oxo-1,4-diazepane-1- carbonyl)thiophen-2-yl)-3-(4-methyl-3- (pyridin-3-yl)phenyl)urea | M + H 506 | A |
| 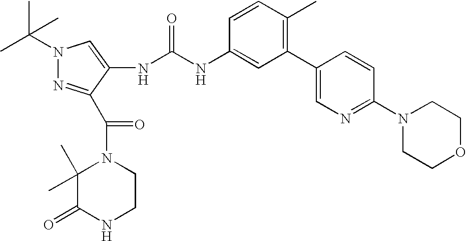 1-(1-tert-butyl-3-(2,2-dimethyl-3- oxopiperazine-1-carbonyl)-1H-pyrazol-4-yl)-3-(4- methyl-3-(6-morpholinopyridin-3-yl)phenyl)urea | M + H 589 | A |
| 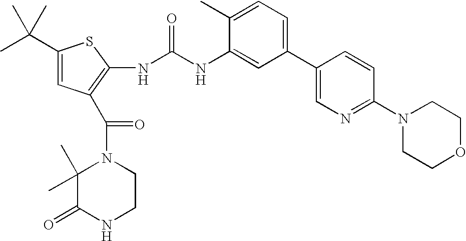 1-(5-tert-butyl-3-(2,2-dimethyl-3- oxopiperazine-1-carbonyl)thiophen-2-yl)-3- (2-methyl-5-(6-morpholinopyridin-3-yl)phenyl)urea | M + H 605 | A |
| 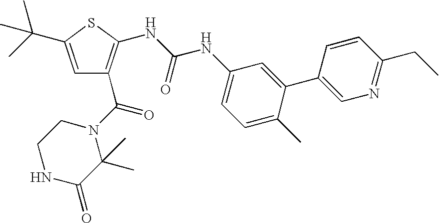 1-(5-tert-butyl-3-(2,2-dimethyl-3- oxopiperazine-1-carbonyl)thiophen-2-yl)-3- (3-(6-ethylpyridin-3-yl)-4-methylphenyl)urea | M + H 548 | A |
| 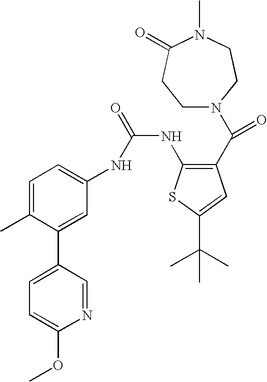 1-(5-tert-butyl-3-(4-methyl-5-oxo-1,4- diazepane-1-carbonyl)thiophen-2-yl)-3-(3- (6-methoxypyridin-3-yl)-4-methylphenyl)urea | M + H 550 | A |
| 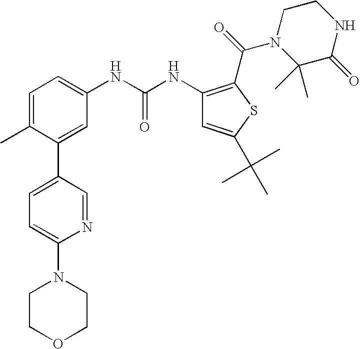 1-(5-tert-butyl-2-(2,2-dimethyl-3- oxopiperazine-1-carbonyl)thiophen-3-yl)-3- (4-methyl-3-(6-morpholinopyridin-3-yl)phenyl)urea | M + H 605 | A |
| 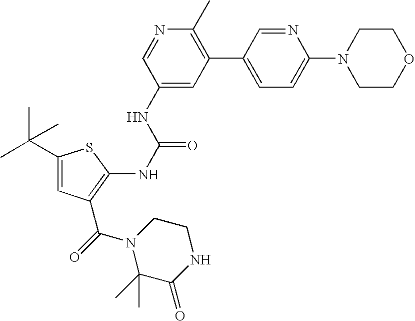 1-(5-tert-butyl-3-(2,2-dimethyl-3- oxopiperazine-1-carbonyl)thiophen-2-yl)-3- (2-methyl-6′-morpholino-3,3′-bipyridin-5-yl)urea | M + H 606 | A |
| 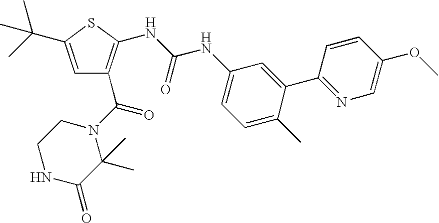 1-(5-tert-butyl-3-(2,2-dimethyl-3- oxopiperazine-1-carbonyl)thiophen-2-yl)-3- (3-(5-methoxypyridin-2-yl)-4-methylphenyl)urea | M + H 550 | A |
| 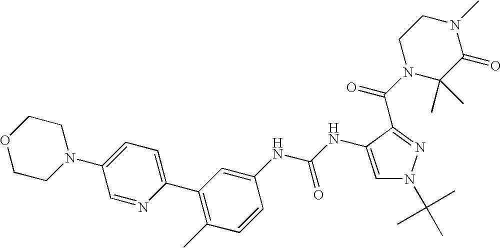 1-(1-tert-butyl-3-(2,2,4-trimethyl-3- oxopiperazine-1-carbonyl)-1H-pyrazol-4-yl)-3- (4-methyl-3-(5-morpholinopyridin-2-yl)phenyl)urea | M + H 603 | A |
| 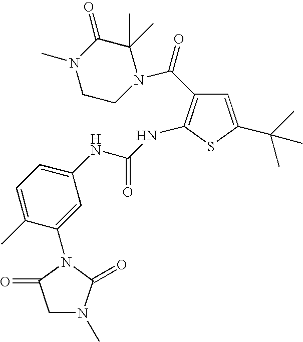 1-(5-tert-butyl-3-(2,2,4-trimethyl-3- oxopiperazine-1-carbonyl)thiophen-2-yl)-3-(4- methyl-3-(3-methyl-2,5-dioxoimidazolidin-1-yl)phenyl)urea | M + H 569 | A |
| 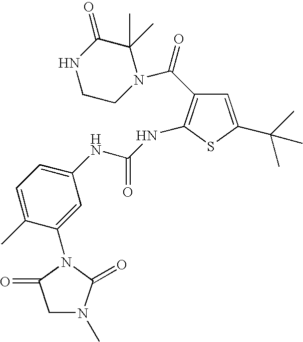 1-(5-tert-butyl-3-(2,2-dimethyl-3- oxopiperazine-1-carbonyl)thiophen-2-yl)-3-(4- methyl-3-(3-methyl-2,5-dioxoimidazolidin-1-yl)phenyl)urea | M + H 555 | A |
| 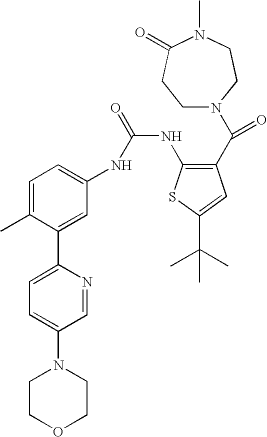 1-(5-tert-butyl-3-(4-methyl-5-oxo-1,4-diazepane- 1-carbonyl)thiophen-2-yl)-3-(4-methyl-3- (5-morpholinopyridin-2-yl)phenyl)urea | | A |
| 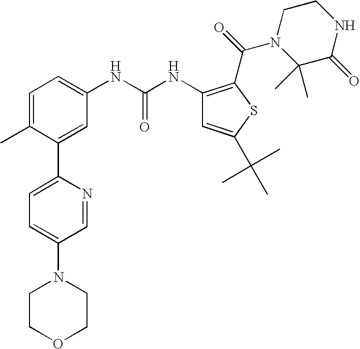 1-(5-tert-butyl-2-(2,2-dimethyl-3- oxopiperazine-1-carbonyl)thiophen-3-yl)-3-(4- methyl-3-(5-morpholinopyridin-2-yl)phenyl)urea | | A |
| 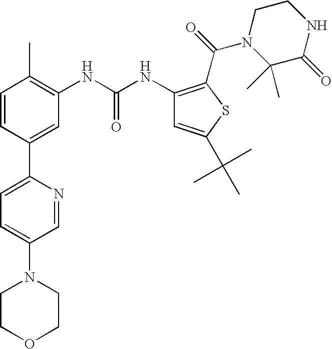 1-(5-tert-butyl-2-(2,2-dimethyl-3- oxopiperazine-1-carbonyl)thiophen-3-yl)-3-(2- methyl-5-(5-morpholinopyridin-2-yl)phenyl)urea | | A |
| 1-(5-tert-butyl-3-(2,2,4-trimethyl-3- oxopiperazine-1-carbonyl)thiophen-2-yl)-3-(3- (5-ethylpyridin-2-yl)-4-methylphenyl)urea 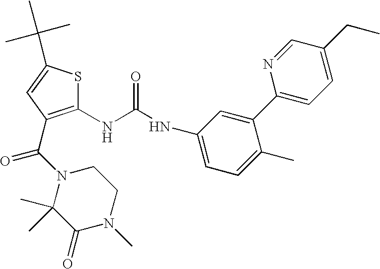 | M + H 562 | A |
| 1-(5-tert-butyl-3-(2,2,4-trimethyl-3- oxopiperazine-1-carbonyl)thiophen-2-yl)-3-(3- (6-ethylpyridin-3-yl)-4-methylphenyl)urea 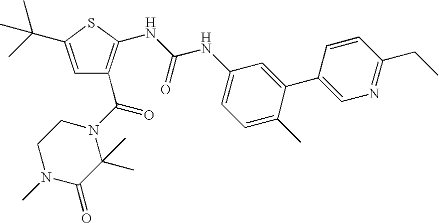 | M + H 562 | A |
| 1-(5-tert-butyl-3-(2,2-dimethyl-3- oxopiperazine-1-carbonyl)thiophen-2-yl)-3- (3-(5-ethylpyridin-2-yl)-4-methylphenyl)urea 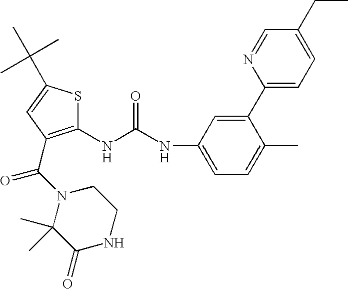 | M + H 548 | A |
| 1-(5-tert-butyl-3-(2,2,4-trimethyl-3- oxopiperazine-1-carbonyl)thiophen-2-yl)-3- (3-(6-methoxypyridin-3-yl)-4-methylphenyl)urea 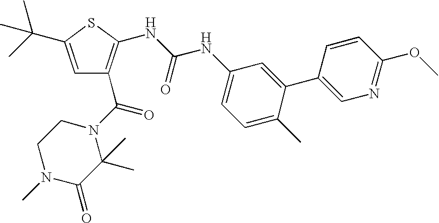 | M + H 564 | A |
| 1-(5-tert-butyl-2-(2,2-dimethyl-3- oxopiperazine-1-carbonyl)thiophen-3-yl)-3- (3-methyl-5-(5-morpholinopyridin-2-yl)phenyl)urea 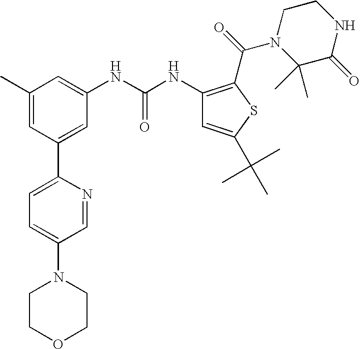 | M + H 605 | A |
| 1-(5-tert-butyl-3-(2,2-dimethyl-3- oxopiperazine-1-carbonyl)thiophen-2-yl)-3- (3-(6-ethynylpyridin-3-yl)-4-methylphenyl)urea 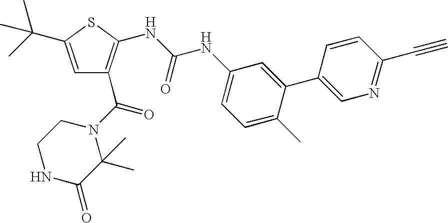 | M + H 544 | A |
| 1-(5-tert-butyl-2-(2,2,4-trimethyl-3- oxopiperazine-1-carbonyl)thiophen-3-yl)-3- (3-(6-methoxypyridin-3-yl)-4-methylphenyl)urea 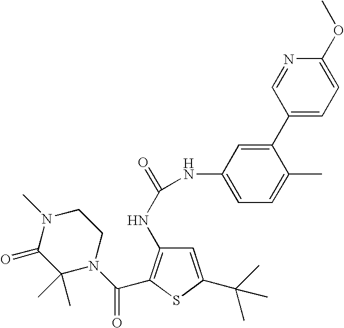 | M + H 564 | A |
| 1-(5-tert-butyl-2-(2,2,4-trimethyl-3- oxopiperazine-1-carbonyl)thiophen-3-yl)-3- (3-(6-ethylpyridin-3-yl)-4-methylphenyl)urea 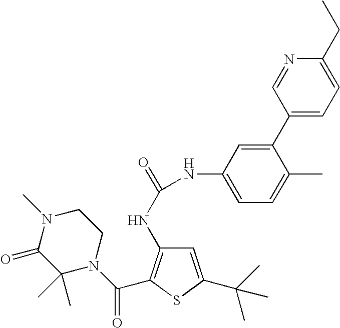 | M + H 562 | A |
| 1-(5-tert-butyl-2-(2,2,4-trimethyl-3- oxopiperazine-1-carbonyl)thiophen-3-yl)-3- (4-methyl-3-(6-morpholinopyridin-3-yl)phenyl)urea 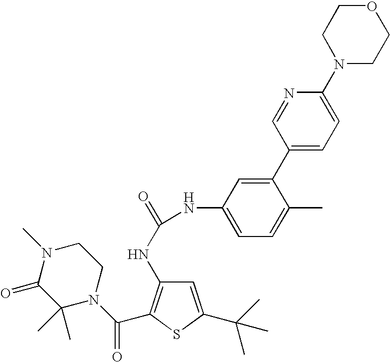 | M + H 619 | A |
| 1-(5-tert-butyl-3-(2,2,4-trimethyl-3- oxopiperazine-1-carbonyl)thiophen-2-yl)-3- (3-(6-ethoxypyridin-3-yl)-4-methylphenyl)urea 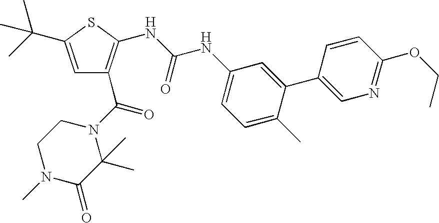 | M + H 578 | A |
| 1-(1-tert-butyl-3-(2,2,4-trimethyl-3- oxopiperazine-1-carbonyl)-1H-pyrazol-4-yl)-3- (3-(6-ethoxypyridin-3-yl)-4-methylphenyl)urea 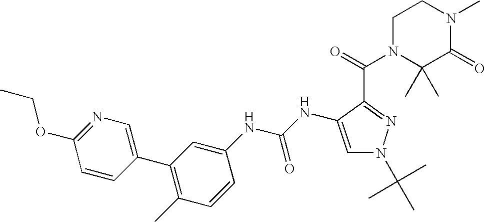 | M + H 562 | A |
| 1-(1-tert-butyl-3-(2,2,4-trimethyl-3- oxopiperazine-1-carbonyl)-1H-pyrazol-4-yl)-3- (3-(6-ethylpyridin-3-yl)-4-methylphenyl)urea 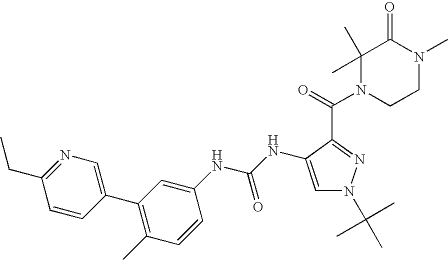 | M + H 546 | A |
| 1-(5-tert-butyl-3-(4-(2-hydroxyethyl)-2,2- dimethyl-3-oxopiperazine-1-carbonyl)thiophen- 2-yl)-3-(3-(6-methoxypyridin-3-yl)-4-methylphenyl)urea 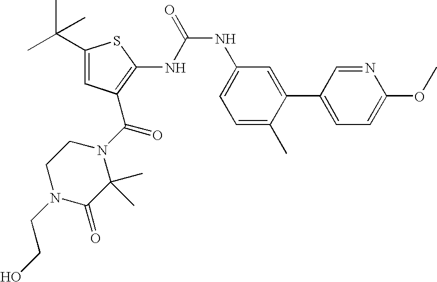 | M + H 594 | A |
| 1-(5-tert-butyl-3-(2,2-dimethyl-3- oxopiperazine-1-carbonyl)thiophen-2-yl)-3- (5-(6-methoxypyridin-3-yl)-2-methylphenyl)urea 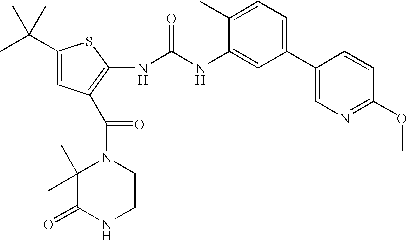 | M + H 550 | A |
| 1-(5-tert-butyl-3-(2,2-dimethyl-3- oxopiperazine-1-carbonyl)thiophen-2-yl)-3- (3-(6-methoxypyridin-3-yl)-5-methylphenyl)urea 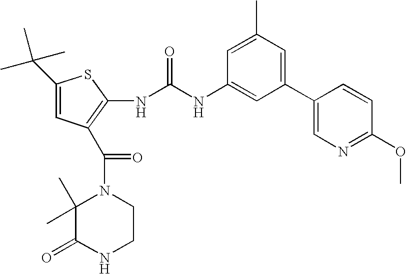 | M + H 550 | A |
| 1-(5-tert-butyl-3-(2,2,4-trimethyl-3- oxopiperazine-1-carbonyl)thiophen-2-yl)-3- (3-(6-isopropoxypyridin-3-yl)-4-methylphenyl)urea 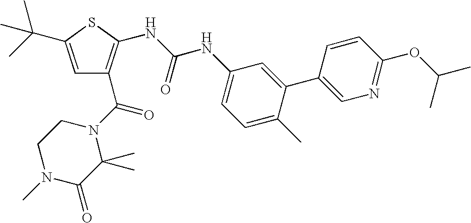 | M + H 592 | A |
| 5-(5-(3-(5-tert-butyl-3-(2,2-dimethyl-3- oxopiperazine-1-carbonyl)thiophen-2-yl)ureido)- 2-methylphenyl)-N-ethylpyridine-2-carboxamide 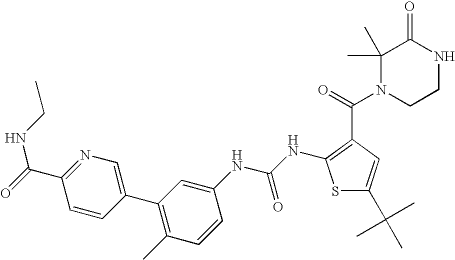 | M + H 591 | A |
| 1-(5-tert-butyl-2-(2,2-dimethyl-3- oxopiperazine-1-carbonyl)thiophen-3-yl)-3- (3-(6-methoxypyridin-3-yl)-4-methylphenyl)urea 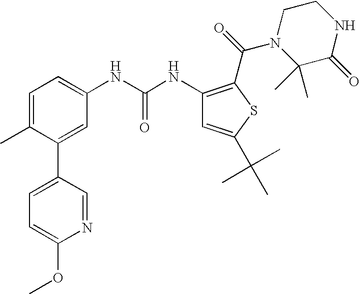 | M + H 550 | A |
| 1-(5-tert-butyl-3-(4-(2- (dimethylamino)ethyl)-2,2-dimethyl-3- oxopiperazine-1-carbonyl)thiophen-2-yl)-3- (3-(6-ethylpyridin-3-yl)-4-methylphenyl)urea 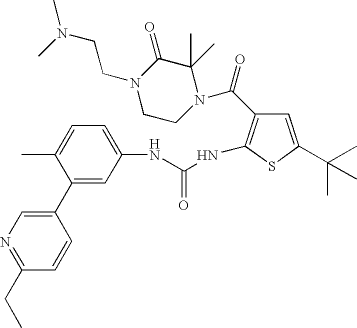 | M + H 619 | B |
| 1-(5-tert-butyl-3-(2,2-dimethyl-3- oxopiperazine-1-carbonyl)thiophen-2-yl)-3- (3-(6-(isobutylamino)pyridin-3-yl)-4-methylphenyl)urea 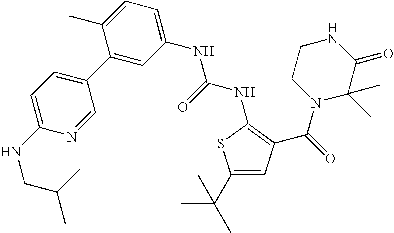 | M + H 591 | A |
| 1-(5-tert-butyl-2-(2,2,4-trimethyl-3- oxopiperazine-1-carbonyl)thiophen-3-yl)-3- (3-fluoro-5-(6-methoxypyridin-3-yl)-4-methylphenyl)urea 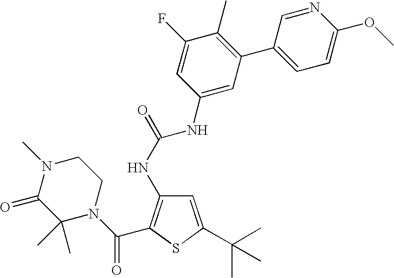 | M + H 582 | A |
| 1-(5-tert-butyl-3-(2,2,4-trimethyl-3- oxopiperazine-1-carbonyl)thiophen-2-yl)-3- (3-fluoro-5-(6-methoxypyridin-3-yl)-4-methylphenyl)urea 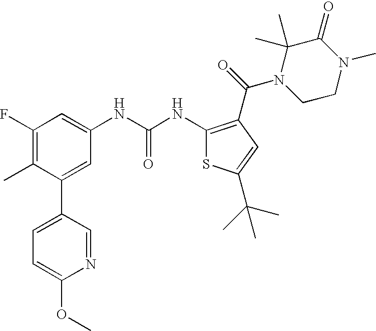 | M + H 582 | A |
| 1-(5-tert-butyl-3-(2,2-dimethyl-3- oxopiperazine-1-carbonyl)thiophen-2-yl)-3- (3-fluoro-5-(6-methoxypyridin-3-yl)-4-methylphenyl)urea 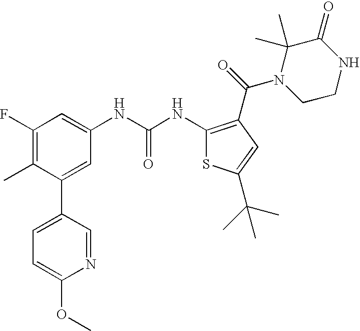 | M + H 568 | A |
| 1-(5-tert-butyl-3-(2,2-dimethyl-3- oxopiperazine-1-carbonyl)thiophen-2-yl)-3- (3-(6-(1-hydroxyethyl)pyridin-3-yl)-4-methylphenyl)urea 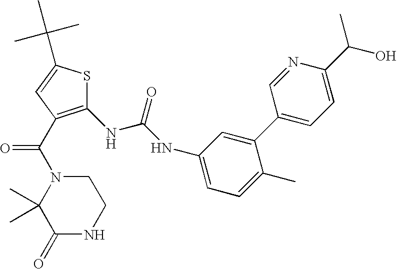 | M + H 564 | A |
| 1-(5-tert-butyl-3-(2,2,4-trimethyl-3- oxopiperazine-1-carbonyl)thiophen-2-yl)-3- (3-(6-(1-hydroxyethyl)pyridin-3-yl)-4-methylphenyl)urea 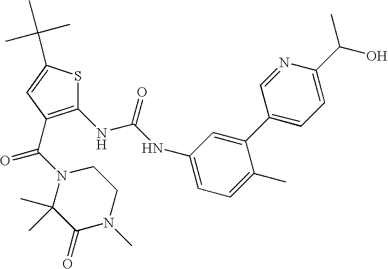 | M + H 578 | A |
| 1-(5-tert-butyl-3-(2,2-dimethyl-3- oxopiperazine-1-carbonyl)thiophen-2-yl)-3- (3-(6-ethylpyridin-3-yl)-5-fluoro-4-methylphenyl)urea 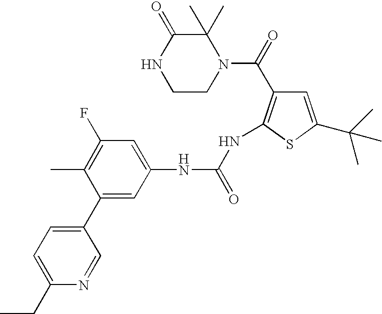 | M + H 566 | A |
| 1-(5-tert-butyl-3-(2,2,4-trimethyl-3- oxopiperazine-1-carbonyl)thiophen-2-yl)-3- (3-(6-ethylpyridin-3-yl)-5-fluoro-4-methylphenyl)urea 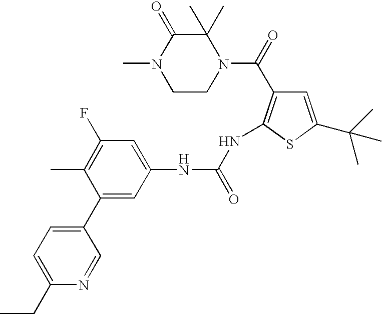 | M + H 580 | A |
| 1-(5-tert-butyl-3-(2,2,4-trimethyl-3- oxopiperazine-1-carbonyl)thiophen-2-yl)-3- (4-methyl-3-(6-methylpyridin-3-yl)phenyl)urea 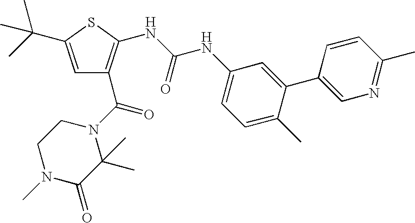 | M + H 548 | A |
| 1-(5-tert-butyl-3-(2,2-dimethyl-3- oxopiperazine-1-carbonyl)thiophen-2-yl)-3-(2- fluoro-5-(6-methoxypyridin-3-yl)-4-methylphenyl)urea 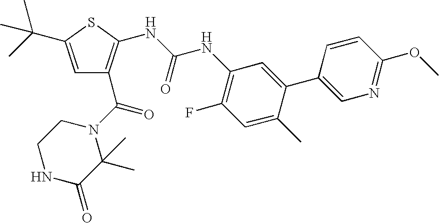 | M + H 568 | A |
| 1-(5-tert-butyl-3-(2,2,4-trimethyl-3- oxopiperazine-1-carbonyl)thiophen-2-yl)-3-(2- fluoro-5-(6-methoxypyridin-3-yl)-4-methylphenyl)urea 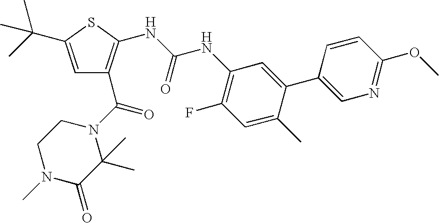 | M + H 582 | A |
| 1-(5-tert-butyl-3-(2,2,4-trimethyl-3- oxopiperazine-1-carbonyl)thiophen-2-yl)-3-(5- (cyclopropylethynyl)-6-methylpyridin-3-yl)urea 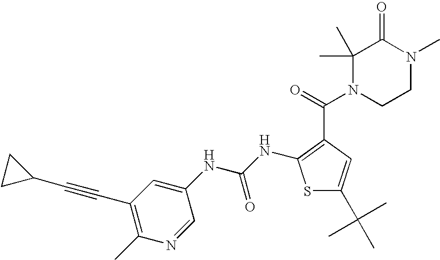 | M + H 522 | A |
| 1-(1-tert-butyl-3-(2,2,4-trimethyl-3- oxopiperazine-1-carbonyl)-1H-pyrazol-4-yl)-3- (3-(6-ethylpyridin-3-yl)-5-fluoro-4-methylphenyl)urea 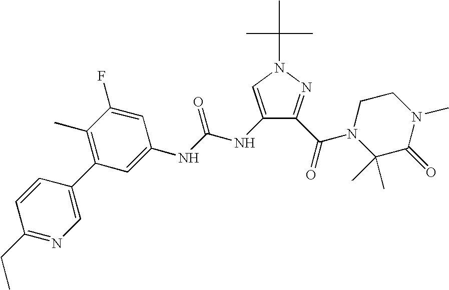 | M + H 564 | A |
| 1-(5-tert-butyl-3-(2,2,4-trimethyl-3- oxopiperazine-1-carbonyl)thiophen-2-yl)-3- (4-methyl-3-(6-morpholinopyridin-3-yl)phenyl)urea 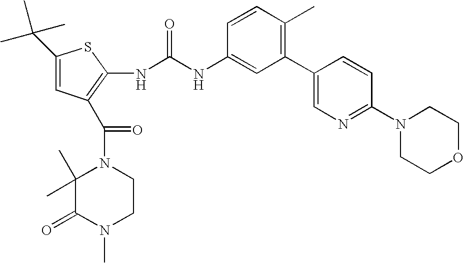 | M + H 619 | A |
| 1-(5-tert-butyl-3-(2,2-dimethyl-3- oxopiperazine-1-carbonyl)thiophen-2-yl)-3- (3-(6-isopropylpyridin-3-yl)-4-methylphenyl)urea 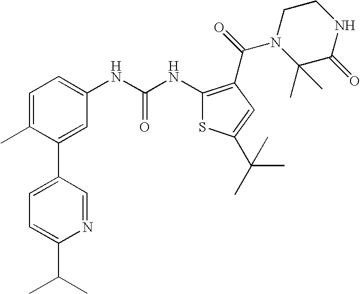 | M + H 562 | A |
| 1-(5-tert-butyl-2-(2,2,4-trimethyl-3- oxopiperazine-1-carbonyl)thiophen-3-yl)-3- (3-(6-ethylpyridin-3-yl)-5-fluoro-4-methylphenyl)urea 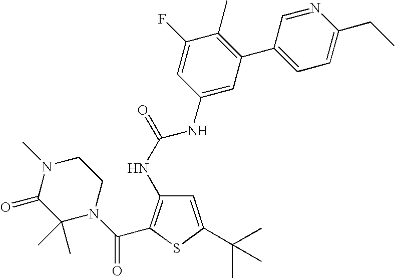 | M + H 580 | A |
| 1-(5-tert-butyl-3-(2,2-dimethyl-3- oxopiperazine-1-carbonyl)thiophen-2-yl)-3- (3-(6-ethanoylpyridin-3-yl)-4-methylphenyl)urea 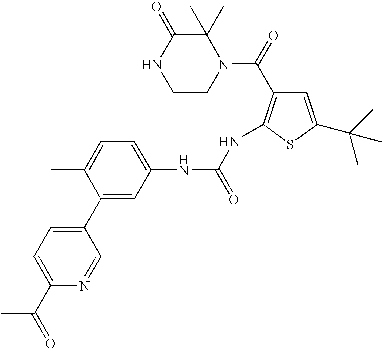 | M + H 562 | A |
| 1-(5-tert-butyl-3-(2,2-dimethyl-3- oxopiperazine-1-carbonyl)thiophen-2-yl)-3- (4-methyl-3-(pyridin-2-ylethynyl)phenyl)urea 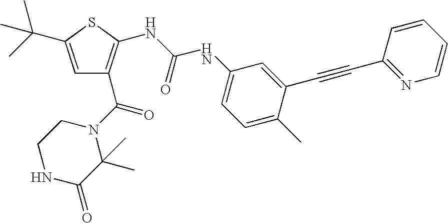 | M + H 544 | A |
| 5-(5-(3-(5-tert-butyl-3-(2,2-dimethyl-3- oxopiperazine-1-carbonyl)thiophen-2-yl)ureido)-2- methylphenyl)-N,N-dimethylpyridine-2-carboxamide 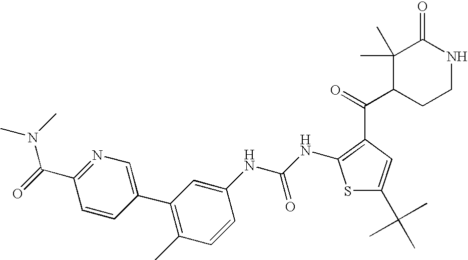 | M + H 591 | A |
| 5-(5-(3-(5-tert-butyl-3-(2,2-dimethyl-3- oxopiperazine-1-carbonyl)thiophen-2-yl)ureido)-2- methylphenyl)-N-methylpyridine-2-carboxamide 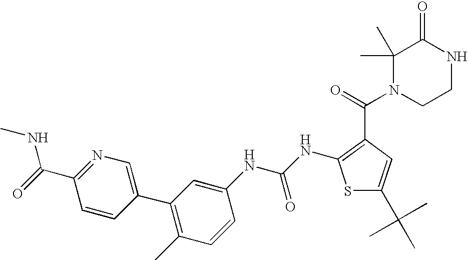 | M + H 577 | A |
| 5-(5-(3-(1-tert-butyl-3-(2,2-dimethyl-3- oxopiperazine-1-carbonyl)-1H-pyrazol-4- yl)ureido)-2-methylphenyl)-N-ethylpyridine-2-carboxamide 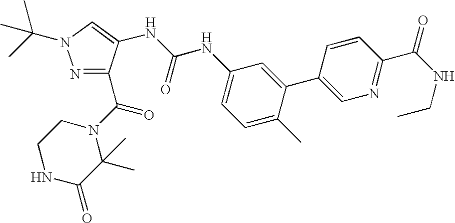 | M + H 575 | A |
| 5-(5-(3-(1-tert-butyl-3-(2,2,4-trimethyl-3- oxopiperazine-1-carbonyl)-1H-pyrazol-4- yl)ureido)-2-methylphenyl)-N-ethylpyridine-2-carboxamide 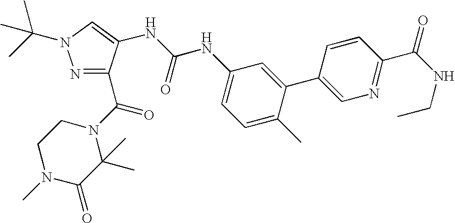 | M + H 589 | A |
| 1-(1-tert-butyl-3-(2,2-dimethyl-3- oxopiperazine-1-carbonyl)-1H-pyrazol-4-yl)-3- (3-(6-ethylpyridin-3-yl)-4-methylphenyl)urea 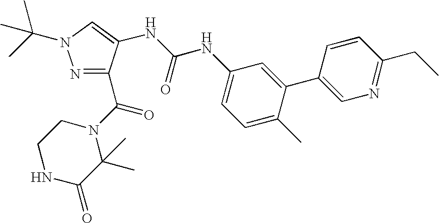 | M + H 532 | A |
| 1-(1-tert-butyl-3-(2,2-dimethyl-3- oxopiperazine-1-carbonyl)-1H-pyrazol-4-yl)-3- (4-methyl-3-(6-(morpholinomethyl)pyridin-3-yl)phenyl)urea 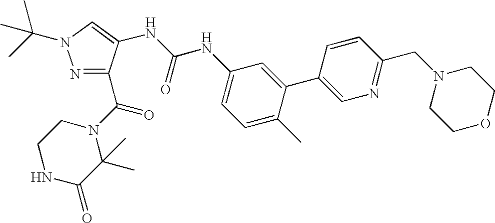 | M + H 603 | A |
| 1-(1-tert-butyl-3-(2,2,4-trimethyl-3- oxopiperazine-1-carbonyl)-1H-pyrazol-4-yl)-3- (4-methyl-3-(6-(morpholinomethyl)pyridin-3-yl)phenyl)urea 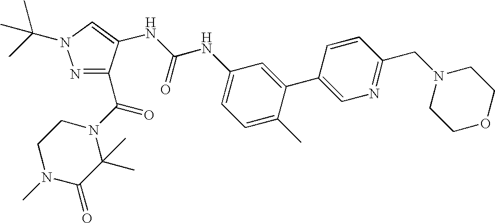 | M + H 617 | A |
| 1-(1-tert-butyl-3-(2,2,4-trimethyl-3- oxopiperazine-1-carbonyl)-1H-pyrazol-4-yl)-3- (3-(6-isopropylpyridin-3-yl)-4-methylphenyl)urea 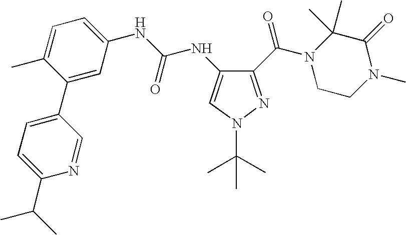 | M + H 560 | A |
| 1-(5-tert-butyl-3-(2,2,4-trimethyl-3- oxopiperazine-1-carbonyl)thiophen-2-yl)-3-(4- methyl-3-(6-(morpholinomethyl)pyridin-3-yl)phenyl)urea 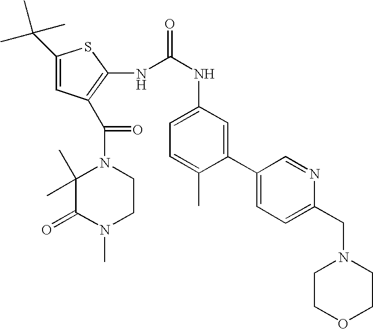 | ¹H NMR (400 MHz, CDCl₃) δ 9.98 (s, 1H), 8.49 (d, J = 2.2 Hz, 1H), 8.25 (s, 1H), 7.61 (dd, J = 7.9, 2.2 Hz, 1H), 7.41 (d, J = 7.9 Hz, 1H), 7.32 (d, J = 2.3 Hz, 1H), 7.24 (dd, J = 8.2, 2.3 Hz, 1H), 7.15 (d, J = 8.3 Hz, 1H), 6.37 (s, 1H), 3.70 (m, 8H), 3.44 (t, J = 5.0 Hz, 2H), 3.00 (s, 3H), 2.52 (t, J = 4.5 Hz, 4H), 2.17 (s, 3H), 1.68 (s, 6H), 1.29 (s, 9H). | A |
| 1-(1-tert-butyl-3-(2,2-dimethyl-3- oxopiperazine-1-carbonyl)-1H-pyrazol-4-yl)-3- (3-(6-isopropylpyridin-3-yl)-4-methylphenyl)urea 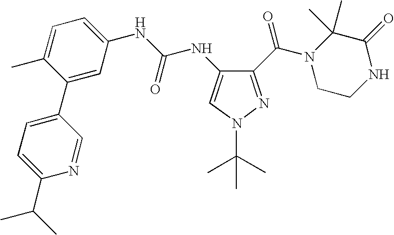 | M + H 546 | A |
| 1-(5-tert-butyl-3-(2,2,4-trimethyl-3- oxopiperazine-1-carbonyl)thiophen-2-yl)-3-(4- methyl-3-(5-(morpholinomethyl)pyridin-2-yl)phenyl)urea 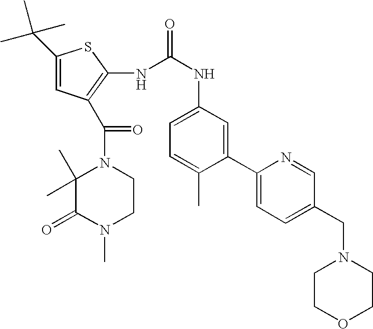 | M + H 633 | A |
| 1-(3-(2,2-dimethyl-3-oxopiperazine-1- carbonyl)-1-(1-methylcyclopentyl)-1H-pyrazol-4- yl)-3-(3-(6-ethylpyridin-3-yl)-4-methylphenyl)urea 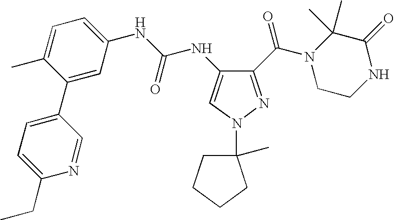 | M + H 558 | A |
| 1-(5-tert-butyl-3-(2,2-dimethyl-3- oxopiperazine-1-carbonyl)thiophen-2-yl)-3- (4-methyl-3-(6-methylpyridazin-3-yl)phenyl)urea 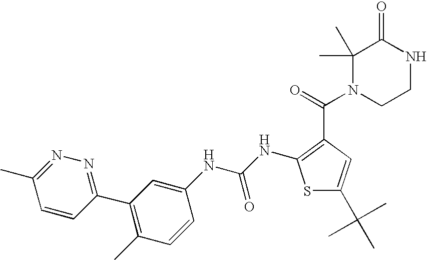 | M + H 535 | A |
| 5-(5-(3-(5-tert-butyl-3-(2,2,4-trimethyl-3- oxopiperazine-1-carbonyl)thiophen-2-yl)ureido)- 2-methylphenyl)-N-ethylpyridine-2-carboxamide 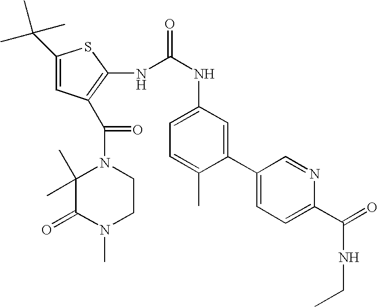 | M + H 605 | A |
| 5-(5-(3-(5-tert-butyl-2-(2,2,4-trimethyl-3- oxopiperazine-1-carbonyl)furan-3-yl)ureido)- 2-methylphenyl)-N-ethylpyridine-2-carboxamide 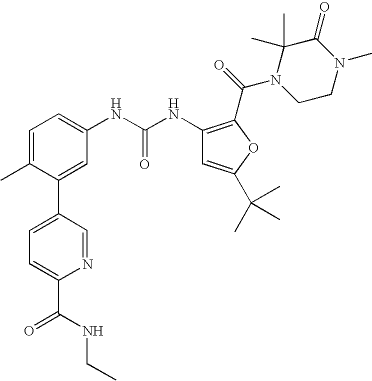 | M + H 589 | A |
| 1-(1-tert-butyl-3-(2,2,4-trimethyl-3- oxopiperazine-1-carbonyl)-1H-pyrazol-4-yl)-3- (4-methyl-3-(5-(morpholinomethyl)pyridin-2-yl)phenyl)urea 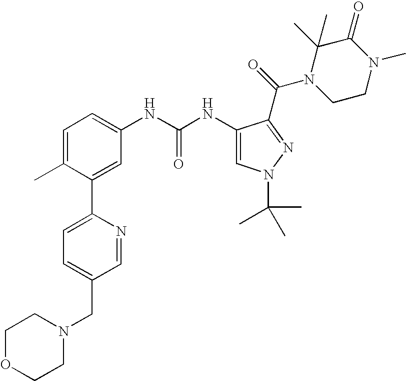 | M + H 617 | A |
| 5-(5-(3-(5-tert-butyl-2-(2,2-dimethyl-3- oxopiperazine-1-carbonyl)thiophen-3-yl)ureido- 2-methylphenyl)-N-ethylpyridine-2-carboxamide 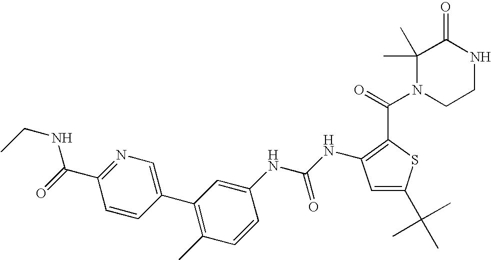 | M + H 591 | A |
| 1-(5-tert-butyl-2-(2,2-dimethyl-3- oxopiperazine-1-carbonyl)thiophen-2-yl)-3-(4- methyl-3-(6-(morpholinomethyl)pyridin-3-yl)phenyl)urea 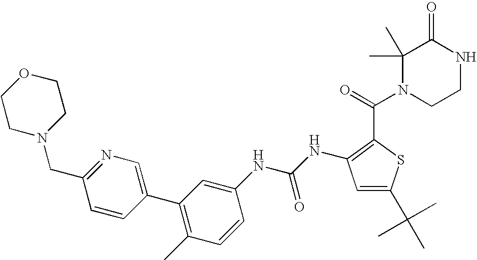 | M + H 619 | A |
| 5-(5-(3-(5-tert-butyl-2-(2,2,4-trimethyl-3- oxopiperazine-1-carbonyl)thiophen-3-yl)ureido)- 2-methylphenyl)-N-ethylpyridine-2-carboxamide 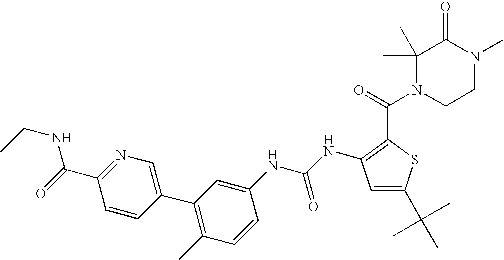 | M + H 605 | A |
| 1-(5-tert-butyl-2-(2,2,4-trimethyl-3- oxopiperazine-1-carbonyl)thiophen-3-yl)-3-(4- methyl-3-(6-(morpholinomethyl)pyridin-3-yl)phenyl)urea 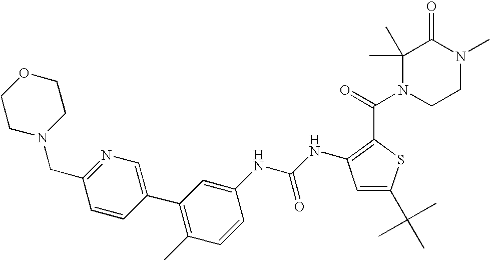 | H-1 NMR (400 MHz, CDCl₃): □(ppm) 9.75 (s, 1H), 8.52 (d, J = 1.6 Hz, 1H), 7.74 (s, 1H), 7.63 (dd, J = 7.8, 2.0 Hz, 1H), 7.42 (d, J = 8.0 Hz, 1H), 7.34 (s, 1H), 7.31 (d, J = 2.1 Hz, 1H), 7.28 (dd, J = 8.2, 2.1 Hz, 1H), 7.20 (d, J = 8.4 Hz, 1H), 3.90 (t, J = 4.9 Hz, 2H), 3.74 (t, J = 4.3 Hz, 4H), 3.69 (s, 2H), 3.50 (t, J = 5.0 Hz, 2H), 3.01 (s, 3H), 2.53 (t, J = 3.9 Hz, 4H), 2.20 (s, 3H), 1.69 (s, 6H), 1.34 (s, 9H). | A |
| 1-(5-tert-butyl-2-(2,2,4-trimethyl-3- oxopiperazine-1-carbonyl)furan-3-yl)-3-(4- methyl-3-(5-(morpholinomethyl)pyridin-2-yl)phenyl)urea 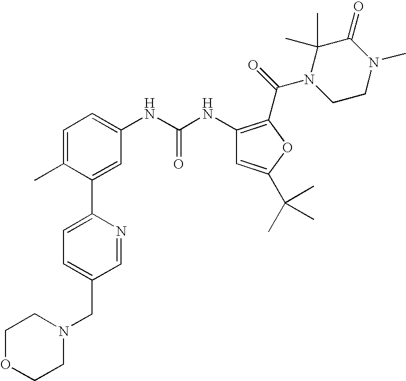 | H-1 NMR (400 MHz, CDCl₃): □(ppm) 9 .25 (s, 1H), 8.59 (d, J = 1.6 Hz, 1H), 7.72 (d, J = 7.9 Hz, 1H), 7.46 (d, J = 2.1 Hz, 1H), 7.41 (d, J = 8.0 Hz, 1H), 7.30 (dd, J = 8.2, 2.3 Hz, 1H), 7.19 (d, J = 8.4 Hz, 1H), 7.05 (s, 1H), 7.00 (s, 1H), 3.89 (t, J = 5.0 Hz, 2H), 3.72 (t, J = 4.1 Hz, 4H), 3.56 (s, 2H), 3.50 (t, J = 4.9 Hz, 2H), 3.02 (s, 3H), 2.49 (s, 4H), 2.29 (s, 3H), 1.72 (s, 6H), 1.26 (s, 9H). | A |
| 1-(1-tert-butyl-3-(2,2-dimethyl-3- oxopiperazine-1-carbonyl)-1H-pyrazol-4-yl)-3- (4-methyl-3-(5-(morpholinomethyl)pyridin-2-yl)phenyl)urea 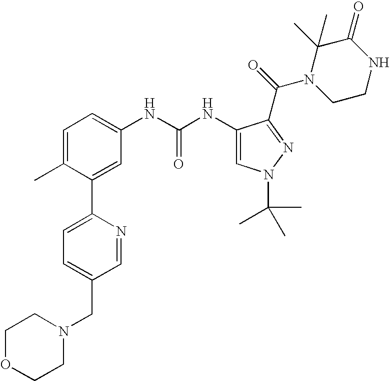 | M + H 603 | A |
| 1-(5-tert-butyl-3-(2,2,4-trimethyl-3- oxopiperazine-1-carbonyl)thiophen-2-yl)-3-(3- (6-(2-hydroxypropan-2-yl)pyridin-3-yl)-4-methylphenyl)urea 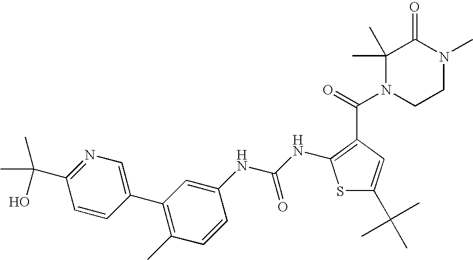 | H-1 NMR (400 MHz, CDCl₃): □(ppm) 10.00 (s, 1H), 8.44 (d, J = 1.6 Hz, 1H), 7.80 (s, 1H), 7.66 (dd, J = 8.0, 2.1 Hz, 1H), 7.36-7.40 (m, 2H), 7.22 (dd, J = 8.4, 2.3 Hz, 1H), 7.17 (d, J = 8.4 Hz, 1H), 6.38 (s, 1H), 5.05 (s, 1H), 3.73 (t, J = 5.0 Hz, 2H), 3.46 (t, J = 5.0 Hz, 2H), 3.02 (s, 3H), 2.20 (s, 3H), 1.73 (s, 6H), 1.58 (s, 6H), 1.30 (s, 9H). | A |
| 1-(5-tert-butyl-3-(2,2,4-trimethyl-3- oxopiperazine-1-carbonyl)thiophen-2-yl)-3-(4- methyl-3-(6-(morpholine-4-carbonyl)pyridin-3-yl)phenyl)urea 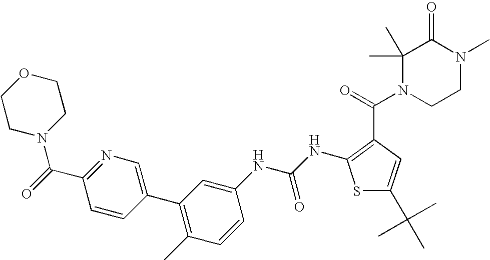 | H-1 NMR (400 MHz, CDCl₃): □(ppm) 10.04 (s, 1H), 8.52 (d, J = 1.4 Hz, 1H), 7.76 (dd, J = 8.0, 2.1 Hz, 1H), 7.71 (d, J = 7.9 Hz, 1H), 7.61 (s, 1H), 7.48 (d, J = 2.0 Hz, 1H), 7.18 (d, J = 8.2 Hz, 1H), 7.13 (dd, J = 8.2, 2.1 Hz, 1H), 6.41 (s, 1H), 3.81-3.85 (m, 4H), 3.73-3.75 (m, 6H), 3.47 (t, J = 5.0 Hz, 2H), 3.03 (s, 3H), 2.19 (s, 3H), 1.75 (s, 6H), 1.31 (s, 9H). | A |
| 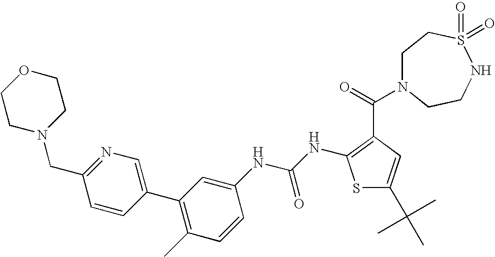 | M + H 641 | A |
| 5-(5-(3-(5-tert-butyl-3-(2,2,4-trimethyl-3- oxopiperazine-1-carbonyl)thiophen-2-yl)ureido)- 2-methylphenyl)-N-(2-methoxyethyl)pyridine-2-carboxamide 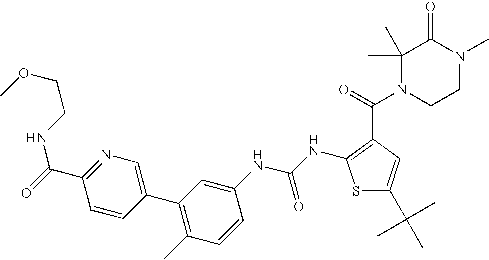 | H-1 NMR (400 MHz, CDCl₃): □(ppm) 9.97 (s, 1H), 8.48 (d, J = 1.4 Hz, 1H), 8.34- 8.38 (m, 2H), 8.18 (d, J = 8.0 Hz, 1H), 7.74 (dd, J = 8.0, 2.1 Hz, 1H), 7.36 (dd, J = 8.2, 2.3 Hz, 1H), 7.30 (d, J = 2.3 Hz, 1H), 7.17 (d, J = 8.4 Hz, 1H), 6.37 (s, 1H), 3.67-3.72 (m, 4H), 3.58 (t, J = 5.1 Hz, 2H), 3.45 (t, J = 4.9 Hz, 2H), 3.38 (s, 3H), 3.01 (s, 3H), 2.16 (s, 3H), 1.69 (s, 6H), 1.29 (s, 9H). | A |
| 5-(5-(3-(5-tert-butyl-3-(2,2,4-trimethyl-3- oxopiperazine-1-carbonyl)thiophen-2-yl)ureido)- 2-methylphenyl)pyridine-2-carboxamide 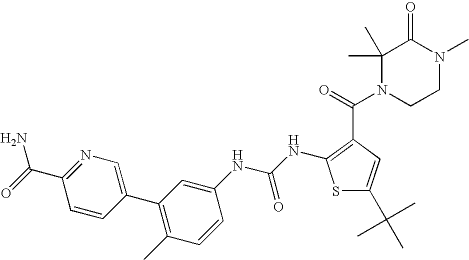 | M + H 577 | A |
| 1-(2-tert-butyl-4-(2,2,4-trimethyl-3- oxopiperazine-1-carbonyl)thiazol-5-yl)-3-(4- methyl-3-(6-(morpholinomethyl)pyridin-3-yl)phenyl)urea 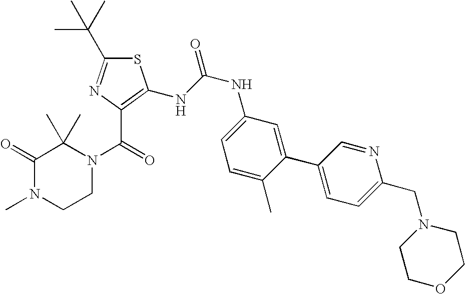 | H-1 NMR (400 MHz, CDCl₃): □(ppm) 10.90 (s, 1H), 8.54 (d, J = 1.8 Hz, 1H), 7.64 (dd, J = 7.9, 2.2 Hz, 1H), 7.44 (d, J = 8.2 Hz, 1H), 7.40 (d, J = 2.1 Hz, 1H), 7.27 (dd, J = 8.2, 2.2 Hz, 1H), 7.24 (d, J = 8.1 Hz, 1H), 7.01 (s, 1H), 4.13 (t, J = 4.8 Hz, 2H), 3.75 (t, J = 4.7 Hz, 4H), 3.70 (s, 2H), 3.55 (t, J = 4.8 Hz, 2H), 3.02 (s, 3H), 2.55 (t, J = 4.5 Hz, 4H), 2.22 (s, 3H), 1.76 (s, 6H), 1.37 (s, 9H). | A |
| 1-(5-tert-butyl-3-(2,2,4-trimethyl-3- oxopiperazine-1-carbonyl)thiophen-2-yl)-3-(4- methyl-3-(6-(5-methyl-1,3,4-oxadiazol-2-yl)pyridin-3-yl)phenyl)urea 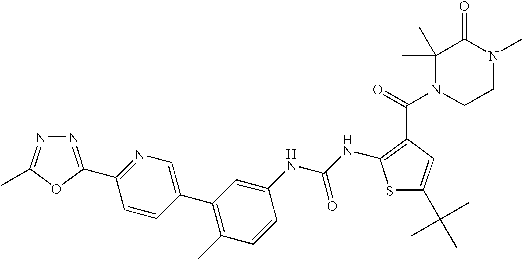 | H-1 NMR (400 MHz, CDCl₃): □(ppm) 10.05 (s, 1H), 8.70 (d, J = 1.4 Hz, 1H), 8.24 (d, J = 8.2 Hz, 1H), 7.85 (dd, J = 8.2, 2.1 Hz, 1H), 7.75 (s, 1H), 7.45 (d, J = 2.1 Hz, 1H), 7.30 (dd, J = 8.2, 2.3 Hz, 1H), 7.21 (d, J = 8.4 Hz, 1H), 6.40 (s, 1H), 3.73 (t, J = 5.0 Hz, 2H), 3.46 (t, J = 5.0 Hz, 2H), 3.02 (s, 3H), 2.67 (s, 3H), 2.22 (s, 3H), 1.72 (s, 6H), 1.31 (s, 9H). | A |
| 1-(3-(6-(1H-imidazol-1-yl)pyridin-3-yl)-4- methylphenyl)-3-(5-tert-butyl-3-(2,2,4-trimethyl- 3-oxopiperazine-1-carbonyl)thiophen-2-yl)urea 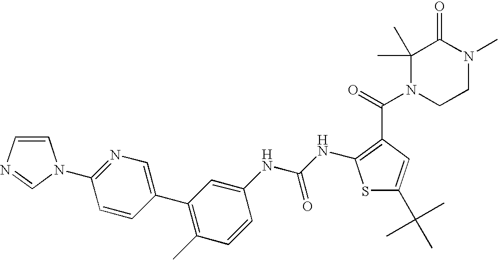 | M + H 600 | A |
| 5-(5-(3-(5-tert-butyl-3-(2,2,4-trimethyl-3- oxopiperazine-1-carbonyl)thiophen-2-yl)ureido)- 2-methylphenyl)-N-methoxy-N-methylpyridine-2-carboxamide 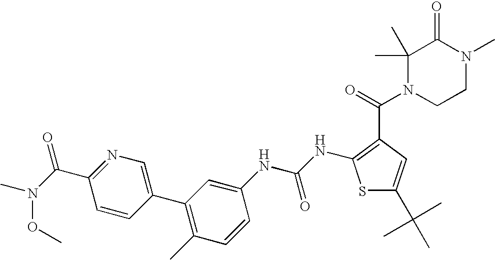 | M + H 621 | A |
| 1-(5-tert-butyl-3-(2,2,4-trimethyl-3- oxopiperazine-1-carbonyl)thiophen-2-yl)-3- (4-methyl-3-(6-(4-methylpiperazin-1- yl)pyridin-3-yl)phenyl)urea 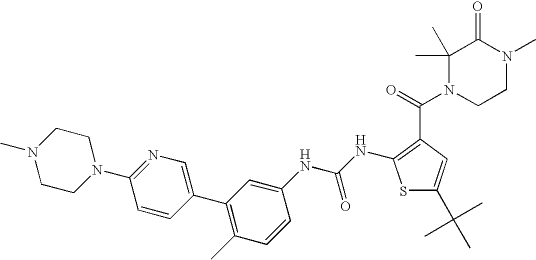 | M + H 632 | A |
| 5-(5-(3-(2-tert-butyl-4-(2,2,4-trimethyl-3- oxopiperazine-1-carbonyl)thiazol-5- yl)ureido)-2-methylphenyl)-N- ethylpyridine-2-carboxamide 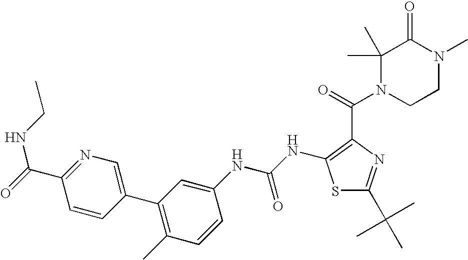 | M + H 606 | A |
| 1-(2-tert-butyl-4-(2,2,4-trimethyl-3- oxopiperazine-1-carbonyl)thiazol-5-yl)-3- (4-methyl-3-(5-(morpholinomethyl)pyridin- 2-yl)phenyl)urea 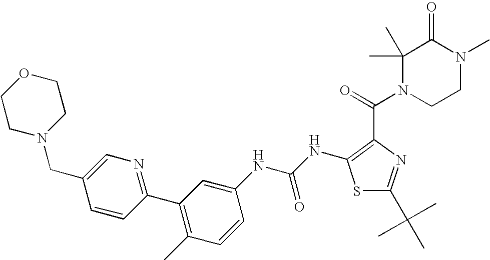 | M + H 634 | A |
| 1-(3-(6-(1H-pyrazol-1-yl)pyridin-3-yl)-4- methylphenyl)-3-(5-tert-butyl-3-(2,2,4- trimethyl-3-oxopiperazine-1- carbonyl)thiophen-2-yl)urea 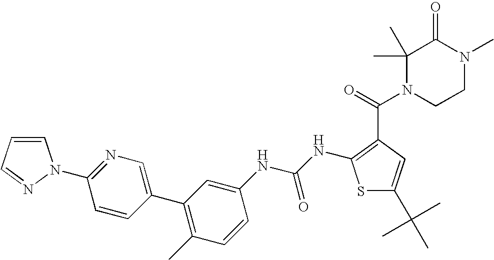 | M + H 600 | A |
| 1-(5-tert-butyl-3-(2,2,4-trimethyl-3- oxopiperazine-1-carbonyl)thiophen-2-yl)-3-(3- (6-(2-methoxyethoxy)pyridin-3-yl)-4-methylphenyl)urea 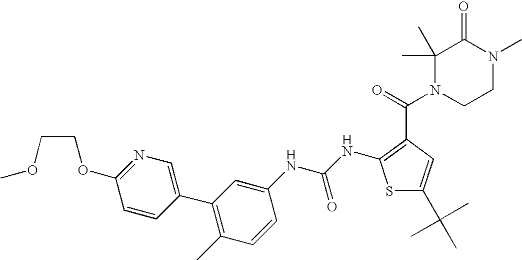 | M + H 608 | A |
| N-(5-(5-(3-(5-tert-butyl-3-(2,2,4-trimethyl- 3-oxopiperazine-1-carbonyl)thiophen-2-yl)ureido)- 2-methylphenyl)pyridin-2-yl)methanesulfonamide 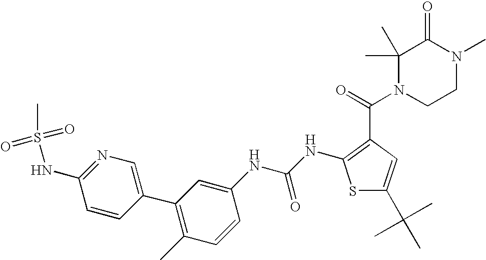 | M + H 627 | A |
| 1-(5-tert-butyl-2-(4-methyl-3- oxopiperazine-1-carbonyl)thiophen-3-yl)-3-(4- methyl-3-(6-(morpholinomethyl)pyridin-3-yl)phenyl)urea 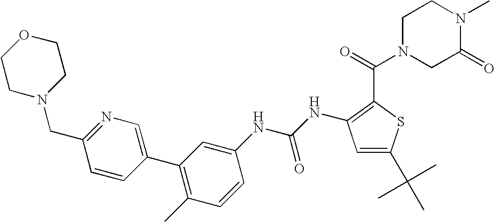 | M + H 605 | A |
| 1-(4-methyl-3-(6- (morpholinomethyl)pyridin-3-yl)phenyl)-3- (3-(2,2,4-trimethyl-3-oxopiperazine-1-carbonyl)- 5,7-dihydro-4H-thieno[2,3-c]pyran-2-yl)urea 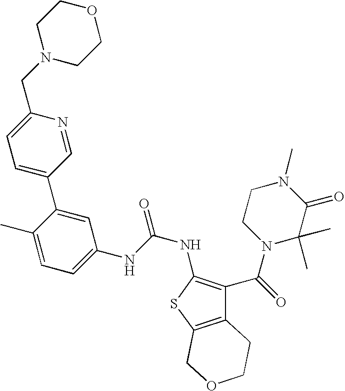 | M + H 633 | B |
| 4-((5-(5-(3-(5-tert-butyl-3-(2,2,4-trimethyl- 3-oxopiperazine-1-carbonyl)thiophen-2-yl)ureido)- 2-methylphenyl)pyridin-2-yl)methyl)morpholine 4-oxide 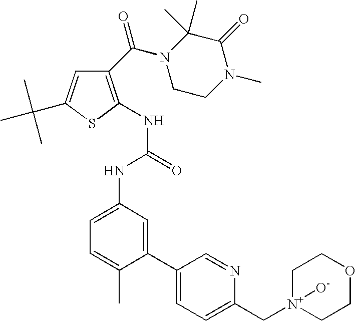 | M + H 649 | A |
| 1-(5-tert-butyl-3-(2,2,4-trimethyl-3- oxopiperazine-1-carbonyl)thiophen-2-yl)-3-(4-methyl-3- (6-((4-methylpiperazin-1-yl)methyl)pyridin-3-yl)phenyl)urea 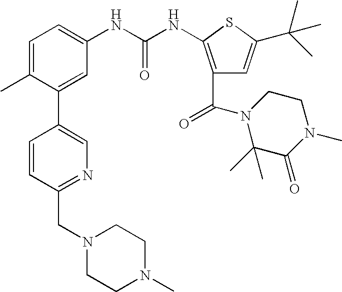 | M + H 646 | A |
| 1-(5-tert-butyl-3-(2,2,4-trimethyl-3- oxopiperazine-1-carbonyl)thiophen-2-yl)-3-(3- (6-((diethylamino)methyl)pyridin-3-yl)-4-methylphenyl)urea 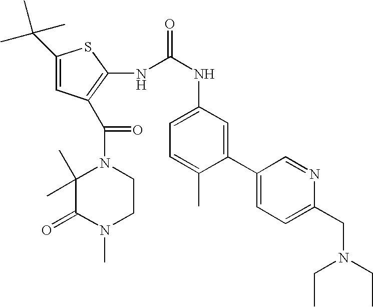 | M + H 619 | A |
| 1-(5-tert-butyl-3-(2,2,4-trimethyl-3- oxopiperazine-1-carbonyl)thiophen-2-yl)-3-(4- methyl-3-(6-(pyrrolidin-1-ylmethyl)pyridin-3-yl)phenyl)urea 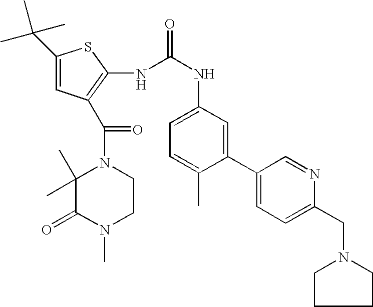 | M + H 617 | A |
| 4-((5-(5-(3-(5-tert-butyl-3-(2,2,4-trimethyl- 3-oxopiperazine-1-carbonyl)thiophen-2-yl)ureido)- 2-methylphenyl)-1-oxidopyridin-2-yl)methyl)morpholine 4-oxide 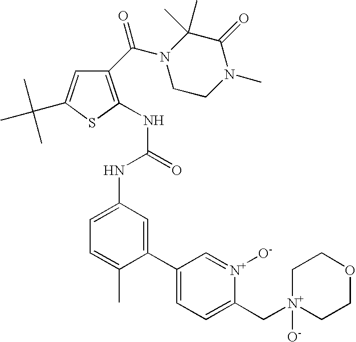 | M + H 665 | B |
| 1-(4-methyl-3-(6- (morpholinomethyl)pyridin-3-yl)phenyl)- 3-(5-tert-pentyl-3-(2,2,4-trimethyl-3- oxopiperazine-1-carbonyl)thiophen-2-yl)urea 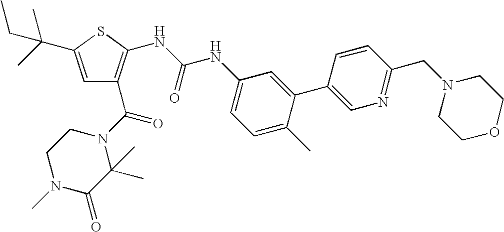 | M + H 647 | A |
| 1-(5-tert-butyl-2-(4-ethyl-2,2-dimethyl-3- oxopiperazine-1-carbonyl)thiophen-3-yl)-3-(4- methyl-3-(6-(morpholinomethyl)pyridin-3-yl)phenyl)urea 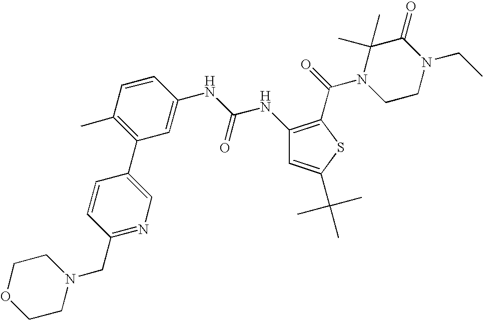 | M + H 647 | A |
| 1-(5-tert-butyl-2-(4-isopropyl-2,2-dimethyl- 3-oxopiperazine-1-carbonyl)thiophen-3-yl)-3- (4-methyl-3-(6-(morpholinomethyl)pyridin-3-yl)phenyl)urea 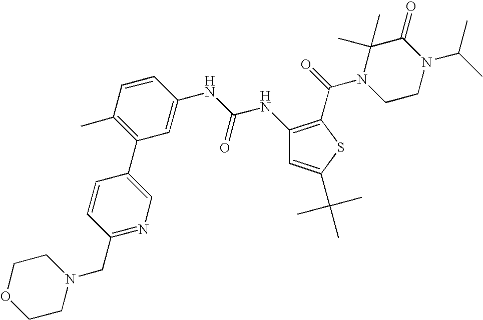 | M + H 661 | A |
| 1-(5-tert-butyl-3-(2,2,4-trimethyl-3- oxopiperazine-1-carbonyl)thiophen-2-yl)-3-(4- methyl-3-(6-(1-morpholinoethyl)pyridin-3-yl)phenyl)urea 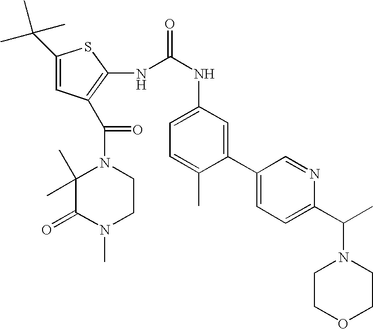 | M + H 647 | A |
| 1-(5-tert-butyl-2-(2,2,4-trimethyl-3- oxopiperazine-1-carbonyl)thiophen-3-yl)-3-(4- methyl-1,3,4-oxadiazol-2-yl)pyridin-3-yl)phenyl)urea 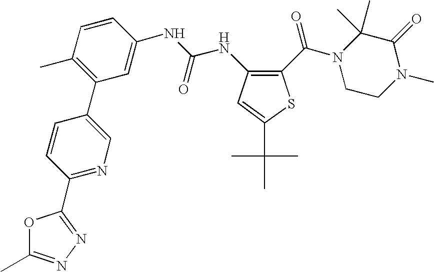 | M + H 616 | A |
| 1-(5-tert-butyl-3-(2,2,4-trimethyl-3- oxopiperazine-1-carbonyl)thiophen-2-yl)-3-(3-(2- (2,3-dihydroxypropylamino)pyrimidin-5-yl)-4-methylphenyl)urea 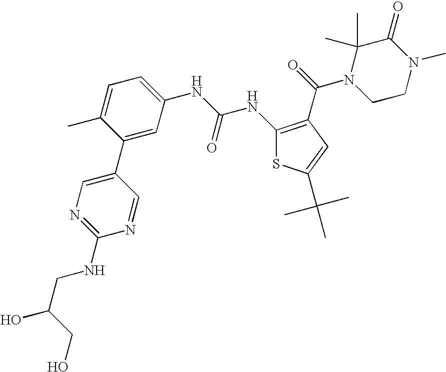 | M + H 624 | A |
| 1-(2-tert-butyl-4-(2,2,4-trimethyl-3- oxopiperazine-1-carbonyl)thiazol-5-yl)-3-(4- methyl-3-(6-(5-methyl-1,3,4-oxadiazol-2-yl)pyridin-3-yl)phenyl)urea 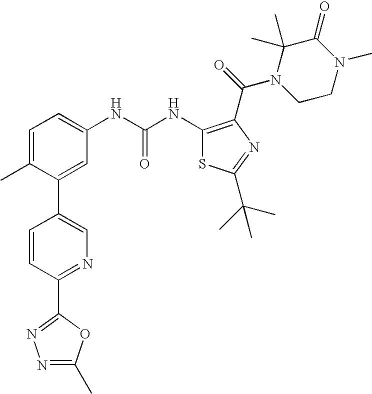 | M + H 617 | A |
| 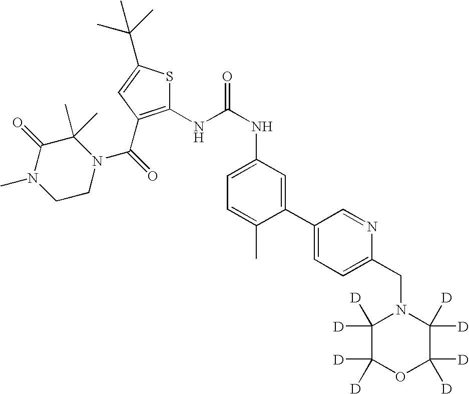 | M + H 641 | |

Exemplary compounds of embodiments of the invention include, but are not limited to, the following:

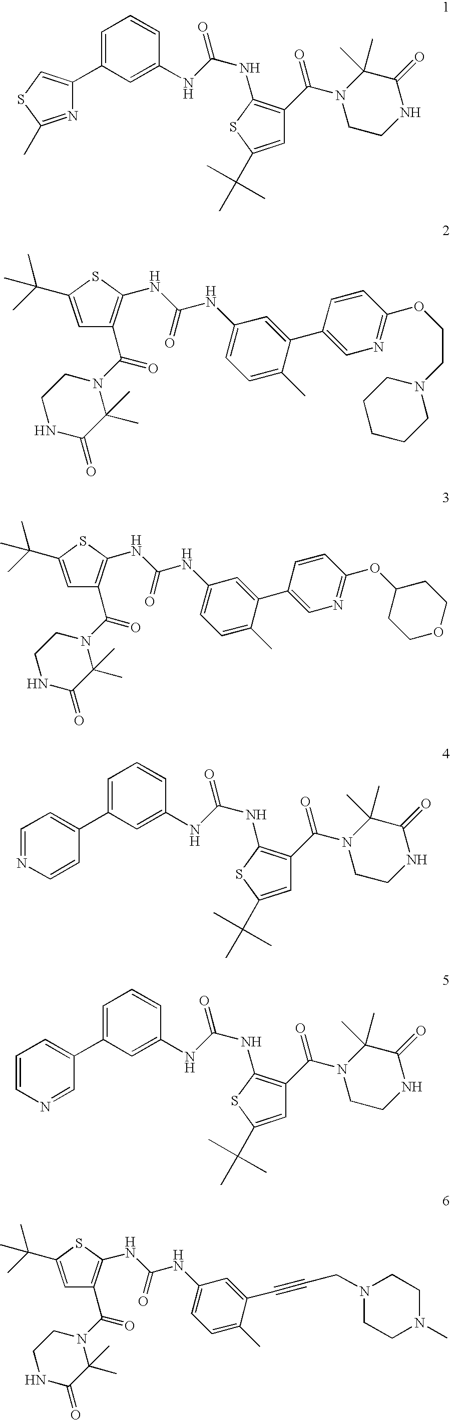

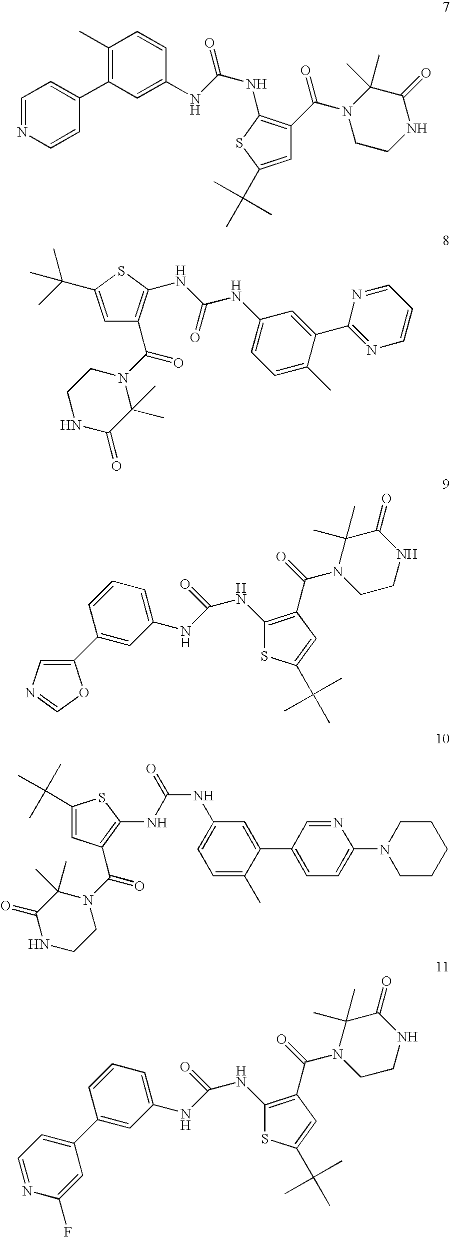

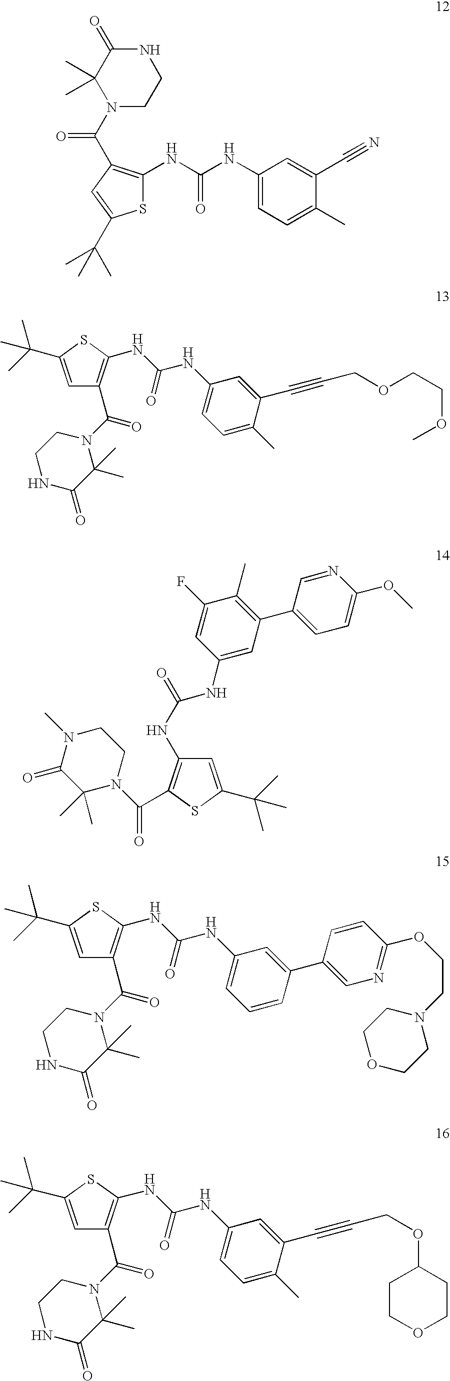

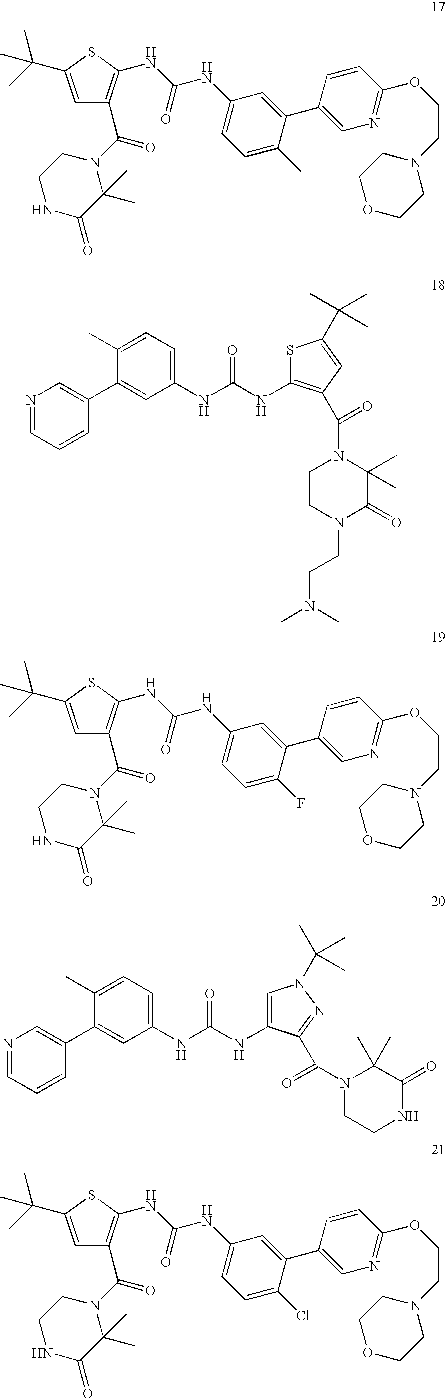

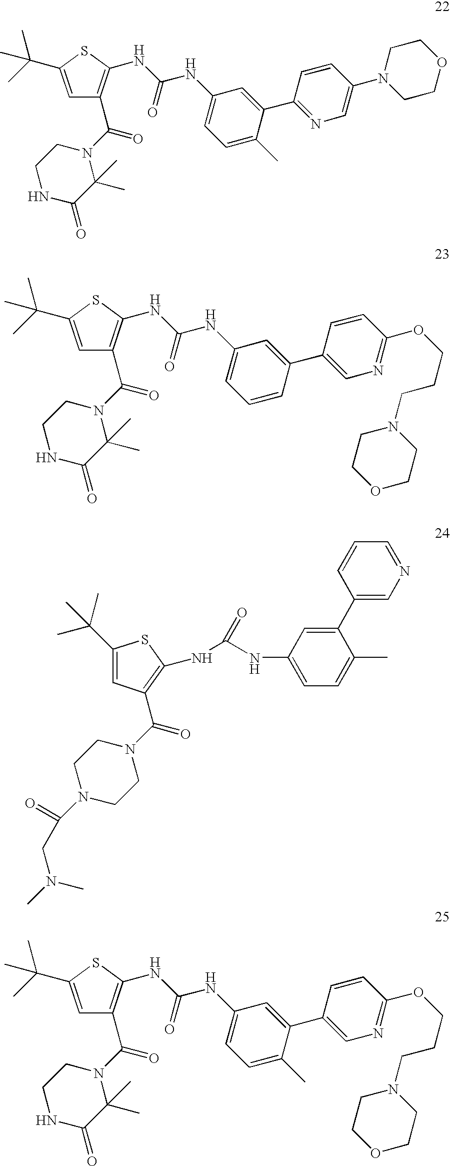

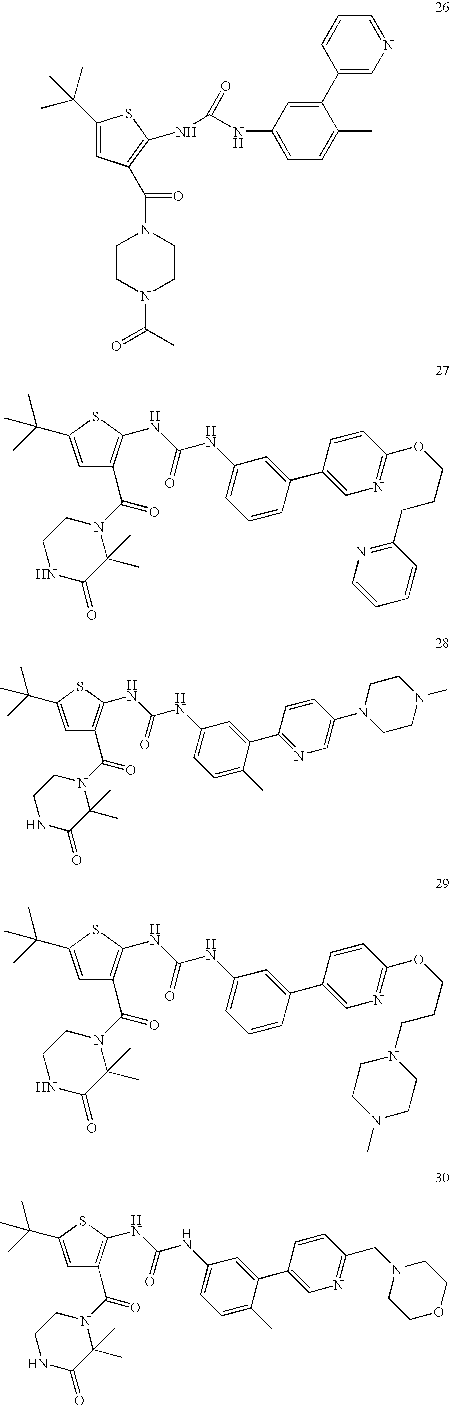

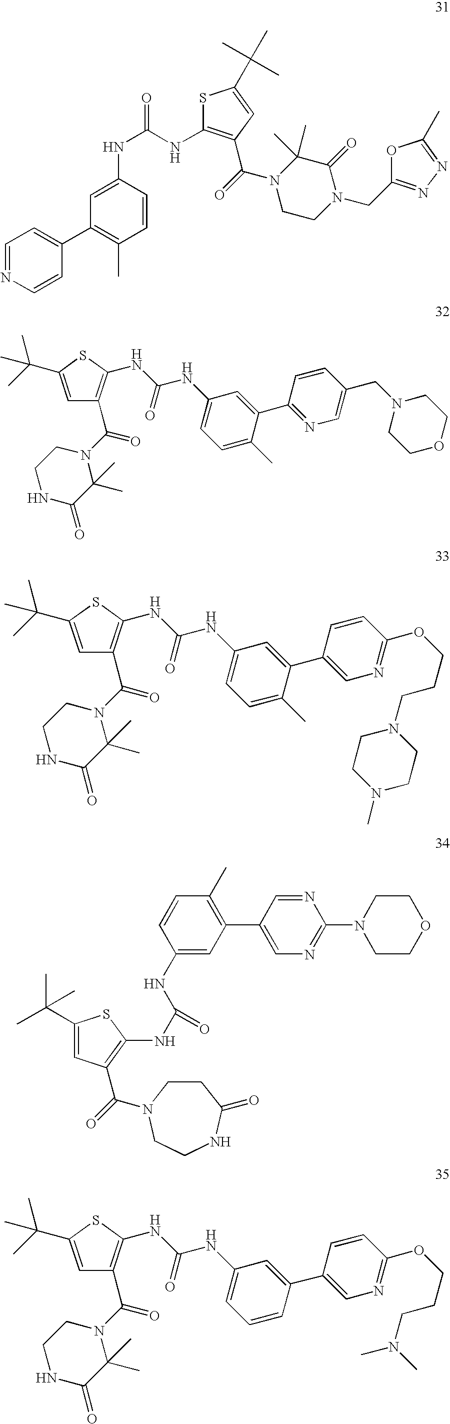

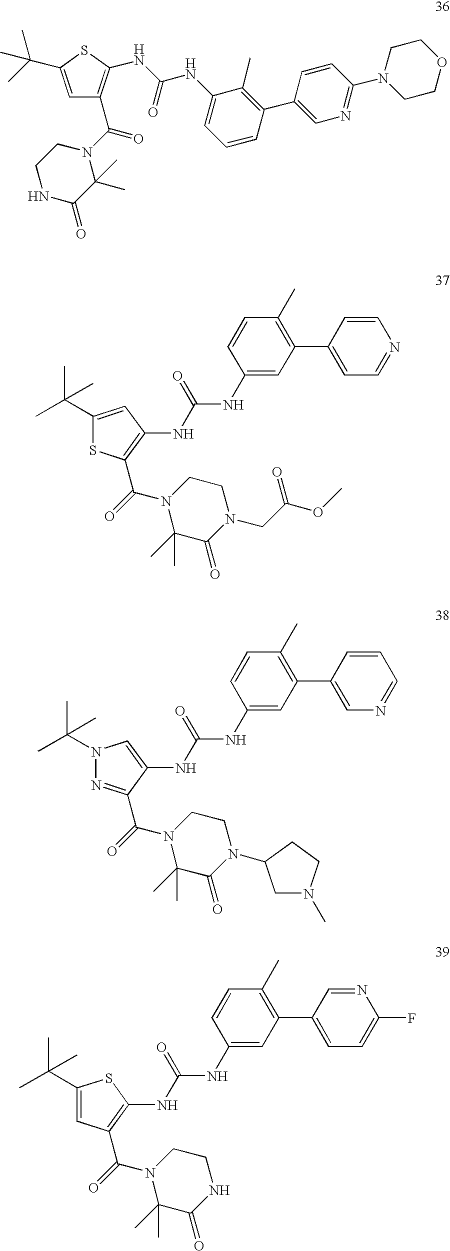

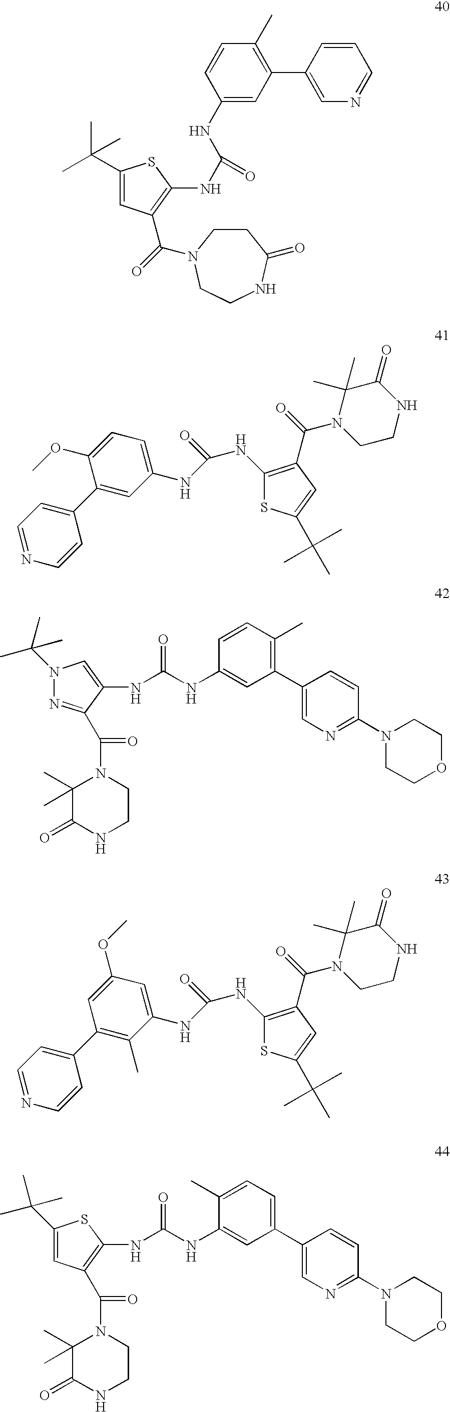

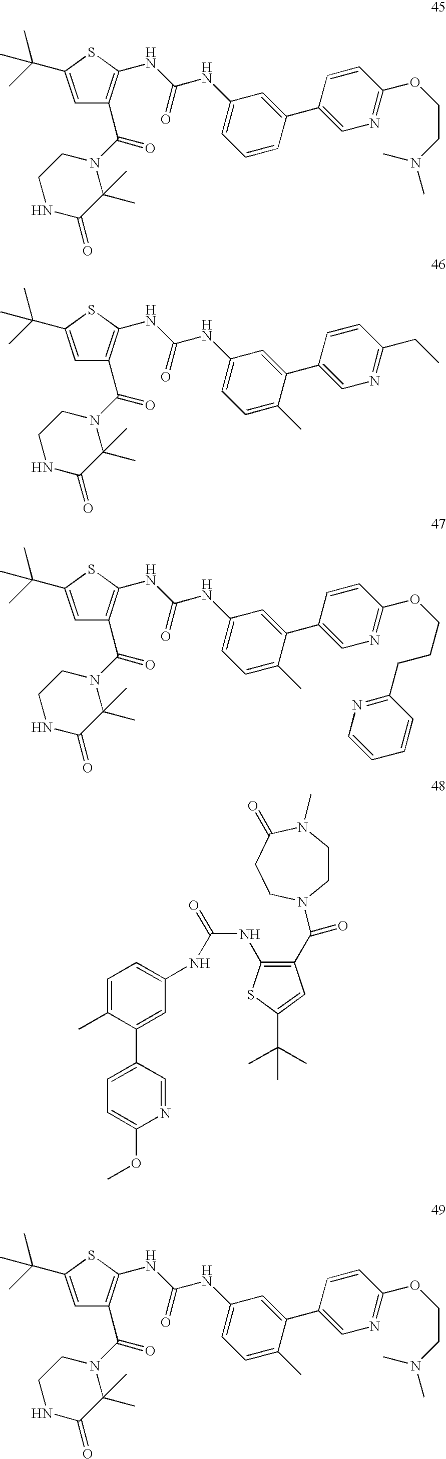

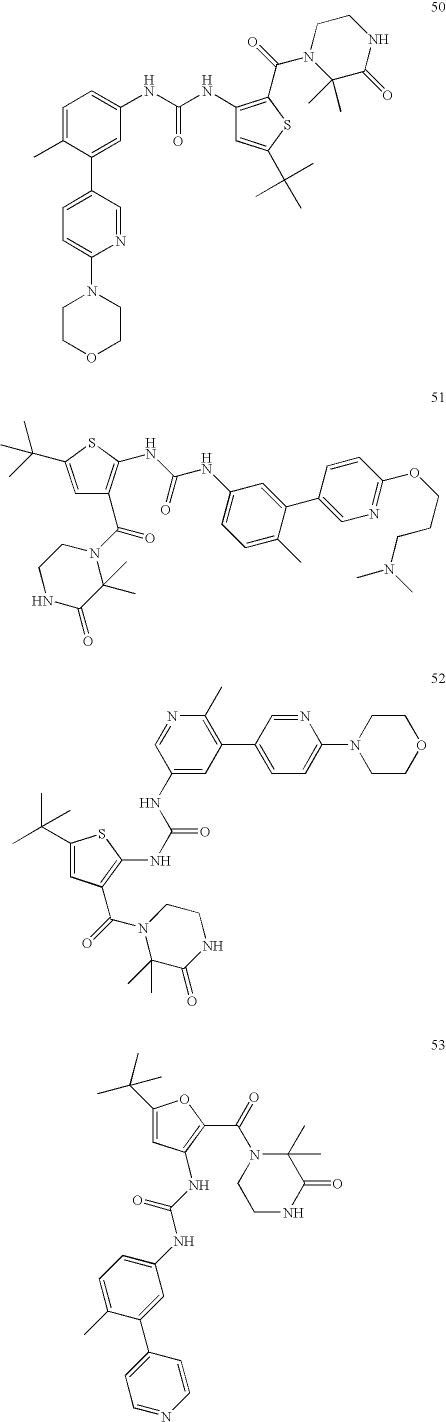

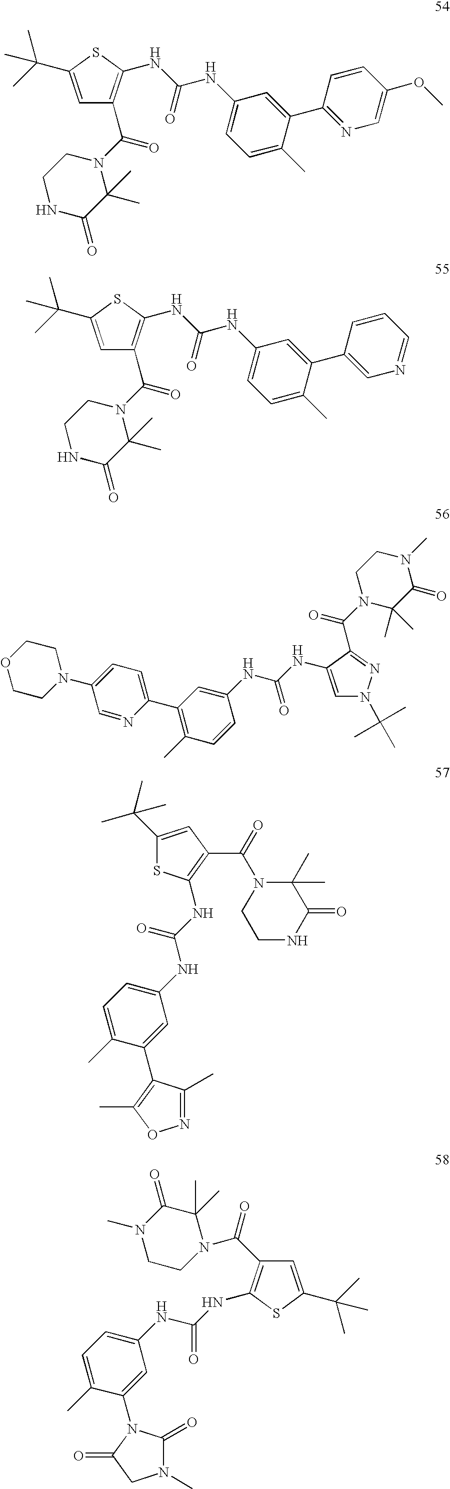

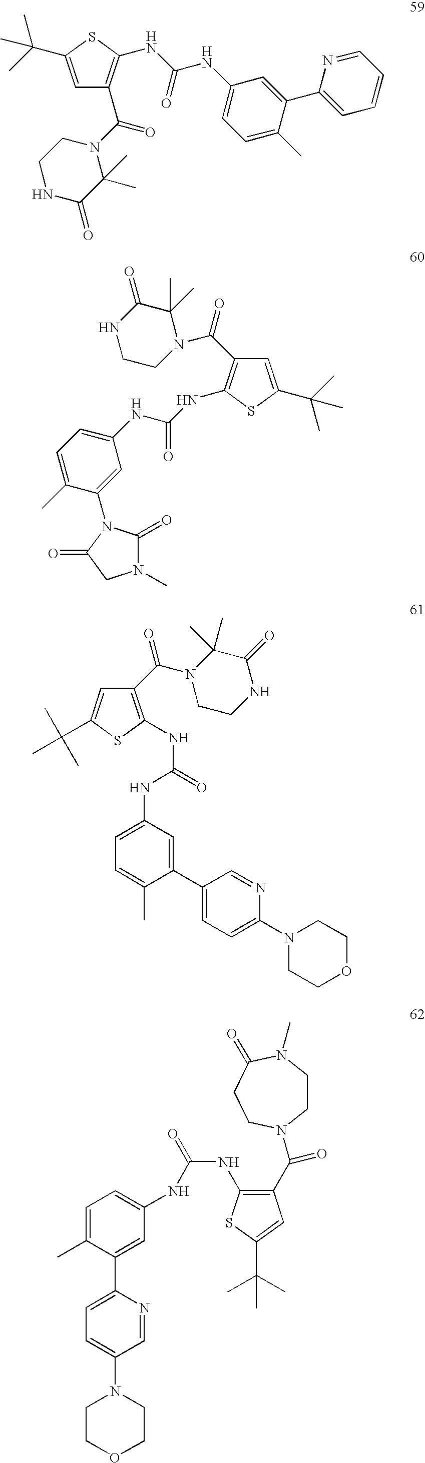

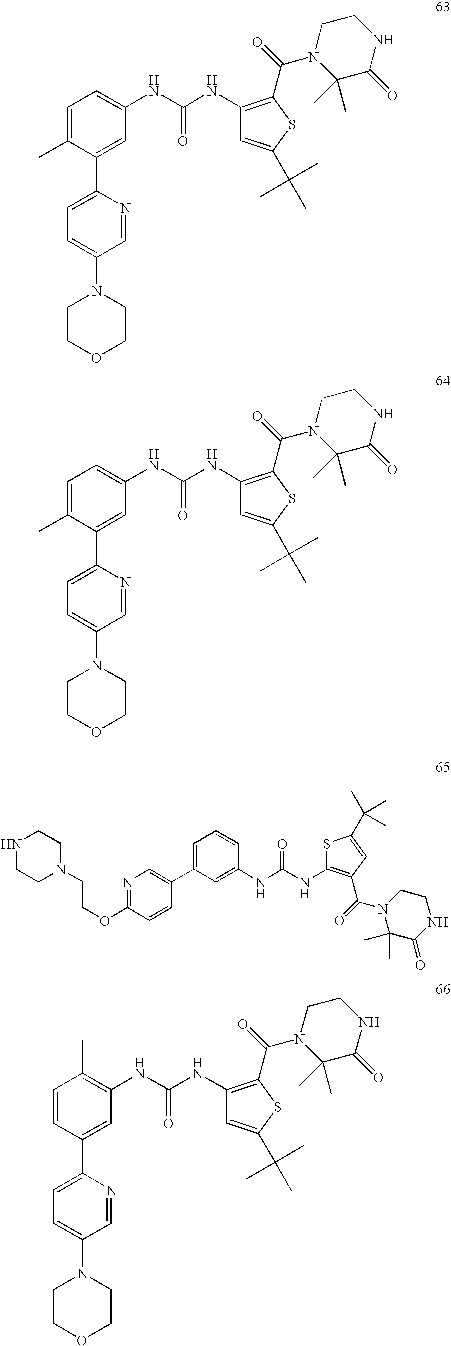

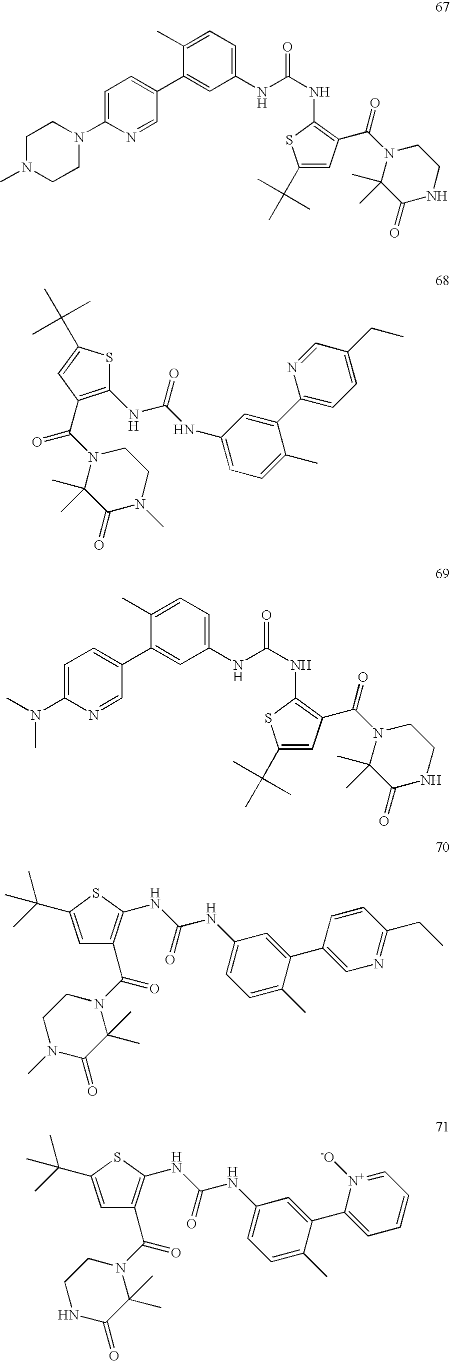

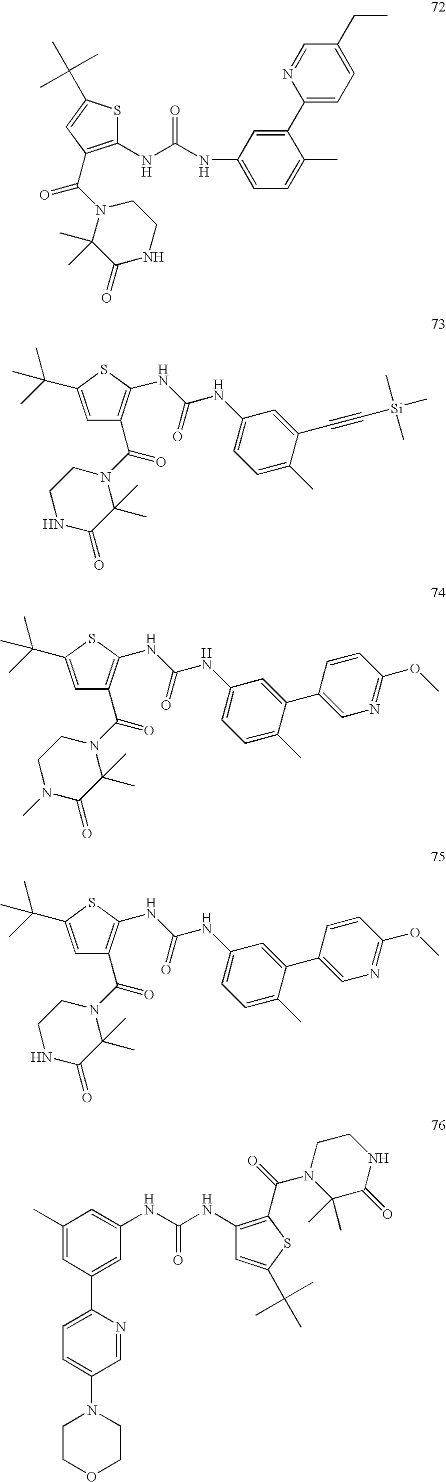

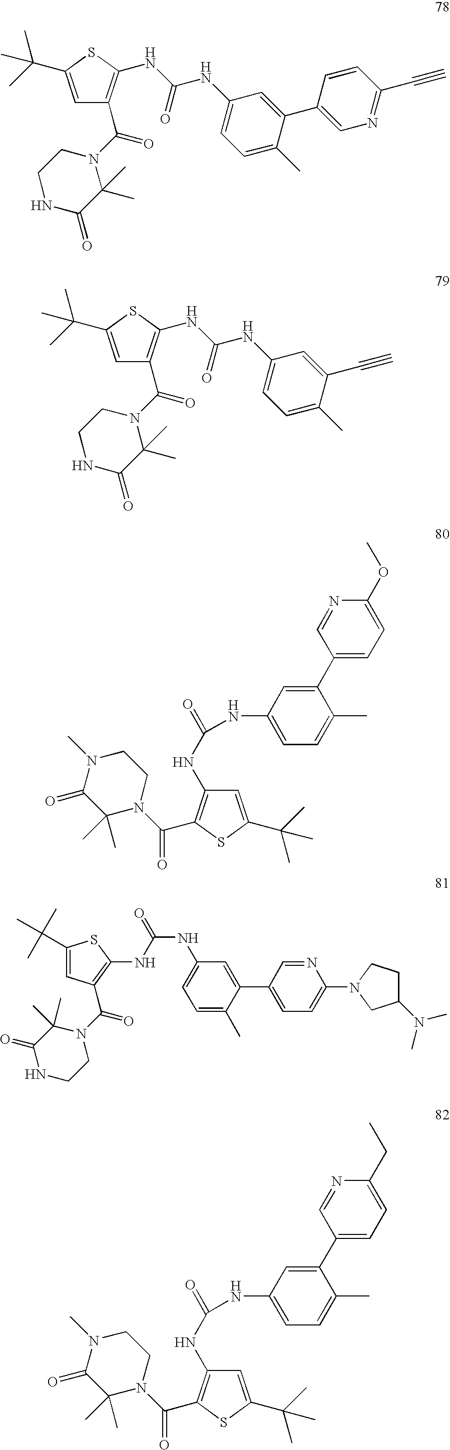

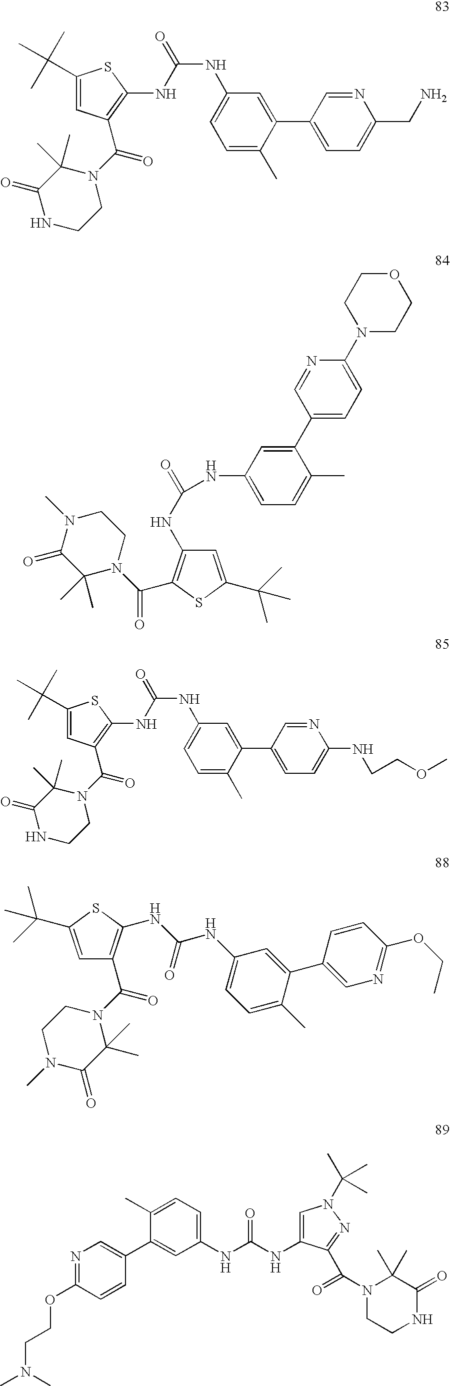

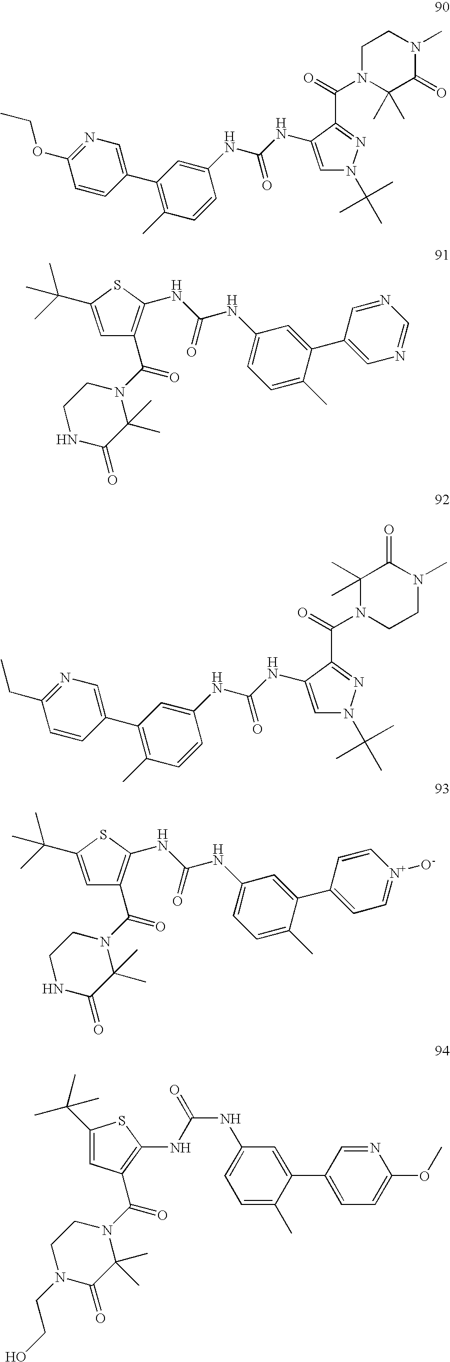

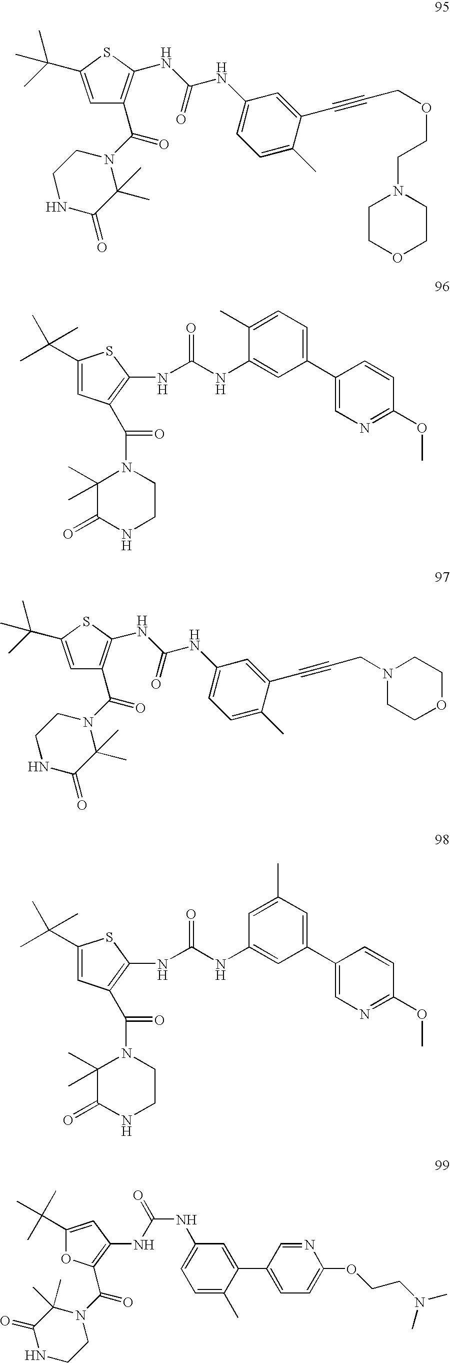

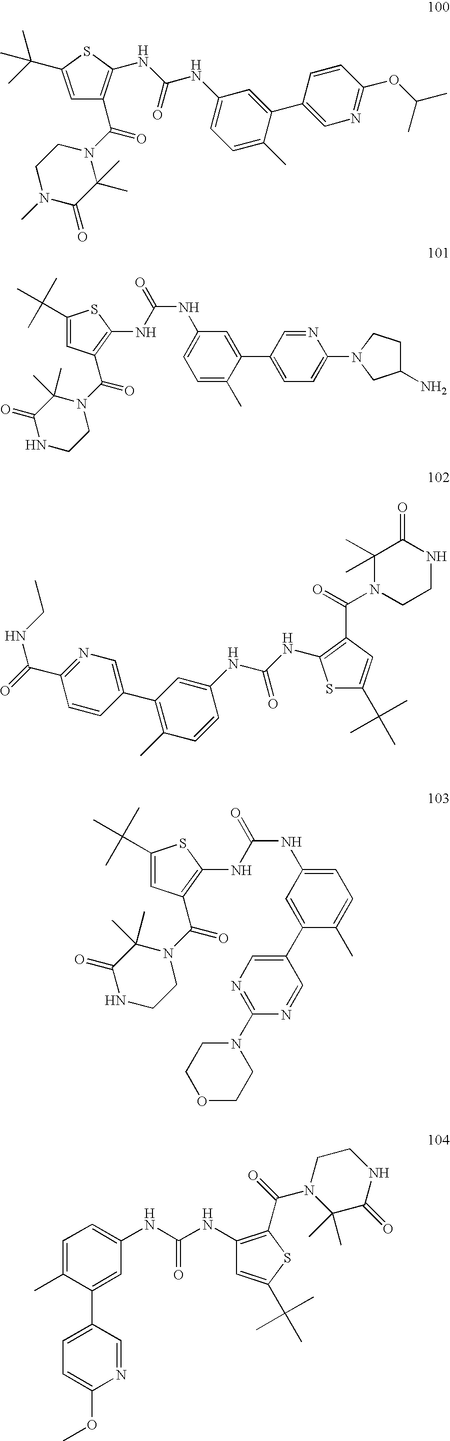

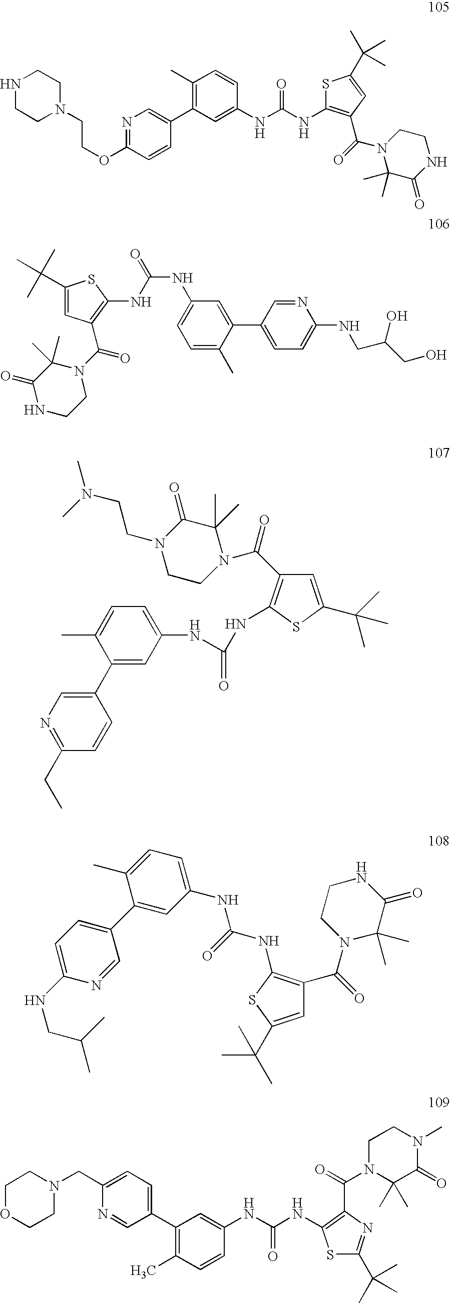

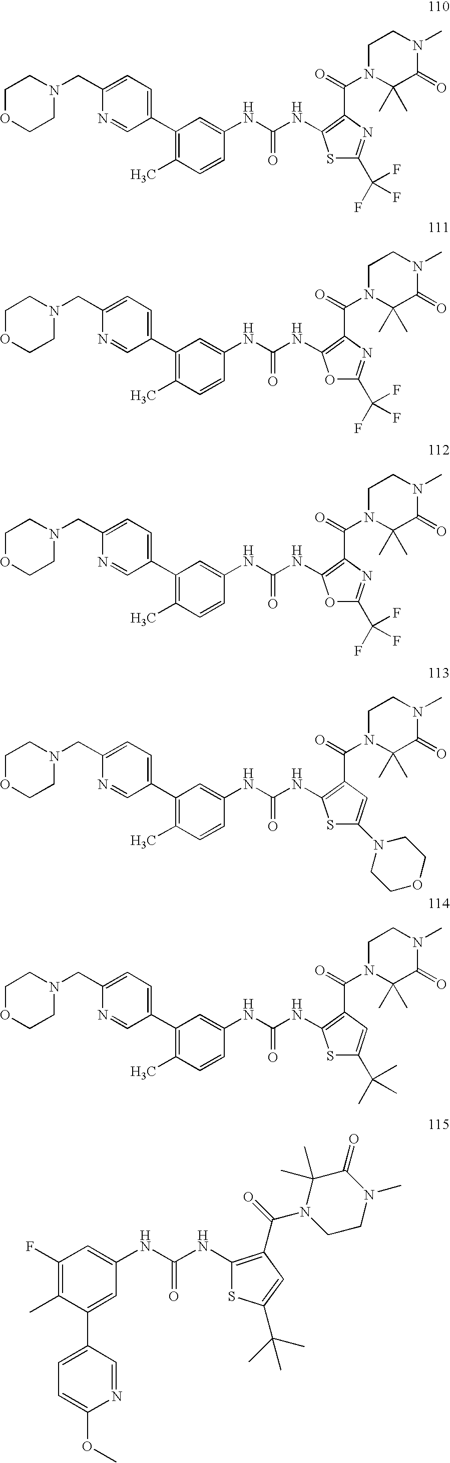

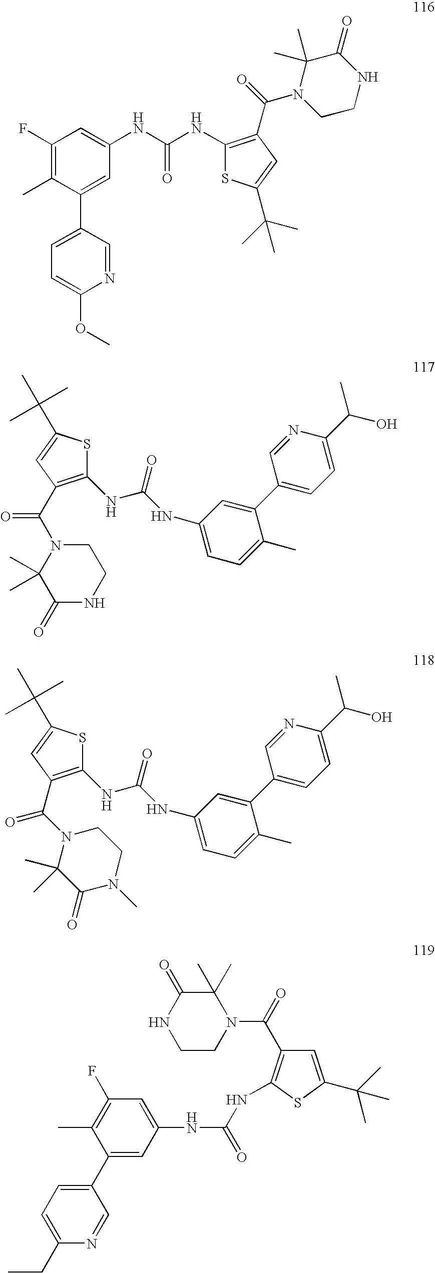

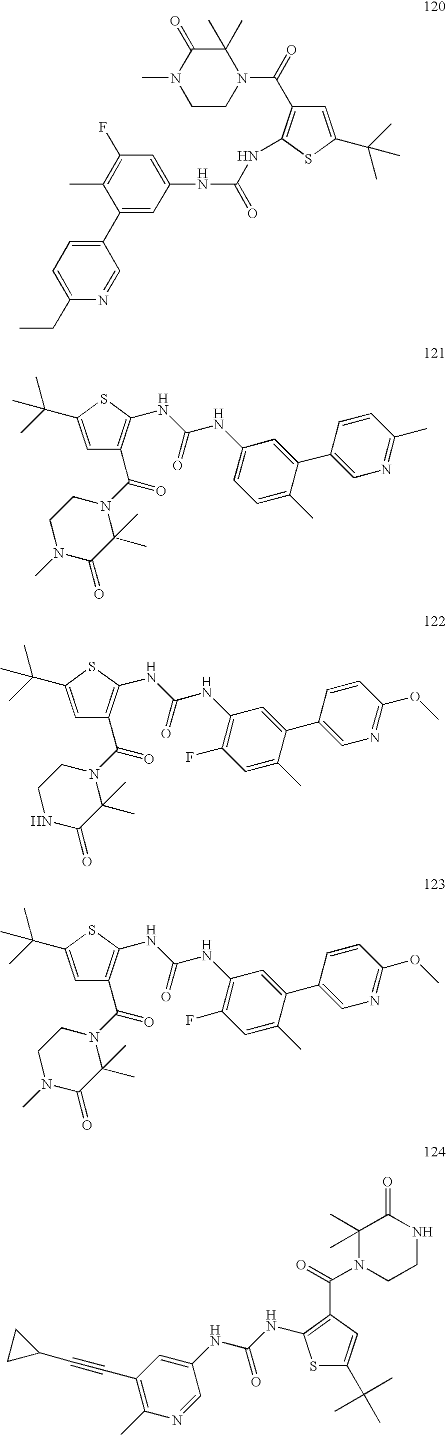

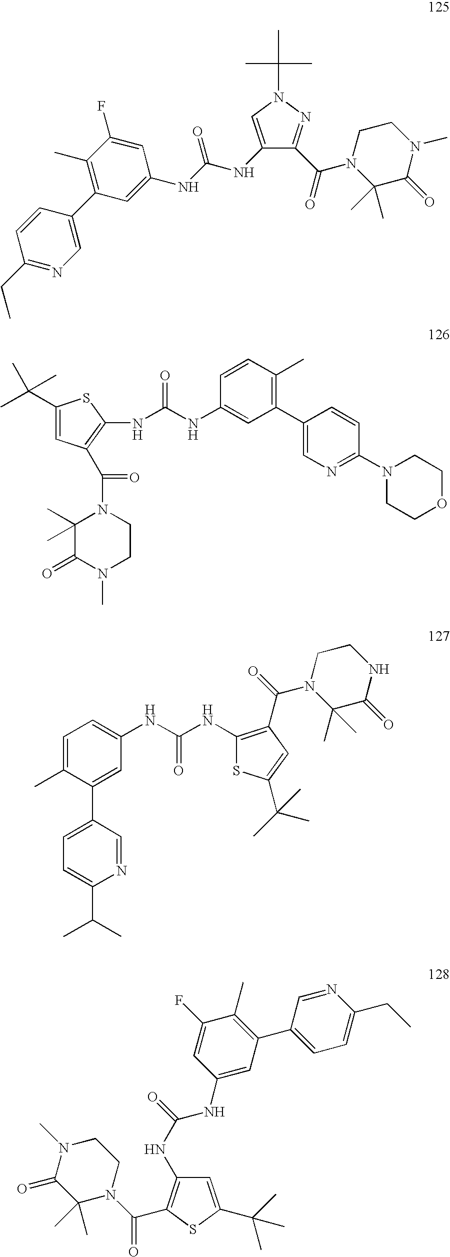

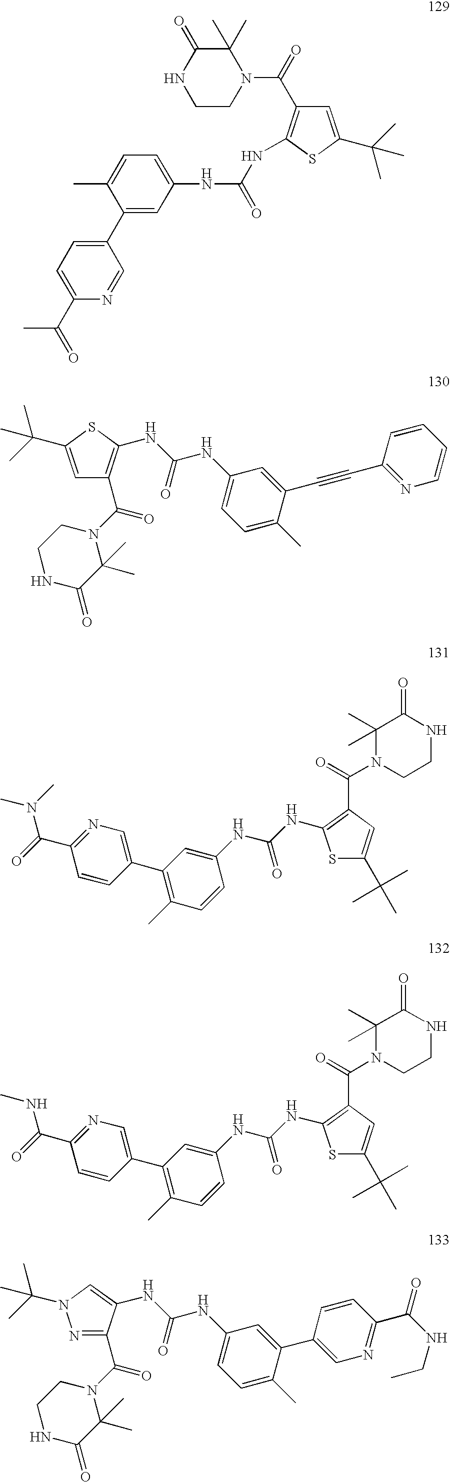

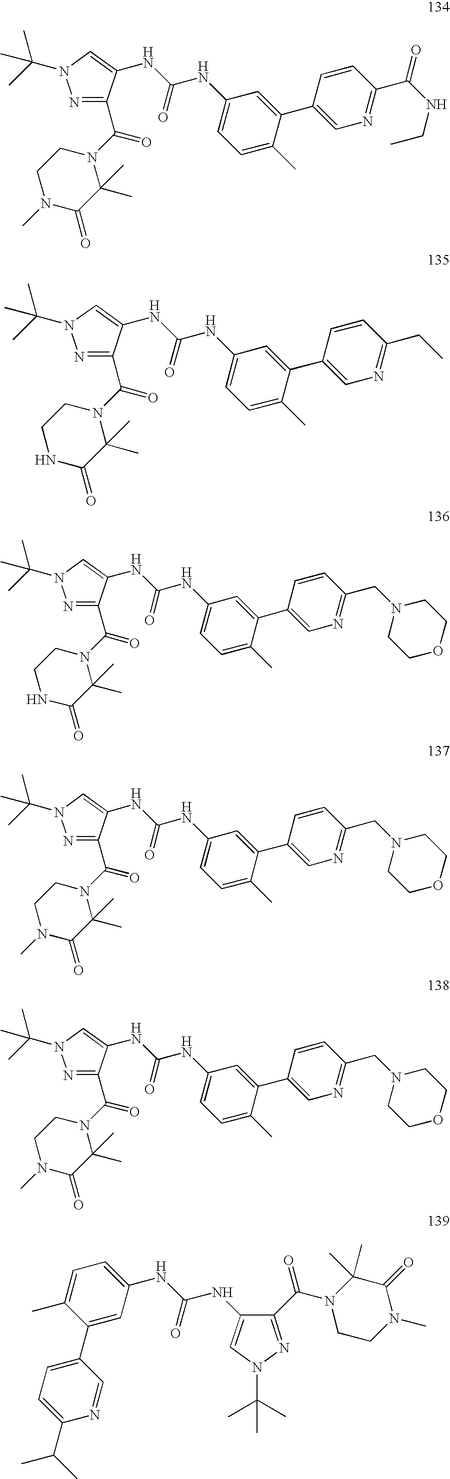

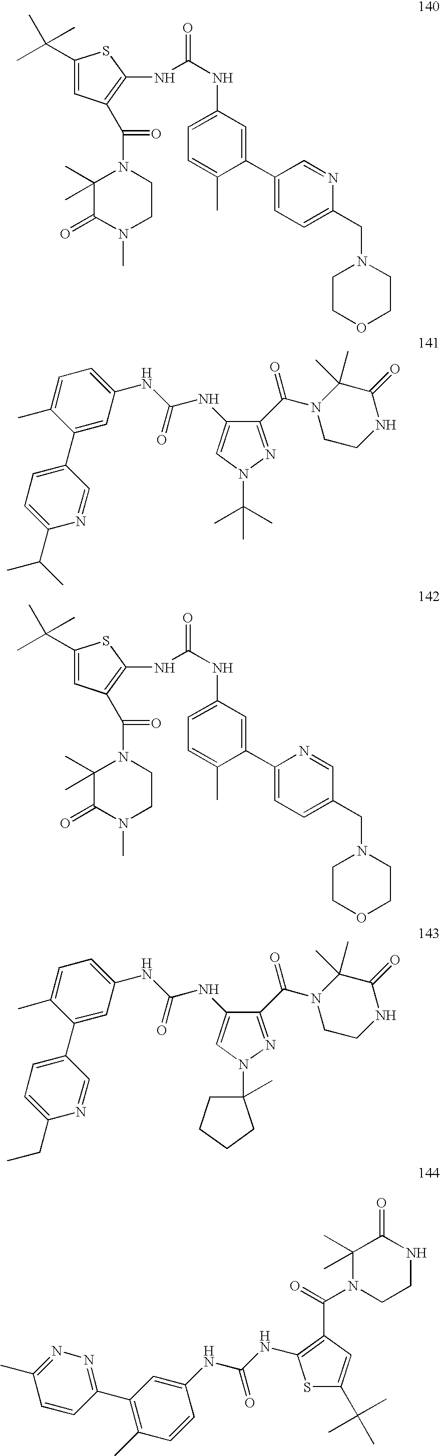

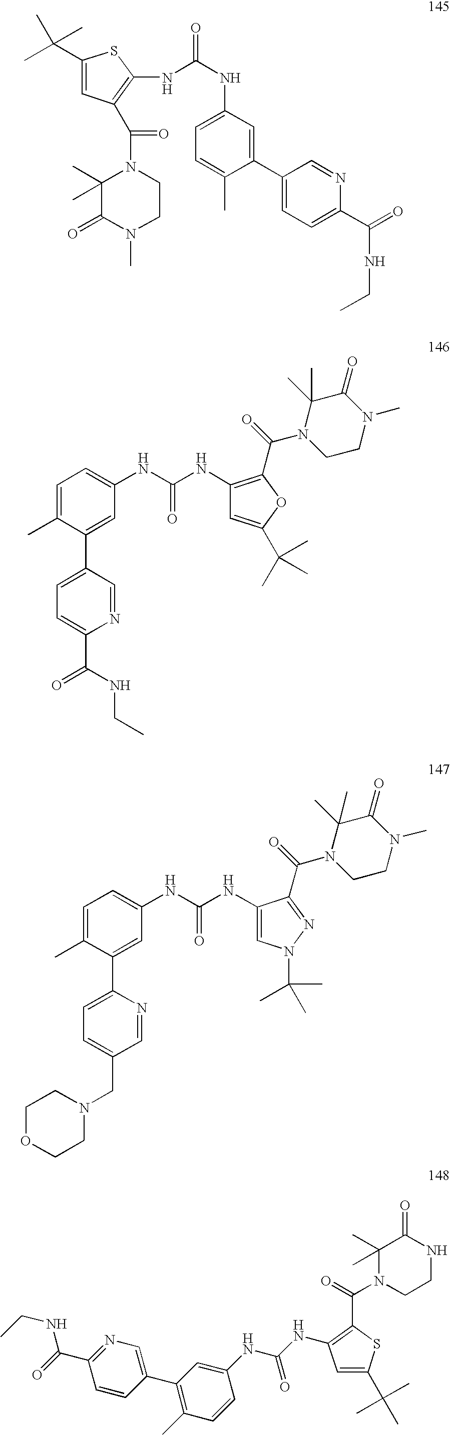

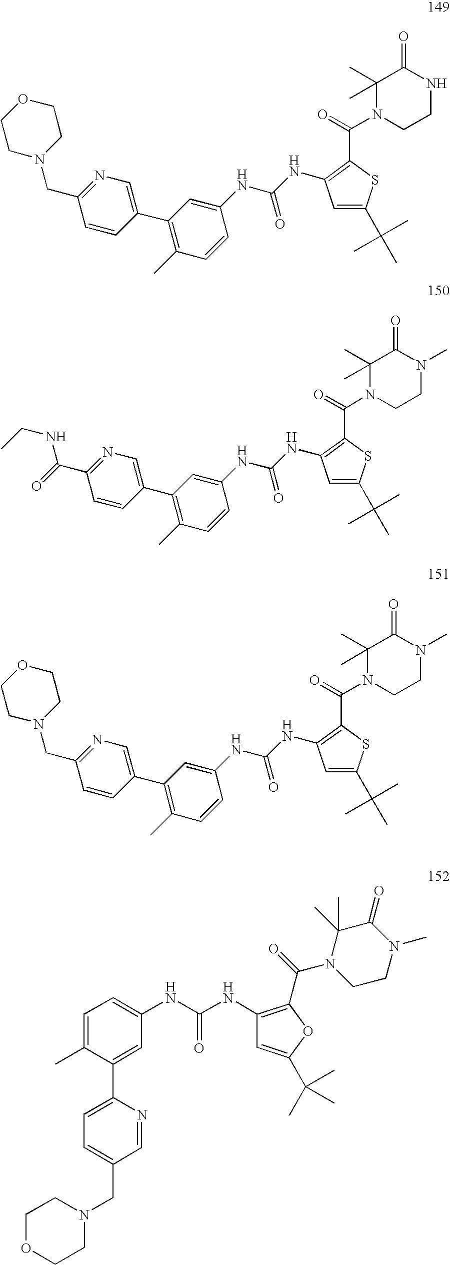

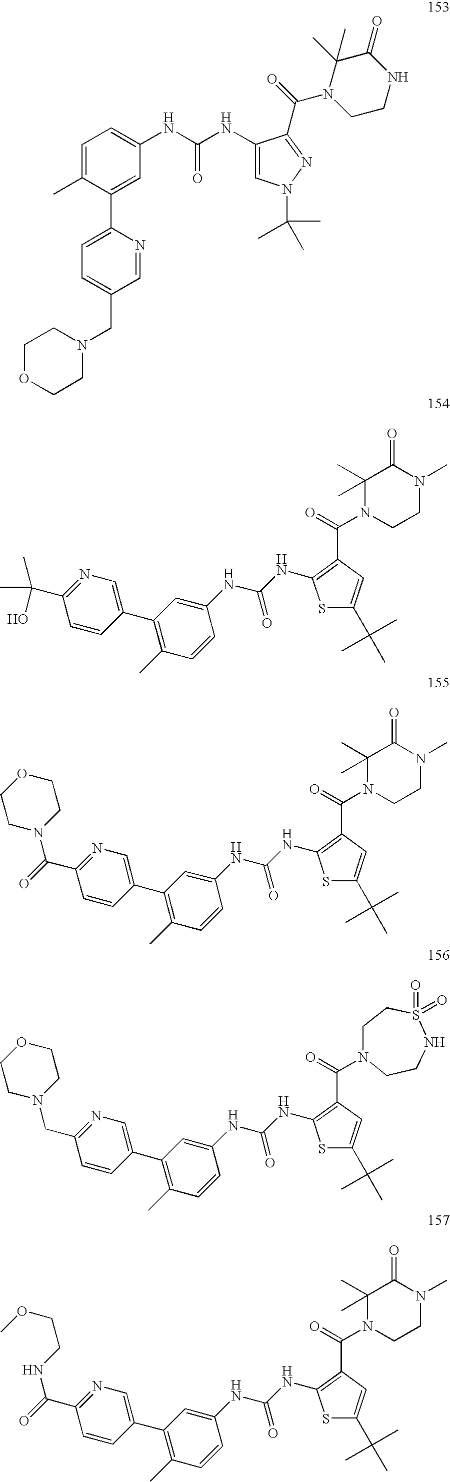

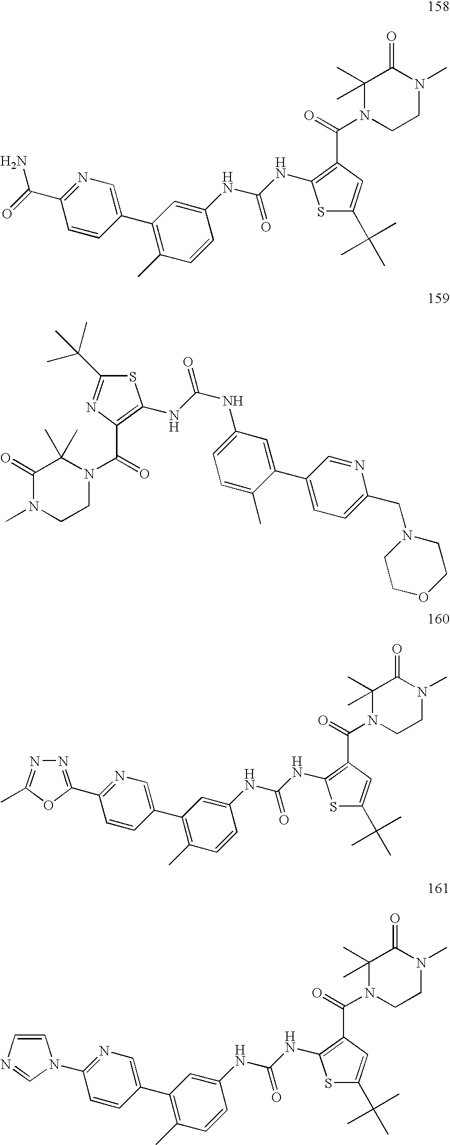

or pharmaceutically acceptable salt thereof, N-oxide thereof, or pharmaceutically acceptable salt of an N-oxide thereof.

What is claimed is:

1. A compound of general formula I: 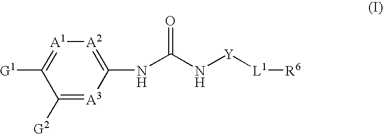
or a pharmaceutically acceptable salt, pharmaceutically acceptable N-oxide or pharmaceutically acceptable salt of an N-oxide, or solvate thereof:
wherein:
A¹ is N or CR¹;
A² is N or CR²;
A³ is N or CR³;
G¹ is hydrogen, halogen, C₁₋₆ alkyl, C₁₋₆ alkoxy or OR^{a};
G² is 5- or 6-membered heteroaryl, 5- or 6-membered heterocycloalkyl, C₂₋₆ alkynyl or cyano optionally substituted by one or more independently selected R^{A} groups;
R¹, R² and R³ are each, independently, hydrogen, halogen, hydroxyl, cyano, nitro, C₁₋₄ alkyl, C₁₋₄ haloalkyl, C₁₋₄ alkoxy, C₁₋₄ haloalkoxy, amino, C₁₋₄alkylamino, di-C₁₋₄alkylamino, or C₁₋₆ alkynyl;
Y is phenyl or 5- or 6-membered heteroaryl, either of which are optionally substituted with one or more independently selected R^{Y} groups;
L¹ is a single bond, CH₂ or —C(O)—;
R⁶ is 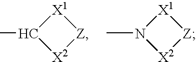
Z is —NR⁷—, —CHR⁷—, —C(O)—, —SR⁷—, —S(O)—, S(O₂)—, —NH—SO₂—;
X¹ and X² are each, independently, C₁₋₃ alkyl optionally substituted with one or more independently selected groups selected from oxo, hydroxyl, cyano, nitro, C₁₋₄ alkyl, C₁₋₄ haloalkyl, C₁₋₄ alkoxy, C₁₋₄ haloalkoxy, amino, C₁₋₄alkylamino and di-C₁₋₄alkylamino;
each R⁷ is, independently, hydrogen, cyano, C(O)R^{m}, C(O)C(O)R^{m}, C(O)C(O)NRⁿR^{o}, C(O)NRⁿR^{o}, C(O)OR^{p}, S(O)R^{p}, S(O)₂R^{p}, S(O)NRⁿR^{o}, S(O)₂R^{p}, C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀ aryl, C₆₋₁₀aryl-C₁₋₆ alkyl, C₅₋₉ heteroaryl, or C₅₋₉ heteroaryl-C₁₋₆ alkyl; wherein said C₁₋₆ alkyl, C₂₋₆ alkenyl, and C₂₋₆ alkynyl are each substituted with 1, 2, 3, or 4 independently selected R^{7′} groups; and wherein said C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀ aryl, C₆₋₁₀ aryl-C₁₋₆ alkyl, C₅₋₉ heteroaryl, or C₅₋₉ heteroaryl-C₁₋₆ alkyl are each optionally substituted with 1, 2, 3, or 4 independently selected R^{7″} groups;
each R^{Y} is, independently, halogen, hydroxyl, cyano, nitro, C₁₋₆ alkyl, C₁₋₆ haloalkyl, C₁₋₆ alkoxy, C₁₋₆ haloalkoxy, amino, amino, C₁₋₆ alkylamino, di-C₁₋₆ alkylamino, amino(C₁₋₆ alkyl), C₁₋₆ alkylamino(C₁₋₆ alkyl), di(C₁₋₆ alkyl)amino(C₁₋₆ alkyl), C₃₋₉ cycloalkyl, C₃₋₉ heterocycloalkyl, C₁₋₆ acyl, formyl, C₁₋₆ carbamyl, C₁₋₆ alkylcarbamoyl, di(C₁₋₆ alkyl)carbamyl, C₁₋₆ alkylcarbamoyloxy, di(C₁₋₆ alkyl)carbamyloxy, C₁₋₆ acyloxy, carboxy, C₁₋₆ alkylsulfonyl, C₁₋₆ alkylsulfonylamido, or di(C₁₋₆ alkyl)sulfonamide; wherein C₁₋₆ alkyl, C₂₋₆ alkenyl, and C₂₋₆ alkynyl each is optionally substituted with 1, 2, 3, or 4 independently selected R^{Y′} groups;
each R^{A} is, independently, halogen, cyano, nitro, tri(C₁₋₆)alkylsilyl, OR^{a}, C(O)R^{a}, C(O)NR^{e}R^{f}, C(O)OR^{a}, OC(O)R^{b}, OC(O)NR^{e}R^{f}, NR^{c}R^{d}, NR^{e}C(O)R^{f}, NR^{e}C(O)OR^{f}, NR^{e}C(O)NR^{f}, S(O)R^{a}, S(O)₂R^{a}, S(O)NR^{e}R^{f}, S(O)₂R^{a}, NR^{e}S(O)₂R^{f}, NR^{g}S(O)₂NR^{e}R^{f}, C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₅₋₁₀ aryl, C₅₋₁₀ aryl-C₁₋₆ alkyl, C₅₋₉ heteroaryl, or C₅₋₉ heteroaryl-C₁₋₆ alkyl; wherein C₁₋₆ alkyl, C₂₋₆ alkenyl, and C₂₋₆ alkynyl are each optionally substituted with 1, 2, 3, or 4 independently selected R^{A′} groups; and wherein said C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₅₋₁₀ aryl, C₅₋₁₀ aryl-C₁₋₆ alkyl, C₅₋₉ heteroaryl, or C₅₋₉ heteroaryl-C₁₋₆ alkyl are each optionally substituted by 1, 2, or 3 independently selected R^{A″} groups;
each R^{Y′}, R^{7′}, R^{7″}, R^{A′}, and R^{A″} ia, independently, halogen, cyano, nitro, hydroxyl, C₁₋₆ alkyl, C₁₋₆ haloalkyl, C₁₋₆ alkoxy, C₁₋₆ haloalkoxy, amino, C₁₋₆ alkylamino, di-C₁₋₆alkylamino, amino(C₁₋₆ alkyl), C₁₋₆ alkylamino(C₁₋₆alkyl), di(C₁₋₆alkyl)amino(C₁₋₆ alkyl), C₅₋₁₀ aryl, C₅₋₁₀ aryl-C₁₋₆ alkyl, C₅₋₉ heteroaryl, or C₅₋₉ heteroaryl-C₁₋₆ alkyl C₁₋₆ acyl, formyl, carboxy, C₁₋₆ alkyloxycarbonyl, C₁₋₆ carbamyl, C₁₋₆ alkylcarbamoyl, di(C₁₋₆ alkyl)carbamyl, C₁₋₆ alkylcarbamoyloxy, di(C₁₋₆ alkyl)carbamyloxy, C₁₋₆ acyloxy, C₁₋₆ alkylsulfonyl, C₁₋₆ alkylsulfonylamido or di(C₁₋₆ alkyl)sulfonamide;
each R^{a}, R^{c}, R^{d}, R^{e}, and R^{f} is, independently, hydrogen, C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₁₆alkyl, C₃₋₇ heterocycloalkyl, C₃₋₇ heterocycloalkyl-C₁₋₆ alkyl, C₆₋₁₀aryl, C₆₋₁₀aryl-C₁₋₆alkyl, C₅₋₉ heteroaryl, or C₅₋₉heteroaryl-C₁₋₆alkyl; wherein said C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀ aryl, C₆₋₁₀ aryl-C₁₋₆alkyl, C₅₋₉ heteroaryl, and C₅₋₉heteroaryl-C₁₋₆alkyl are each optionally substituted with 1, 2 or 3 independently selected R^{g} groups;
each R^{b} is, independently, C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀aryl, C₆₋₁₀ aryl-C₁₋₆ alkyl, C₅₋₉ heteroaryl, or C₅₋₉ heteroaryl-C₁₋₆ alkyl; wherein said C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀ aryl, C₆₋₁₀ aryl-C₁₋₆ alkyl, C₅₋₉ heteroaryl, and C₅₋₉ heteroaryl-C₁₋₆ alkyl are each optionally substituted with 1, 2 or 3 independently selected R^{h} groups;
each R^{m}, Rⁿ, R^{o}, and R^{p} is, independently, hydrogen, C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀ aryl, C₆₋₁₀ aryl-C₁₋₆ alkyl, C₅₋₉ heteroaryl, or C₅₋₉ heteroaryl-C₁₋₆ alkyl; wherein said C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀ aryl, C₆₋₁₀ aryl-C₁₋₆ alkyl, C₅₋₉ heteroaryl, and C₅₋₉ heteroaryl-C₁₋₆ alkyl are each optionally substituted with 1, 2 or 3 independently selected Rⁱ groups;
each R^{g}, R^{h}, and Rⁱ is, independently, halogen, C₁₋₆ alkyl, C₁₋₆ haloalkyl, hydroxyl, C₁₋₆ alkoxy, C₁₋₆ haloalkoxy, cyano, nitro, amino, C₁₋₆alkylamino, or di-C₁₋₆alkylamino.

2. The compound of claim 1, wherein Y is 5- or 6-membered heteroaryl, either of which are optionally substituted with one or more independently selected R^{Y} groups.

3. A pharmaceutical composition comprising:
a compound of general formula I: 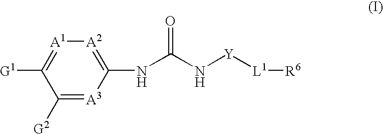
or a pharmaceutically acceptable salt, pharmaceutically acceptable N-oxide or pharmaceutically acceptable salt of an N-oxide, or solvate thereof:
wherein:
A¹ is N or CR¹;
A² is N or CR²;
A³ is N or CR³;
G¹ is hydrogen, halogen, C₁₋₆ alkyl, C₁₋₆ alkoxy or OR^{a};
G² is 5- or 6-membered heteroaryl, 5- or 6-membered heterocycloalkyl, C₂₋₆ alkynyl or cyano optionally substituted by one or more independently selected R^{A} groups;
R¹, R² and R³ are each, independently, hydrogen, halogen, hydroxyl, cyano, nitro, C₁₋₄ alkyl, C₁₋₄ haloalkyl, C₁₋₄ alkoxy, C₁₋₄ haloalkoxy, amino, C₁₋₄alkylamino, di-C₁₋₄alkylamino, or C₁₋₆ alkynyl;
Y is phenyl or 5- or 6-membered heteroaryl, either of which are optionally substituted with one or more independently selected R^{Y} groups;
L¹ is a single bond, CH₂ or —C(O)—;
R⁶ is 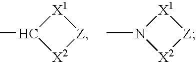
Z is —NR⁷—, —CHR⁷—, —C(O)—, —SR⁷—, —S(O)—, S(O₂)—, —NH—SO₂—;
X¹ and X² are each, independently, C₁₋₃ alkyl optionally substituted with one or more independently selected groups selected from oxo, hydroxyl, cyano, nitro, C₁₋₄ alkyl, C₁₋₄ haloalkyl, C₁₋₄ alkoxy, C₁₋₄ haloalkoxy, amino, C₁₋₄alkylamino and di-C₁₋₄alkylamino;
each R⁷ is, independently, hydrogen, cyano, C(O)R^{m}, C(O)C(O)R^{m}, C(O)C(O)NRⁿR^{o}, C(O)NRⁿR^{o}, C(O)OR^{p}, S(O)R^{p}, S(O)₂R^{p}, S(O)NRⁿR^{o}, S(O)₂R^{p}, C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀ aryl, C₆₋₁₀aryl-C₁₋₆ alkyl, C₅₋₉ heteroaryl, or C₅₋₉ heteroaryl-C₁₋₆ alkyl; wherein said C₁₋₆ alkyl, C₂₋₆ alkenyl, and C₂₋₆ alkynyl are each substituted with 1, 2, 3, or 4 independently selected R^{7′} groups; and wherein said C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀ aryl, C₆₋₁₀ aryl-C₁₋₆ alkyl, C₅₋₉ heteroaryl, or C₅₋₉ heteroaryl-C₁₋₆ alkyl are each optionally substituted with 1, 2, 3, or 4 independently selected R^{7″} groups;
each R^{Y} is, independently, halogen, hydroxyl, cyano, nitro, C₁₋₆ alkyl, C₁₋₆ haloalkyl, C₁₋₆ alkoxy, C₁₋₆ haloalkoxy, amino, amino, C₁₋₆ alkylamino, di-C₁₋₆ alkylamino, amino(C₁₋₆ alkyl), C₁₋₆ alkylamino(C₁₋₆ alkyl), di(C₁₋₆ alkyl)amino(C₁₋₆ alkyl), C₃₋₉ cycloalkyl, C₃₋₉ heterocycloalkyl, C₁₋₆ acyl, formyl, C₁₋₆ carbamyl, C₁₋₆ alkylcarbamoyl, di(C₁₋₆ alkyl)carbamyl, C₁₋₆ alkylcarbamoyloxy, di(C₁₋₆ alkyl)carbamyloxy, C₁₋₆ acyloxy, carboxy, C₁₋₆ alkylsulfonyl, C₁₋₆ alkylsulfonylamido, or di(C₁₋₆ alkyl)sulfonamide; wherein C₁₋₆ alkyl, C₂₋₆ alkenyl, and C₂₋₆ alkynyl each is optionally substituted with 1, 2, 3, or 4 independently selected R^{Y′} groups;
each R^{A} is, independently, halogen, cyano, nitro, tri(C₁₋₆)alkylsilyl, OR^{a}, C(O)R^{a}, C(O)NR^{e}R^{f}, C(O)OR^{a}, OC(O)R^{b}, OC(O)NR^{e}R^{f}, NR^{c}R^{d}, NR^{e}C(O)R^{f}, NR^{e}C(O)OR^{f}, NR^{e}C(O)NR^{f}, S(O)R^{a}, S(O)₂R^{a}, S(O)NR^{e}R^{f}, S(O)₂R^{a}, NR^{e}S(O)₂R^{f}, NR^{g}S(O)₂NR^{e}R^{f}, C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₅₋₁₀ aryl, C₅₋₁₀ aryl-C₁₋₆ alkyl, C₅₋₉ heteroaryl, or C₅₋₉ heteroaryl-C₁₋₆ alkyl; wherein C₁₋₆ alkyl, C₂₋₆ alkenyl, and C₂₋₆ alkynyl are each optionally substituted with 1, 2, 3, or 4 independently selected R^{A′} groups; and wherein said C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₅₋₁₀ aryl, C₅₋₁₀ aryl-C₁₋₆ alkyl, C₅₋₉ heteroaryl, or C₅₋₉ heteroaryl-C₁₋₆ alkyl are each optionally substituted by 1, 2, or 3 independently selected R^{A″} groups;
each R^{Y′}, R^{7′}, R^{7″}, R^{A′}, and R^{A″} ia, independently, halogen, cyano, nitro, hydroxyl, C₁₋₆ alkyl, C₁₋₆ haloalkyl, C₁₋₆ alkoxy, C₁₋₆ haloalkoxy, amino, C₁₋₆ alkylamino, di-C₁₋₆alkylamino, amino(C₁₋₆ alkyl), C₁₋₆ alkylamino(C₁₋₆alkyl), di(C₁₋₆alkyl)amino(C₁₋₆ alkyl), C₅₋₁₀ aryl, C₅₋₁₀ aryl-C₁₋₆ alkyl, C₅₋₉ heteroaryl, or C₅₋₉ heteroaryl-C₁₋₆ alkyl C₁₋₆ acyl, formyl, carboxy, C₁₋₆ alkyloxycarbonyl, C₁₋₆ carbamyl, C₁₋₆ alkylcarbamoyl, di(C₁₋₆ alkyl)carbamyl, C₁₋₆ alkylcarbamoyloxy, di(C₁₋₆ alkyl)carbamyloxy, C₁₋₆ acyloxy, C₁₋₆ alkylsulfonyl, C₁₋₆ alkylsulfonylamido or di(C₁₋₆ alkyl)sulfonamide;
each R^{a}, R^{c}, R^{d}, R^{e}, and R^{f} is, independently, hydrogen, C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀aryl, C₆₋₁₀aryl-C₁₋₆alkyl, C₅₋₉ heteroaryl, or C₅₋₉heteroaryl-C₁₋₆alkyl; wherein said C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀ aryl, C₆₋₁₀ aryl-C₁₋₆alkyl, C₅₋₉ heteroaryl, and C₅₋₉heteroaryl-C₁₋₆alkyl are each optionally substituted with 1, 2 or 3 independently selected R^{g} groups;
each R^{b} is, independently, C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀aryl, C₆₋₁₀ aryl-C₁₋₆ alkyl, C₅₋₉ heteroaryl, or C₅₋₉ heteroaryl-C₁₋₆ alkyl; wherein said C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀ aryl, C₆₋₁₀ aryl-C₁₋₆ alkyl, C₅₋₉ heteroaryl, and C₅₋₉ heteroaryl-C₁₋₆ alkyl are each optionally substituted with 1, 2 or 3 independently selected R^{h} groups;
each R^{m}, Rⁿ, R^{o}, and R^{p} is, independently, hydrogen, C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀ aryl, C₆₋₁₀ aryl-C₁₋₆ alkyl, C₅₋₉ heteroaryl, or C₅₋₉ heteroaryl-C₁₋₆ alkyl; wherein said C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀ aryl, C₆₋₁₀ aryl-C₁₋₆ alkyl, C₅₋₉ heteroaryl, and C₅₋₉ heteroaryl-C₁₋₆ alkyl are each optionally substituted with 1, 2 or 3 independently selected Rⁱ groups;
each R^{g}, R^{h}, and Rⁱ is, independently, halogen, C₁₋₆ alkyl, C₁₋₆ haloalkyl, hydroxyl, C₁₋₆ alkoxy, C₁₋₆ haloalkoxy, cyano, nitro, amino, C₁₋₆alkylamino, or di-C₁₋₆alkylamino; and
a pharmaceutically acceptable carrier or excipient.

4. A method for inhibiting p38 MAP kinase in a subject:
administering to a subject in need of treatment a pharmaceutical composition comprising:
a compound of general formula I: 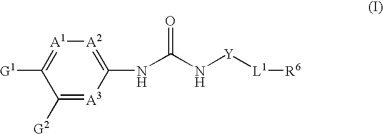
or a pharmaceutically acceptable salt, pharmaceutically acceptable N-oxide or pharmaceutically acceptable salt of an N-oxide, or solvate thereof:
wherein:
A¹ is N or CR¹;
A² is N or CR²;
A³ is N or CR³;
G¹ is hydrogen, halogen, C₁₋₆ alkyl, C₁₋₆ alkoxy or OR^{a};
G² is 5- or 6-membered heteroaryl, 5- or 6-membered heterocycloalkyl, C₂₋₆ alkynyl or cyano optionally substituted by one or more independently selected R^{A} groups;
R¹, R² and R³ are each, independently, hydrogen, halogen, hydroxyl, cyano, nitro, C₁₋₄ alkyl, C₁₋₄ haloalkyl, C₁₋₄ alkoxy, C₁₋₄ haloalkoxy, amino, C₁₋₄alkylamino, di-C₁₋₄alkylamino, or C₁₋₆ alkynyl;
Y is phenyl or 5- or 6-membered heteroaryl, either of which are optionally substituted with one or more independently selected R^{Y} groups;
L¹ is a single bond, CH₂ or —C(O)—;
R⁶ is 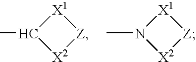
Z is —NR⁷—, —CHR⁷—, —C(O)—, —SR⁷—, —S(O)—, S(O₂)—, —NH—SO₂—;
X¹ and X² are each, independently, C₁₋₃ alkyl optionally substituted with one or more independently selected groups selected from oxo, hydroxyl, cyano, nitro, C₁₋₄ alkyl, C₁₋₄ haloalkyl, C₁₋₄ alkoxy, C₁₋₄ haloalkoxy, amino, C₁₋₄alkylamino and di-C₁₋₄alkylamino;
each R⁷ is, independently, hydrogen, cyano, C(O)R^{m}, C(O)C(O)R^{m}, C(O)C(O)NRⁿR^{o}, C(O)NRⁿR^{o}, C(O)OR^{p}, S(O)R^{p}, S(O)₂R^{p}, S(O)NRⁿR^{o}, S(O)₂R^{p}, C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀ aryl, C₆₋₁₀aryl-C₁₋₆ alkyl, C₅₋₉ heteroaryl, or C₅₋₉ heteroaryl-C₁₋₆ alkyl; wherein said C₁₋₆ alkyl, C₂₋₆ alkenyl, and C₂₋₆ alkynyl are each substituted with 1, 2, 3, or 4 independently selected R^{7′} groups; and wherein said C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀ aryl, C₆₋₁₀ aryl-C₁₋₆ alkyl, C₅₋₉ heteroaryl, or C₅₋₉ heteroaryl-C₁₋₆ alkyl are each optionally substituted with 1, 2, 3, or 4 independently selected R^{7″} groups;
each R^{Y} is, independently, halogen, hydroxyl, cyano, nitro, C₁₋₆ alkyl, C₁₋₆ haloalkyl, C₁₋₆ alkoxy, C₁₋₆ haloalkoxy, amino, amino, C₁₋₆ alkylamino, di-C₁₋₆ alkylamino, amino(C₁₋₆ alkyl), C₁₋₆ alkylamino(C₁₋₆ alkyl), di(C₁₋₆ alkyl)amino(C₁₋₆ alkyl), C₃₋₉ cycloalkyl, C₃₋₉ heterocycloalkyl, C₁₋₆ acyl, formyl, C₁₋₆ carbamyl, C₁₋₆ alkylcarbamoyl, di(C₁₋₆ alkyl)carbamyl, C₁₋₆ alkylcarbamoyloxy, di(C₁₋₆ alkyl)carbamyloxy, C₁₋₆ acyloxy, carboxy, C₁₋₆ alkylsulfonyl, C₁₋₆ alkylsulfonylamido, or di(C₁₋₆ alkyl)sulfonamide; wherein C₁₋₆ alkyl, C₂₋₆ alkenyl, and C₂₋₆ alkynyl each is optionally substituted with 1, 2, 3, or 4 independently selected R^{Y′} groups;
each R^{A} is, independently, halogen, cyano, nitro, tri(C₁₋₆)alkylsilyl, OR^{a}, C(O)R^{a}, C(O)NR^{e}R^{f}, C(O)OR^{a}, OC(O)R^{b}, OC(O)NR^{e}R^{f}, NR^{c}R^{d}, NR^{e}C(O)R^{f}, NR^{e}C(O)OR^{f}, NR^{e}C(O)NR^{f}, S(O)R^{a}, S(O)₂R^{a}, S(O)NR^{e}R^{f}, S(O)₂R^{a}, NR^{e}S(O)₂R^{f}, NR^{g}S(O)₂NR^{e}R^{f}, C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₅₋₁₀ aryl, C₅₋₁₀ aryl-C₁₋₆ alkyl, C₅₋₉ heteroaryl, or C₅₋₉ heteroaryl-C₁₋₆ alkyl; wherein C₁₋₆ alkyl, C₂₋₆ alkenyl, and C₂₋₆ alkynyl are each optionally substituted with 1, 2, 3, or 4 independently selected R^{A′} groups; and wherein said C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₅₋₁₀ aryl, C₅₋₁₀ aryl-C₁₋₆ alkyl, C₅₋₉ heteroaryl, or C₅₋₉ heteroaryl-C₁₋₆ alkyl are each optionally substituted by 1, 2, or 3 independently selected R^{A″} groups;
each R^{Y′}, R^{7′}, R^{7″}, R^{A′}, and R^{A″} ia, independently, halogen, cyano, nitro, hydroxyl, C₁₋₆ alkyl, C₁₋₆ haloalkyl, C₁₋₆ alkoxy, C₁₋₆ haloalkoxy, amino, C₁₋₆ alkylamino, di-C₁₋₆alkylamino, amino(C₁₋₆ alkyl), C₁₋₆ alkylamino(C₁₋₆alkyl), di(C₁₋₆alkyl)amino(C₁₋₆ alkyl), C₅₋₁₀ aryl, C₅₋₁₀ aryl-C₁₋₆ alkyl, C₅₋₉ heteroaryl, or C₅₋₉ heteroaryl-C₁₋₆ alkyl C₁₋₆ acyl, formyl, carboxy, C₁₋₆ alkyloxycarbonyl, C₁₋₆ carbamyl, C₁₋₆ alkylcarbamoyl, di(C₁₋₆ alkyl)carbamyl, C₁₋₆ alkylcarbamoyloxy, di(C₁₋₆ alkyl)carbamyloxy, C₁₋₆ acyloxy, C₁₋₆ alkylsulfonyl, C₁₋₆ alkylsulfonylamido or di(C₁₋₆ alkyl)sulfonamide;
each R^{a}, R^{c}, R^{d}, R^{e}, and R^{f} is, independently, hydrogen, C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀aryl, C₆₋₁₀aryl-C₁₋₆alkyl, C₅₋₉ heteroaryl, or C₅₋₉heteroaryl-C₁₋₆alkyl; wherein said C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀ aryl, C₆₋₁₀ aryl-C₁₋₆alkyl, C₅₋₉ heteroaryl, and C₅₋₉heteroaryl-C₁₋₆alkyl are each optionally substituted with 1, 2 or 3 independently selected R^{g} groups;
each R^{b} is, independently, C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀aryl, C₆₋₁₀ aryl-C₁₋₆ alkyl, C₅₋₉ heteroaryl, or C₅₋₉ heteroaryl-C₁₋₆ alkyl; wherein said C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀ aryl, C₆₋₁₀ aryl-C₁₋₆ alkyl, C₅₋₉ heteroaryl, and C₅₋₉ heteroaryl-C₁₋₆ alkyl are each optionally substituted with 1, 2 or 3 independently selected R^{h} groups;
each R^{m}, Rⁿ, R^{o}, and R^{p} is, independently, hydrogen, C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆alkyl, C₃₋₇ heterocycloalkyl, C₃₋₇ heterocycloalkyl-C₁₋₆ alkyl, C₆₋₁₀ aryl, C₆₋₁₀ aryl-C₁₋₆ alkyl, C₅₋₉ heteroaryl, or C₅₋₉ heteroaryl-C₁₋₆ alkyl; wherein said C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₆ alkyl, C₃₋₇ heterocycloalkyl, C₃₋₇ heterocycloalkyl-C₁₋₆ alkyl, C₆₋₁₀ aryl, C₆₋₁₀ aryl-C₁₋₆ alkyl, C₅₋₉ heteroaryl, and C₅₋₉ heteroaryl-C₁₋₆ alkyl are each optionally substituted with 1, 2 or 3 independently selected Rⁱ groups;
each R^{g}, R^{h}, and Rⁱ is, independently, halogen, C₁₋₆ alkyl, C₁₋₆ haloalkyl, hydroxyl, C₁₋₆ alkoxy, C₁₋₆ haloalkoxy, cyano, nitro, amino, C₁₋₆alkylamino, or di-C₁₋₆alkylamino; and
a pharmaceutically acceptable carrier or excipient.

5. A method for preparing a compound of Formula I comprising:
performing the steps of at least one of Methods I to XIV.

6. The compound of claim 1, wherein
G¹ is methyl;
G² is 6-membered heteroaryl substituted by one or more independently selected C₅₋₉ heteroaryl-C₁₋₆ alkyl;
L is —C(O)—;
Y is 5-heteroaryl, optionally substituted with one or more independently selected C₁₋₆ alkyl;
R⁶ is 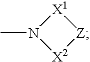
Z is —NR⁷—;
X¹ and X² are each, independently, C₁₋₃ alkyl optionally substituted with one or more independently selected groups selected from oxo, hydroxyl, cyano, nitro, C₁₋₄ alkyl, C₁₋₄ haloalkyl, C₁₋₄ alkoxy, C₁₋₄ haloalkoxy, amino, C₁₋₄alkylamino and di-C₁₋₄alkylamino;
or a pharmaceutically acceptable salt thereof.

7. A compound in accordance with claim 1, selected from 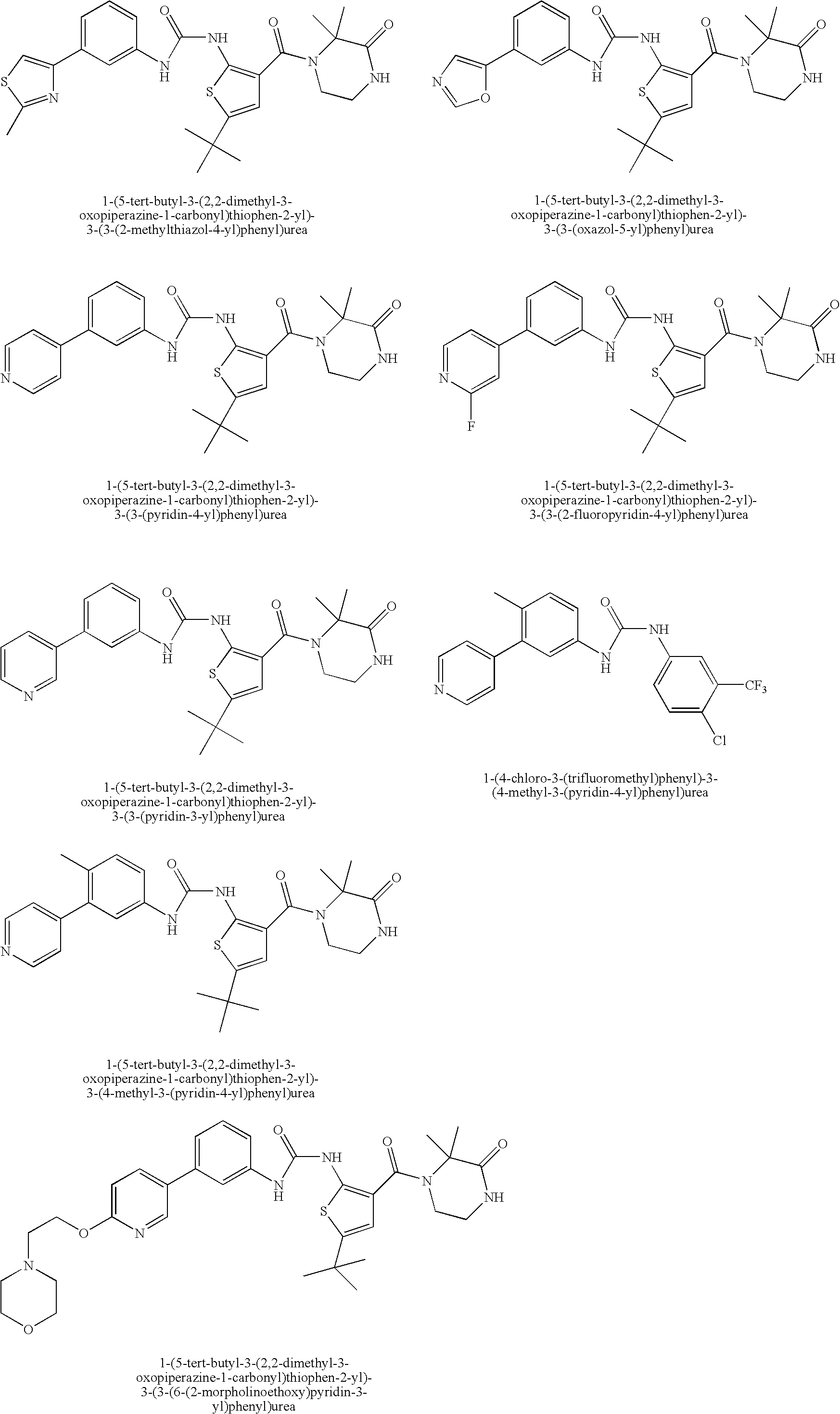 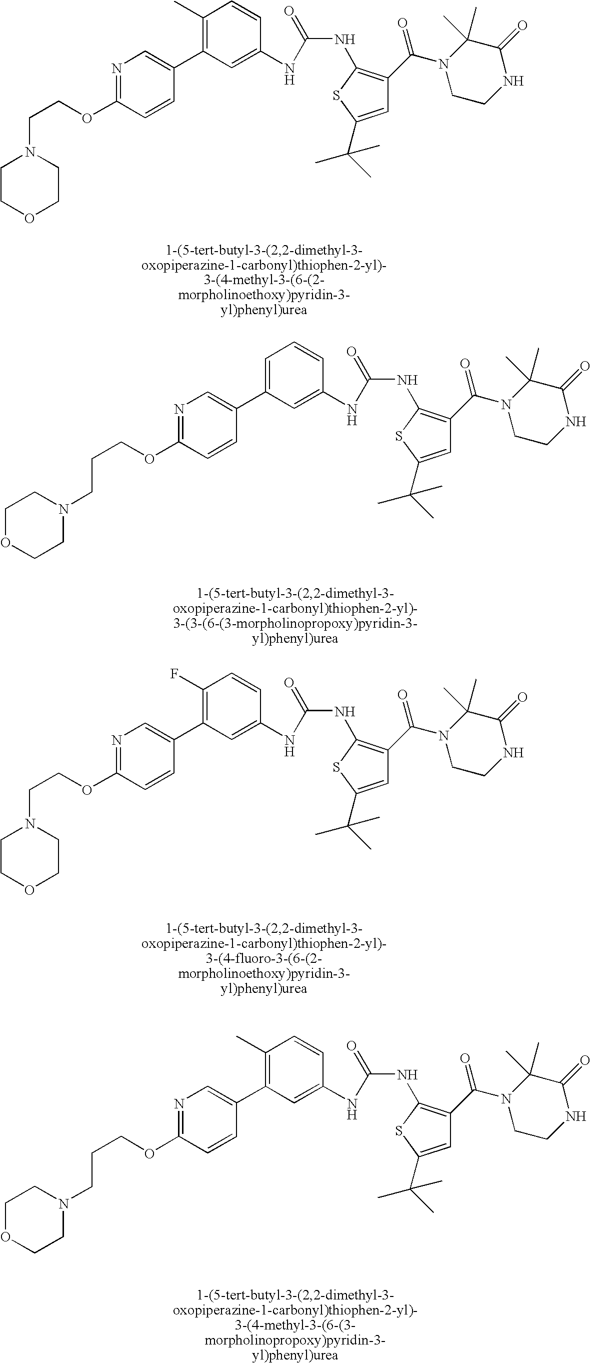 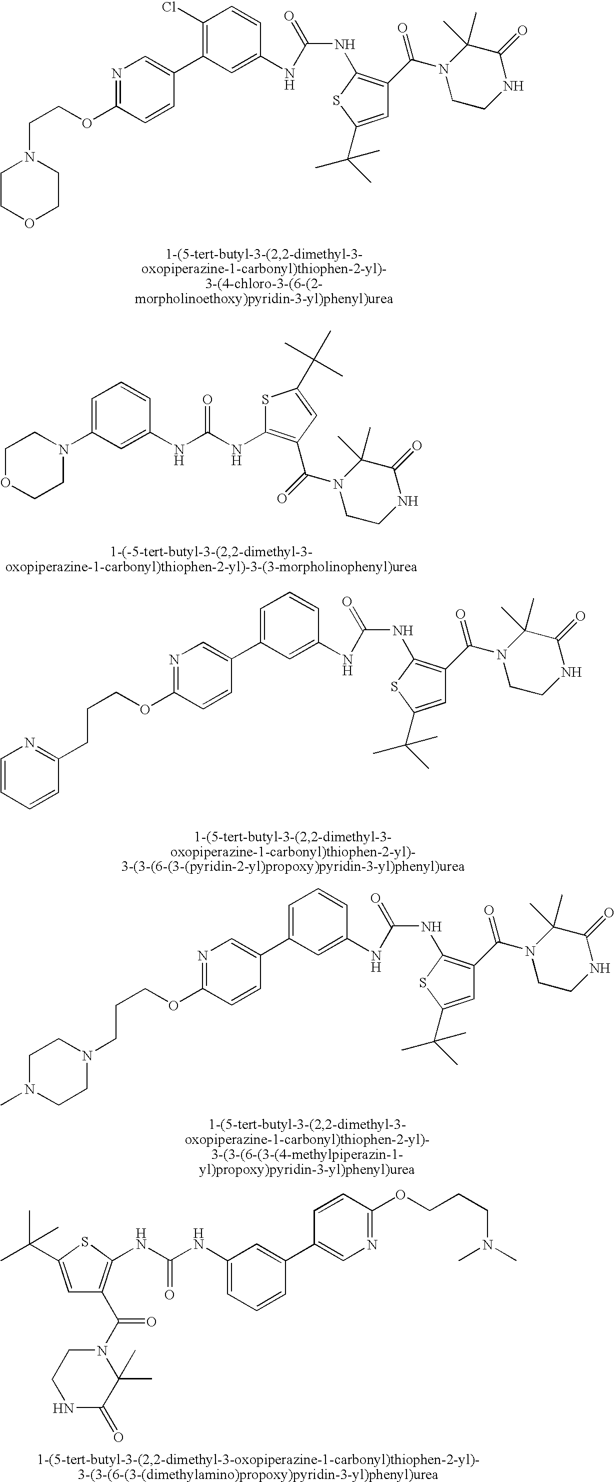 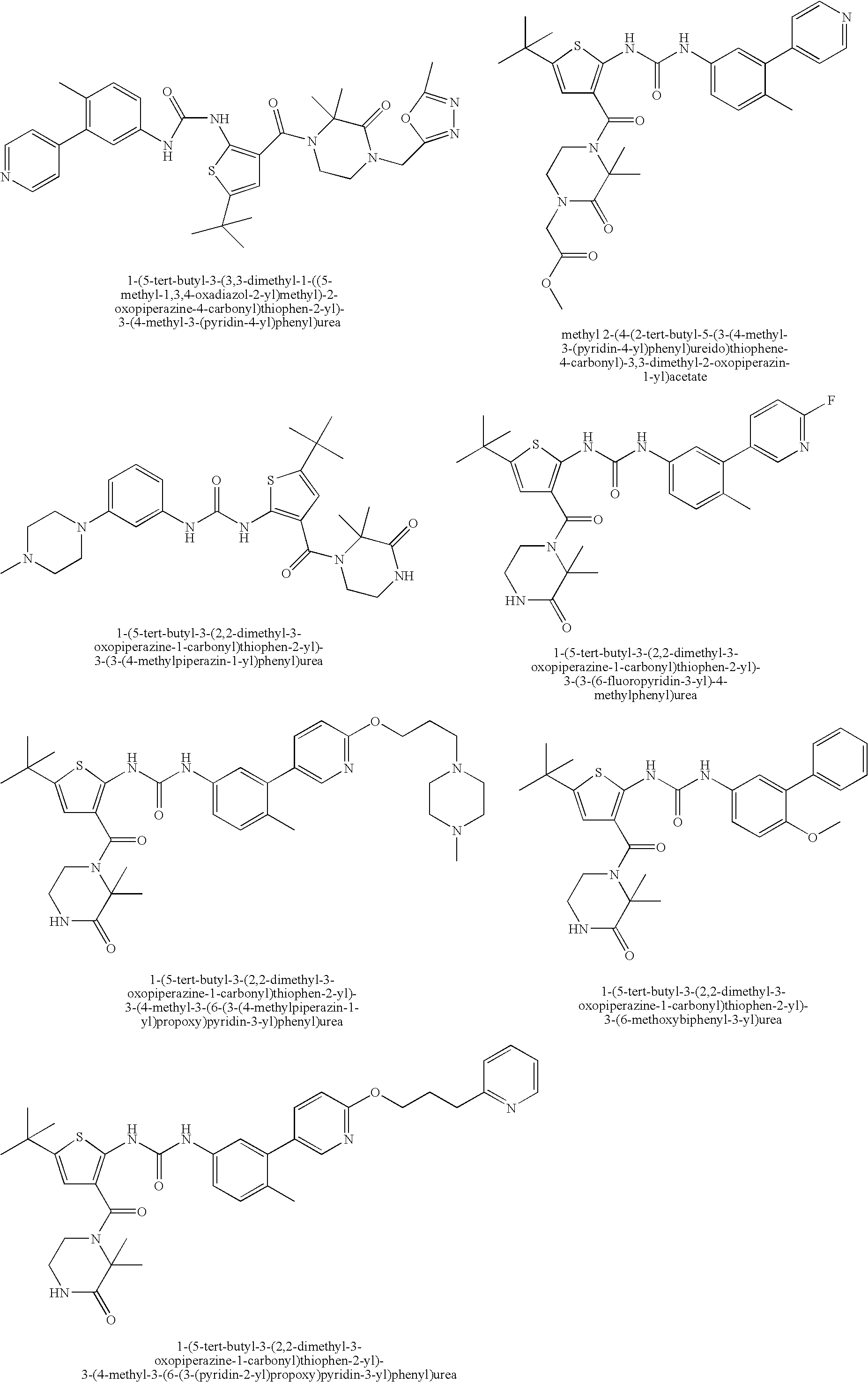 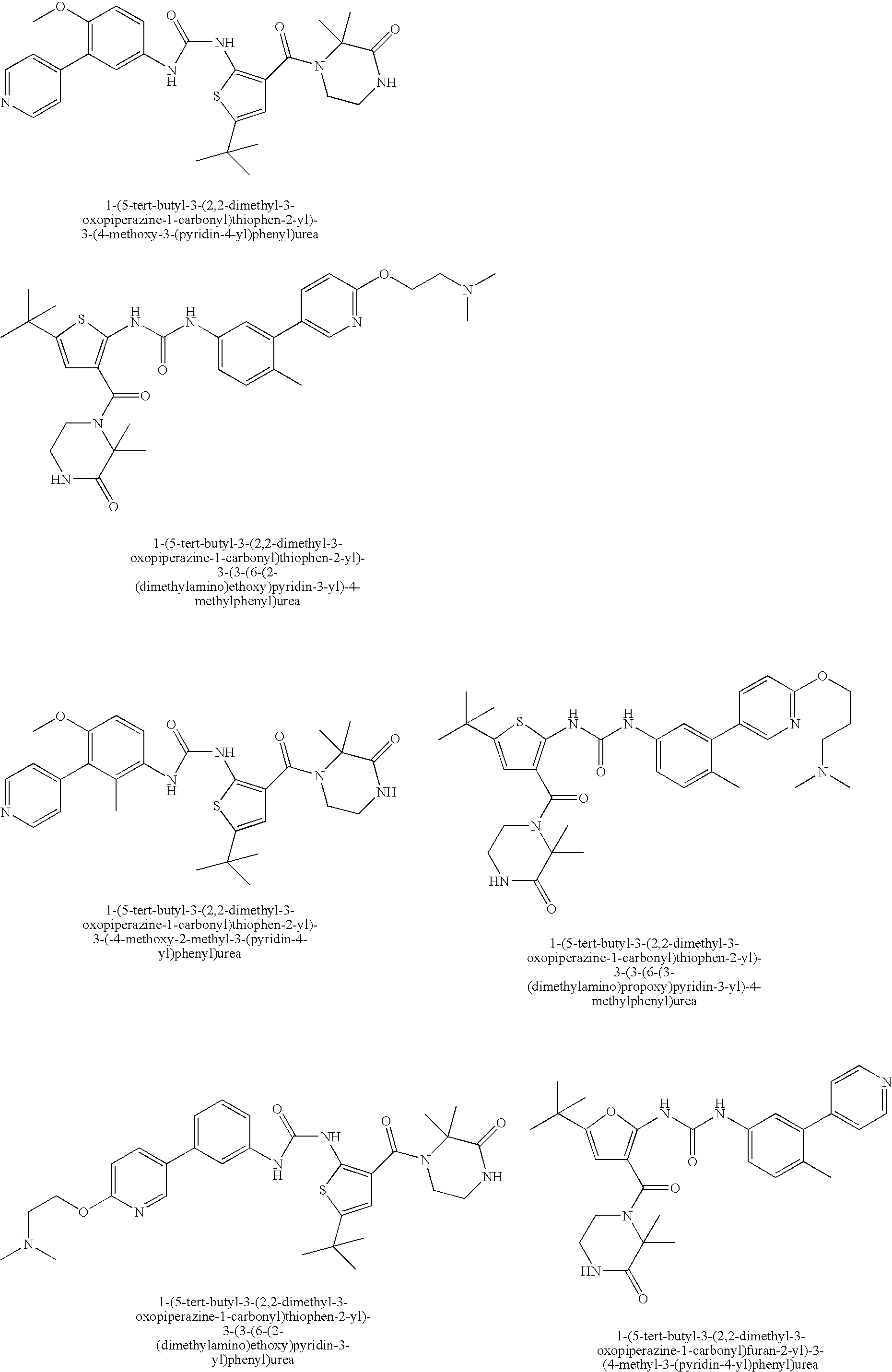 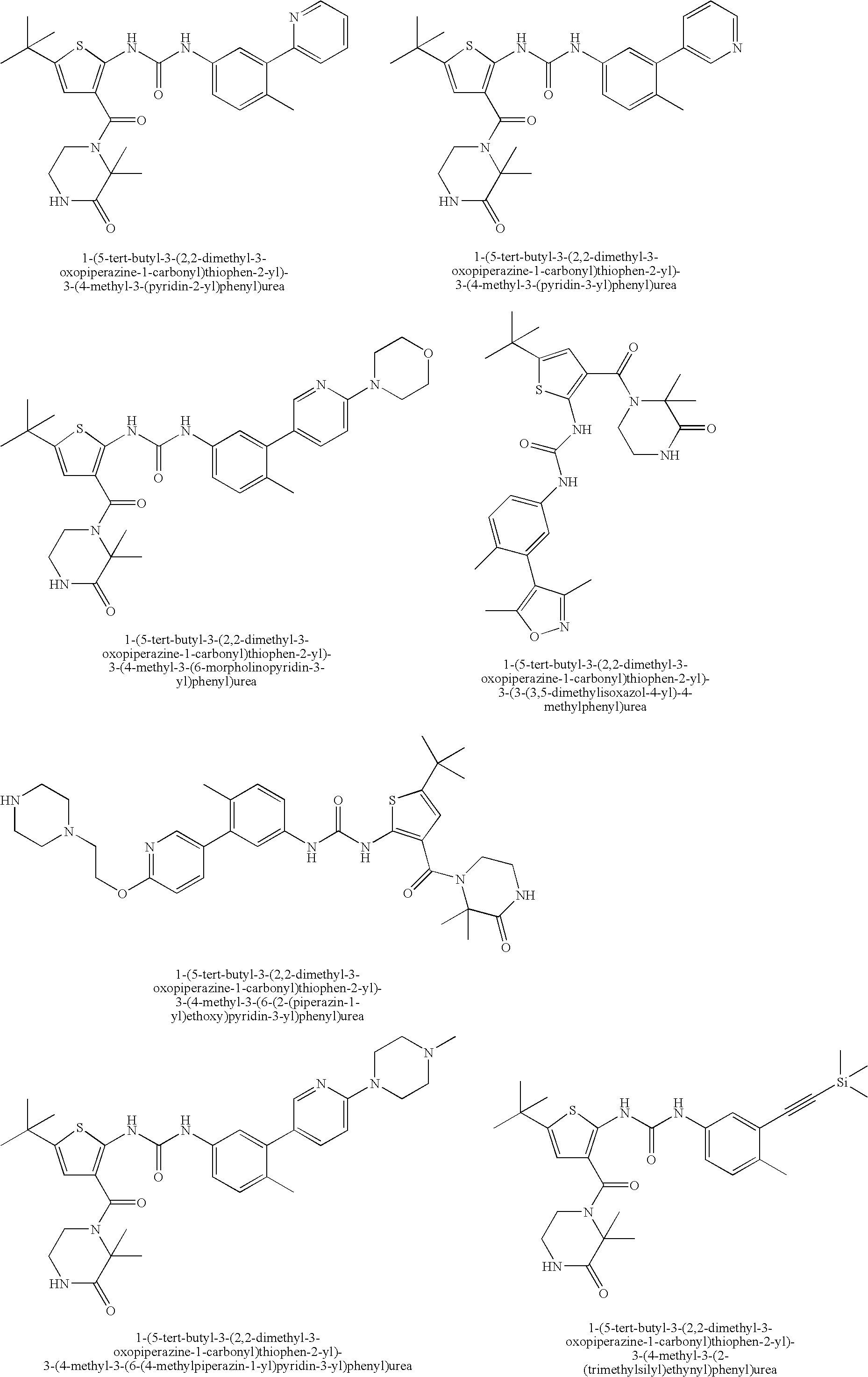 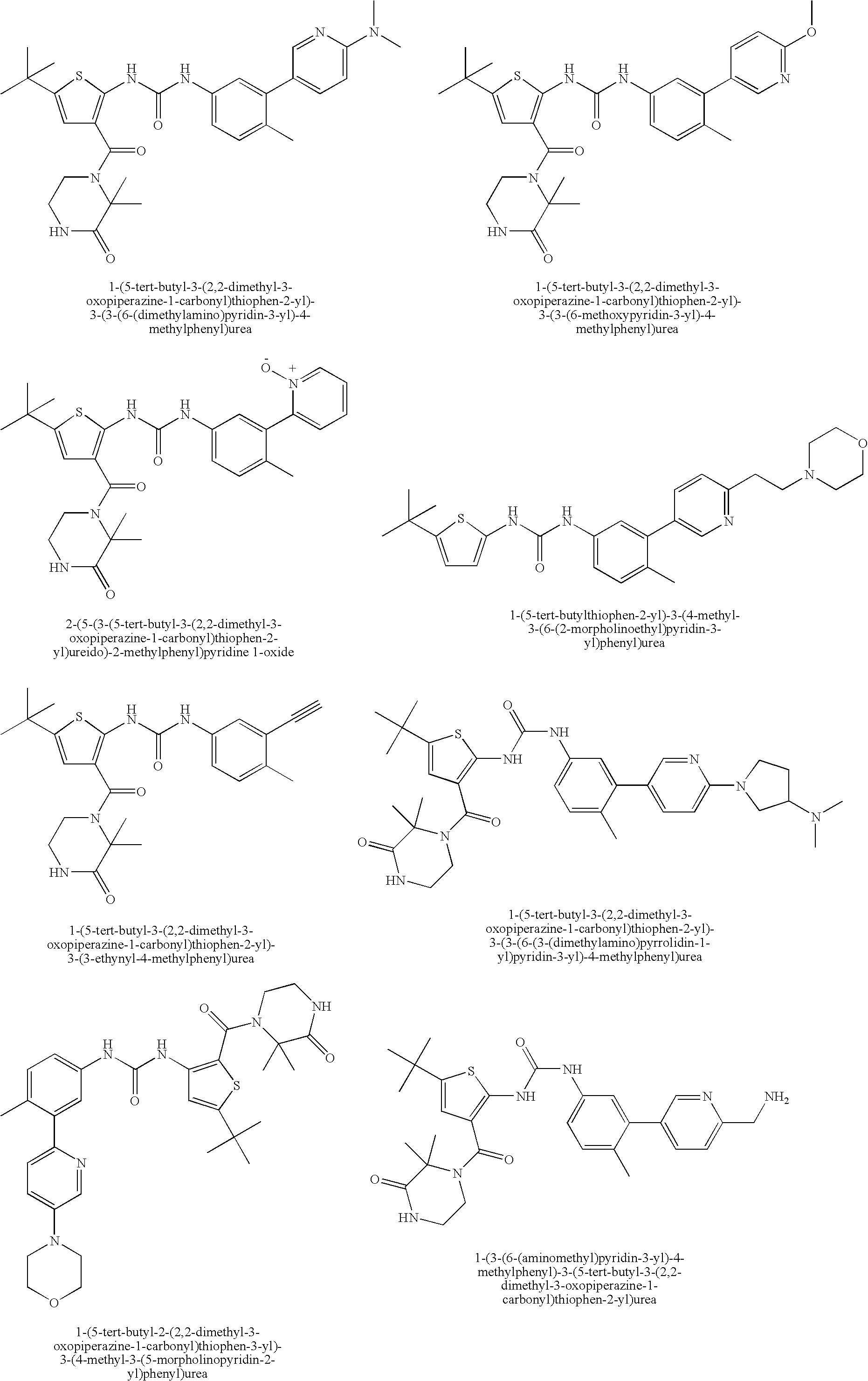 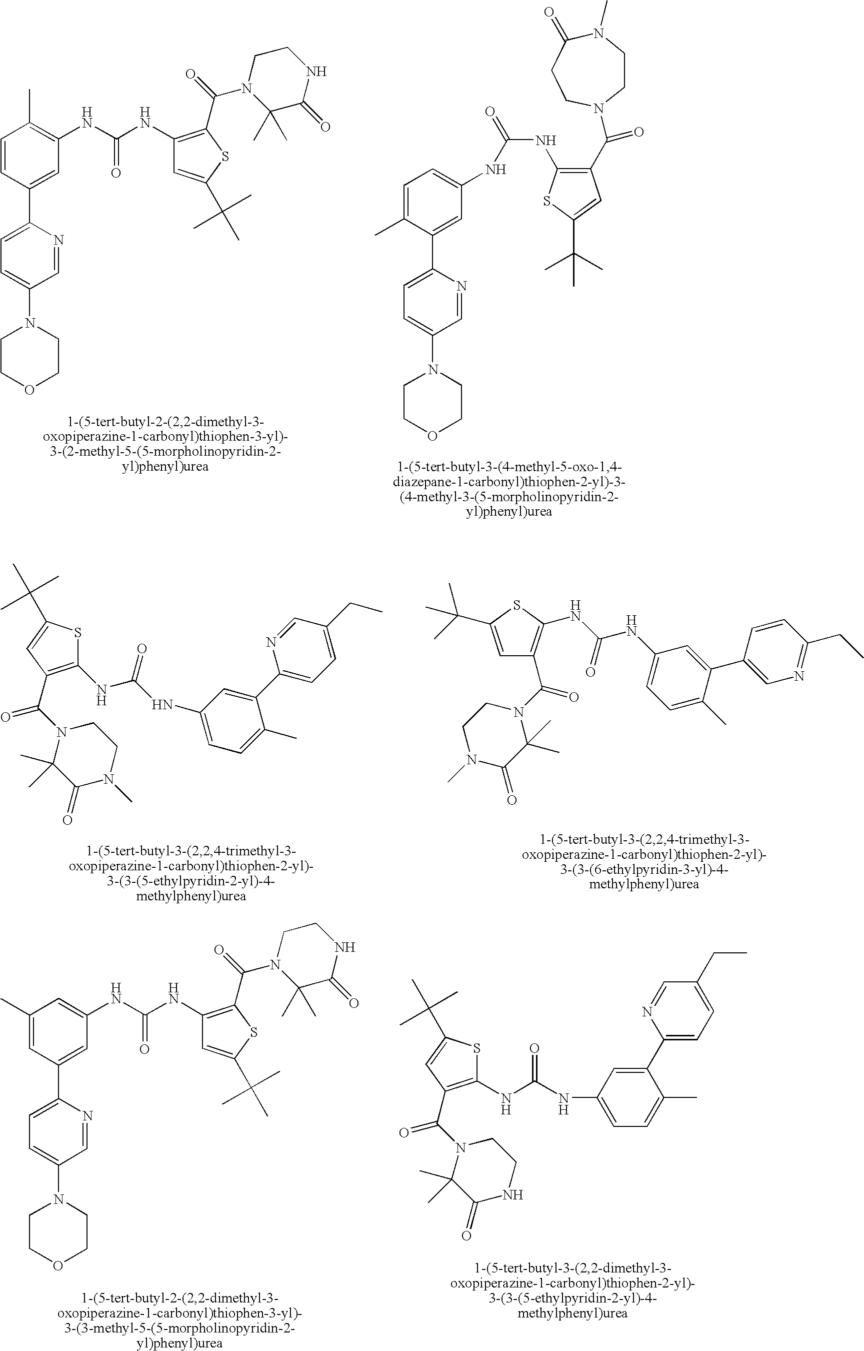 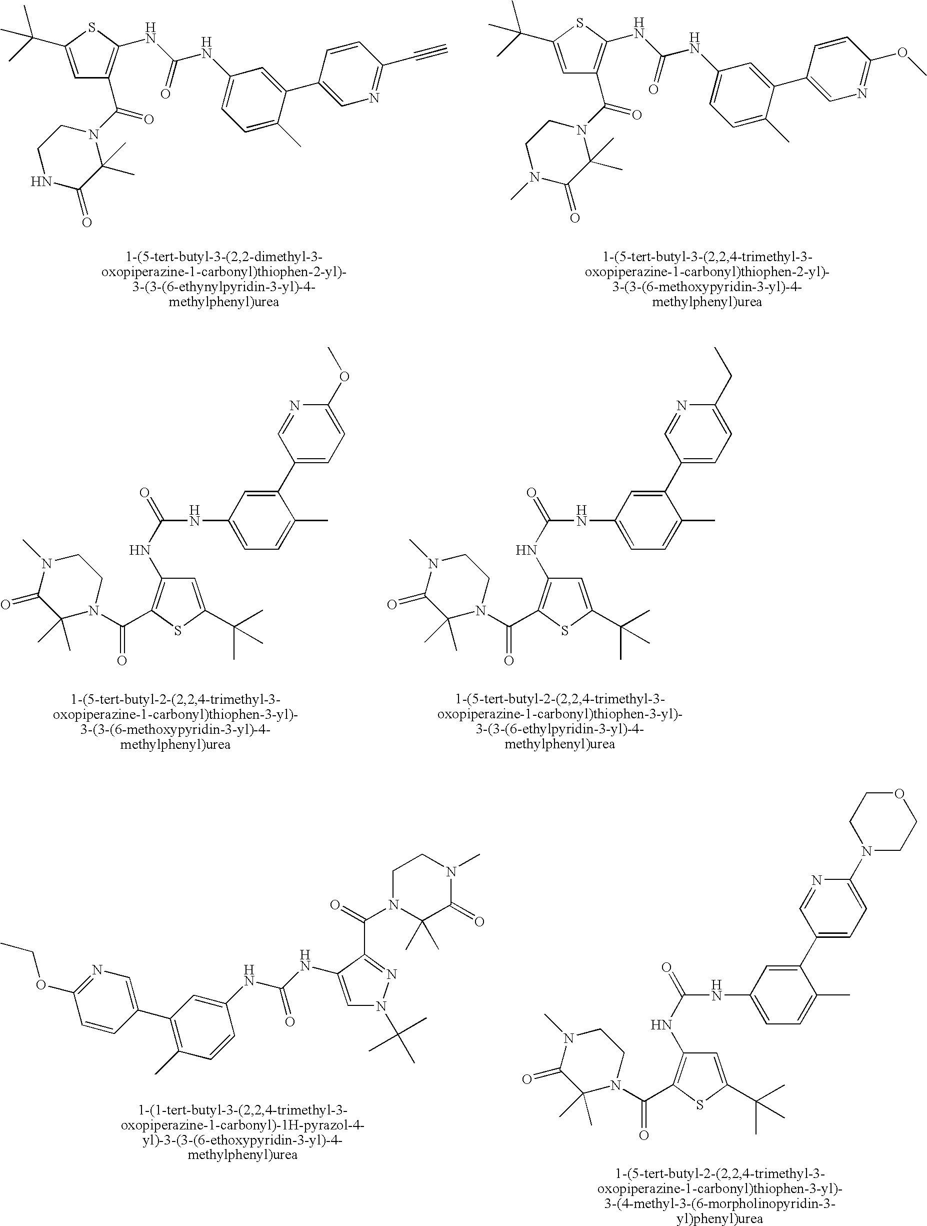 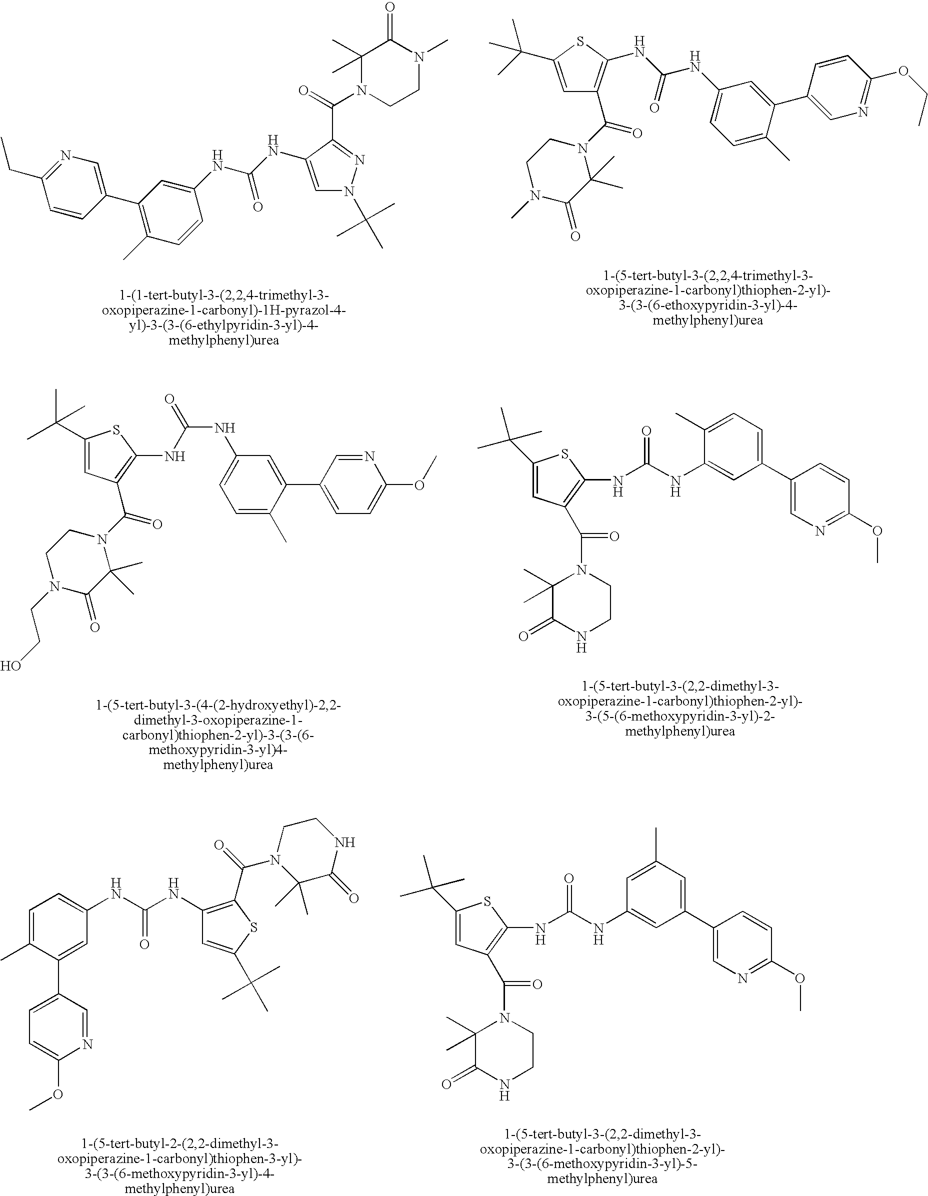 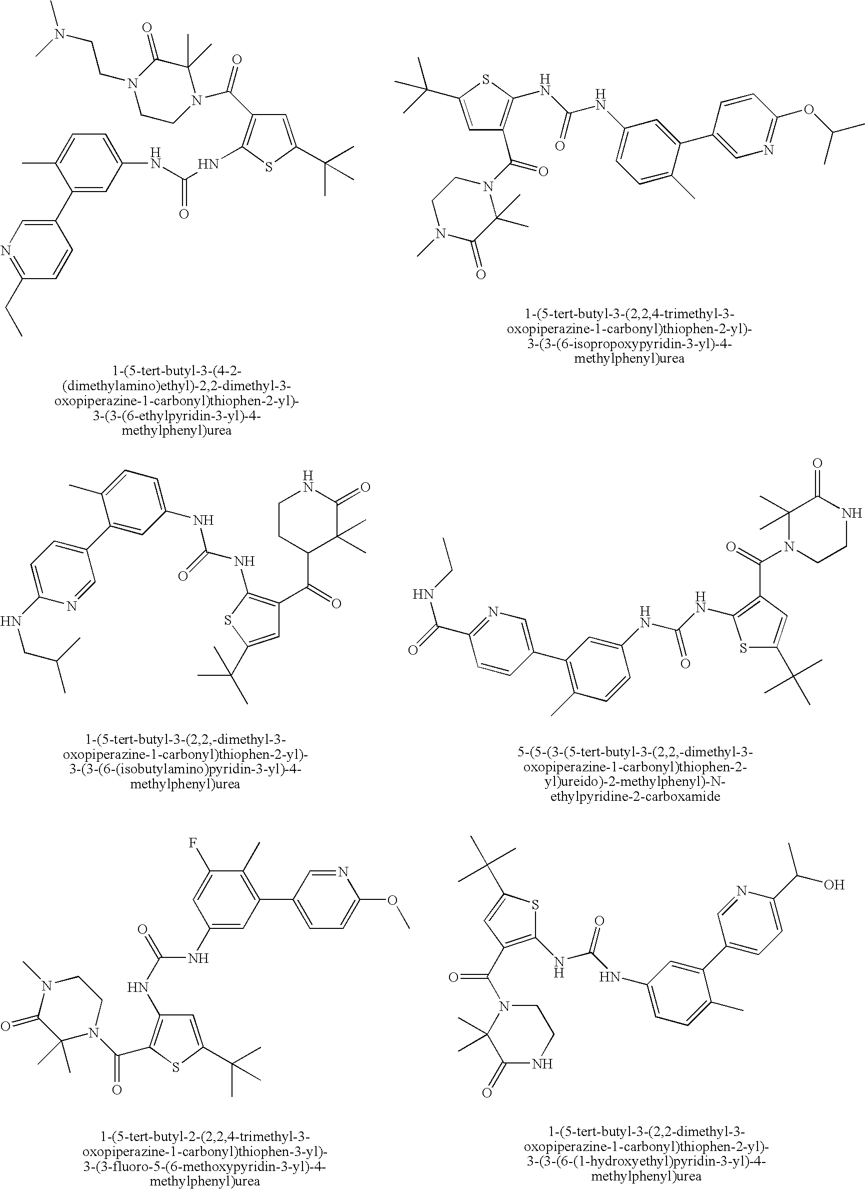 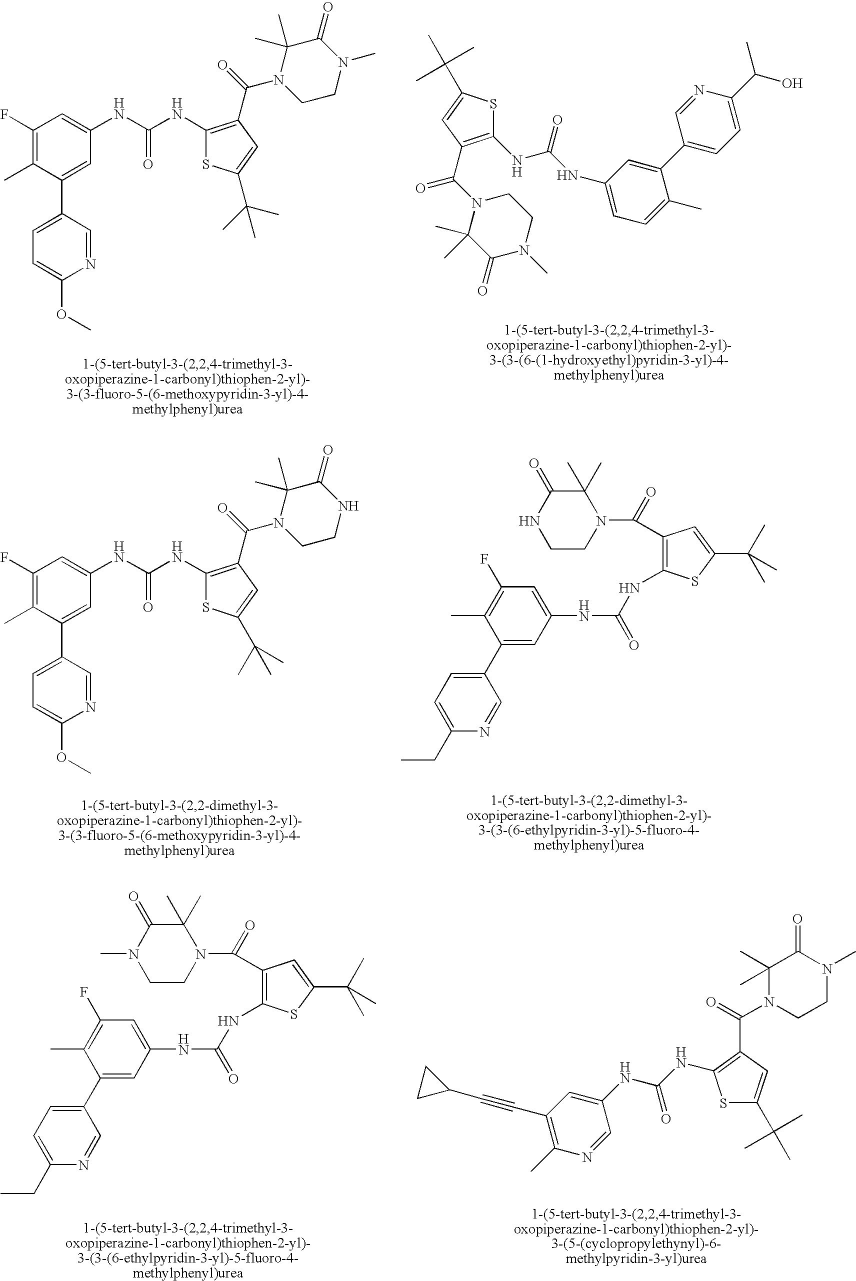 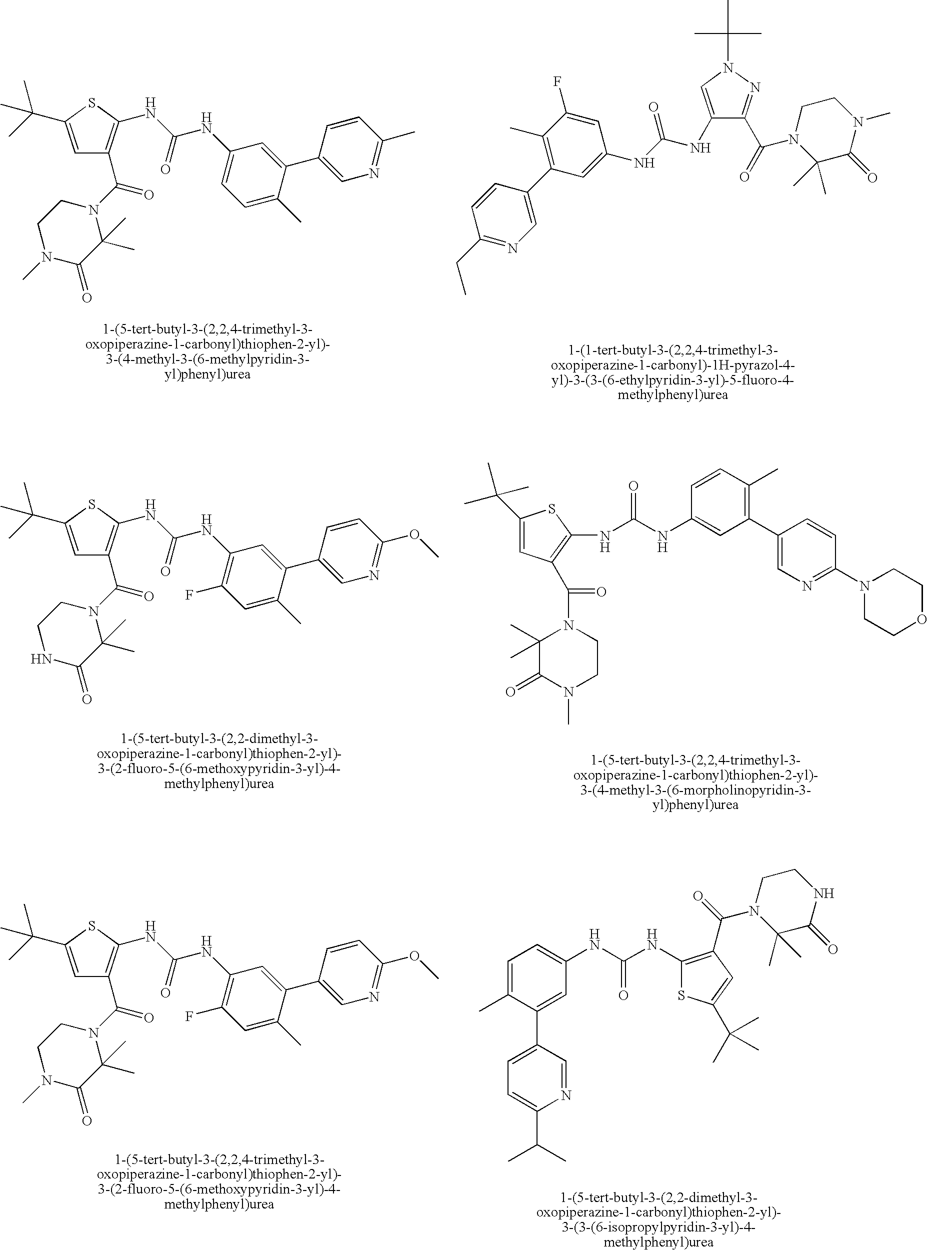 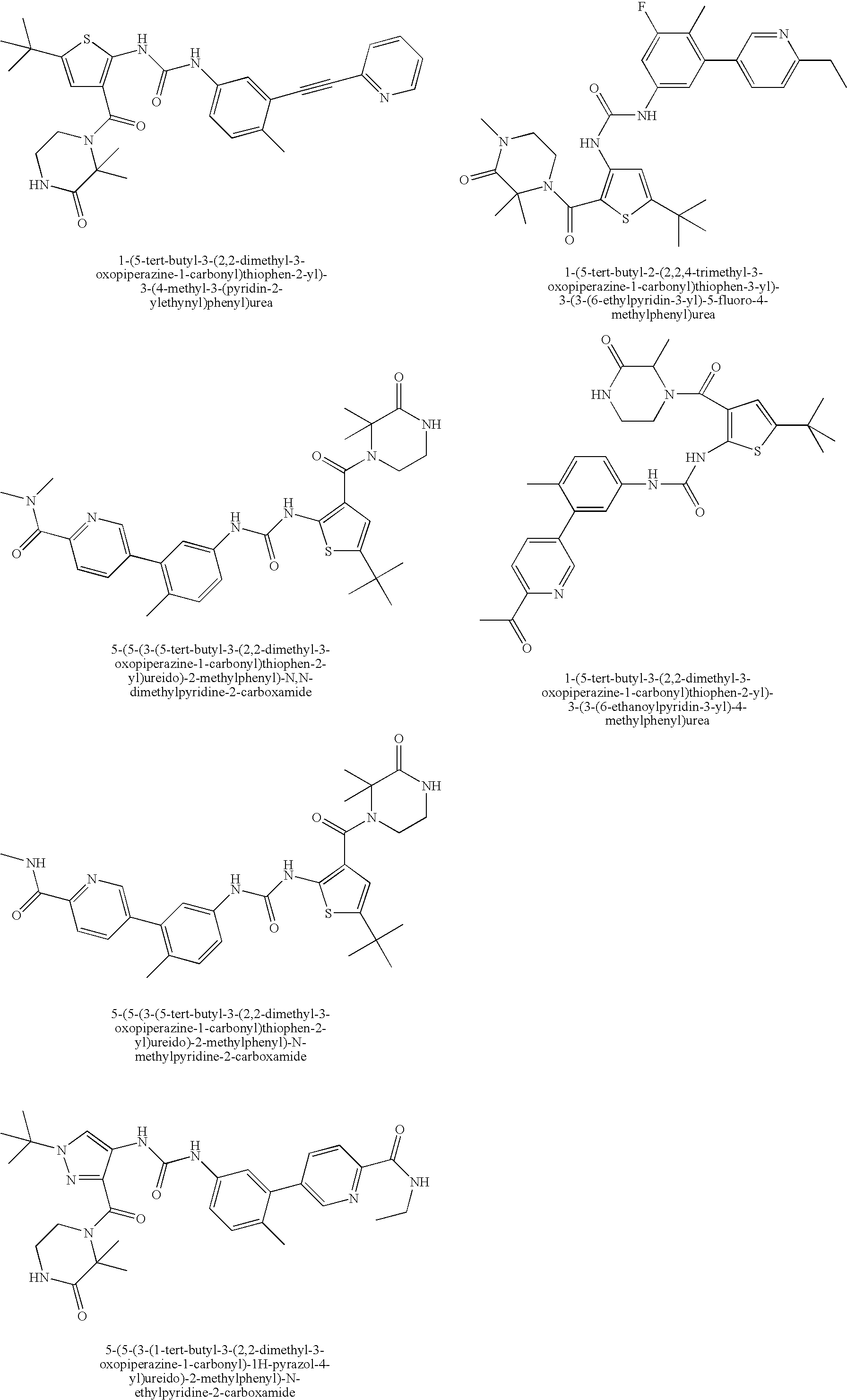 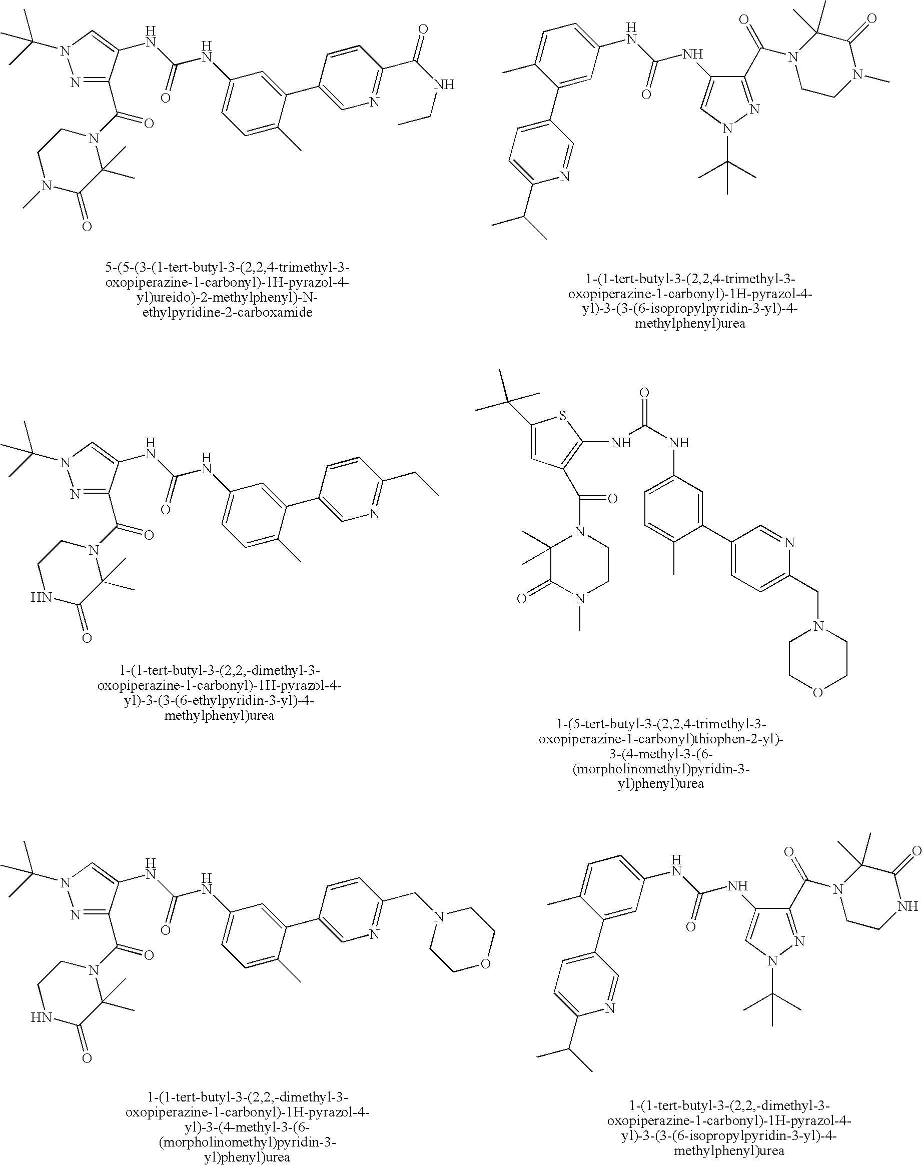 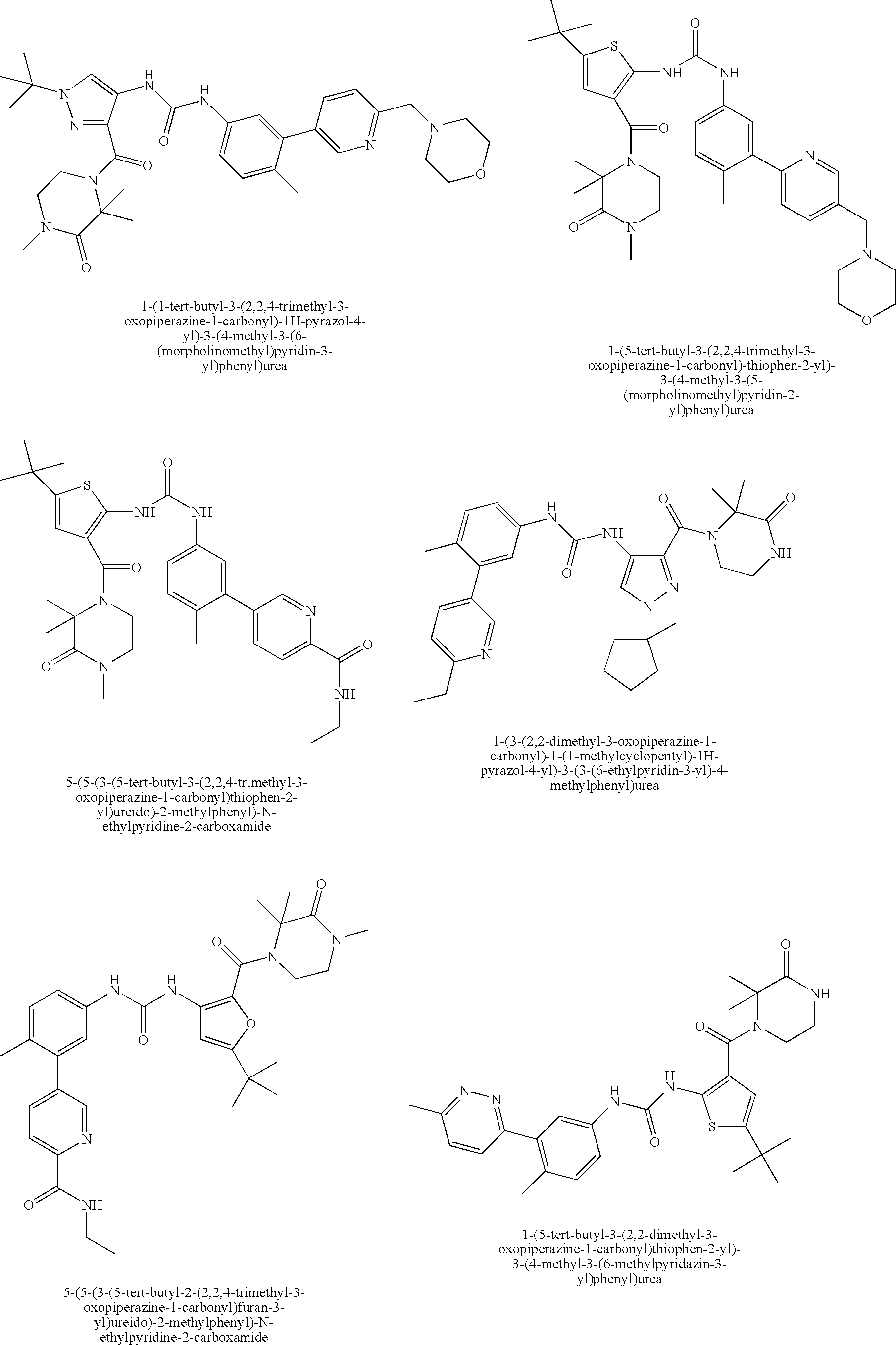 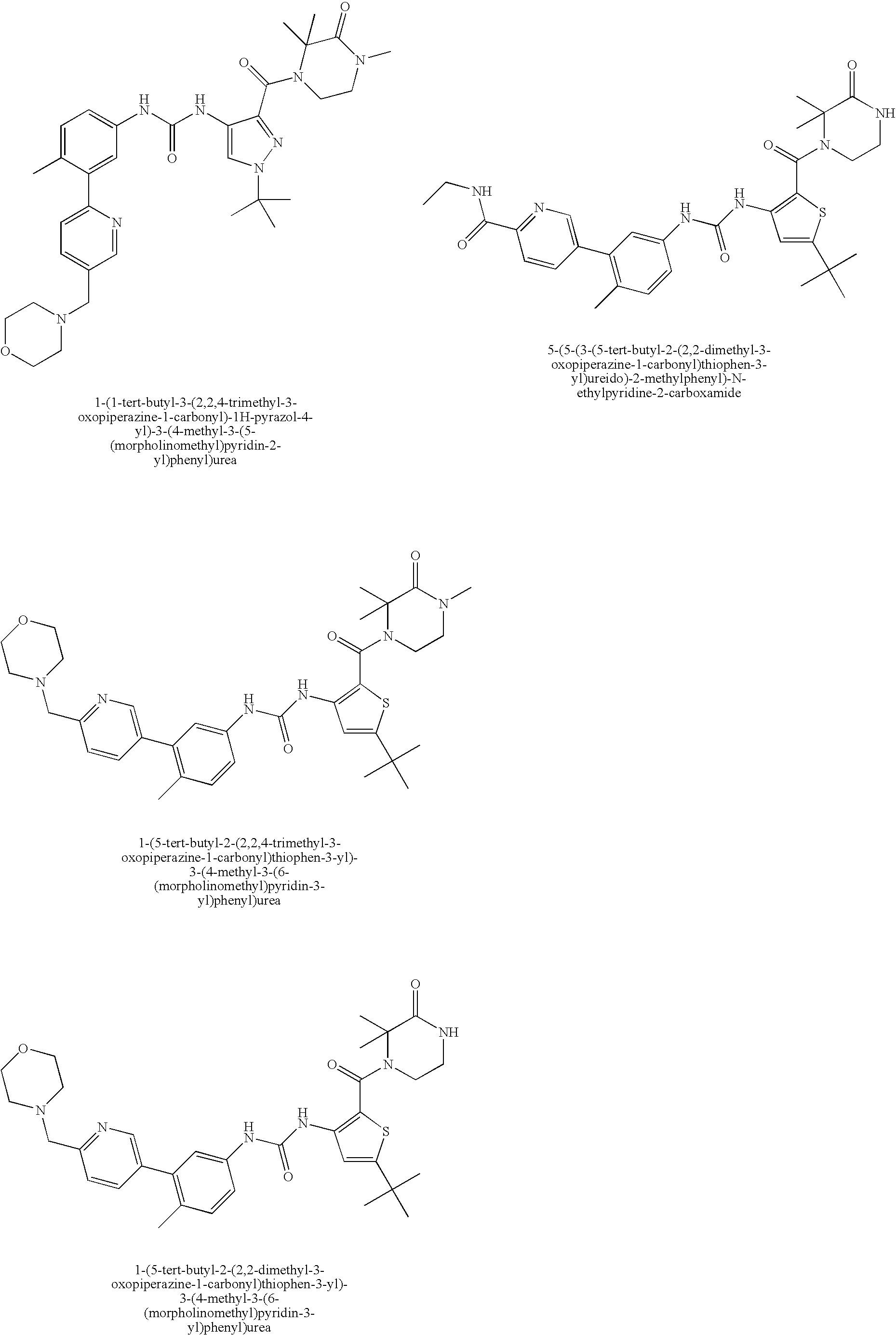 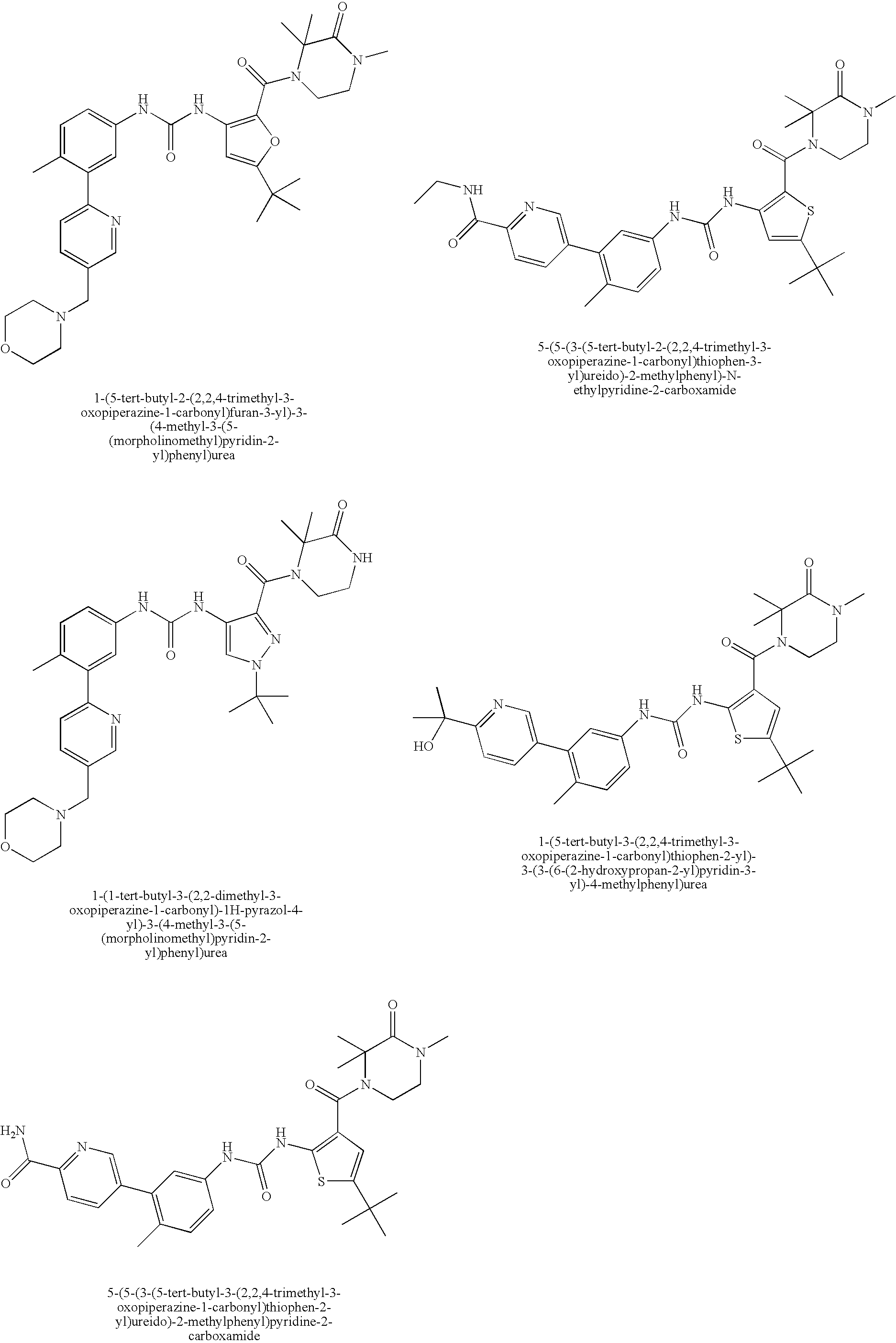 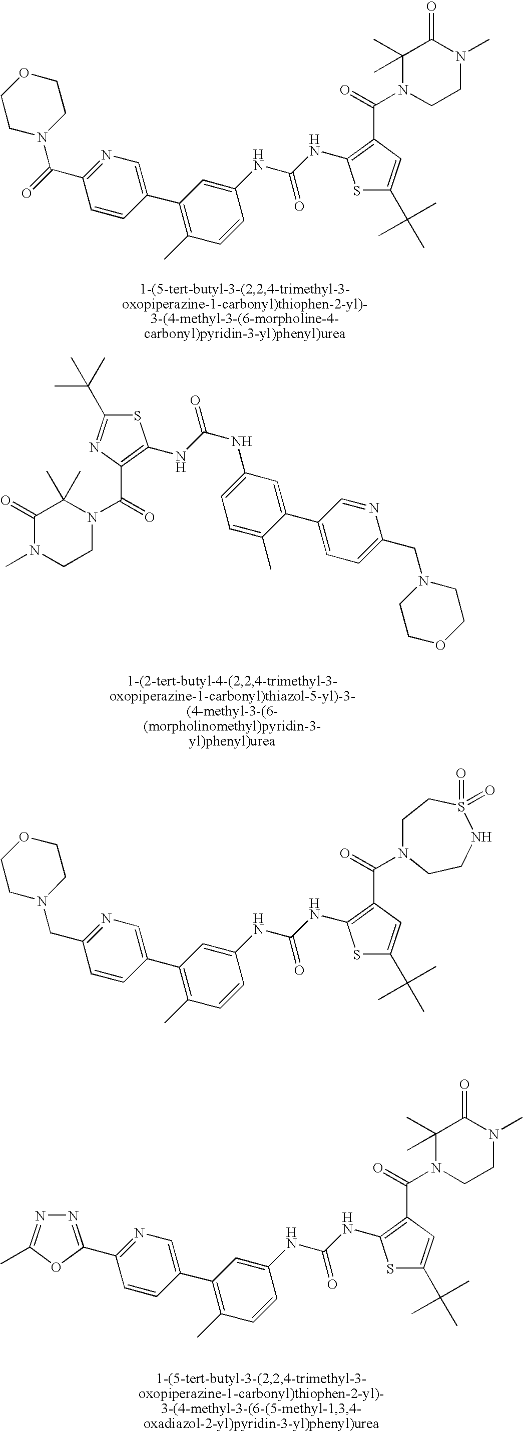 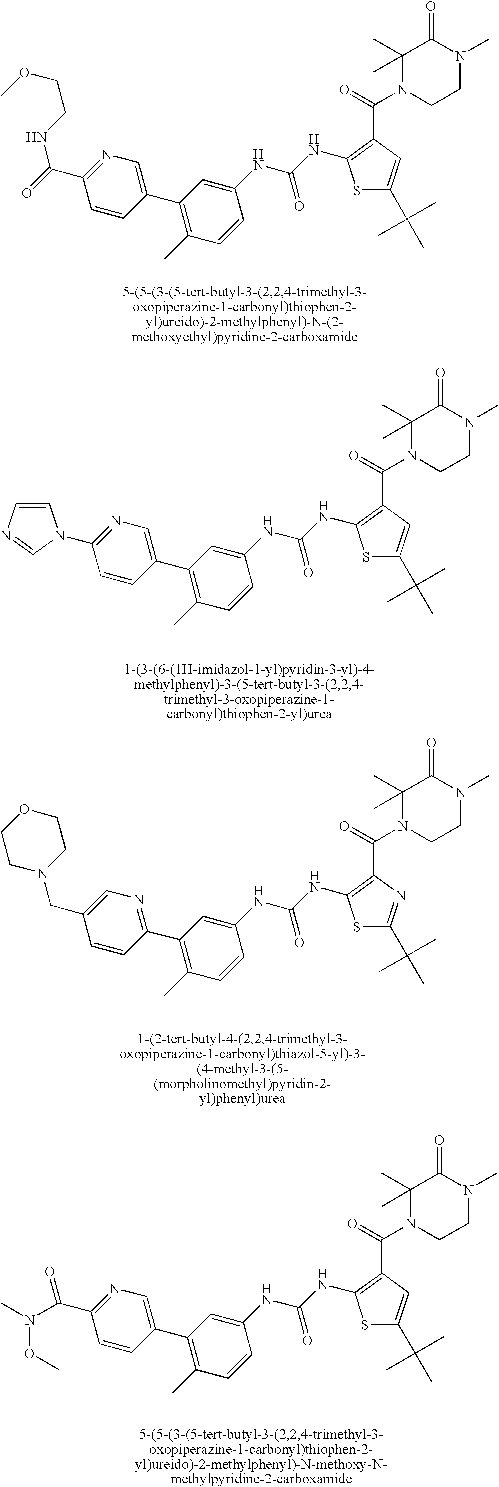 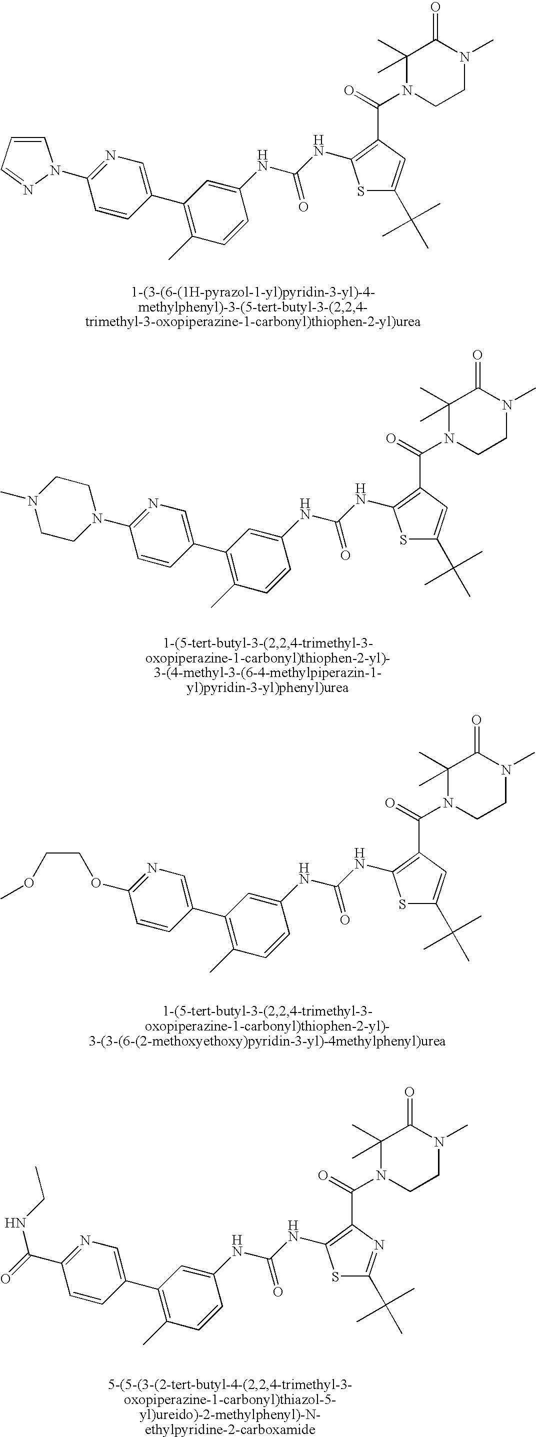 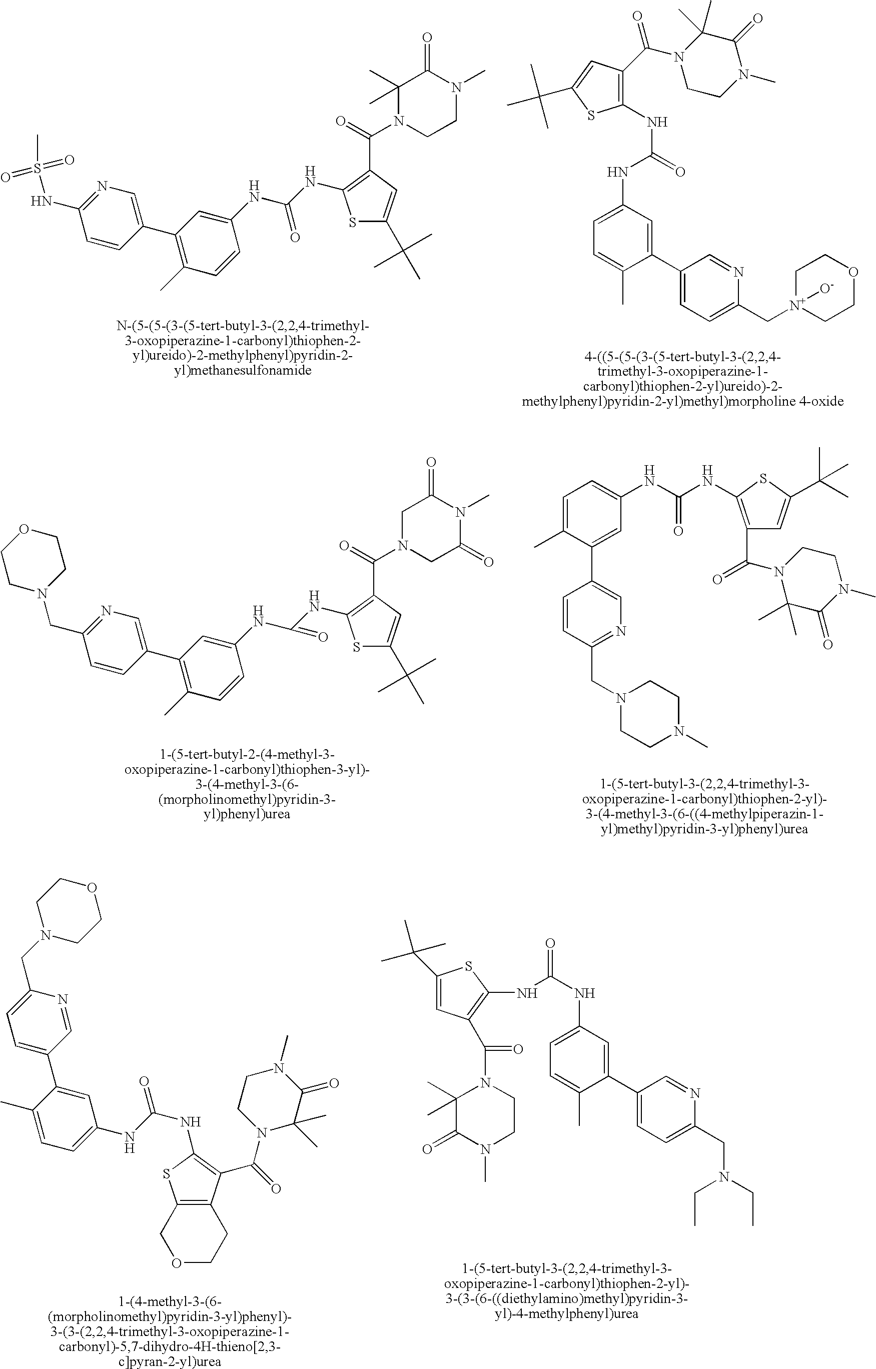 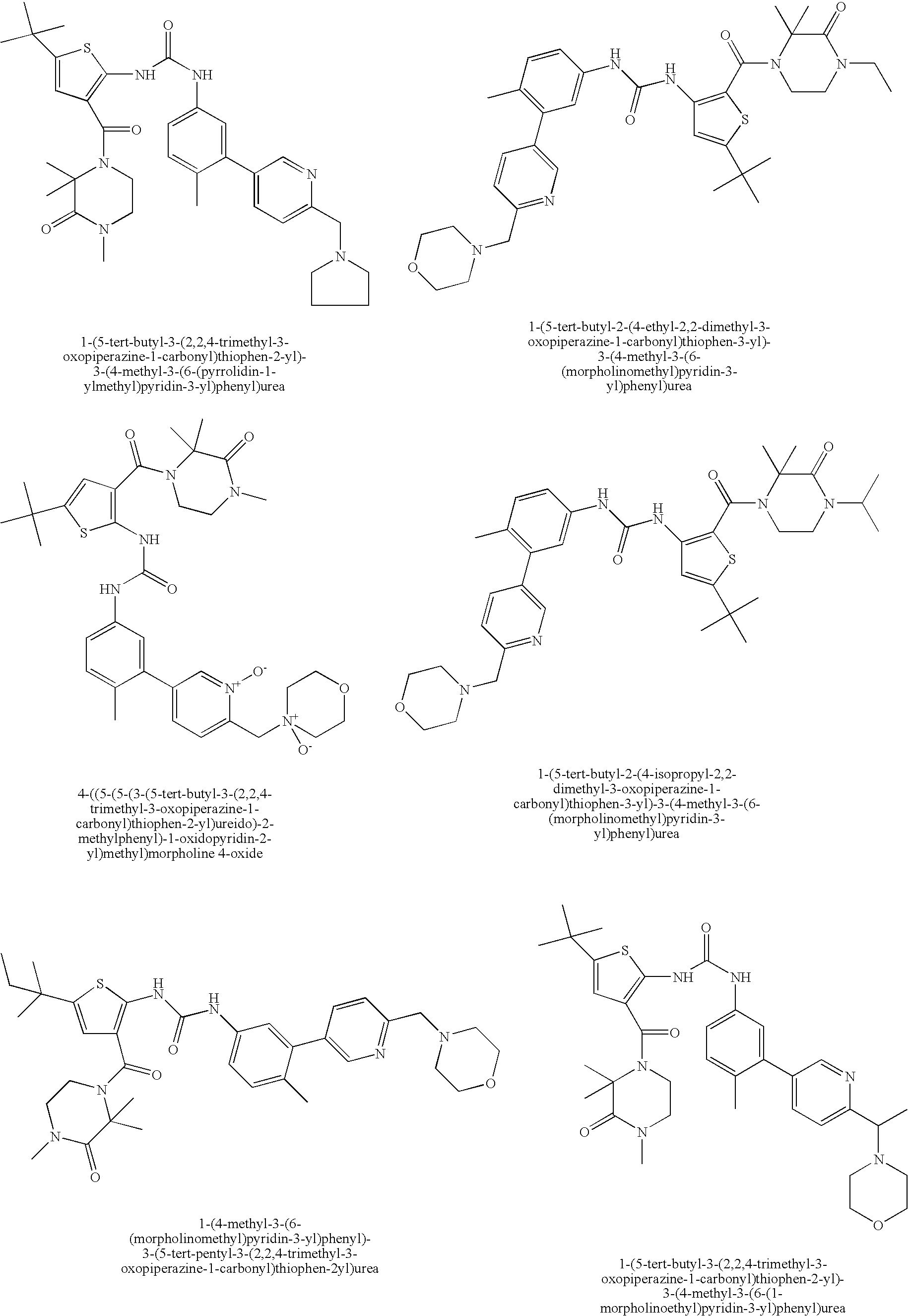 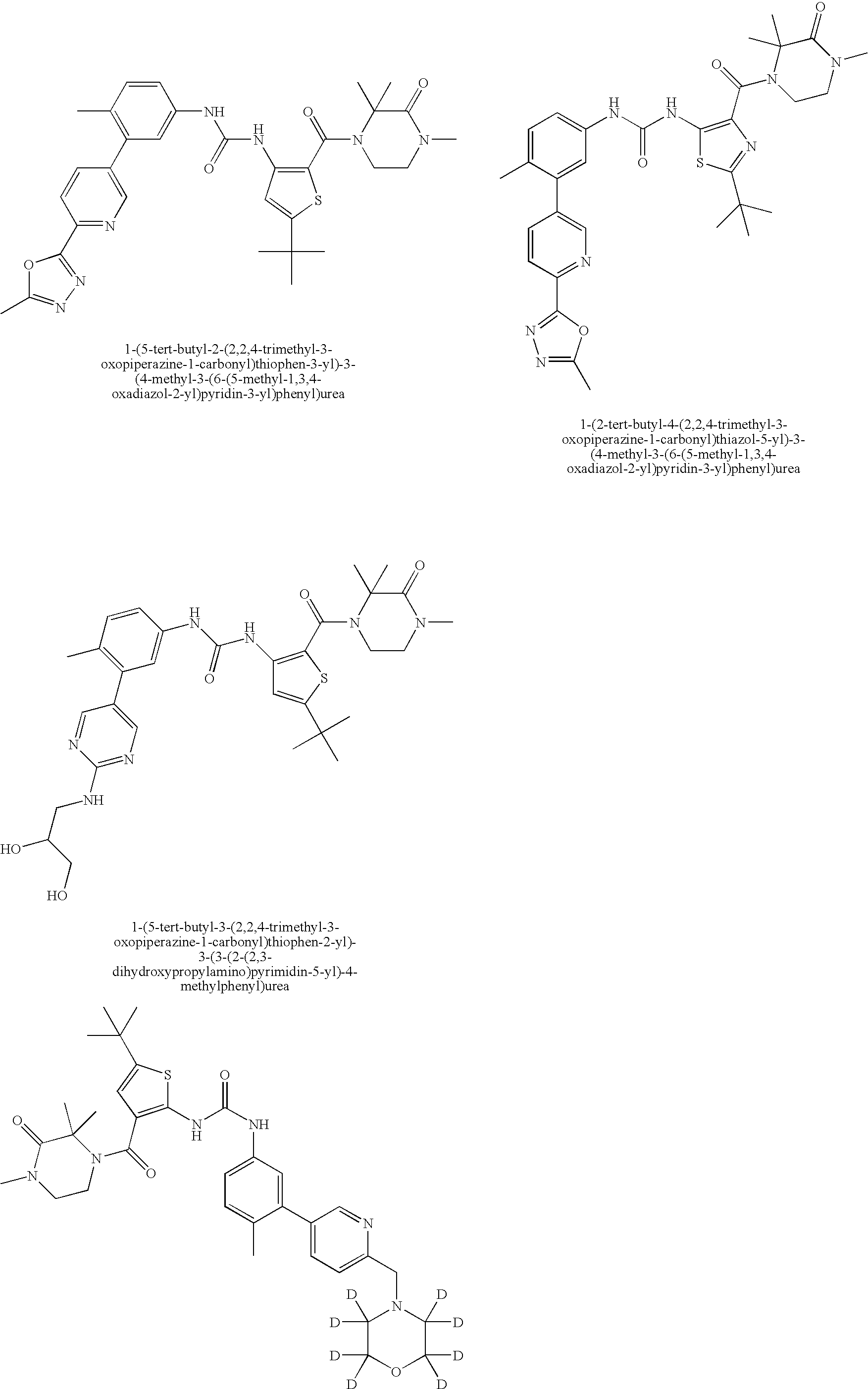
or a pharmaceutically acceptable salt thereof.

8. A compound of claim 1, selected from:
1-(5-tert-butyl-3-(2,2-dimethyl-3-oxopiperazine-1-carbonyl)thiophen-2-yl)-3-(4-methyl-3-(5-(morpholinomethyl)pyridin-2-yl)phenyl)urea;
1-(5-tert-butyl-3-(2,2,4-trimethyl-3-oxopiperazine-1-carbonyl)thiophen-2-yl)-3-(4-methyl-3-(6-(morpholinomethyl)pyridin-3-yl)phenyl)urea;
1-(1-tert-butyl-3-(2,2-dimethyl-3-oxopiperazine-1-carbonyl)-1H-pyrazol-4-yl)-3-(3-(6-isopropylpyridin-3-yl)-4-methylphenyl)urea;
1-(5-tert-butyl-3-(2,2,4-trimethyl-3-oxopiperazine-1-carbonyl)thiophen-2-yl)-3-(3-(6-(2-hydroxypropan-2-yl)pyridin-3-yl)-4-methylphenyl)urea;
1-(5-tert-butyl-2-(2,2,4-trimethyl-3-oxopiperazine-1-carbonyl)thiophen-3-yl)-3-(4-methyl-3-(6-(morpholinomethyl)pyridin-3-yl)phenyl)urea;
1-(5-tert-butyl-2-(2,2,4-trimethyl-3-oxopiperazine-1-carbonyl)furan-3-yl)-3-(4-methyl-3-(5-(morpholinomethyl)pyridin-2-yl)phenyl)urea;
1-(5-tert-butyl-3-(2,2,4-trimethyl-3-oxopiperazine-1-carbonyl)thiophen-2-yl)-3-(4-methyl-3-(6-(morpholine-4-carbonyl)pyridin-3-yl)phenyl)urea;
5-(5-(3-(5-tert-butyl-3-(2,2,4-trimethyl-3-oxopiperazine-1-carbonyl)thiophen-2-yl)ureido)-2-methylphenyl)-N-(2-methoxyethyl)pyridine-2-carboxamide;
1-(5-tert-butyl-3-(2,2,4-trimethyl-3-oxopiperazine-1-carbonyl)thiophen-2-yl)-3-(4-methyl-3-(6-(5-methyl-1,3,4-oxadiazol-2-yl)pyridin-3-yl)phenyl)urea;
1-(2-tert-butyl-4-(2,2,4-trimethyl-3-oxopiperazine-1-carbonyl)thiazol-5-yl)-3-(4-methyl-3-(6-(morpholinomethyl)pyridin-3-yl)phenyl)urea; or
a pharmaceutically acceptable salt thereof.

9. A compound having the structure: 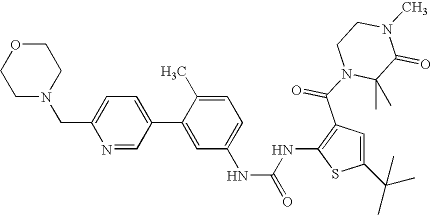
1-(5-tert-butyl-3-(2,2,4-trimethyl-3-oxopiperazine-1-carbonyl)thiophen-2-yl)-3-(4-methyl-3-(6-(morpholinomethyl)pyridin-3-yl)phenyl)urea
or a pharmaceutically acceptable salt thereof.
